(19) United States
(12) Patent Application Publication
(10) Pub. No.: US 2023/0320205 A1
(43) Pub. Date: Oct. 5, 2023

(54) NITROGEN-CONTAINING COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Tiantian MA, Xi'an (CN); Kongyan ZHANG, Xi'an (CN); Xinxuan LI, Xi'an (CN)

(21) Appl. No.: 18/007,811

(22) PCT Filed: Aug. 20, 2021
(86) PCT No.: PCT/CN2021/113867

§ 371 Date: Dec. 2, 2022

(30) Foreign Application Priority Data
Nov. 10, 2020 (CN) 202011249452.5

(51) Int. Cl. H10K 85/60; C07D 405/14; C09K 11/06; C07D 405/04; C07D 491/048; C07D 409/14

(52) U.S. Cl. CPC H10K 85/654; C07D 405/14; C09K 11/06; H10K 85/615; H10K 85/6574; C07D 405/04; H10K 50/11; C07D 491/048; H10K 85/657; H10K 85/6576; C07D 409/14; H10K 85/626; H10K 85/6572

(57) ABSTRACT

The present disclosure belongs to the technical field of organic materials, and provides a nitrogen-containing compound, an organic electroluminescent device, and an electronic apparatus. The nitrogen-containing compound has a structure as shown in a formula 1,

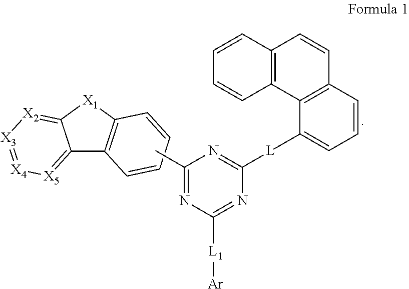

### CROSS REFERENCE TO RELATED APPLICATIONS

The present application claims the priority of Chinese Patent Application No. 202011249452.5 filed on Nov. 10, 2020, the contents of which are incorporated herein by reference in their entirety as part of the present application.

### FIELD

The present disclosure relates to the technical field of organic materials, in particular to a nitrogen-containing compound, an organic electroluminescent device using the nitrogen-containing compound, and an electronic apparatus using the organic electroluminescent device.

### BACKGROUND

An organic electroluminescent device, also referred to as an organic light-emitting diode, refers to a phenomenon that an organic light-emitting material emits light when excited by a current under the action of an electric field. It is a process of converting an electrical energy into a light energy. Compared with inorganic light-emitting materials, organic light-emitting diodes (OLEDs) have the advantages of active light emission, large optical path range, low driving voltage, high brightness, high efficiency, low energy consumption, and simple manufacturing process. Because of these advantages, organic light-emitting materials and devices have become one of the most popular research topics of the scientific community and the industrial community.

The organic electroluminescent device generally includes an anode, a hole transport layer, an electroluminescent layer as an energy conversion layer, an electron transport layer, and a cathode which are stacked in sequence. When a voltage is applied to the cathode and the anode, an electric field is generated between the two electrodes, the electrons on the cathode side move towards the electroluminescent layer and the holes on the anode side also move towards the electroluminescent layer under the action of the electric field, the electrons and the holes are combined in the electroluminescent layer to form excitons, the excitons are in an excited state and release energy outwards, which in turn makes the electroluminescent layer emit light outwards.

In the prior art, CN107445910A, CN108884059A, CN109641840A, CN110540527A and the like disclose light-emitting layer materials that can be used in an organic electroluminescent device. However, it is still necessary to continue to develop new materials to further improve the performance of electronic components.

The above information disclosed in the background is merely used to enhance an understanding of the background of the present disclosure, and thus it may include information that does not constitute the prior art known to those of ordinary skill in the art.

### SUMMARY

The present disclosure aims to provide a nitrogen-containing compound, an organic electroluminescent device, and an electronic device to improve the performance of the organic electroluminescent device and the electronic apparatus.

In order to achieve the above-mentioned inventive purpose, the present disclosure adopts the following technical solutions.

In a first aspect, the present disclosure provides a nitrogen-containing compound, having a structure as shown in a formula 1:

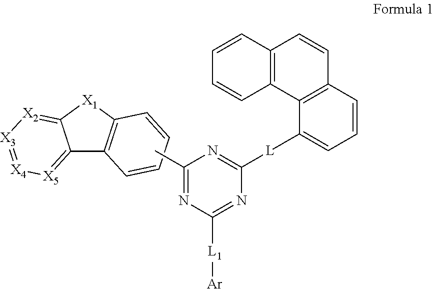

Wherein X₁ is selected from O or S;

X₂, X₃, X₄, and X₅ are the same as or different from each other, and are each independently selected from C(H) or N;

L and L₁ are respectively and independently selected from a single bond, substituted or unsubstituted arylene with 6 to 30 carbon atoms, and substituted or unsubstituted heteroarylene with 3 to 30 carbon atoms;

Ar is selected from substituted or unsubstituted aryl with 6 to 30 carbon atoms, substituted or unsubstituted heteroaryl with 3 to 30 carbon atoms, substituted or unsubstituted alkyl with 1 to 20 carbon atoms, and substituted or unsubstituted cycloalkyl with 3 to 20 carbon atoms;

substituents in L, L₁ and Ar are the same as or different from each other, and are each independently selected from deuterium, a halogen group, cyano, heteroaryl with 3 to 20 carbon atoms, aryl with 6 to 20 carbon atoms which can be optionally substituted by 0, 1, 2, 3, 4, or 5 substituents independently selected from deuterium, fluorine, cyano, methyl, and tert-butyl, trialkylsilyl with 3 to 12 carbon atoms, triarylsilyl with 18 to 24 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms; and

in L, L₁ and Ar, optionally, any two adjacent substituents form a ring.

The nitrogen-containing compound provided by the present disclosure is based on a triazine derivative and phenanthrene as a core structure. Wherein a fused ring group of phenanthrene is an aromatic structure having 10 π electrons, which has a stable planar structure. The triazine derivative is substituted at the 4-position of phenanthrene, and compounds substituted at the 4-position have a larger twisted dihedral angle compared with those substituted at other positions, which reduces the degree of conjugation of a nitrogen-containing compound structure, thus making the material have a high T1 value; and meanwhile, the steric hindrance of the material is increased, the intermolecular force is decreased, the evaporation temperature of the material is decreased under the same molecular weight, and the performance degradation of the organic electroluminescent device caused by crystallization can be effectively reduced. Furthermore, the triazine derivative can effectively enhance the electronegativity of the compound and improve the electron transport properties of the compound. When the nitrogen-containing compound of the present disclosure is used as a host material of an organic electroluminescent layer of an organic electroluminescent device, the luminous efficiency and the service life of the device can be effectively improved.

In a second aspect, the present disclosure provides an organic electroluminescent device, including an anode and a cathode which are disposed oppositely, and a functional layer disposed between the anode and the cathode; and the functional layer includes the nitrogen-containing compound in the first aspect; and

preferably, the functional layer includes an organic electroluminescent layer, and the organic electroluminescent layer includes the nitrogen-containing compound.

In a third aspect, the present disclosure provides an electronic apparatus, including the organic electroluminescent device in the second aspect.

### BRIEF DESCRIPTION OF THE DRAWINGS

The above and other features and advantages of the present disclosure will become more apparent by describing the examples in detail with reference to the accompanying drawings.

FIG. 1 is a structural schematic diagram of an organic electroluminescent device according to an embodiment of the present disclosure.

FIG. 2 is a structural schematic diagram of an electronic device according to an embodiment of the present disclosure.

### DESCRIPTION OF REFERENCE SIGNS

100, anode; 200, cathode; 300, functional layer; 310, hole injection layer; 321, hole transport layer; 322, electron blocking layer; 330, organic electroluminescent layer; 340, hole blocking layer; 350, electron transport layer; 360, electron injection layer; and 400, electronic apparatus.

### DETAILED DESCRIPTION

Exemplary embodiments will now be described more fully with reference to the accompanying drawings. However, the exemplary embodiments can be implemented in various forms and should not be construed as limited to the examples set forth here; and on the contrary, these embodiments are provided so that the present disclosure will be thorough and complete, and the concept of the exemplary embodiments is fully conveyed to those skilled in the art. The described features, structures, or characteristics may be combined in any suitable manner in one or more embodiments. In the following description, many specific details are provided to provide a thorough understanding of the embodiments of the present disclosure.

In the drawings, the thicknesses of regions and layers may be exaggerated for clarity. The same reference signs denote the same or similar structures in the drawings, and thus their detailed description will be omitted.

In a first aspect, the present disclosure provides a nitrogen-containing compound, having a structure as shown in a formula 1:

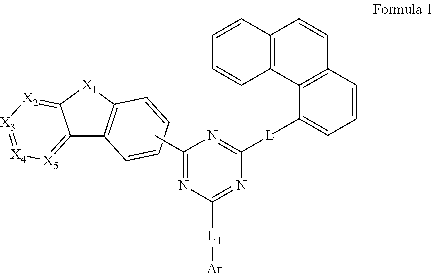

wherein X₁ is selected from O or S;

X₂, X₃, X₄, and X₅ are the same as or different from each other, and are each independently selected from C(H) or N;

L and L₁ are respectively and independently selected from a single bond, substituted or unsubstituted arylene with 6 to 30 carbon atoms, and substituted or unsubstituted heteroarylene with 3 to 30 carbon atoms;

Ar is selected from substituted or unsubstituted aryl with 6 to 30 carbon atoms, substituted or unsubstituted heteroaryl with 3 to 30 carbon atoms, substituted or unsubstituted alkyl with 1 to 20 carbon atoms, and substituted or unsubstituted cycloalkyl with 3 to 20 carbon atoms;

substituents in L, L₁ and Ar are the same as or different from each other, and are each independently selected from deuterium, a halogen group, cyano, heteroaryl with 3 to 20 carbon atoms, aryl with 6 to 20 carbon atoms which can be optionally substituted by 0, 1, 2, 3, 4, or 5 substituents independently selected from deuterium, fluorine, cyano, methyl, and tert-butyl, trialkylsilyl with 3 to 12 carbon atoms, triarylsilyl with 18 to 24 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms; and

in L, L₁ and Ar, optionally, any two adjacent substituents form a ring.

In the present disclosure, the terms “optional” and “optionally” mean that the subsequently described event or circumstance can but need not occur, and that the description includes occasions where the event or circumstance occurs or does not occur. For example, “optionally, any two adjacent substituents form a ring;”, which means that the two substituents may, but need not, form a ring, including a scenario in which two adjacent substituents form a ring and a scenario in which two adjacent substituents do not form a ring.

In the present disclosure, “aryl with 6 to 20 carbon atoms which can be optionally substituted by 0, 1, 2, 3, 4, or 5 substituents independently selected from deuterium, fluorine, cyano, methyl, and tert-butyl” means that the aryl may be substituted with one or more of deuterium, fluorine, cyano, methyl, and tert-butyl, and may also be not substituted with deuterium, fluorine, cyano, methyl, or tert-butyl, and when the number of substituents on the aryl is greater than or equal to 2, the substituents may be the same or different.

In the present disclosure, in the case that “any two adjacent substituents form a ring”, “any adjacent” can include both substituents on a same atom and can also include one substituent on each of two adjacent atoms; when there are two substituents on the same atom, the two substituents may form a saturated or unsaturated ring with the atom to which they are jointly connected; and when two adjacent atoms each have one substituent, the two substituents may be fused to form a ring. For example, when Ar has two or more substituents and any adjacent substituents form a ring, a saturated or unsaturated ring having 5 to 14 ring-forming carbon atoms may be formed, for example, a benzene ring, a naphthalene ring, a phenanthrene ring, an anthracene ring, cyclopentane, cyclohexane, adamantane, and the like.

In the present disclosure, the descriptions of “each . . . is independently”, “ . . . is respectively and independently“and” . . . is independently selected from” can be exchanged, which should be understood in a broad sense, and may mean that specific options expressed by a same signs in different groups do not influence each other, or may also mean that specific options expressed by a same signs in a same group do not influence each other. For example, the meaning of

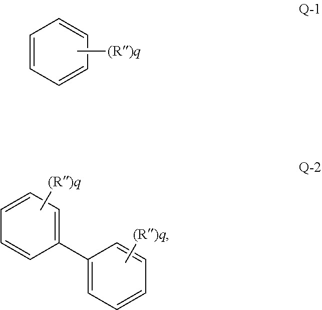

where each q is independently 0, 1, 2 or 3 and each R″ is independently selected from hydrogen, deuterium, fluorine, and chlorine” is as follows: a formula Q-1 represents that a benzene ring has q substituents R″, each R″ can be the same or different, and options of each R″ do not influence each other; and a formula Q-2 represents that each benzene ring of biphenyl has q substituents R″, the number q of the substituents R″ on the two benzene rings can be the same or different, each R″ can be the same or different, and options of each R″ do not influence each other.

In the present disclosure, the number of carbon atoms of L, L₁, and Ar refers to the number of all carbon atoms. For example, if L is selected from substituted arylene with 12 carbon atoms, the number of all carbon atoms of the arylene and the substituents on the arylene is 12. For example: if Ar is

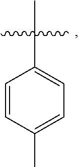 [SMILES: *c1ccc(C)cc1]

then the number of carbon atoms is 7; and if L is

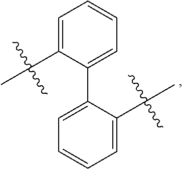 [SMILES: *c1ccccc1-c1ccccc1*]

the total number of carbon atoms is 12.

In the present disclosure, when a specific definition is not otherwise provided, “hetero” means that at least one heteroatom such as B, N, O, S, Se, Si, or P is included in one functional group and the remaining atoms are carbon and hydrogen. The unsubstituted alkyl may be a “saturated alkyl group” without any double bond or triple bond.

In the present disclosure, “alkyl” may include linear alkyl or branched alkyl. The alkyl may have 1 to 20 carbon atoms, and in the present disclosure, a numerical range such as “1 to 20” refers to each integer in a given range; for example, “1 to 20 carbon atoms” refers to alkyl that may include 1, 2, 3, 4, 5, 6, 7, 8, 9, 10, 11, 12, 13, 14, 15, 16, 17, 18, 19, or 20 carbon atoms. In addition, the alkyl may be substituted or unsubstituted.

Optionally, the alkyl is selected from alkyl with 1 to 5 carbon atoms, and specific examples include, but are not limited to, methyl, ethyl, n-propyl, isopropyl, n-butyl, isobutyl, sec-butyl, tert-butyl, and pentyl.

In the present disclosure, aryl refers to an optional functional group or substituent derived from an aromatic carbocyclic ring. The aryl can be monocyclic aryl (e.g., phenyl) or polycyclic aryl, in other words, the aryl can be monocyclic aryl, fused aryl, two or more monocyclic aryl conjugatedly connected by carbon-carbon bond, monocyclic aryl and fused aryl which are conjugatedly connected by a carbon-carbon bond, and two or more fused aryl conjugatedly connected by carbon-carbon bonds. That is, unless specified otherwise, two or more aromatic groups conjugatedly connected by carbon-carbon bonds can also be regarded as aryl of the present disclosure. The fused aryl may, for example, include bicyclic fused aryl (e.g., naphthyl), tricyclic fused aryl (e.g., phenanthryl, fluorenyl, and anthryl), and the like. For example, in the present disclosure, biphenyl, terphenyl, and the like are aryl. Examples of the aryl can include, but are not limited to, phenyl, naphthyl, fluorenyl, anthryl, phenanthryl, biphenyl, terphenyl, quaterphenyl, quinquephenyl, benzo[9,10]phenanthryl, pyrenyl, benzofluoranthenyl, chrysenyl, and the like. “Substituted or unsubstituted aryl” in the present disclosure can contain 6 to 30 carbon atoms, in some embodiments, the number of carbon atoms in the substituted or unsubstituted aryl is 6 to 25, in other embodiments, the number of carbon atoms in the substituted or unsubstituted aryl is 6 to 18, and in other embodiments, the number of carbon atoms in the substituted or unsubstituted aryl is 6 to 13. For example, in the present disclosure, the number of carbon atoms in the substituted or unsubstituted aryl can be 6, 12, 13, 14, 15, 18, 20, 24, 25, or 30, and of course, the number of carbon atoms can also be other numbers, which will not be listed here. In the present disclosure, biphenyl can be understood as phenyl-substituted aryl and can also be understood as unsubstituted aryl.

In the present disclosure, the related arylene refers to a divalent group formed by further loss of one hydrogen atom of the aryl.

In the present disclosure, the substituted aryl can be that one or two or more hydrogen atoms in the aryl are substituted by groups such as a deuterium atom, a halogen group, cyano, aryl, heteroaryl, trialkylsilyl, alkyl, cycloalkyl, alkoxy, alkylthio, and the like. Specific examples of heteroaryl-substituted aryl include, but are not limited to, dibenzofuranyl-substituted phenyl, dibenzothiophene-substituted phenyl, pyridine-substituted phenyl, and the like. It should be understood that the number of carbon atoms of the substituted aryl refers to the total number of carbon atoms of the aryl and substituents on the aryl, for example, substituted aryl with 18 carbon atoms means that the total number of carbon atoms of the aryl and its substituents is 18.

In the present disclosure, specific examples of aryl as a substituent include, but are not limited to, phenyl, naphthyl, anthryl, phenanthryl, dimethylfluorenyl, biphenyl, diphenylfluorenyl, spirobifluorenyl, and the like.

In the present disclosure, fluorenyl may be substituted and the two substituents may be bonded to each other to form a spiro structure, and specific embodiments include, but are not limited to, the following structures:

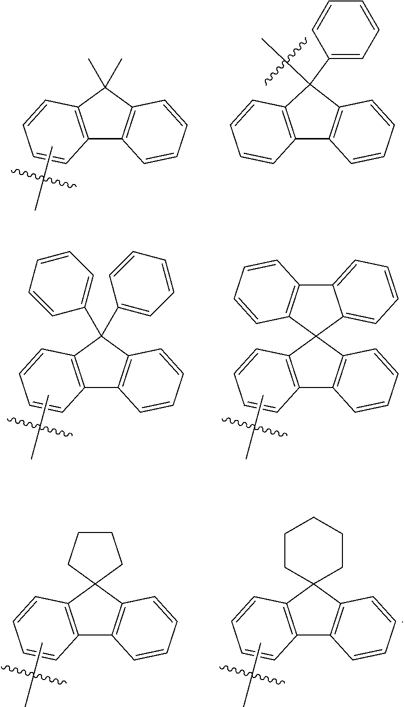

In the present disclosure, heteroaryl refers to a monovalent aromatic ring containing 1, 2, 3, 4, 5, 6, or 7 heteroatoms in the ring, or its derivative, and the heteroatom may be at least one of B, O, N, P, Si, Se, and S. The heteroaryl may be monocyclic heteroaryl or polycyclic heteroaryl, in other words, the heteroaryl may be a single aromatic ring system or a plurality of aromatic ring systems conjugatedly connected by carbon-carbon bond, and any one aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, benzothiazolyl, phenothiazinyl, silafluorenyl, dibenzofuranyl, and N-arylcarbazolyl (e.g., N-phenylcarbazolyl), N-heteroarylcarbazolyl (e.g., N-pyridylcarbazolyl), N-alkylcarbazolyl (e.g., N-methylcarbazolyl), and the like, but is not limited to this. Wherein thienyl, furyl, phenanthrolinyl, etc. are heteroaryl of the single aromatic ring system, and N-arylcarbazolyl, and N-heteroarylcarbazolyl are heteroaryl of the plurality of aromatic ring systems conjugatedly connected by carbon-carbon bonds. “Substituted or unsubstituted heteroaryl” in the present disclosure contains 3 to 30 carbon atoms, in some embodiments, the number of carbon atoms in the substituted or unsubstituted heteroaryl is 3 to 25, in other embodiments, the number of carbon atoms in the substituted or unsubstituted heteroaryl is 3 to 20, and in other embodiments, the number of carbon atoms in the substituted or unsubstituted heteroaryl is 12 to 20. For example, the number of carbon atoms of the substituted or unsubstituted heteroaryl can also be 3, 4, 5, 7, 12, 13, 18, 20, 24, 25 or 30, and of course, the number of carbon atoms can also be other numbers, which will not be listed here.

In the present disclosure, the related heteroarylene refers to a divalent group formed by further loss of one hydrogen atom of the heteroaryl.

In the present disclosure, the substituted heteroaryl may be that one or two or more hydrogen atoms in the heteroaryl are substituted with groups such as a deuterium atom, a halogen group, cyano, aryl, heteroaryl, trialkylsilyl, alkyl, cycloalkyl, alkoxy, alkylthio, and the like. Specific examples of aryl-substituted heteroaryl include, but are not limited to, phenyl-substituted dibenzofuranyl, phenyl-substituted dibenzothienyl, N-phenylcarbazolyl, and the like. It should be understood that the number of carbon atoms of the substituted heteroaryl refers to the total number of carbon atoms of the heteroaryl and substituents on the heteroaryl.

In the present disclosure, specific examples of heteroaryl as a substituent include, but are not limited to, dibenzofuranyl, dibenzothienyl, carbazolyl, N-phenylcarbazolyl, phenanthrolinyl, and the like.

In the present disclosure, the halogen group may be fluorine, chlorine, bromine, or iodine.

According to one embodiment of the present disclosure, X₂, X₃, X₄, and X₅ are each C(H).

According to another embodiment of the present disclosure, one of X₂, X₃, X₄, and X₅ is N, and the rest are C(H). For example, X₂ is N, and X₃, X₄, and X₅ are each C(H); or X₃ is N, and X₂, X₄, and X₅ are each C(H); or X₄ is N, and X₃, X₂, and X₅ are each C(H); or X₅ is N, and X₃, X₂, and X₄ are each C(H).

According to another embodiment of the present disclosure, two of X₂, X₃, X₄, and X₅ are N, and the rest are C(H). For example, X₂ and X₄ are N, and X₃ and X₅ are each C(H); or X₃ and X₅ are N, and X₂ and X₄ are each C(H).

According to another embodiment of the present disclosure, three of X₂, X₃, X₄, and X₅ are N, and the rest is C(H).

According to one embodiment of the present disclosure, L and L₁ are respectively and independently selected from a single bond, substituted or unsubstituted arylene with 6 to 20 carbon atoms, and substituted or unsubstituted heteroarylene with 5 to 20 carbon atoms.

According to another embodiment of the present disclosure, L and L₁ are respectively and independently selected from a single bond, substituted or unsubstituted phenylene, substituted or unsubstituted naphthylene, substituted or unsubstituted biphenylene, substituted or unsubstituted pyridylidene, substituted or unsubstituted quinolylidene, substituted or unsubstituted fluorenylidene, substituted or unsubstituted carbazolylidene, substituted or unsubstituted dibenzofurylidene, substituted or unsubstituted dibenzothienylene, substituted or unsubstituted phenanthrylene, substituted or unsubstituted anthrylene, and substituted or unsubstituted N-phenylcarbazolylidene;

or, a group formed by connecting any two of the above groups by a single bond.

Wherein, “a group formed by connecting any two of the above groups by a single bond” means that: L and L₁ may also be selected from a group formed by connecting any two of substituted or unsubstituted phenylene, substituted or unsubstituted naphthylene, substituted or unsubstituted biphenylene, substituted or not pyridylidene, substituted or unsubstituted quinolylidene, substituted or unsubstituted fluorenylidene, substituted or unsubstituted carbazolylidene, substituted or unsubstituted dibenzofurylidene, substituted or unsubstituted dibenzothienylene, substituted or unsubstituted phenanthrylene, substituted or unsubstituted anthrylene, and substituted or unsubstituted N-phenylcarbazolylidene, and connecting by a single bond means that any two groups are connected to each other by their chemical bonds

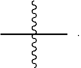

For example, the substituted or unsubstituted phenylene

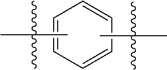

is connected to

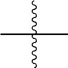

of the substituted or unsubstituted carbazolylidene

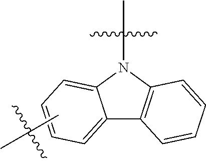

by its

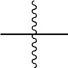

to form a group

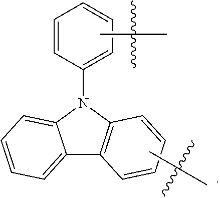

Optionally, substituents in the L and the L₁ are respectively and independently selected from deuterium, a halogen group, cyano, alkyl with 1 to 5 carbon atoms, aryl with 6 to 12 carbon atoms, and heteroaryl with 5 to 12 carbon atoms.

Specifically, specific examples of the substituents in the L and the L₁ include, but are not limited to, deuterium, fluorine, cyano, methyl, ethyl, n-propyl, isopropyl, tert-butyl, phenyl, naphthyl, biphenyl, and carbazolyl.

According to one embodiment of the present disclosure, L and L₁ are respectively and independently selected from a single bond or the group consisting of groups represented by formulae j-1 to j-14:

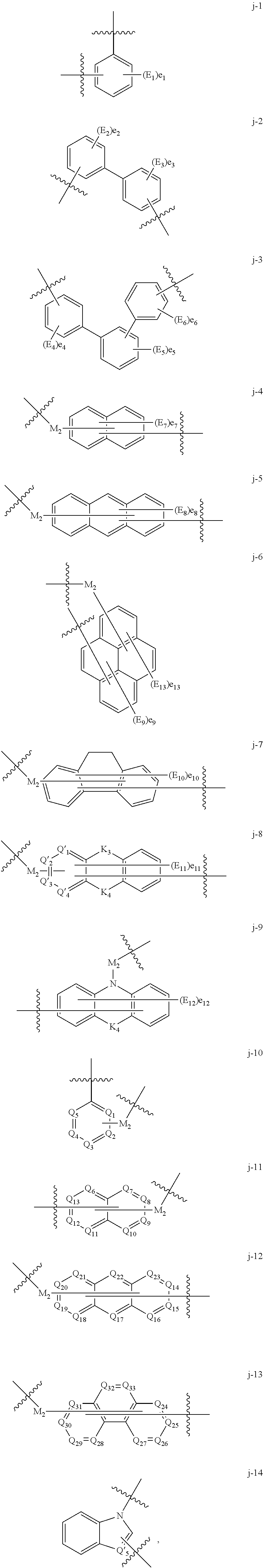

wherein M₂ is selected from a single bond or

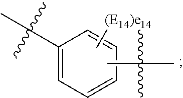

Q₁-Q₅ and Q′₁-Q′₅ are each independently selected from N or C(J₅), and at least one of Q₁-Q₅ is selected from N; and when two or more of Q₁-Q₅ are selected from C(J₅), any two J₅ are the same or different, and when two or more of Q′₁-Q′₄ are selected from C(J₅), any two J₅ are the same or different;

Q₆-Q₁₃ are each independently selected from N, C or C(J₆), and at least one of Q₆-Q₁₃ is selected from N; and when two or more of Q₆-Q₁₃ are selected from C(J₆), any two J₆ are the same or different;

Q₁₄-Q₂₃ are each independently selected from N, C or C(J₇), and at least one of Q₁₄-Q₂₃ is selected from N; and when two or more of Q₁₄-Q₂₃ are selected from C(J₇), any two J₇ are the same or different;

Q₂₄-Q₃₃ are each independently selected from N, C or C(J₈), and at least one of Q₂₄-Q₃₃ is selected from N; and when two or more of Q₂₄-Q₃₃ are selected from C(J₈), any two J₈ are the same or different;

E₁-E₁₄, and J₅-J₉ are each independently selected from: hydrogen, deuterium, a halogen group, cyano, heteroaryl with 3 to 20 carbon atoms, aryl with 6 to 20 carbon atoms which can be optionally substituted by 0, 1, 2, 3, 4, or 5 substituents independently selected from deuterium, fluorine, chlorine, cyano, methyl, ethyl, and tert-butyl, trialkylsilyl with 3 to 12 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, phosphinyloxy with 6 to 18 carbon atoms, and triarylsilyl with 18 to 24 carbon atoms; and the aryl with 6 to 20 carbon atoms which can be optionally substituted by 0, 1, 2, 3, 4, or 5 substituents independently selected from deuterium, fluorine, chlorine, cyano, methyl, ethyl, and tert-butyl means that the aryl may or may not be substituted by one or more of deuterium, fluorine, chlorine, cyano, methyl, and tert-butyl, and when the number of substituents on the aryl is greater than or equal to 2, the substituents may be the same or different.

When any one of E₁-E₁₄ is independently selected from aryl with 6 to 20 carbon atoms, E₁-E₃ and E₁₄ are not aryl;

e₁-e₁₄ are represented by eᵣ, E₁-E₁₄ are represented by Eᵣ, r is a variable, and represents any integer from 1 to 14, and eᵣ represents the number of a substituent Eᵣ; when r is selected from 1, 2, 3, 4, 5, 6, 9, 13 or 14, eᵣ is selected from 1, 2, 3 or 4; when r is selected from 7 or 11, eᵣ is selected from 1, 2, 3, 4, 5 or 6; when r is 12, eᵣ is selected from 1, 2, 3, 4, 5, 6, or 7; when r is selected from 8 or 10, eᵣ is selected from 1, 2, 3, 4, 5, 6, 7, or 8; and when eᵣ is greater than 1, any two Eᵣ are the same or different;

K₃ is selected from O, S, Se, N(E₁₅), C(E₁₆E₁₇), and Si(E₁₈E₁₉); where E₁₅, E₁₆, E₁₇, E₁₈ and E₁₉ are each independently selected from: aryl with 6 to 20 carbon atoms, heteroaryl with 3 to 20 carbon atoms, alkyl with 1 to 10 carbon atoms, cycloalkyl with 3 to 10 carbon atoms, and heterocycloalkyl with 2 to 10 carbon atoms, or E₁₆ and E₁₇ are connected to each other to form a saturated or unsaturated ring with 3 to 15 carbon atoms together with the atoms to which they are jointly connected, or E₁₈ and E₁₉ are connected to each other to form a saturated or unsaturated ring with 3 to 15 carbon atoms together with the atoms to which they are jointly connected;

K₄ is selected from a single bond, O, S, Se, N(E₂₀), C(E₂₁E₂₂), and Si(E₂₃E₂₄); where E₂₀-E₂₄ are each independently selected from: aryl with 6 to 20 carbon atoms, heteroaryl with 3 to 20 carbon atoms, alkyl with 1 to 10 carbon atoms, cycloalkyl with 3 to 10 carbon atoms, and heterocycloalkyl with 2 to 10 carbon atoms, or E₂₁ and E₂₂ are connected to each other to form a saturated or unsaturated ring with 3 to 15 carbon atoms together with the atoms to which they are jointly connected, or E₂₃ and E₂₄ are connected to each other to form a saturated or unsaturated ring with 3 to 15 carbon atoms together with the atoms to which they are jointly connected.

Optionally, L and L₁ are respectively and independently selected from a single bond, and a substituted or unsubstituted group V; the unsubstituted group V is selected from the group consisting of:

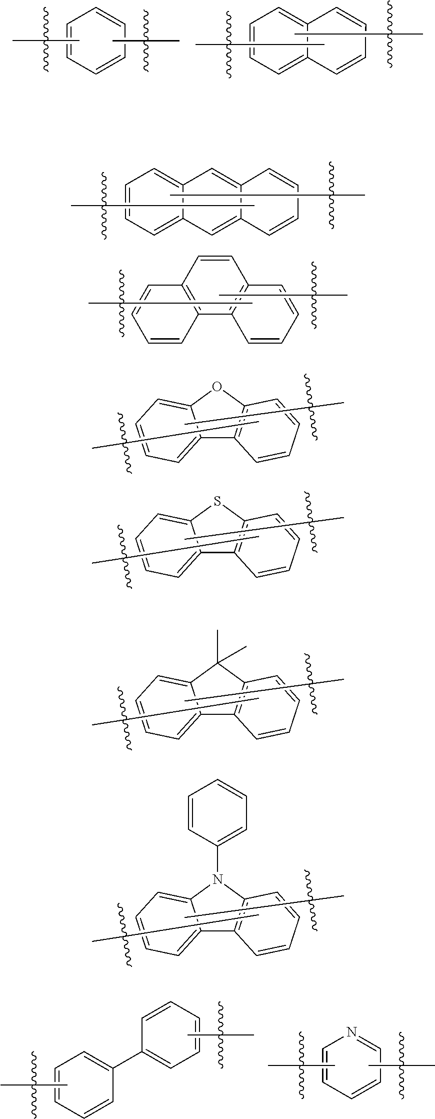

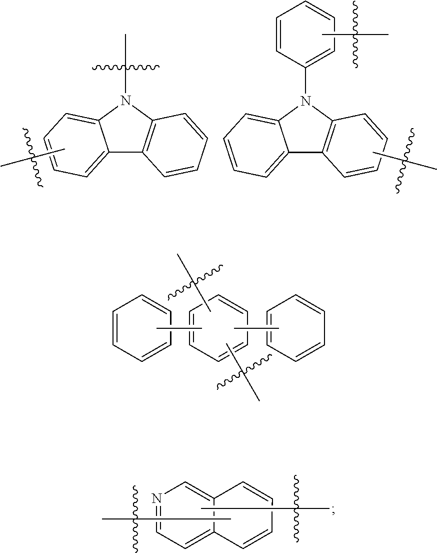

wherein

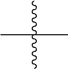

represents a chemical bond; the substituted group V has one or more substituents, and the substituents are each independently selected from deuterium, fluorine, cyano, a halogen group, methyl, ethyl, n-propyl, isopropyl, tert-butyl, phenyl, naphthyl, biphenyl, and carbazolyl; when the group V has two or more substituents, the two or more substituents are the same or different.

Optionally, L and L₁ are respectively and independently selected from a single bond or the group consisting of the following groups, but are not limited to this:

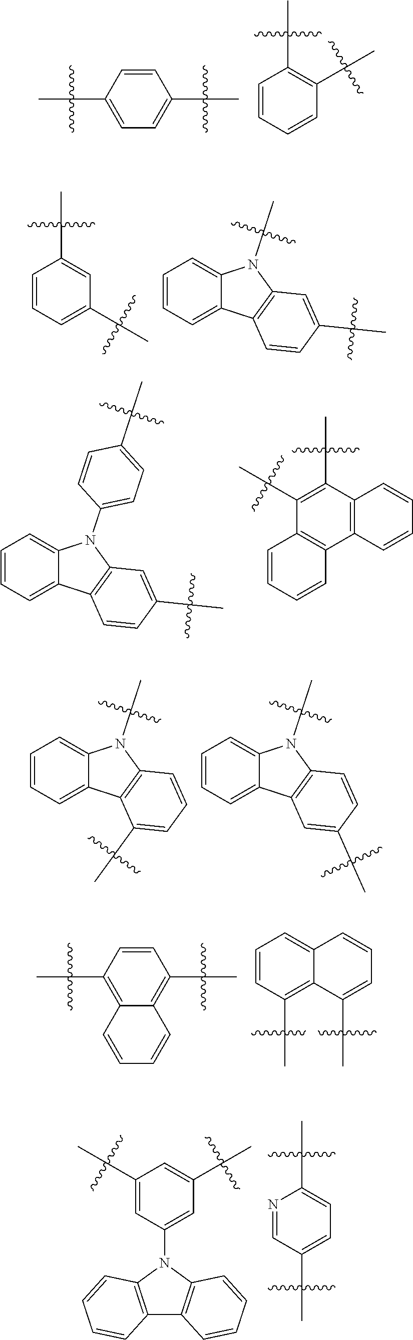

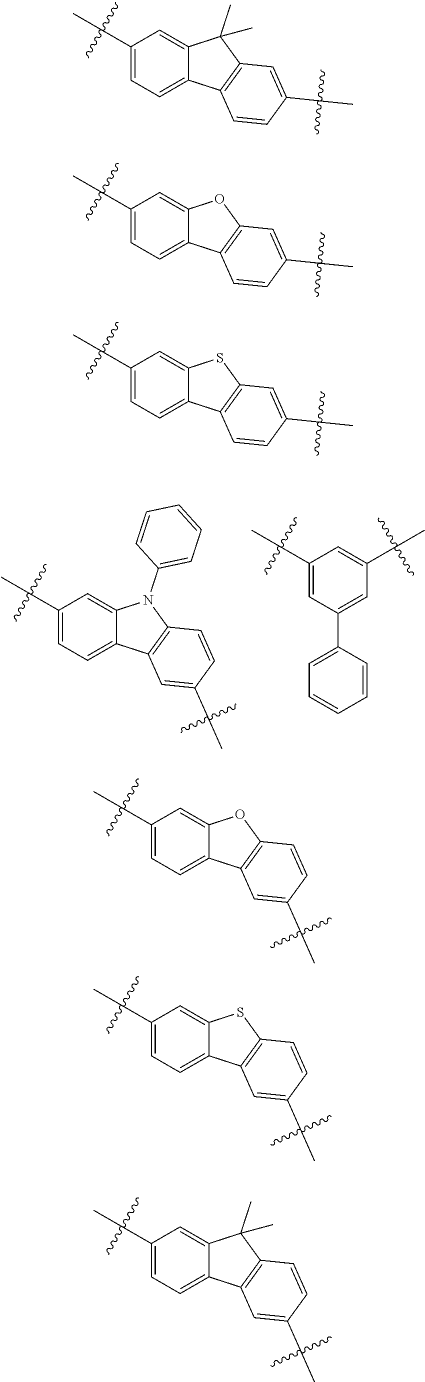

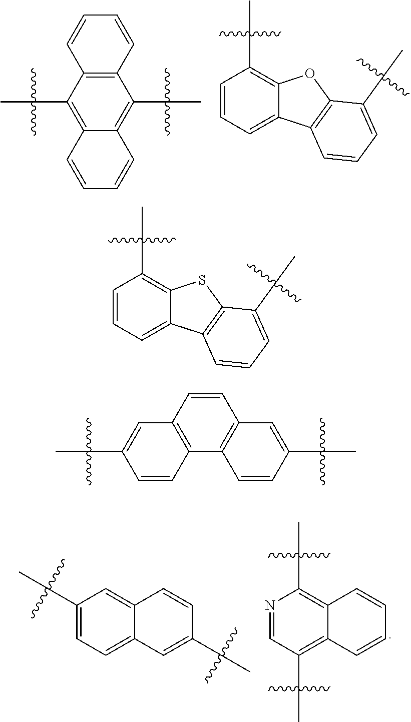

According to one embodiment of the present disclosure, Ar is selected from substituted or unsubstituted aryl with 6 to 26 carbon atoms and substituted or unsubstituted heteroaryl with 5 to 20 carbon atoms.

Optionally, substituents in the Ar are selected from deuterium, fluorine, cyano, alkyl with 1 to 5 carbon atoms, aryl with 6 to 20 carbon atoms, heteroaryl with 12 to 18 carbon atoms, and cycloalkyl with 5 to 10 carbon atoms;

or, any two adjacent substituents form a saturated or unsaturated ring having 5 to 8 carbon atoms.

Specifically, specific embodiments of substituents in Ar include, but are not limited to, deuterium, fluorine, cyano, methyl, ethyl, n-propyl, isopropyl, tert-butyl, cyclopentyl, cyclohexyl, phenyl, phenanthryl, naphthyl, dibenzofuranyl, dibenzothienyl, 9,9-dimethylfluorenyl, carbazolyl, N-phenylcarbazolyl, cyclohexyl, cyclopentyl, adamantyl, and the like.

In one embodiment of the present disclosure, the substituents in Ar form cyclopentane or cyclohexane.

According to one embodiment of the present disclosure, Ar is selected from the group consisting of groups represented by formulae i-1 to i-15 below:

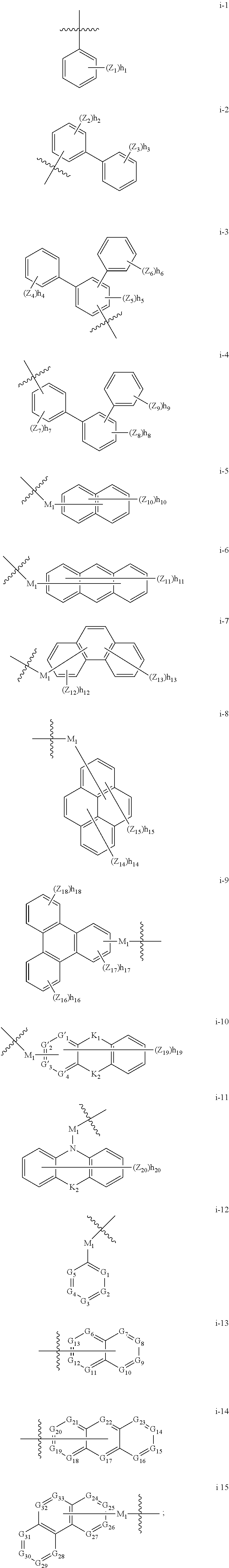

wherein M₁ is selected from a single bond or

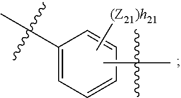

G₁-G₅ and G′₁-G′₄ are each independently selected from N, C, or C(J₁), and at least one of G₁-G₅ is selected from N; and when two or more of G₁-G₅ are selected from C(J₁), any two J₁ are the same or different;

G₆-G₁₃ are each independently selected from N, C or C(J₂), and at least one of G₆-G₁₃ is selected from N; and when two or more of G₆-G₁₃ are selected from C(J₂), any two J₂ are the same or different;

G₁₄-G₂₃ are each independently selected from N, C or C(J₃), and at least one of G₁₄-G₂₃ is selected from N; and when two or more of G₁₄-G₂₃ are selected from C(J₃), any two J₃ are the same or different;

G₂₄-G₃₃ are each independently selected from N, C or C(J₄), and at least one of G₂₄-G₃₃ is selected from N; and when two or more of G₂₄-G₃₃ are selected from C(J₄), any two J₄ are the same or different;

Z₁ is selected from hydrogen, deuterium, a halogen group, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, and triarylsilyl with 18 to 24 carbon atoms;

Z₂-Z₉, and Z₂₁ are each independently selected from: hydrogen, deuterium, a halogen group, cyano, trialkylsilyl with 3 to 12 carbon atoms, triarylsilyl with 18 to 24 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, and heteroaryl with 3 to 18 carbon atoms;

Z₁₀-Z₂₀, and J₁-J₄ are each independently selected from: hydrogen, deuterium, a halogen group, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, aryl with 6 to 18 carbon atoms which can be optionally substituted by 0, 1, 2, 3, 4, or 5 substituents independently selected from deuterium, fluorine, chlorine, cyano, methyl, ethyl, and tert-butyl, heteroaryl with 3 to 18 carbon atoms, and triarylsilyl with 18 to 24 carbon atoms; in the present disclosure, “aryl with 6 to 18 carbon atoms which can be optionally substituted by 0, 1, 2, 3, 4, or 5 substituents independently selected from deuterium, fluorine, chlorine, cyano, methyl, ethyl, and tert-butyl” means that the aryl may or may not be substituted by one or more of deuterium, fluorine, chlorine, cyano, methyl, ethyl, and tert-butyl, and when the number of substituents in the aryl is greater than or equal to 2, the substituents may be the same or different.

h₁-h₂₁ are represented by hₖ, Z₁-Z₂₁ are represented by Zₖ, k is a variable, and represents any integer from 1 to 21, and hₖ represents the number of a substituent Zₖ; when k is selected from 5 or 17, hₖ is selected from 1, 2 or 3; when k is selected from 2, 7, 8, 12, 15, 16, 18 or 21, hₖ is selected from 1, 2, 3 or 4; when k is selected from 1, 3, 4, 6, 9 or 14, hₖ is selected from 1, 2, 3, 4 or 5; when k is 13, hₖ is selected from 1, 2, 3, 4, 5, or 6; when k is selected from 10 or 19, hₖ is selected from 1, 2, 3, 4, 5, 6 or 7; when k is 20, hₖ is selected from 1, 2, 3, 4, 5, 6, 7, or 8; when k is 11, hₖ is selected from 1, 2, 3, 4, 5, 6, 7, 8 or 9; and when hₖ is greater than 1, any two Zₖ are the same or different;

K₁ is selected from O, S, N(Z₂₂), C(Z₂₃Z₂₄), and Si(Z₂₈Z₂₉); where Z₂₂, Z₂₃, Z₂₄, Z₂₈, and Z₂₉ are each independently selected from: aryl with 6 to 18 carbon atoms, heteroaryl with 3 to 18 carbon atoms, alkyl with 1 to 10 carbon atoms, or cycloalkyl with 3 to 10 carbon atoms, or the Z₂₃ and the Z₂₄ are connected to each other to form a saturated or unsaturated ring with 3 to 15 carbon atoms together with the atoms to which they are jointly connected, or the Z₂₈ and the Z₂₉ are connected to each other to form a saturated or unsaturated ring with 3 to 15 carbon atoms together with the atoms to which they are jointly connected; and

K₂ is selected from a single bond, O, S, N(Z₂₅), C(Z₂₆Z₂₇), and Si(Z₃₀Z₃₁); where Z₂₅, Z₂₆, Z₂₇, Z₃₀, and Z₃₁ are each independently selected from: aryl with 6 to 18 carbon atoms, heteroaryl with 3 to 18 carbon atoms, alkyl with 1 to 10 carbon atoms, or cycloalkyl with 3 to 10 carbon atoms, or the Z₂₆ and the Z₂₇ are connected to each other to form a saturated or unsaturated ring with 3 to 15 carbon atoms together with the atoms to which they are jointly connected, or the Z₃₀ and the Z₃₁ are connected to each other to form a saturated or unsaturated ring with 3 to 15 carbon atoms together with the atoms to which they are jointly connected. In the present disclosure, the ring refers to a saturated or unsaturated ring, for example,

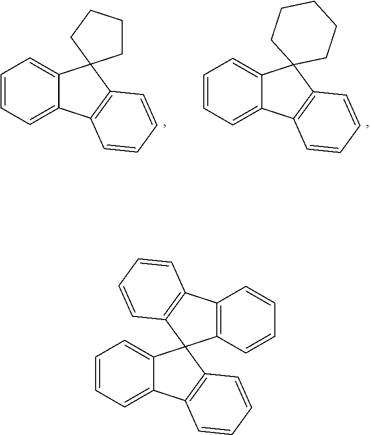

and the like, but is not limited to this.

Optionally, Ar is selected from a substituted or unsubstituted group W, and the unsubstituted group W is selected from the group consisting of:

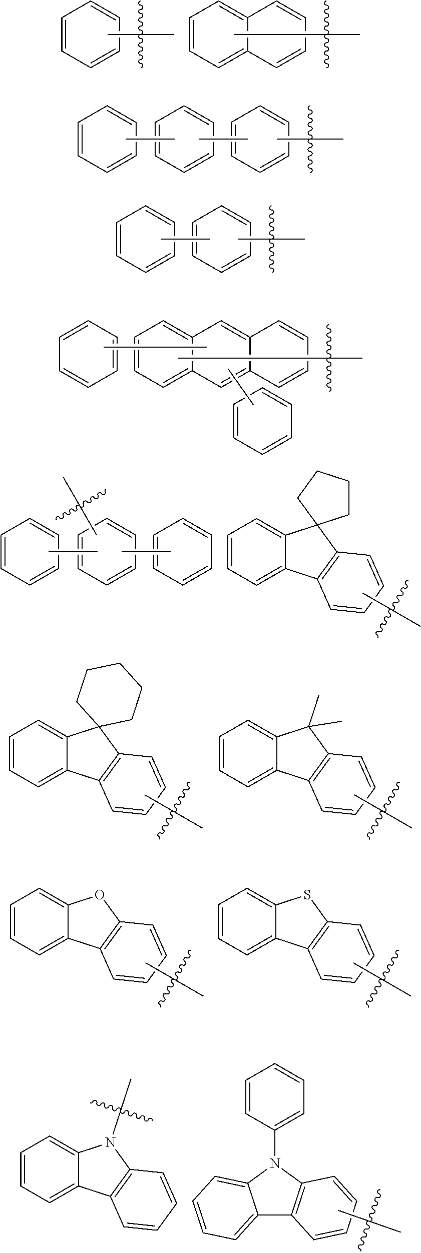

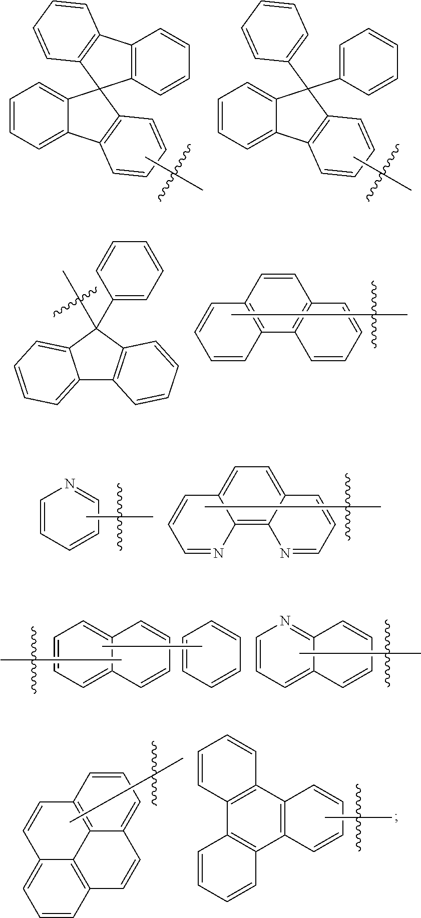

wherein

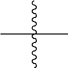

represents a chemical bond; and the substituted group W has one or more substituents, and the substituents are each independently selected from deuterium, fluorine, cyano, a halogen group, methyl, ethyl, n-propyl, isopropyl, tert-butyl, phenyl, naphthyl, biphenyl, cyclopentyl, and cyclohexyl; when the group W has two or more substituents, the two or more substituents are the same or different.

Optionally, Ar is selected from the group consisting of the following groups, but is not limited to this:

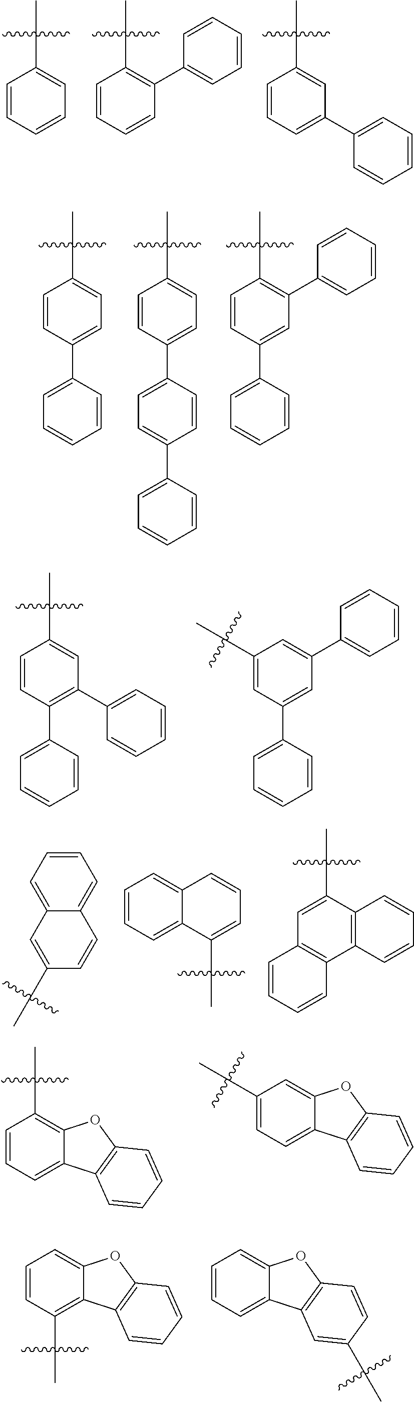

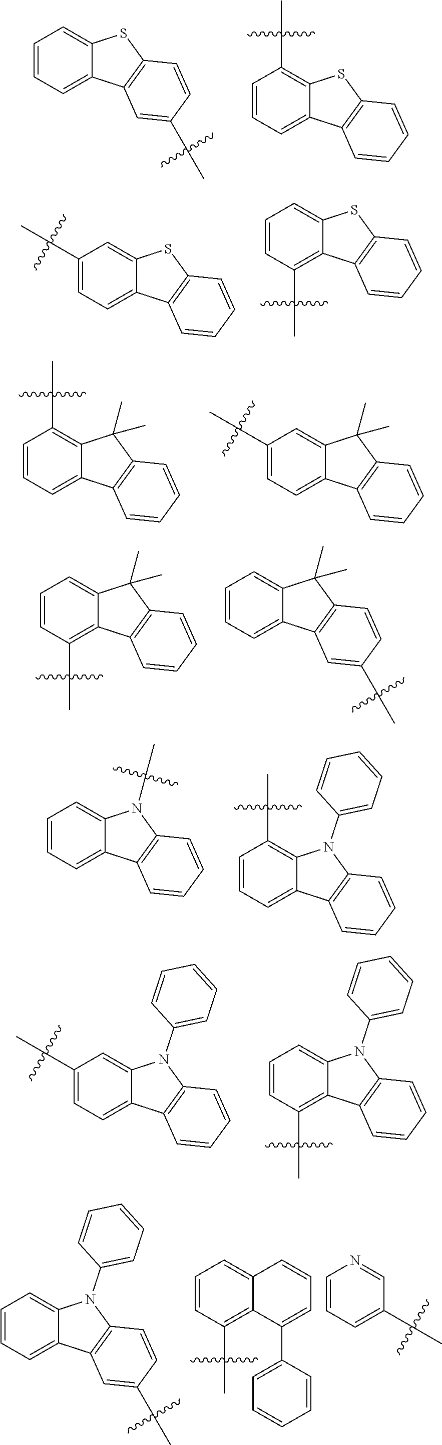

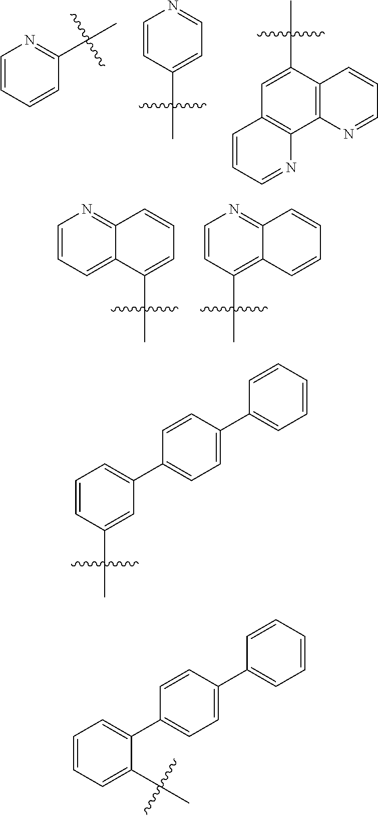

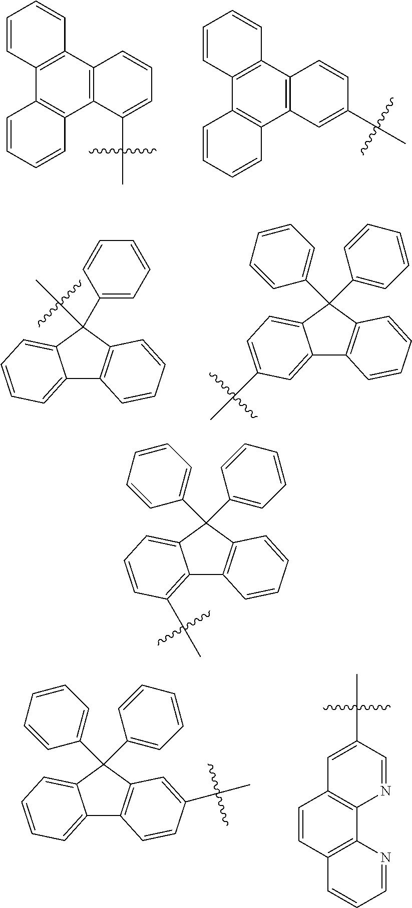

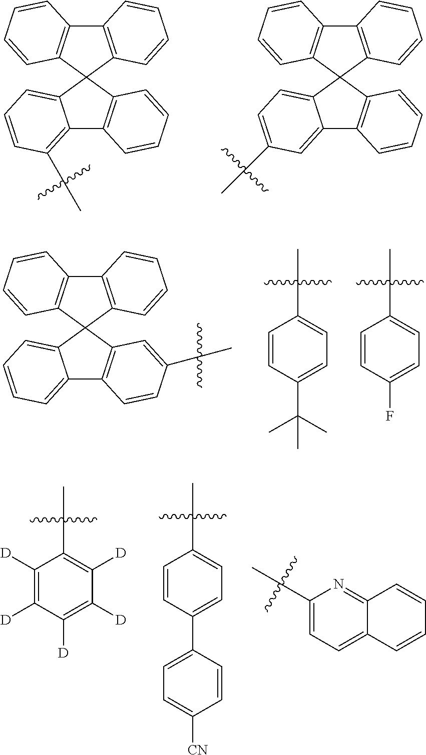

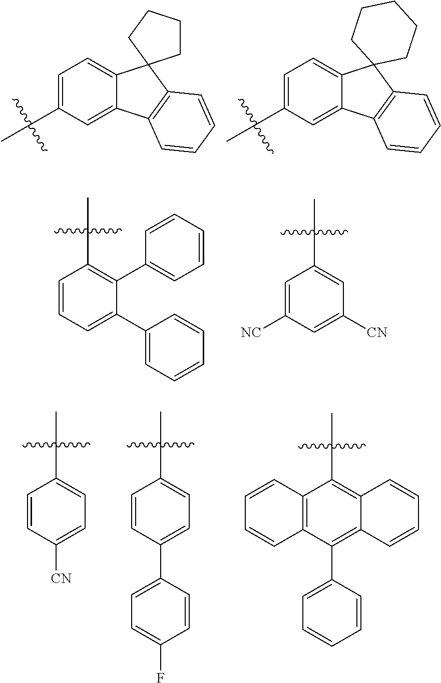

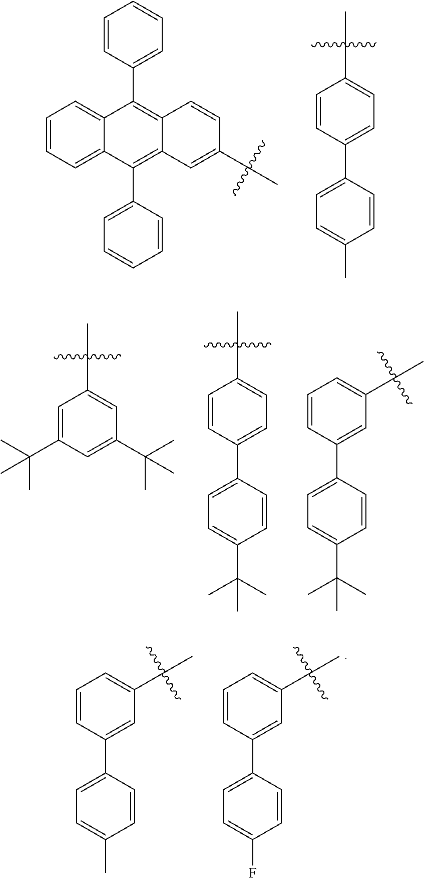

Optionally, the nitrogen-containing compound is selected from the group of consisting of the following compounds, but is not limited to this:

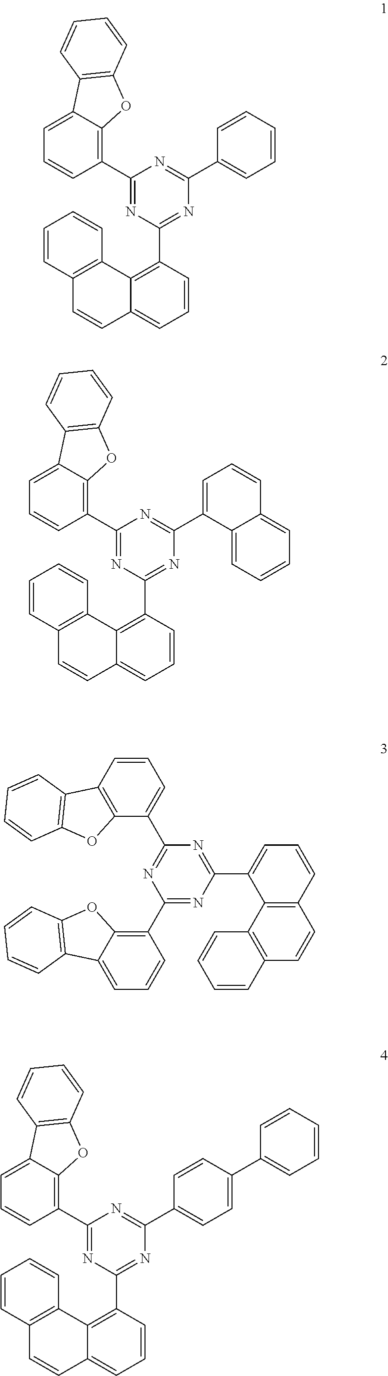

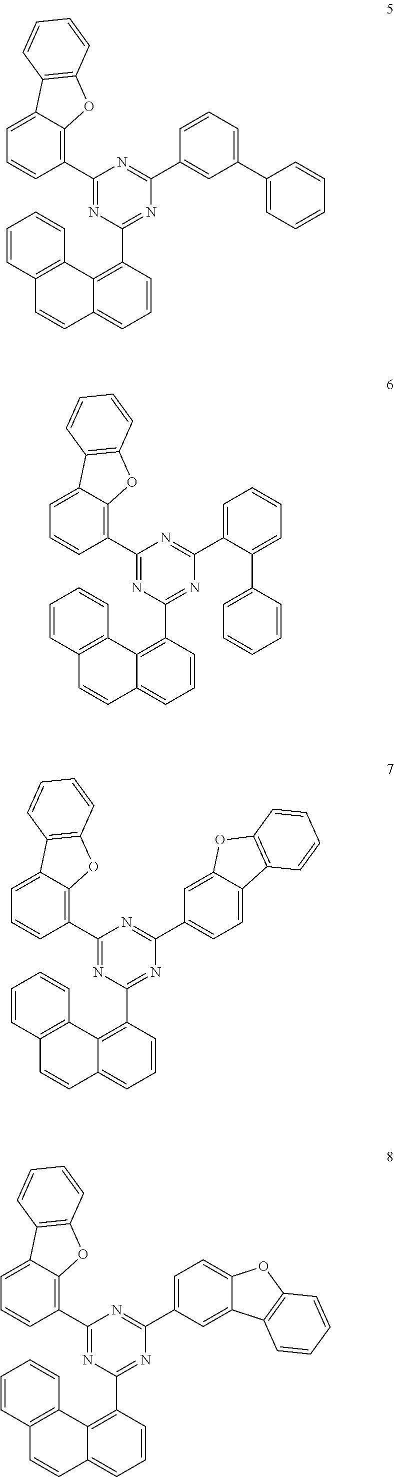 [SMILES: c1ccc(-c2cccc(-c3nc(-c4cccc5c4oc4ccccc45)nc(-c4cccc5ccc6ccccc6c45)n3)c2)cc1 ; c1ccc(-c2ccccc2-c2nc(-c3cccc4c3oc3ccccc34)nc(-c3cccc4ccc5ccccc5c34)n2)cc1 ; c1ccc2c(c1)ccc1cccc(-c3nc(-c4ccc5c(c4)oc4ccccc45)nc(-c4cccc5c4oc4ccccc45)n3)c12 ; c1ccc2c(c1)ccc1cccc(-c3nc(-c4ccc5oc6ccccc6c5c4)nc(-c4cccc5c4oc4ccccc45)n3)c12]

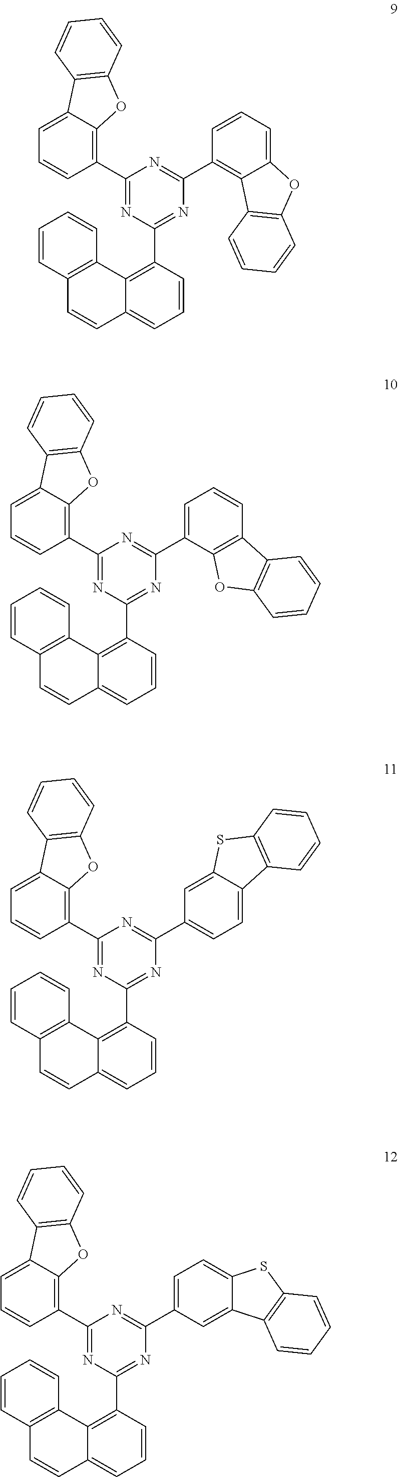 [SMILES: c1ccc2c(c1)ccc1cccc(-c3nc(-c4cccc5c4oc4ccccc45)nc(-c4cccc5oc6ccccc6c45)n3)c12 ; c1ccc2c(c1)ccc1cccc(-c3nc(-c4cccc5c4oc4ccccc45)nc(-c4cccc5c4oc4ccccc45)n3)c12 ; c1ccc2c(c1)ccc1cccc(-c3nc(-c4ccc5c(c4)sc4ccccc45)nc(-c4cccc5c4oc4ccccc45)n3)c12 ; c1ccc2c(c1)ccc1cccc(-c3nc(-c4ccc5sc6ccccc6c5c4)nc(-c4cccc5c4oc4ccccc45)n3)c12]

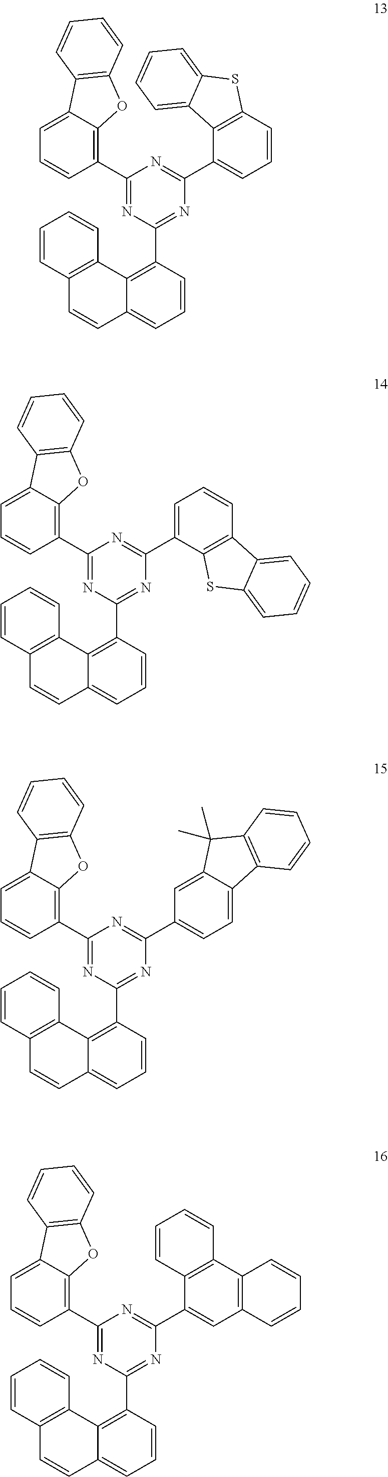 [SMILES: c1ccc2c(c1)ccc1cccc(-c3nc(-c4cccc5c4oc4ccccc45)nc(-c4cccc5sc6ccccc6c45)n3)c12 ; c1ccc2c(c1)ccc1cccc(-c3nc(-c4cccc5c4oc4ccccc45)nc(-c4cccc5c4sc4ccccc45)n3)c12 ; CC1(C)c2ccccc2-c2ccc(-c3nc(-c4cccc5c4oc4ccccc45)nc(-c4cccc5ccc6ccccc6c45)n3)cc21 ; c1ccc2c(c1)cc(-c1nc(-c3cccc4c3oc3ccccc34)nc(-c3cccc4ccc5ccccc5c34)n1)c1ccccc12]

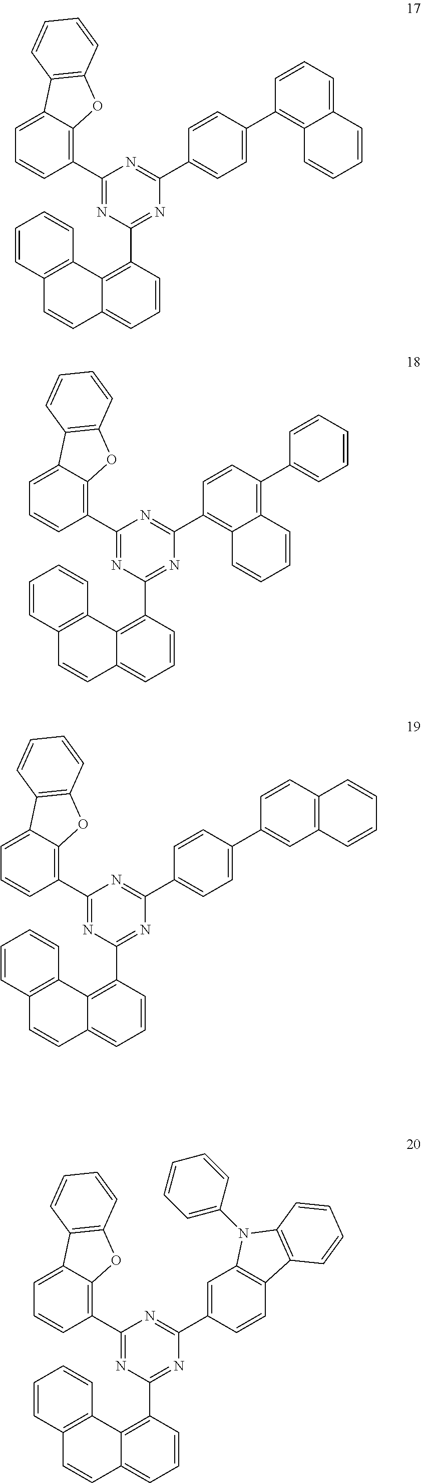 [SMILES: c1ccc2c(-c3ccc(-c4nc(-c5cccc6c5oc5ccccc56)nc(-c5cccc6ccc7ccccc7c56)n4)cc3)cccc2c1 ; c1ccc(-c2ccc(-c3nc(-c4cccc5c4oc4ccccc45)nc(-c4cccc5ccc6ccccc6c45)n3)c3ccccc23)cc1 ; c1ccc2cc(-c3ccc(-c4nc(-c5cccc6c5oc5ccccc56)nc(-c5cccc6ccc7ccccc7c56)n4)cc3)ccc2c1 ; c1ccc(-n2c3ccccc3c3ccc(-c4nc(-c5cccc6c5oc5ccccc56)nc(-c5cccc6ccc7ccccc7c56)n4)cc32)cc1]

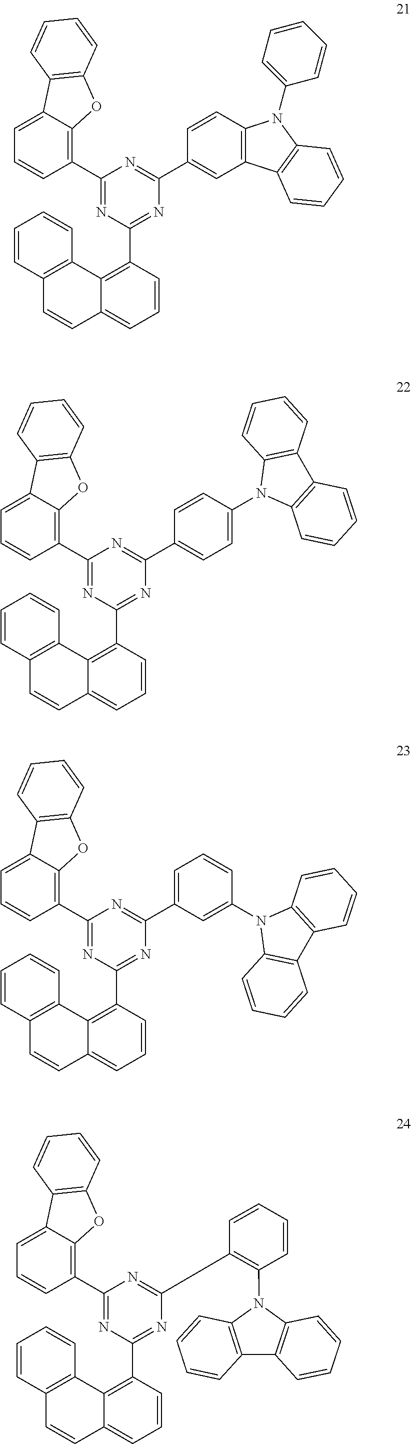 [SMILES: c1ccc(-n2c3ccccc3c3cc(-c4nc(-c5cccc6c5oc5ccccc56)nc(-c5cccc6ccc7ccccc7c56)n4)ccc32)cc1 ; c1ccc2c(c1)ccc1cccc(-c3nc(-c4ccc(-n5c6ccccc6c6ccccc65)cc4)nc(-c4cccc5c4oc4ccccc45)n3)c12 ; c1cc(-c2nc(-c3cccc4c3oc3ccccc34)nc(-c3cccc4ccc5ccccc5c34)n2)cc(-n2c3ccccc3c3ccccc32)c1 ; c1ccc(-n2c3ccccc3c3ccccc32)c(-c2nc(-c3cccc4c3oc3ccccc34)nc(-c3cccc4ccc5ccccc5c34)n2)c1]

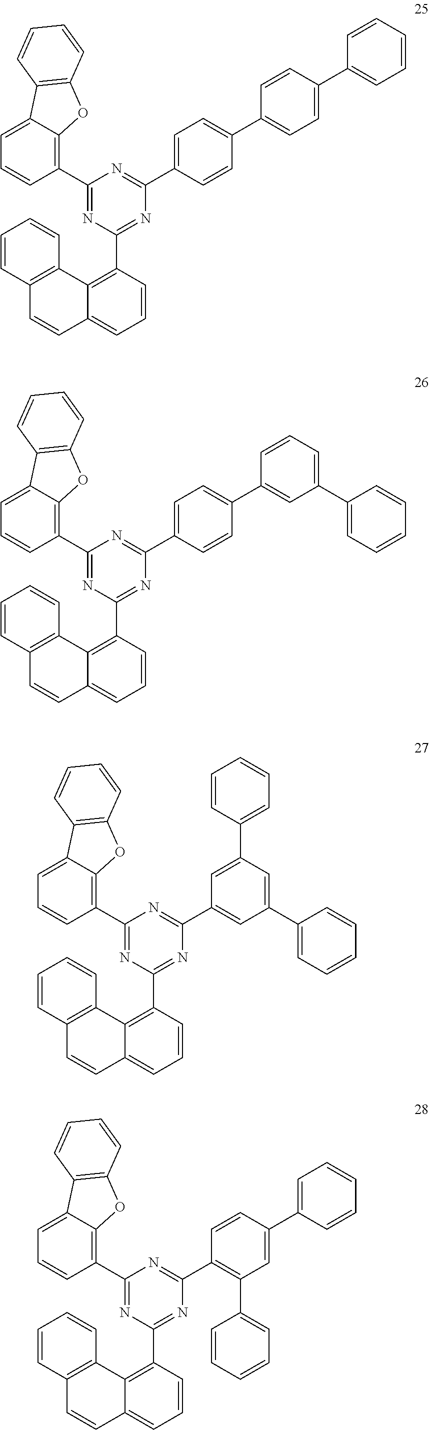

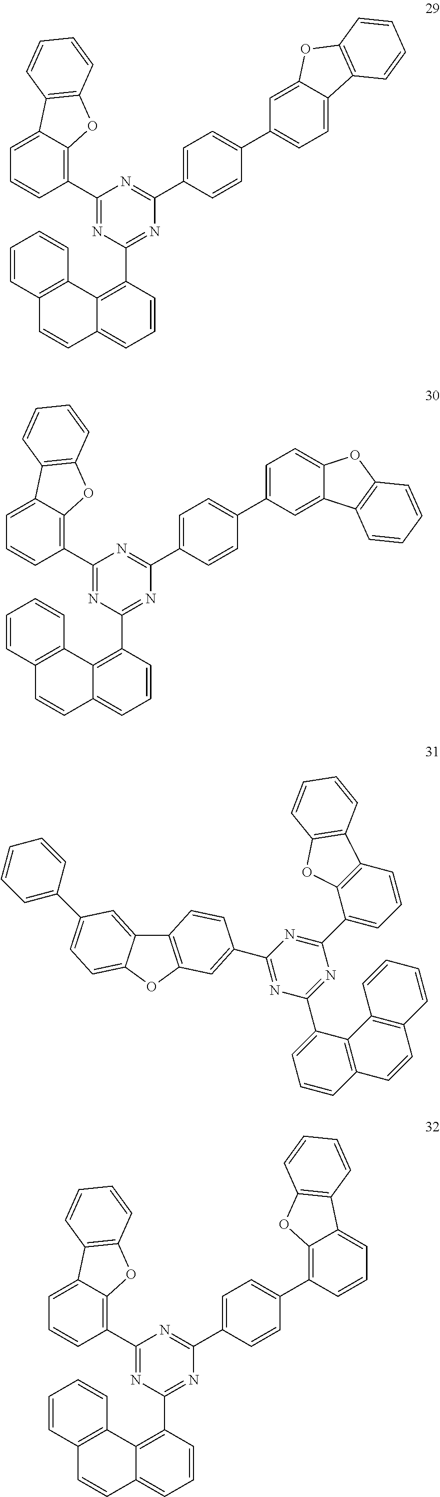

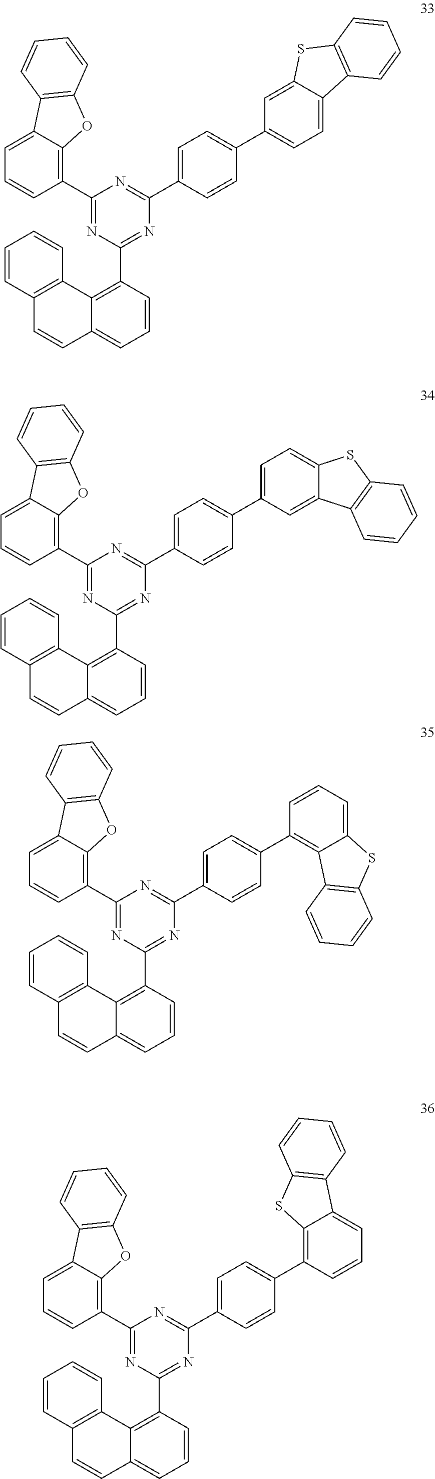

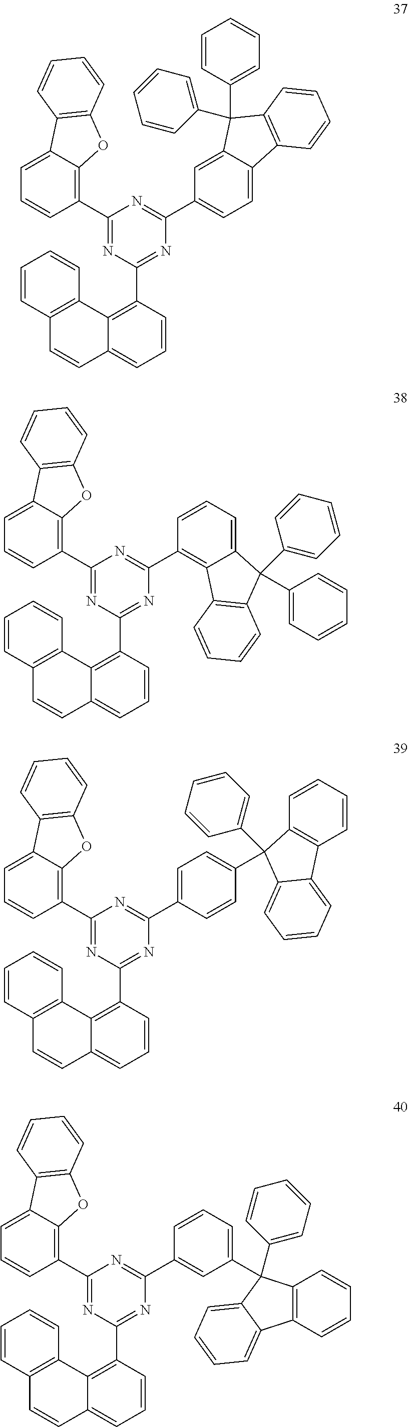

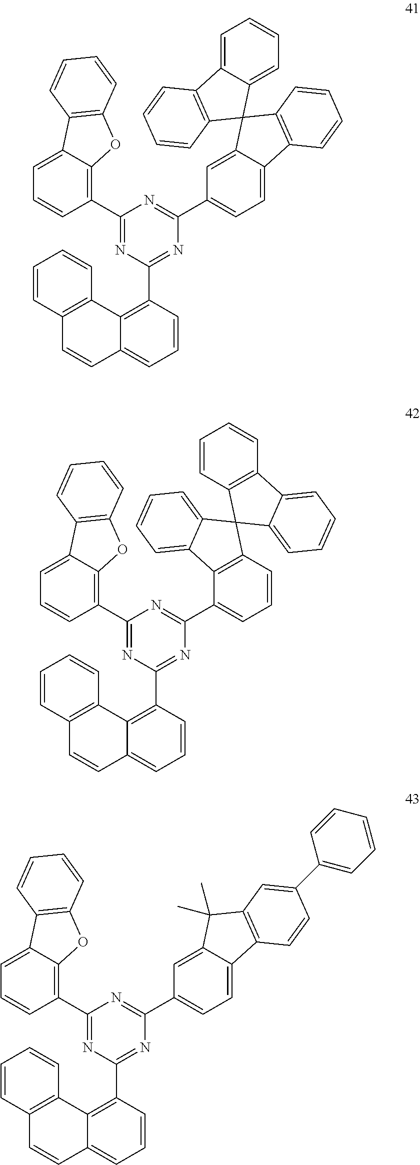 [SMILES: c1ccc2c(c1)-c1ccccc1C21c2ccccc2-c2ccc(-c3nc(-c4cccc5c4oc4ccccc45)nc(-c4cccc5ccc6ccccc6c45)n3)cc21 ; c1ccc2c(c1)-c1ccccc1C21c2ccccc2-c2c(-c3nc(-c4cccc5c4oc4ccccc45)nc(-c4cccc5ccc6ccccc6c45)n3)cccc21 ; CC1(C)c2cc(-c3ccccc3)ccc2-c2ccc(-c3nc(-c4cccc5c4oc4ccccc45)nc(-c4cccc5ccc6ccccc6c45)n3)cc21]

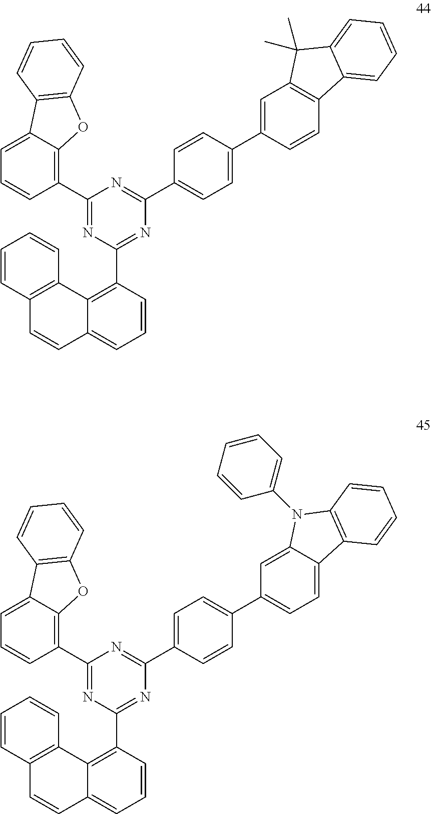 [SMILES: CC1(C)c2ccccc2-c2ccc(-c3ccc(-c4nc(-c5cccc6c5oc5ccccc56)nc(-c5cccc6ccc7ccccc7c56)n4)cc3)cc21 ; c1ccc(-n2c3ccccc3c3ccc(-c4ccc(-c5nc(-c6cccc7c6oc6ccccc67)nc(-c6cccc7ccc8ccccc8c67)n5)cc4)cc32)cc1]

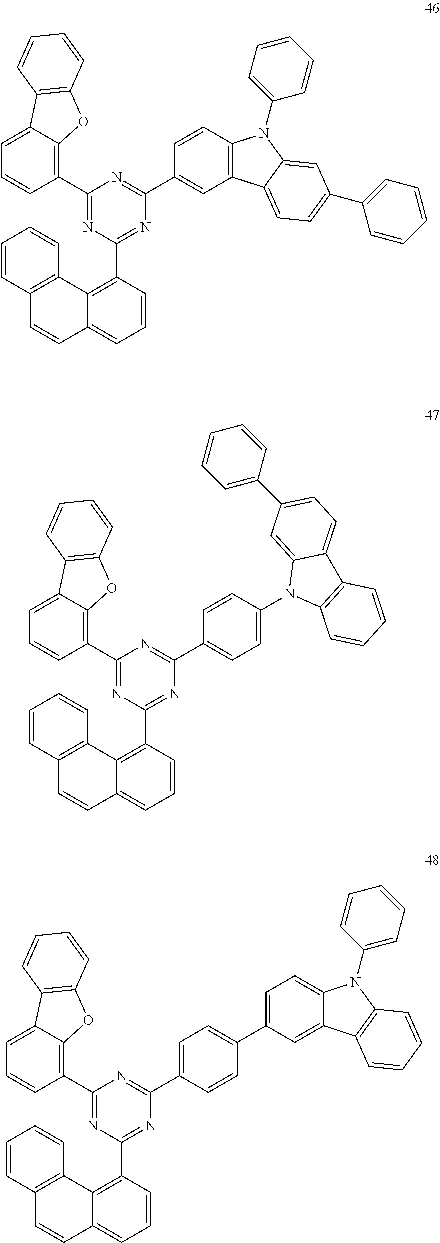 [SMILES: c1ccc(-c2ccc3c4cc(-c5nc(-c6cccc7c6oc6ccccc67)nc(-c6cccc7ccc8ccccc8c67)n5)ccc4n(-c4ccccc4)c3c2)cc1 ; c1ccc(-c2ccc3c4ccccc4n(-c4ccc(-c5nc(-c6cccc7c6oc6ccccc67)nc(-c6cccc7ccc8ccccc8c67)n5)cc4)c3c2)cc1 ; c1ccc(-n2c3ccccc3c3cc(-c4ccc(-c5nc(-c6cccc7c6oc6ccccc67)nc(-c6cccc7ccc8ccccc8c67)n5)cc4)ccc32)cc1]

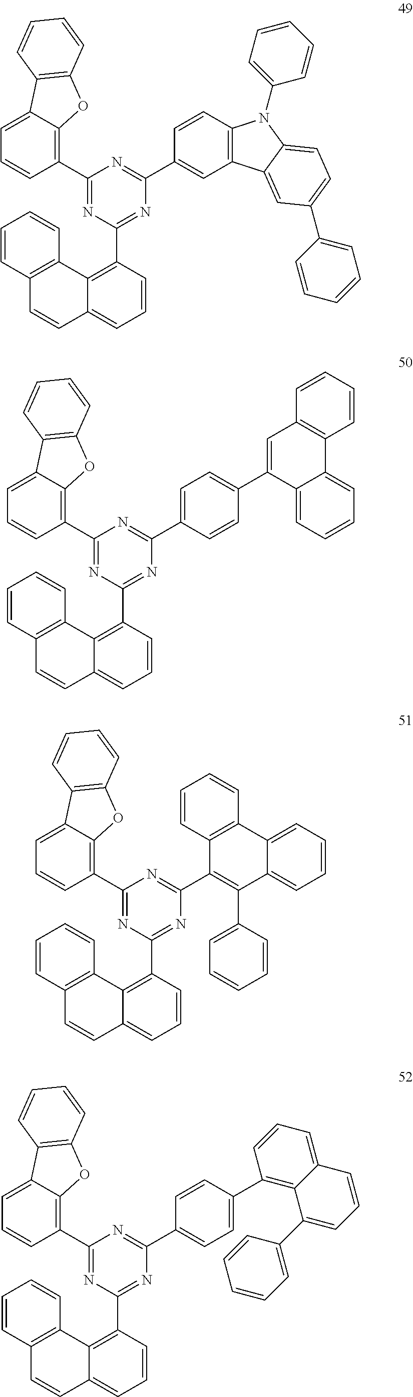

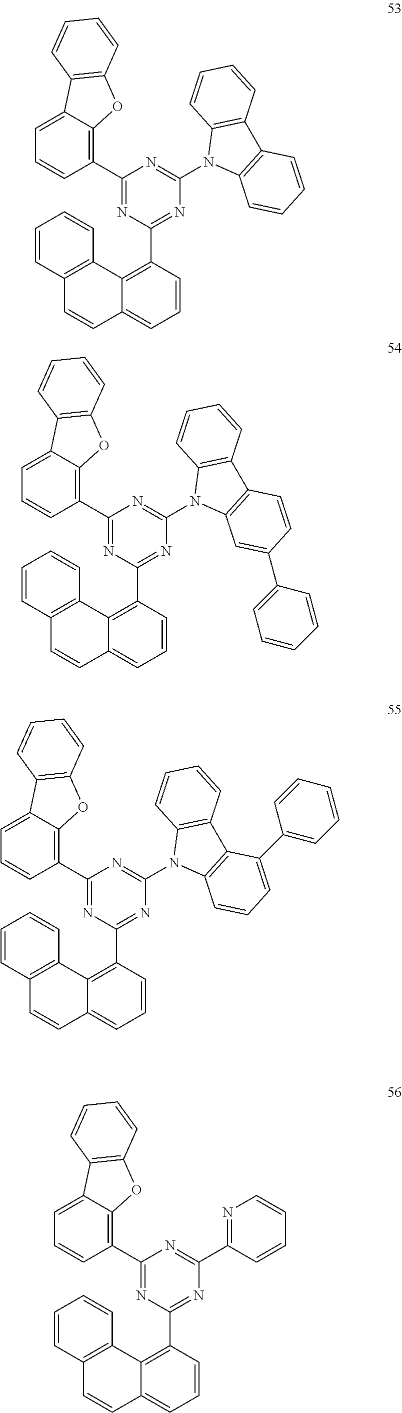

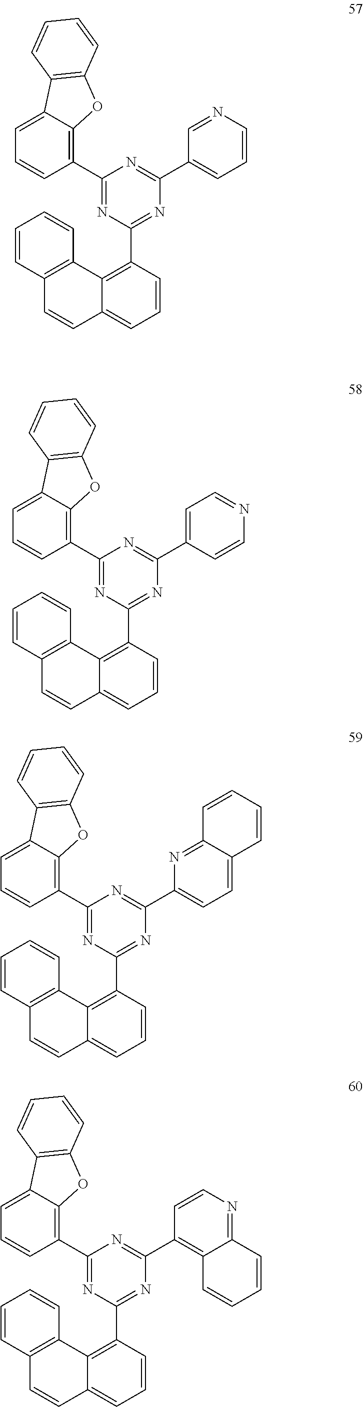

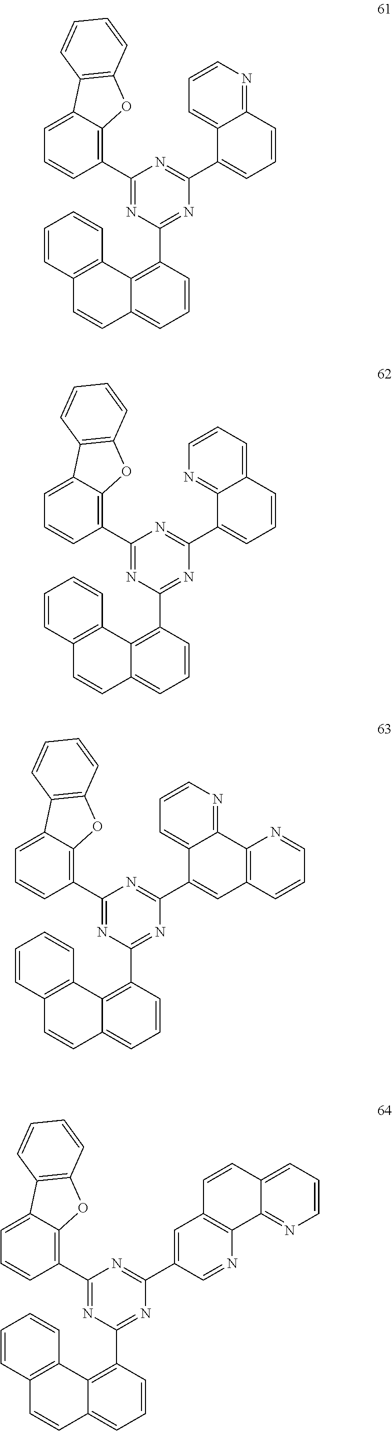 [SMILES: c1ccc2c(c1)ccc1cccc(-c3nc(-c4cccc5ncccc45)nc(-c4cccc5c4oc4ccccc45)n3)c12 ; c1cnc2c(-c3nc(-c4cccc5c4oc4ccccc45)nc(-c4cccc5ccc6ccccc6c45)n3)cccc2c1 ; c1ccc2c(c1)ccc1cccc(-c3nc(-c4cc5cccnc5c5ncccc45)nc(-c4cccc5c4oc4ccccc45)n3)c12 ; c1ccc2c(c1)ccc1cccc(-c3nc(-c4cnc5c(ccc6cccnc65)c4)nc(-c4cccc5c4oc4ccccc45)n3)c12]

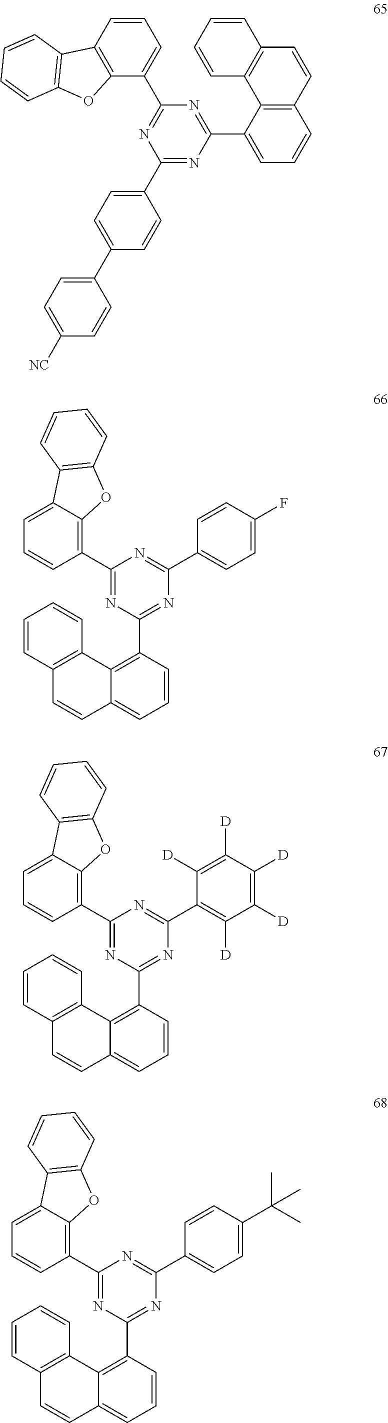

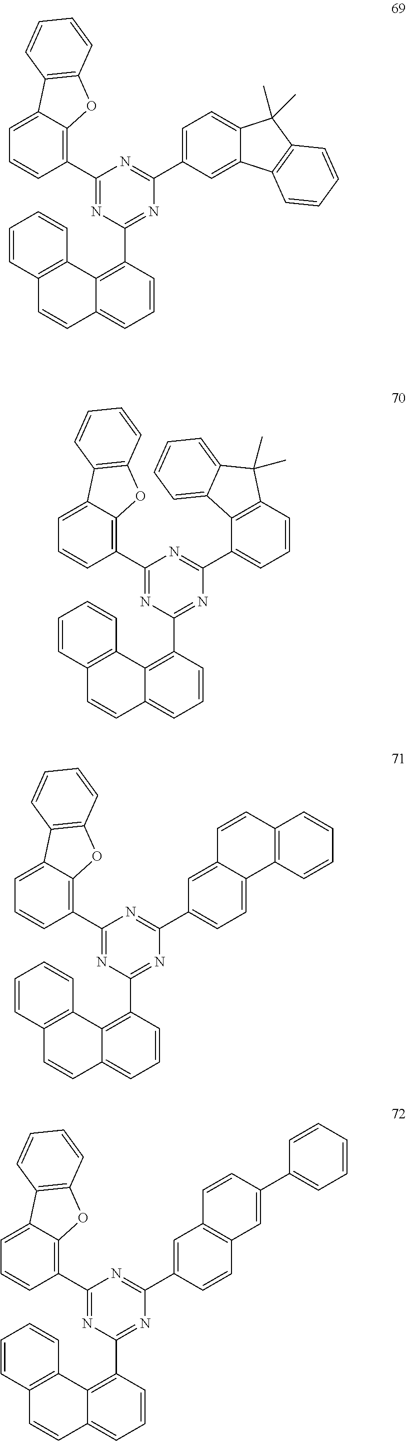 [SMILES: CC1(C)c2ccccc2-c2cc(-c3nc(-c4cccc5c4oc4ccccc45)nc(-c4cccc5ccc6ccccc6c45)n3)ccc21 ; CC1(C)c2ccccc2-c2c(-c3nc(-c4cccc5c4oc4ccccc45)nc(-c4cccc5ccc6ccccc6c45)n3)cccc21 ; c1ccc2c(c1)ccc1cc(-c3nc(-c4cccc5c4oc4ccccc45)nc(-c4cccc5ccc6ccccc6c45)n3)ccc12 ; c1ccc(-c2ccc3cc(-c4nc(-c5cccc6c5oc5ccccc56)nc(-c5cccc6ccc7ccccc7c56)n4)ccc3c2)cc1]

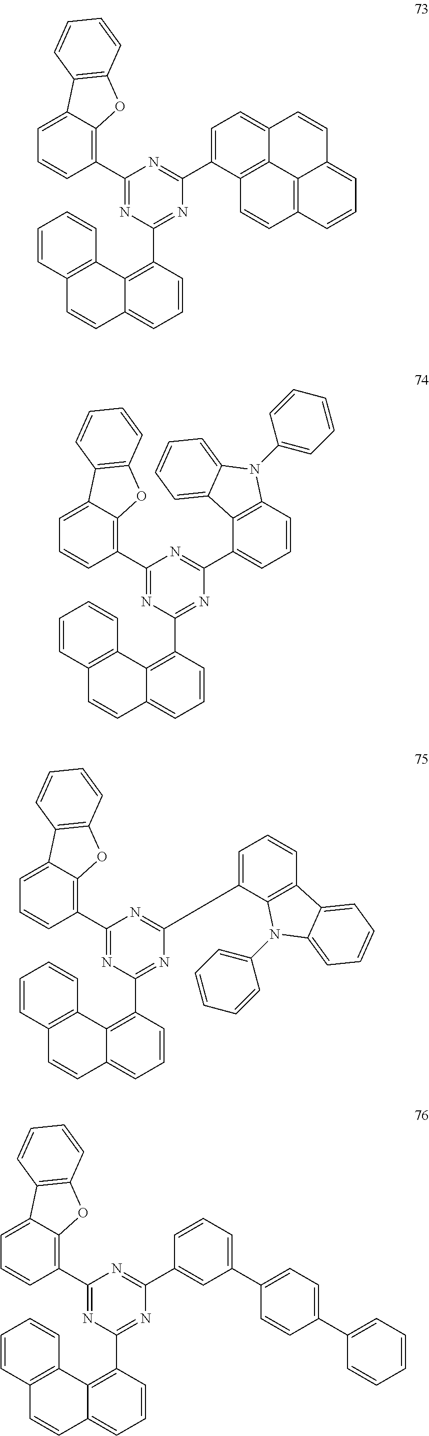 [SMILES: c1ccc2c(c1)ccc1cccc(-c3nc(-c4ccc5ccc6cccc7ccc4c5c67)nc(-c4cccc5c4oc4ccccc45)n3)c12 ; c1ccc(-n2c3ccccc3c3c(-c4nc(-c5cccc6c5oc5ccccc56)nc(-c5cccc6ccc7ccccc7c56)n4)cccc32)cc1 ; c1ccc(-n2c3ccccc3c3cccc(-c4nc(-c5cccc6c5oc5ccccc56)nc(-c5cccc6ccc7ccccc7c56)n4)c32)cc1 ; c1ccc(-c2ccc(-c3cccc(-c4nc(-c5cccc6c5oc5ccccc56)nc(-c5cccc6ccc7ccccc7c56)n4)c3)cc2)cc1]

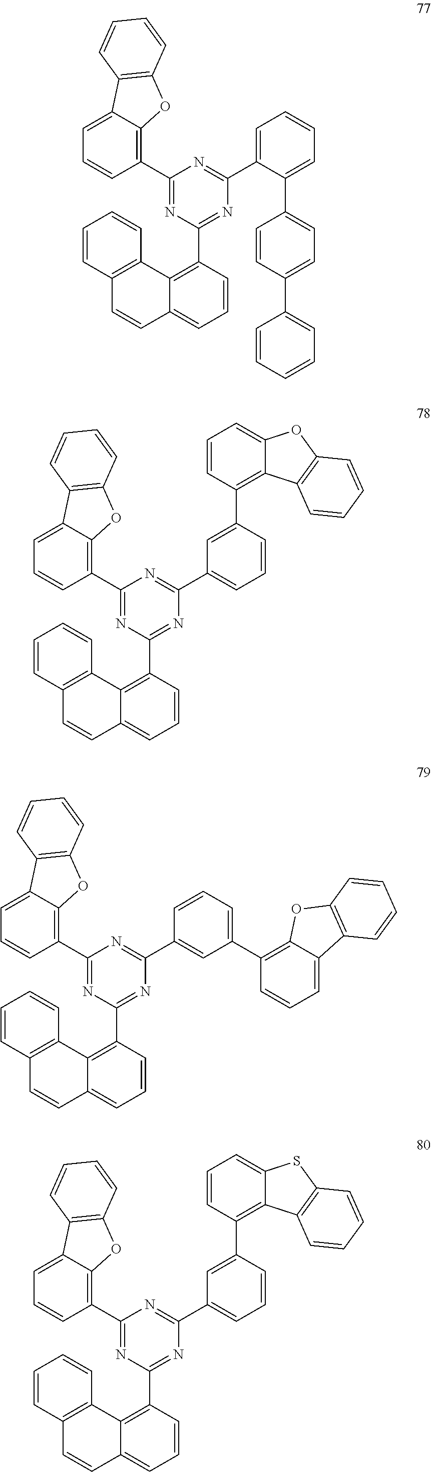 [SMILES: c1ccc(-c2ccc(-c3ccccc3-c3nc(-c4cccc5c4oc4ccccc45)nc(-c4cccc5ccc6ccccc6c45)n3)cc2)cc1 ; c1cc(-c2nc(-c3cccc4c3oc3ccccc34)nc(-c3cccc4ccc5ccccc5c34)n2)cc(-c2cccc3oc4ccccc4c23)c1 ; c1cc(-c2nc(-c3cccc4c3oc3ccccc34)nc(-c3cccc4ccc5ccccc5c34)n2)cc(-c2cccc3c2oc2ccccc23)c1 ; c1cc(-c2nc(-c3cccc4c3oc3ccccc34)nc(-c3cccc4ccc5ccccc5c34)n2)cc(-c2cccc3sc4ccccc4c23)c1]

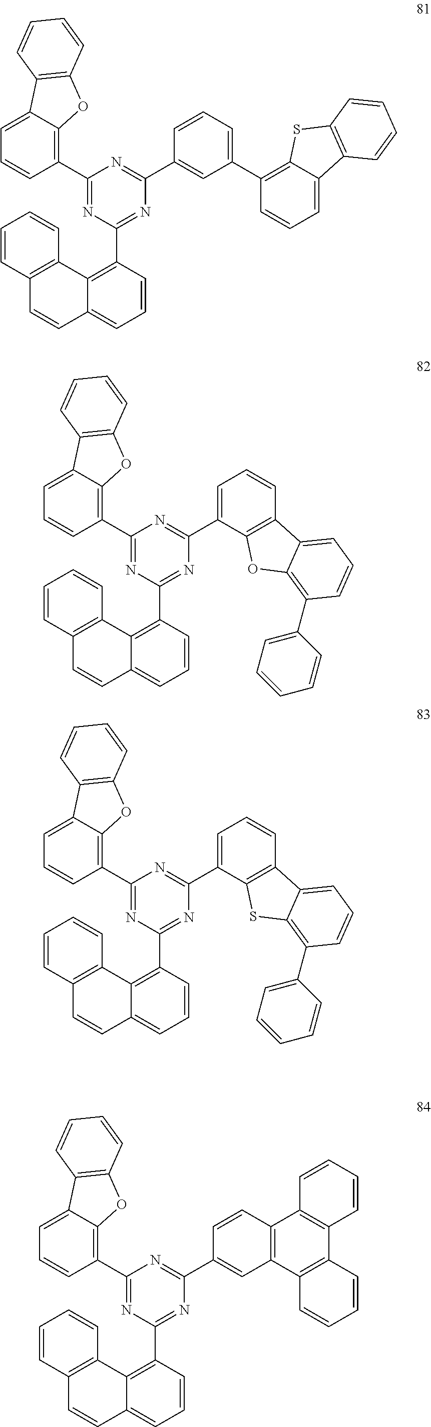

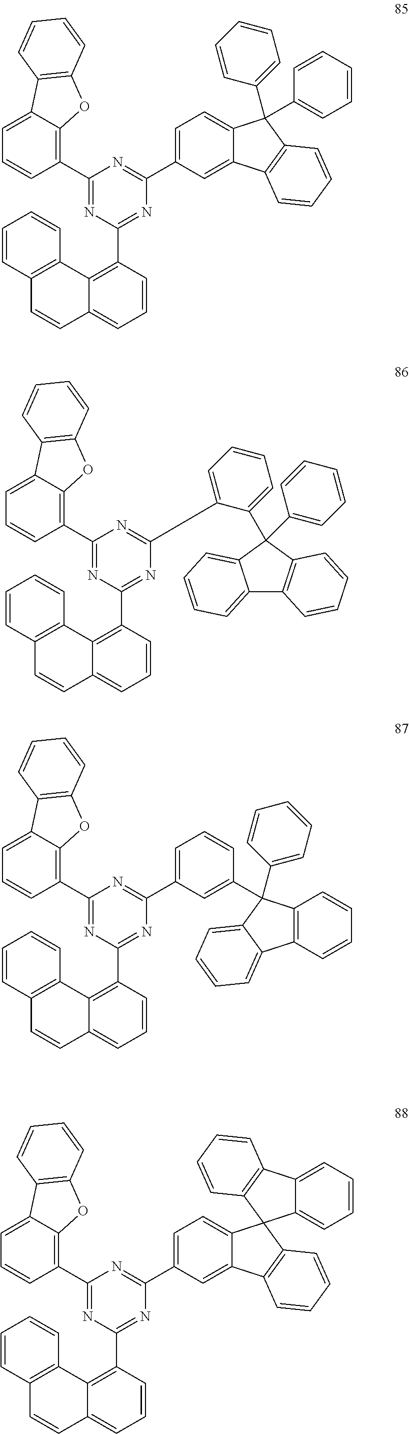

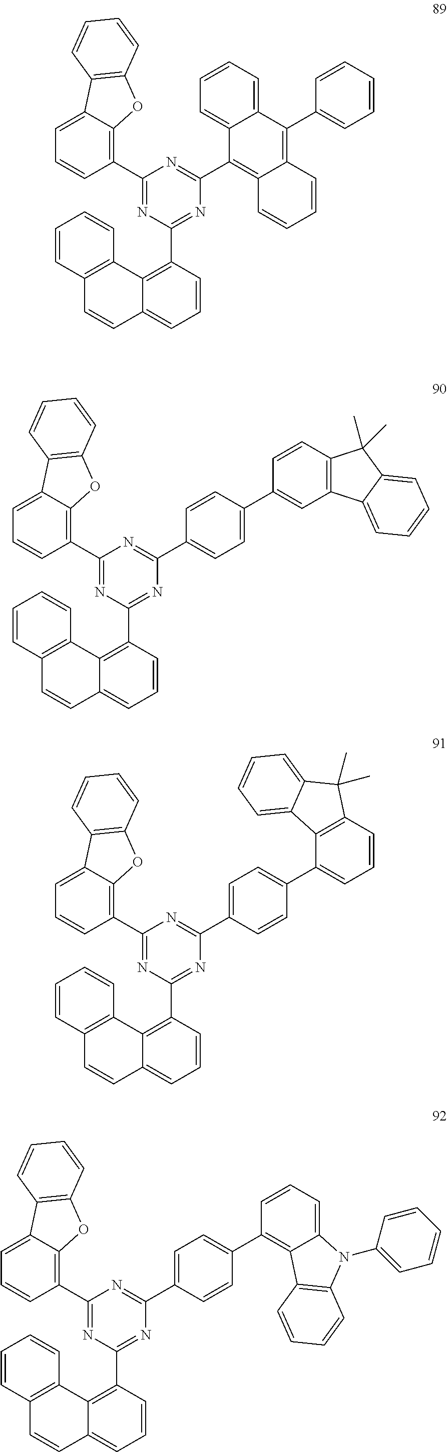 [SMILES: c1ccc(-c2c3ccccc3c(-c3nc(-c4cccc5c4oc4ccccc45)nc(-c4cccc5ccc6ccccc6c45)n3)c3ccccc23)cc1 ; CC1(C)c2ccccc2-c2cc(-c3ccc(-c4nc(-c5cccc6c5oc5ccccc56)nc(-c5cccc6ccc7ccccc7c56)n4)cc3)ccc21 ; CC1(C)c2ccccc2-c2c(-c3ccc(-c4nc(-c5cccc6c5oc5ccccc56)nc(-c5cccc6ccc7ccccc7c56)n4)cc3)cccc21 ; c1ccc(-n2c3ccccc3c3c(-c4ccc(-c5nc(-c6cccc7c6oc6ccccc67)nc(-c6cccc7ccc8ccccc8c67)n5)cc4)cccc32)cc1]

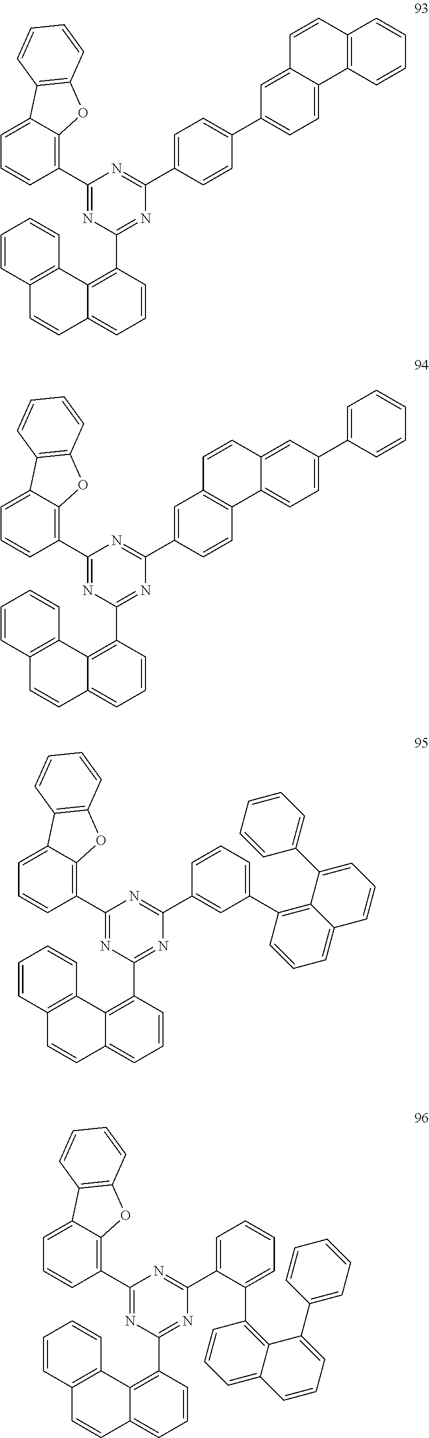 [SMILES: c1ccc2c(c1)ccc1cc(-c3ccc(-c4nc(-c5cccc6c5oc5ccccc56)nc(-c5cccc6ccc7ccccc7c56)n4)cc3)ccc12 ; c1ccc(-c2ccc3c(ccc4cc(-c5nc(-c6cccc7c6oc6ccccc67)nc(-c6cccc7ccc8ccccc8c67)n5)ccc43)c2)cc1 ; c1ccc(-c2cccc3cccc(-c4cccc(-c5nc(-c6cccc7c6oc6ccccc67)nc(-c6cccc7ccc8ccccc8c67)n5)c4)c23)cc1 ; c1ccc(-c2cccc3cccc(-c4ccccc4-c4nc(-c5cccc6c5oc5ccccc56)nc(-c5cccc6ccc7ccccc7c56)n4)c23)cc1]

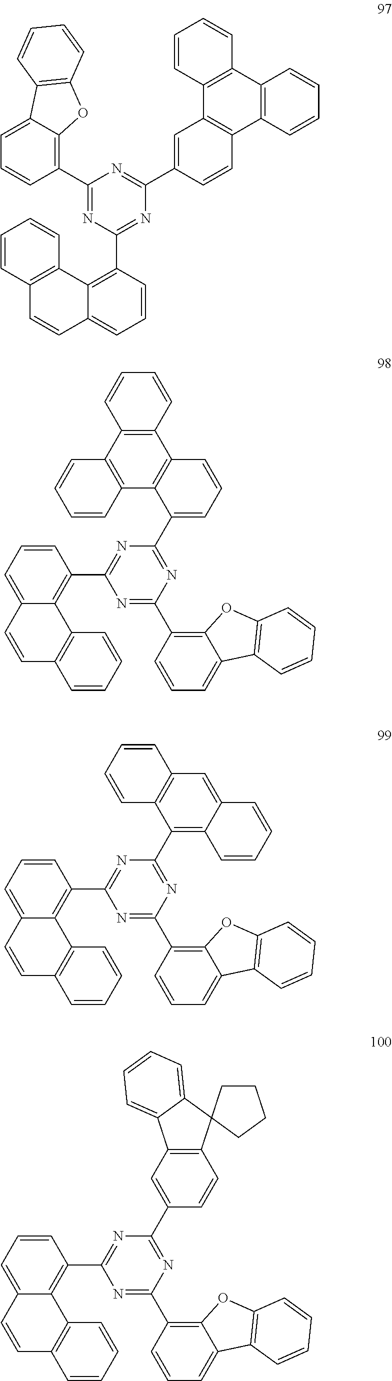

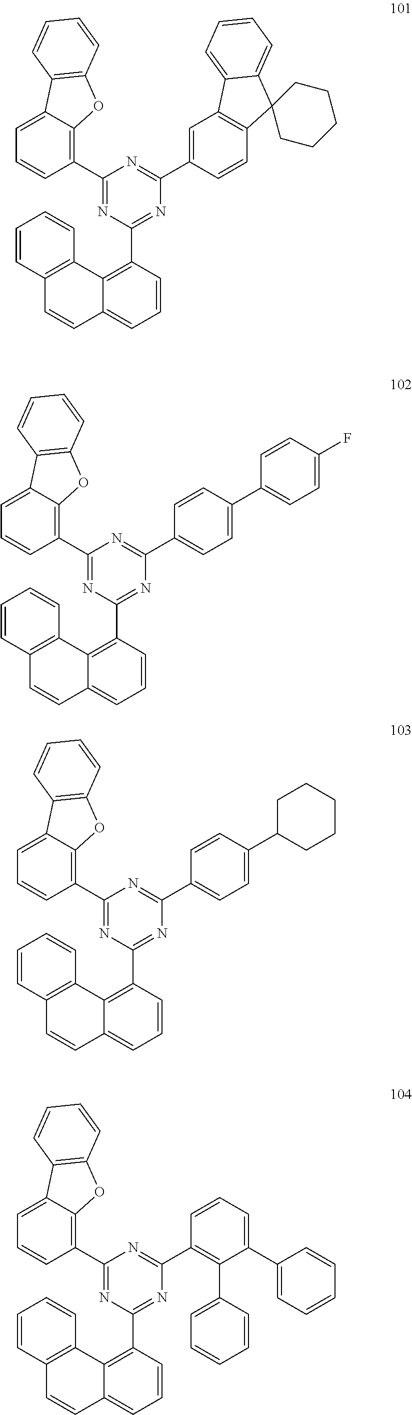

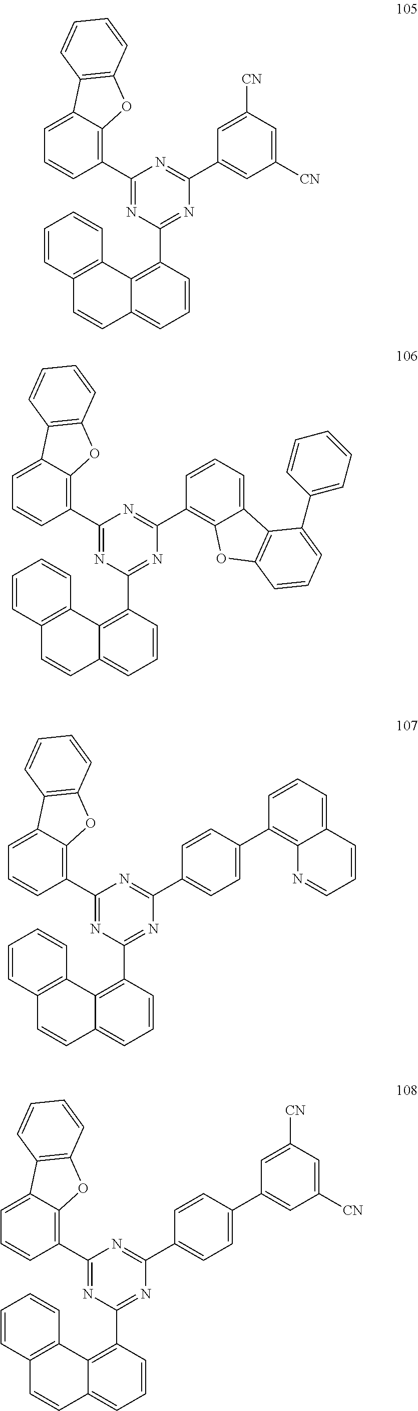

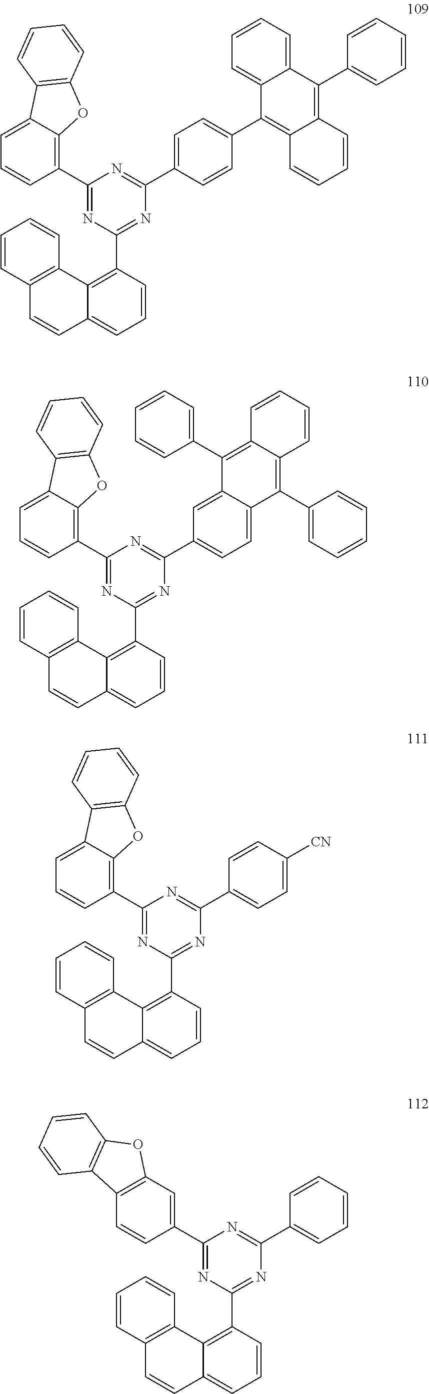

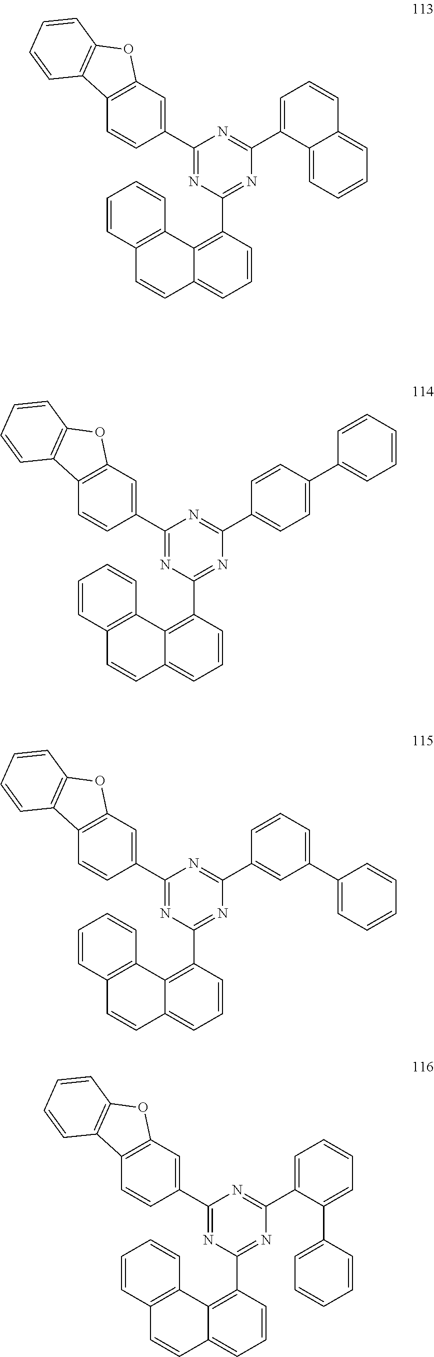 [SMILES: c1ccc2c(-c3nc(-c4ccc5c(c4)oc4ccccc45)nc(-c4cccc5ccc6ccccc6c45)n3)cccc2c1 ; c1ccc(-c2ccc(-c3nc(-c4ccc5c(c4)oc4ccccc45)nc(-c4cccc5ccc6ccccc6c45)n3)cc2)cc1 ; c1ccc(-c2cccc(-c3nc(-c4ccc5c(c4)oc4ccccc45)nc(-c4cccc5ccc6ccccc6c45)n3)c2)cc1 ; c1ccc(-c2ccccc2-c2nc(-c3ccc4c(c3)oc3ccccc34)nc(-c3cccc4ccc5ccccc5c34)n2)cc1]

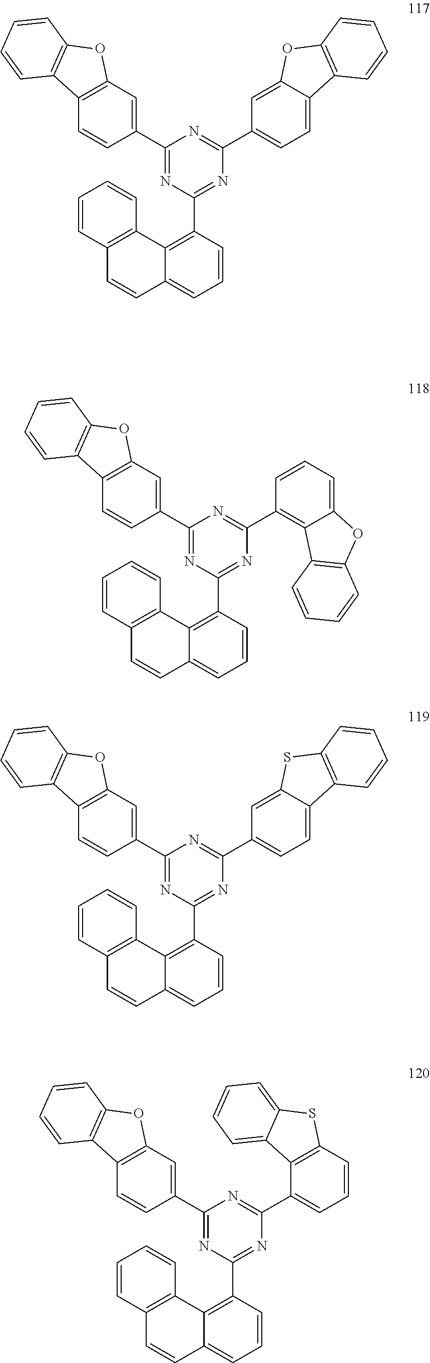 [SMILES: c1ccc2c(c1)ccc1cccc(-c3nc(-c4ccc5c(c4)oc4ccccc45)nc(-c4ccc5c(c4)oc4ccccc45)n3)c12 ; c1ccc2c(c1)ccc1cccc(-c3nc(-c4ccc5c(c4)oc4ccccc45)nc(-c4cccc5oc6ccccc6c45)n3)c12 ; c1ccc2c(c1)ccc1cccc(-c3nc(-c4ccc5c(c4)oc4ccccc45)nc(-c4ccc5c(c4)sc4ccccc45)n3)c12 ; c1ccc2c(c1)ccc1cccc(-c3nc(-c4ccc5c(c4)oc4ccccc45)nc(-c4cccc5sc6ccccc6c45)n3)c12]

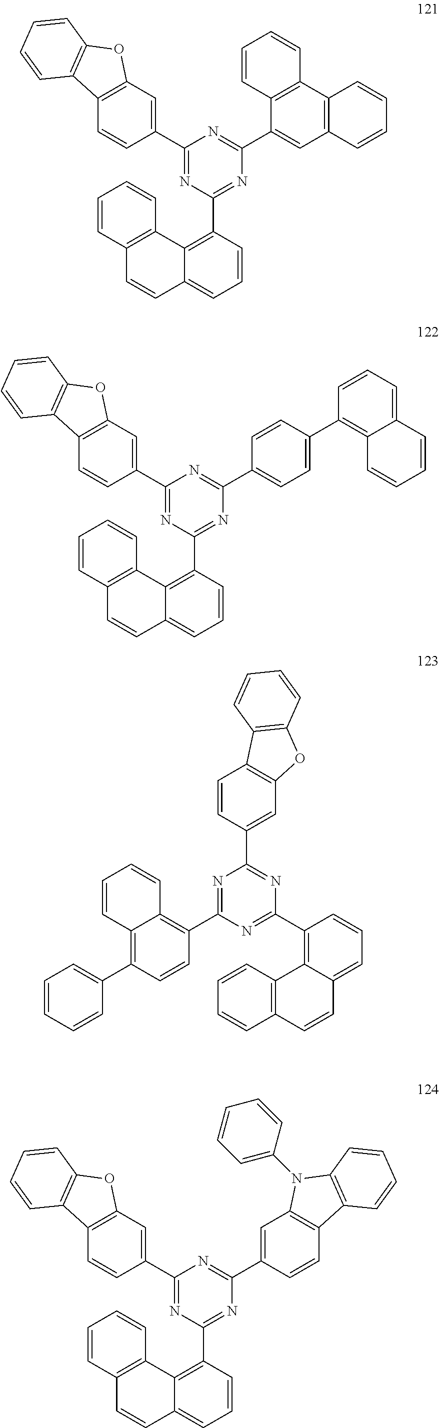 [SMILES: c1ccc2c(c1)cc(-c1nc(-c3ccc4c(c3)oc3ccccc34)nc(-c3cccc4ccc5ccccc5c34)n1)c1ccccc12 ; c1ccc2c(-c3ccc(-c4nc(-c5ccc6c(c5)oc5ccccc56)nc(-c5cccc6ccc7ccccc7c56)n4)cc3)cccc2c1 ; c1ccc(-c2ccc(-c3nc(-c4ccc5c(c4)oc4ccccc45)nc(-c4cccc5ccc6ccccc6c45)n3)c3ccccc23)cc1 ; c1ccc(-n2c3ccccc3c3ccc(-c4nc(-c5ccc6c(c5)oc5ccccc56)nc(-c5cccc6ccc7ccccc7c56)n4)cc32)cc1]

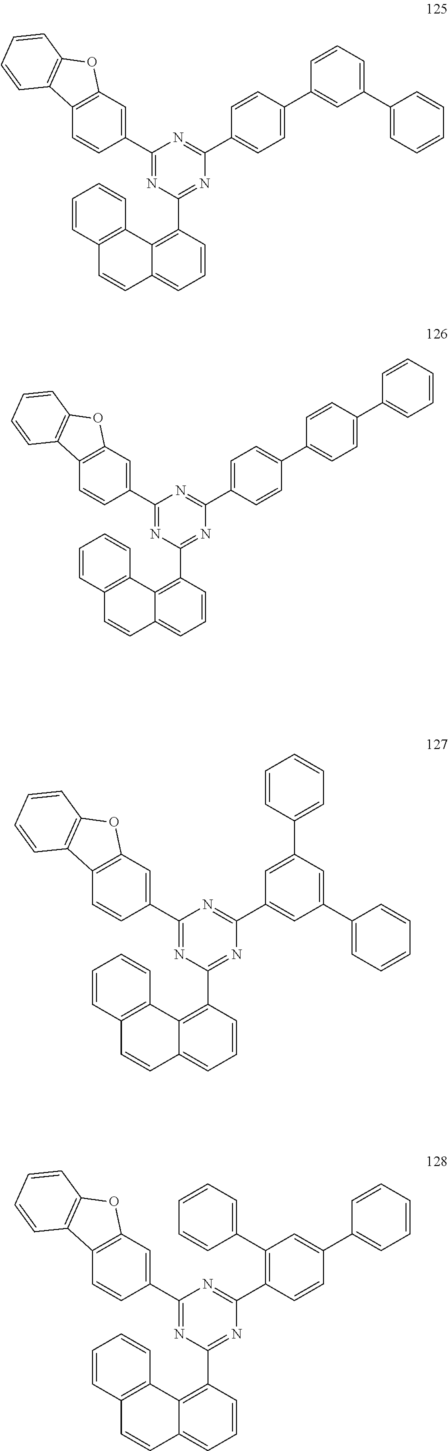 [SMILES: c1ccc(-c2cccc(-c3ccc(-c4nc(-c5ccc6c(c5)oc5ccccc56)nc(-c5cccc6ccc7ccccc7c56)n4)cc3)c2)cc1 ; c1ccc(-c2ccc(-c3ccc(-c4nc(-c5ccc6c(c5)oc5ccccc56)nc(-c5cccc6ccc7ccccc7c56)n4)cc3)cc2)cc1 ; c1ccc(-c2cc(-c3ccccc3)cc(-c3nc(-c4ccc5c(c4)oc4ccccc45)nc(-c4cccc5ccc6ccccc6c45)n3)c2)cc1 ; c1ccc(-c2ccc(-c3nc(-c4ccc5c(c4)oc4ccccc45)nc(-c4cccc5ccc6ccccc6c45)n3)c(-c3ccccc3)c2)cc1]

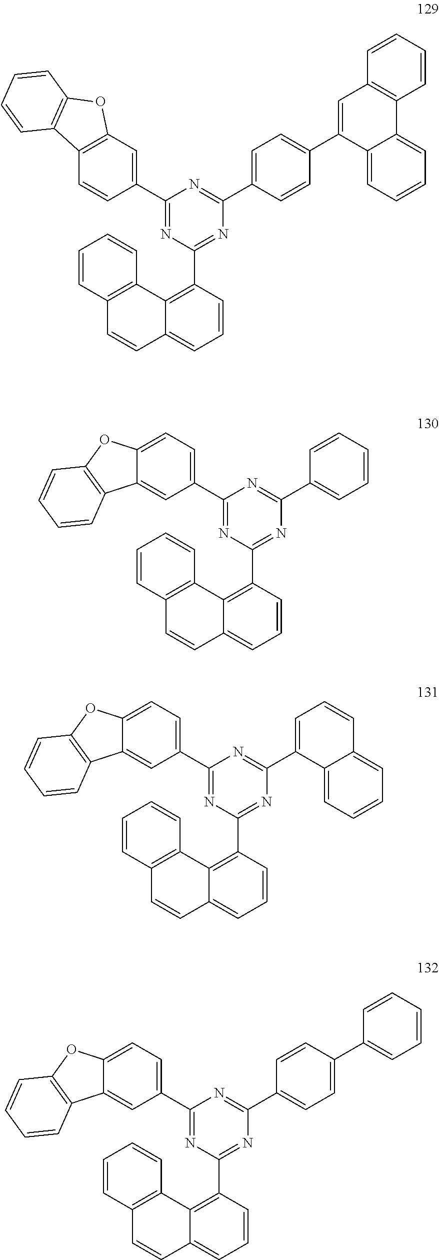 [SMILES: c1ccc2c(c1)cc(-c1ccc(-c3nc(-c4ccc5c(c4)oc4ccccc45)nc(-c4cccc5ccc6ccccc6c45)n3)cc1)c1ccccc12 ; c1ccc(-c2nc(-c3ccc4oc5ccccc5c4c3)nc(-c3cccc4ccc5ccccc5c34)n2)cc1 ; c1ccc2c(-c3nc(-c4ccc5oc6ccccc6c5c4)nc(-c4cccc5ccc6ccccc6c45)n3)cccc2c1 ; c1ccc(-c2ccc(-c3nc(-c4ccc5oc6ccccc6c5c4)nc(-c4cccc5ccc6ccccc6c45)n3)cc2)cc1]

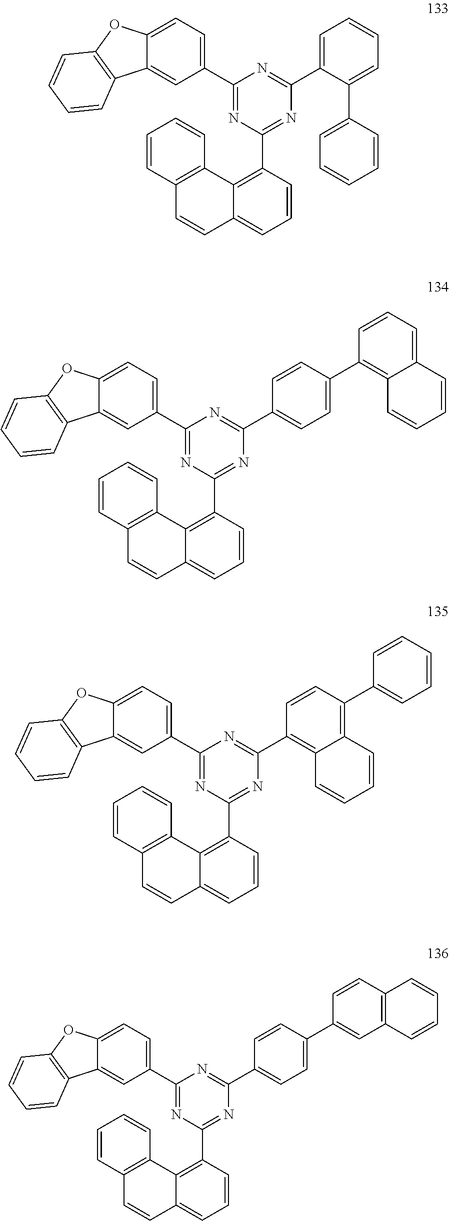 [SMILES: c1ccc(-c2ccccc2-c2nc(-c3ccc4oc5ccccc5c4c3)nc(-c3cccc4ccc5ccccc5c34)n2)cc1 ; c1ccc2c(-c3ccc(-c4nc(-c5ccc6oc7ccccc7c6c5)nc(-c5cccc6ccc7ccccc7c56)n4)cc3)cccc2c1 ; c1ccc(-c2ccc(-c3nc(-c4ccc5oc6ccccc6c5c4)nc(-c4cccc5ccc6ccccc6c45)n3)c3ccccc23)cc1 ; c1ccc2cc(-c3ccc(-c4nc(-c5ccc6oc7ccccc7c6c5)nc(-c5cccc6ccc7ccccc7c56)n4)cc3)ccc2c1]

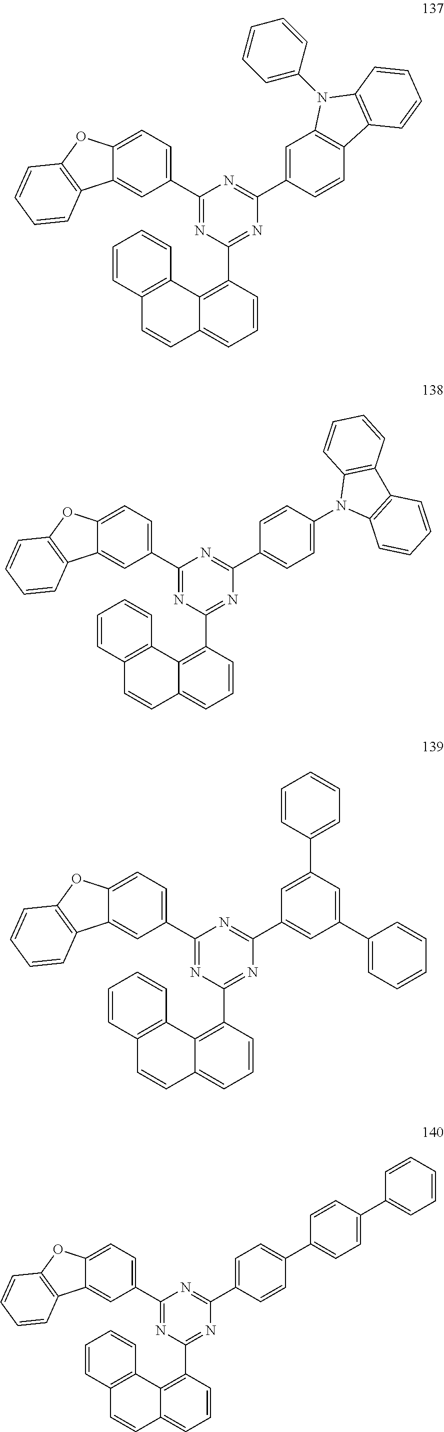 [SMILES: c1ccc(-n2c3ccccc3c3ccc(-c4nc(-c5ccc6oc7ccccc7c6c5)nc(-c5cccc6ccc7ccccc7c56)n4)cc32)cc1 ; c1ccc2c(c1)ccc1cccc(-c3nc(-c4ccc(-n5c6ccccc6c6ccccc65)cc4)nc(-c4ccc5oc6ccccc6c5c4)n3)c12 ; c1ccc(-c2cc(-c3ccccc3)cc(-c3nc(-c4ccc5oc6ccccc6c5c4)nc(-c4cccc5ccc6ccccc6c45)n3)c2)cc1 ; c1ccc(-c2ccc(-c3ccc(-c4nc(-c5ccc6oc7ccccc7c6c5)nc(-c5cccc6ccc7ccccc7c56)n4)cc3)cc2)cc1]

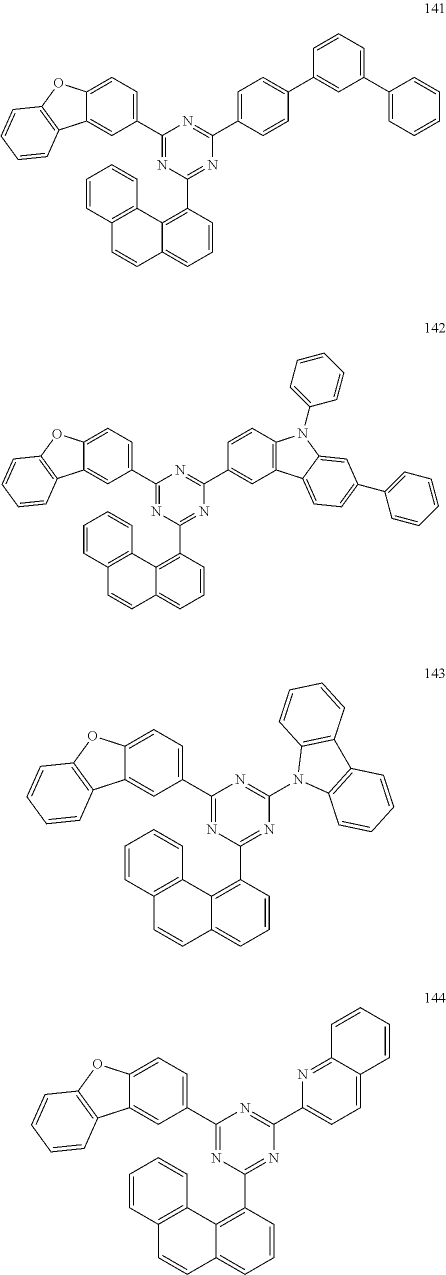 [SMILES: c1ccc(-c2cccc(-c3ccc(-c4nc(-c5ccc6oc7ccccc7c6c5)nc(-c5cccc6ccc7ccccc7c56)n4)cc3)c2)cc1 ; c1ccc(-c2ccc3c4cc(-c5nc(-c6ccc7oc8ccccc8c7c6)nc(-c6cccc7ccc8ccccc8c67)n5)ccc4n(-c4ccccc4)c3c2)cc1 ; c1ccc2c(c1)ccc1cccc(-c3nc(-c4ccc5oc6ccccc6c5c4)nc(-n4c5ccccc5c5ccccc54)n3)c12 ; c1ccc2nc(-c3nc(-c4ccc5oc6ccccc6c5c4)nc(-c4cccc5ccc6ccccc6c45)n3)ccc2c1]

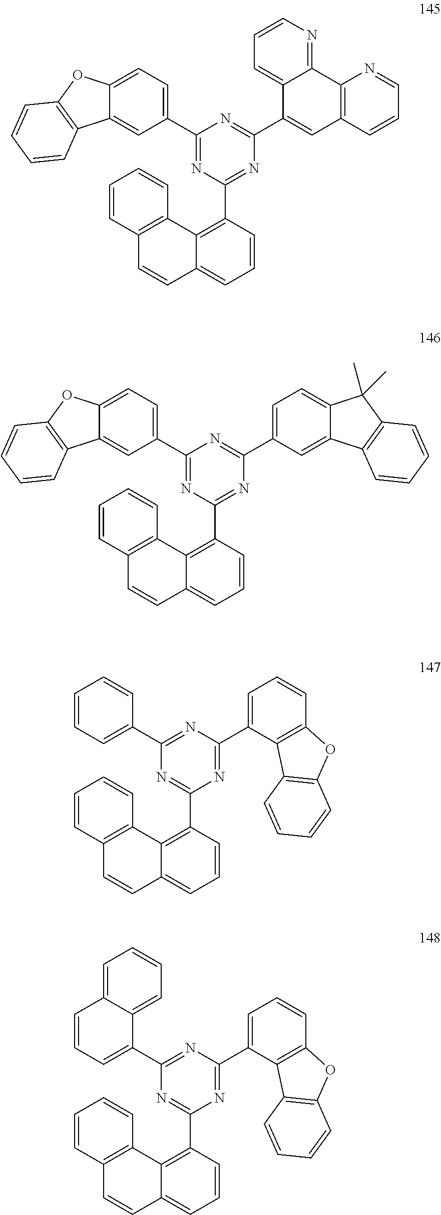 [SMILES: c1ccc2c(c1)ccc1cccc(-c3nc(-c4ccc5oc6ccccc6c5c4)nc(-c4cc5cccnc5c5ncccc45)n3)c12 ; CC1(C)c2ccccc2-c2cc(-c3nc(-c4ccc5oc6ccccc6c5c4)nc(-c4cccc5ccc6ccccc6c45)n3)ccc21 ; c1ccc(-c2nc(-c3cccc4ccc5ccccc5c34)nc(-c3cccc4oc5ccccc5c34)n2)cc1 ; c1ccc2c(-c3nc(-c4cccc5ccc6ccccc6c45)nc(-c4cccc5oc6ccccc6c45)n3)cccc2c1]

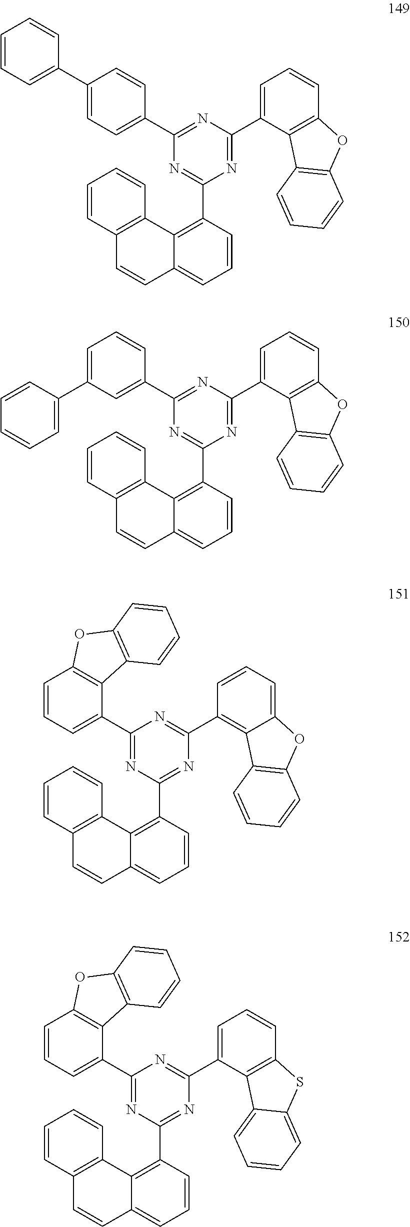 [SMILES: c1ccc(-c2ccc(-c3nc(-c4cccc5ccc6ccccc6c45)nc(-c4cccc5oc6ccccc6c45)n3)cc2)cc1 ; c1ccc(-c2cccc(-c3nc(-c4cccc5ccc6ccccc6c45)nc(-c4cccc5oc6ccccc6c45)n3)c2)cc1 ; c1ccc2c(c1)ccc1cccc(-c3nc(-c4cccc5oc6ccccc6c45)nc(-c4cccc5oc6ccccc6c45)n3)c12 ; c1ccc2c(c1)ccc1cccc(-c3nc(-c4cccc5oc6ccccc6c45)nc(-c4cccc5sc6ccccc6c45)n3)c12]

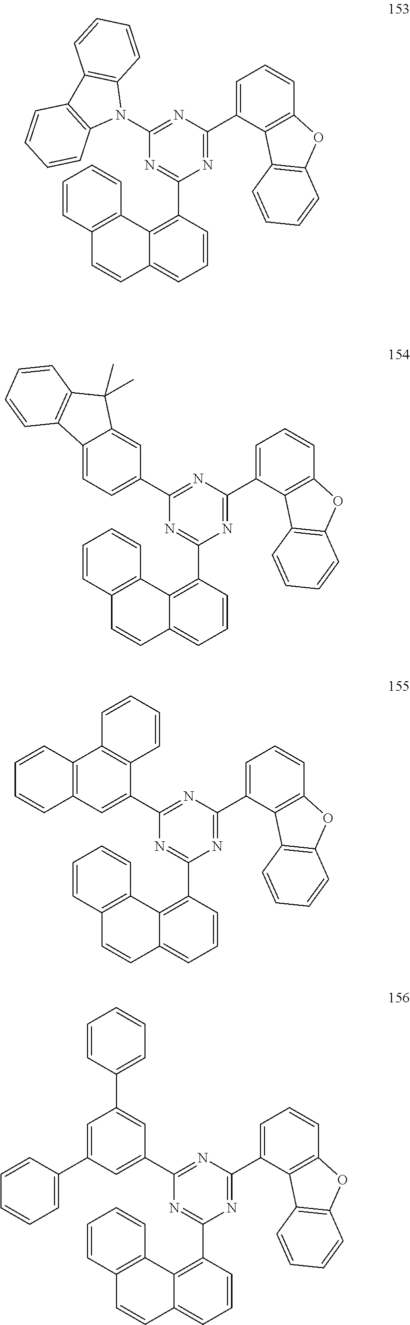 [SMILES: c1ccc2c(c1)ccc1cccc(-c3nc(-c4cccc5oc6ccccc6c45)nc(-n4c5ccccc5c5ccccc54)n3)c12 ; CC1(C)c2ccccc2-c2ccc(-c3nc(-c4cccc5ccc6ccccc6c45)nc(-c4cccc5oc6ccccc6c45)n3)cc21 ; c1ccc2c(c1)cc(-c1nc(-c3cccc4ccc5ccccc5c34)nc(-c3cccc4oc5ccccc5c34)n1)c1ccccc12 ; c1ccc(-c2cc(-c3ccccc3)cc(-c3nc(-c4cccc5ccc6ccccc6c45)nc(-c4cccc5oc6ccccc6c45)n3)c2)cc1]

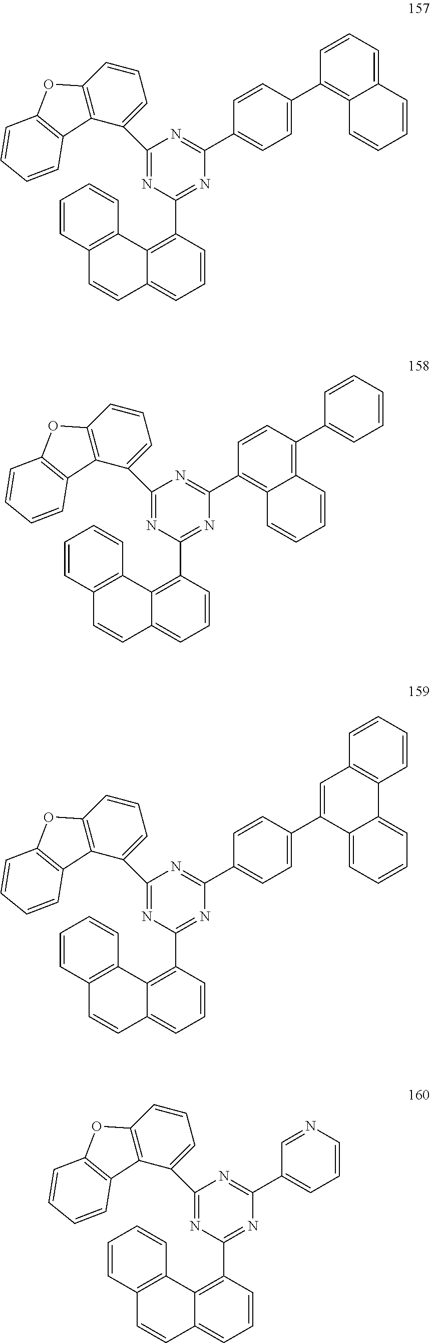 [SMILES: c1ccc2c(-c3ccc(-c4nc(-c5cccc6ccc7ccccc7c56)nc(-c5cccc6oc7ccccc7c56)n4)cc3)cccc2c1 ; c1ccc(-c2ccc(-c3nc(-c4cccc5ccc6ccccc6c45)nc(-c4cccc5oc6ccccc6c45)n3)c3ccccc23)cc1 ; c1ccc2c(c1)cc(-c1ccc(-c3nc(-c4cccc5ccc6ccccc6c45)nc(-c4cccc5oc6ccccc6c45)n3)cc1)c1ccccc12 ; c1cncc(-c2nc(-c3cccc4ccc5ccccc5c34)nc(-c3cccc4oc5ccccc5c34)n2)c1]

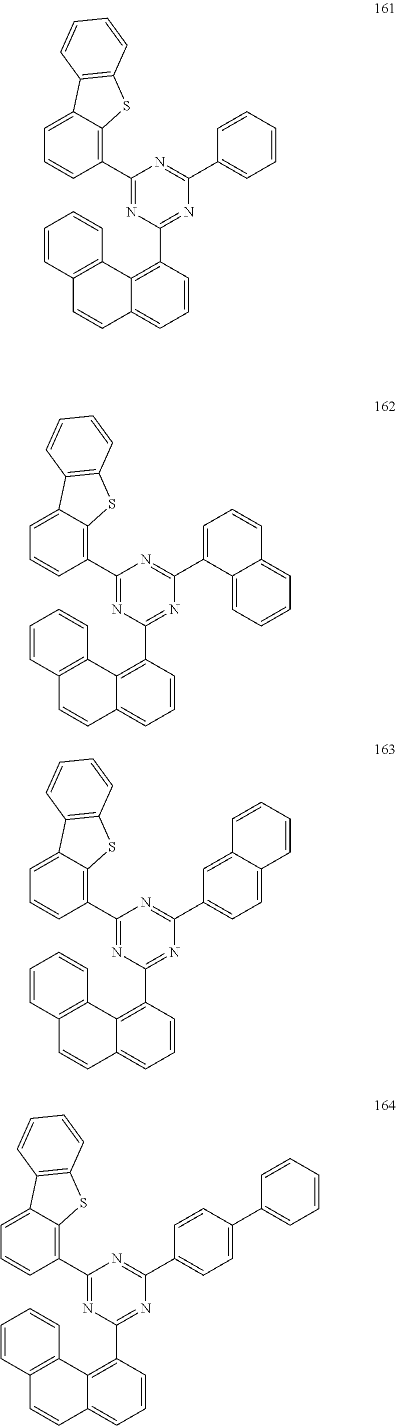

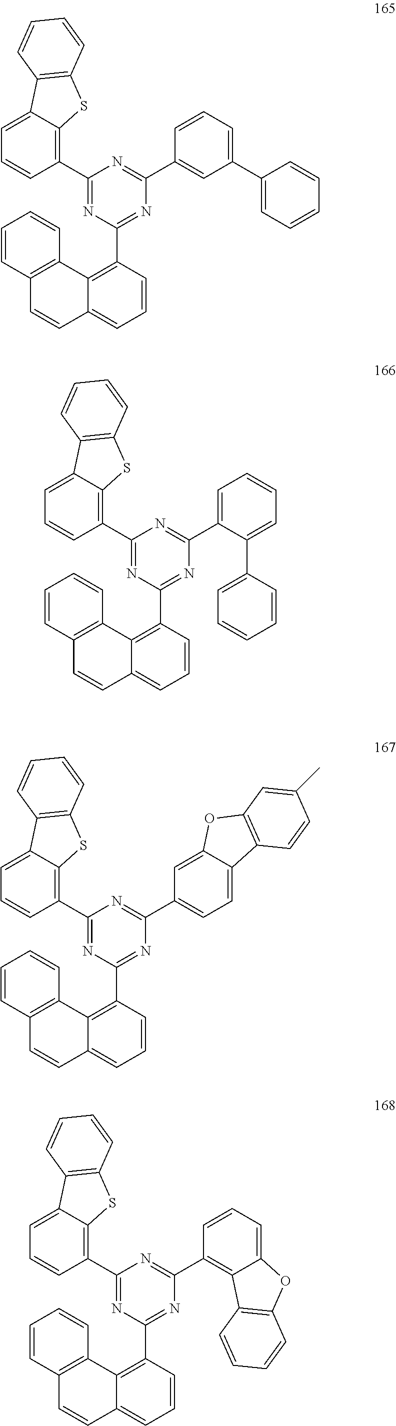 [SMILES: c1ccc(-c2cccc(-c3nc(-c4cccc5c4sc4ccccc45)nc(-c4cccc5ccc6ccccc6c45)n3)c2)cc1 ; c1ccc(-c2ccccc2-c2nc(-c3cccc4c3sc3ccccc34)nc(-c3cccc4ccc5ccccc5c34)n2)cc1 ; Cc1ccc2c(c1)oc1cc(-c3nc(-c4cccc5c4sc4ccccc45)nc(-c4cccc5ccc6ccccc6c45)n3)ccc12 ; c1ccc2c(c1)ccc1cccc(-c3nc(-c4cccc5c4sc4ccccc45)nc(-c4cccc5oc6ccccc6c45)n3)c12]

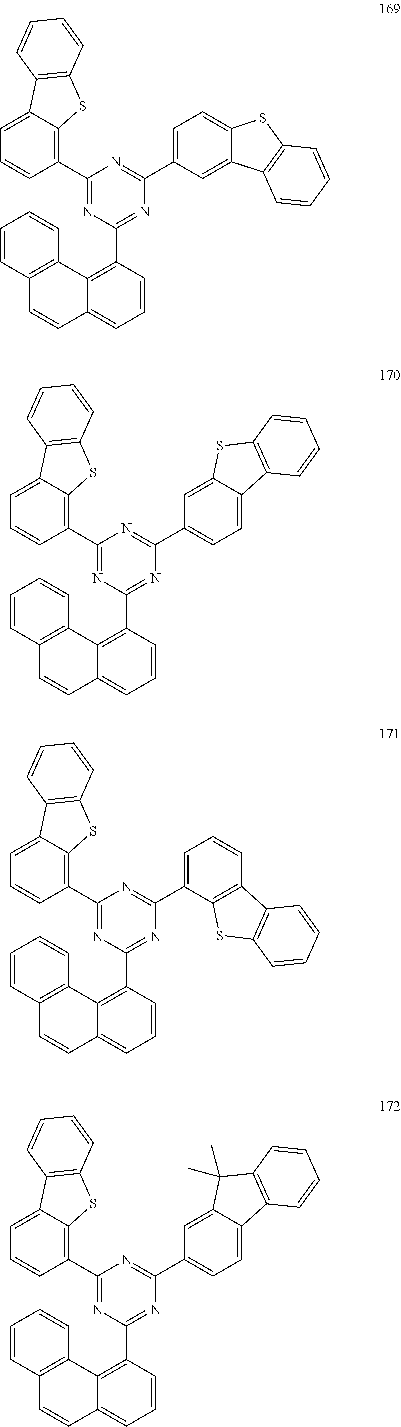 [SMILES: c1ccc2c(c1)ccc1cccc(-c3nc(-c4ccc5sc6ccccc6c5c4)nc(-c4cccc5c4sc4ccccc45)n3)c12 ; c1ccc2c(c1)ccc1cccc(-c3nc(-c4ccc5c(c4)sc4ccccc45)nc(-c4cccc5c4sc4ccccc45)n3)c12 ; c1ccc2c(c1)ccc1cccc(-c3nc(-c4cccc5c4sc4ccccc45)nc(-c4cccc5c4sc4ccccc45)n3)c12 ; CC1(C)c2ccccc2-c2ccc(-c3nc(-c4cccc5c4sc4ccccc45)nc(-c4cccc5ccc6ccccc6c45)n3)cc21]

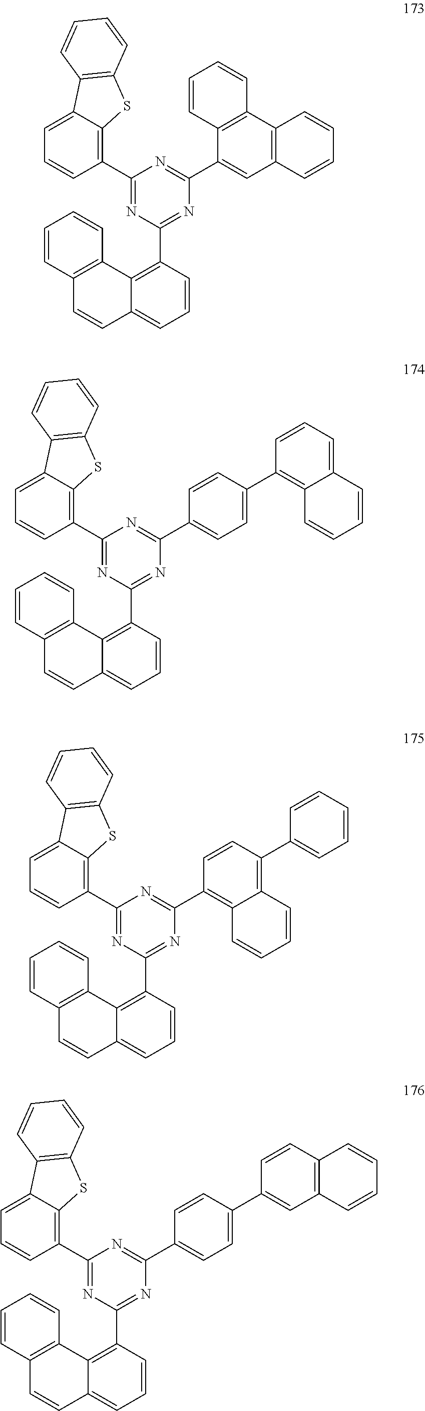 [SMILES: c1ccc2c(c1)cc(-c1nc(-c3cccc4c3sc3ccccc34)nc(-c3cccc4ccc5ccccc5c34)n1)c1ccccc12 ; c1ccc2c(-c3ccc(-c4nc(-c5cccc6c5sc5ccccc56)nc(-c5cccc6ccc7ccccc7c56)n4)cc3)cccc2c1 ; c1ccc(-c2ccc(-c3nc(-c4cccc5c4sc4ccccc45)nc(-c4cccc5ccc6ccccc6c45)n3)c3ccccc23)cc1 ; c1ccc2cc(-c3ccc(-c4nc(-c5cccc6c5sc5ccccc56)nc(-c5cccc6ccc7ccccc7c56)n4)cc3)ccc2c1]

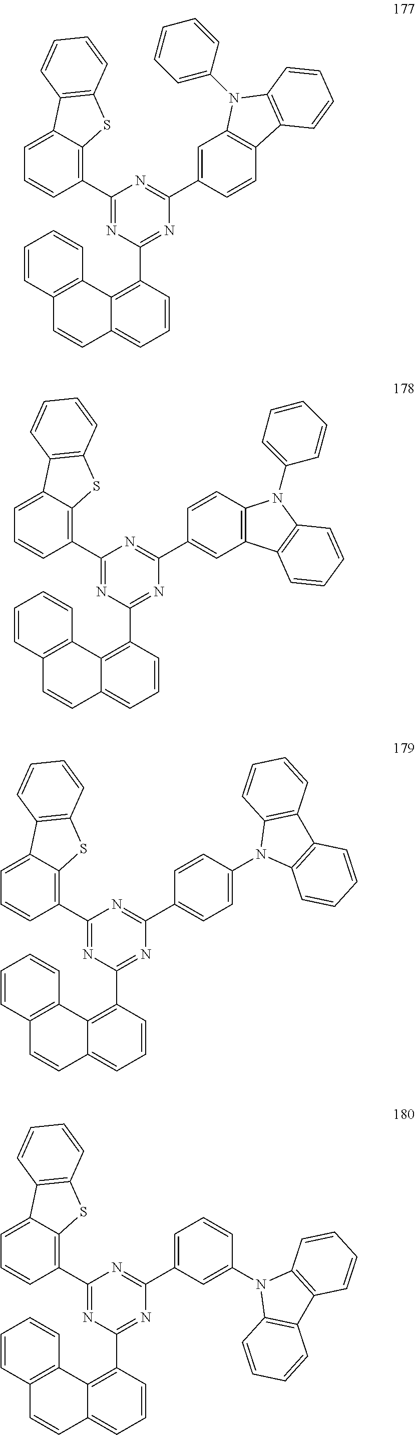 [SMILES: c1ccc(-n2c3ccccc3c3ccc(-c4nc(-c5cccc6c5sc5ccccc56)nc(-c5cccc6ccc7ccccc7c56)n4)cc32)cc1 ; c1ccc(-n2c3ccccc3c3cc(-c4nc(-c5cccc6c5sc5ccccc56)nc(-c5cccc6ccc7ccccc7c56)n4)ccc32)cc1 ; c1ccc2c(c1)ccc1cccc(-c3nc(-c4ccc(-n5c6ccccc6c6ccccc65)cc4)nc(-c4cccc5c4sc4ccccc45)n3)c12 ; c1cc(-c2nc(-c3cccc4c3sc3ccccc34)nc(-c3cccc4ccc5ccccc5c34)n2)cc(-n2c3ccccc3c3ccccc32)c1]

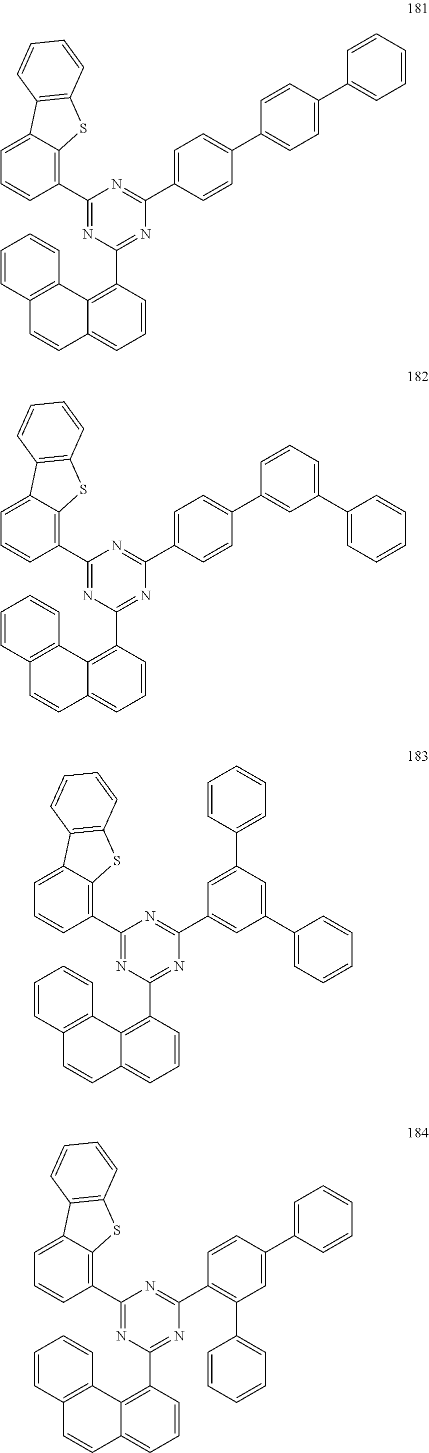 [SMILES: c1ccc(-c2ccc(-c3ccc(-c4nc(-c5cccc6c5sc5ccccc56)nc(-c5cccc6ccc7ccccc7c56)n4)cc3)cc2)cc1 ; c1ccc(-c2cccc(-c3ccc(-c4nc(-c5cccc6c5sc5ccccc56)nc(-c5cccc6ccc7ccccc7c56)n4)cc3)c2)cc1 ; c1ccc(-c2cc(-c3ccccc3)cc(-c3nc(-c4cccc5c4sc4ccccc45)nc(-c4cccc5ccc6ccccc6c45)n3)c2)cc1 ; c1ccc(-c2ccc(-c3nc(-c4cccc5c4sc4ccccc45)nc(-c4cccc5ccc6ccccc6c45)n3)c(-c3ccccc3)c2)cc1]

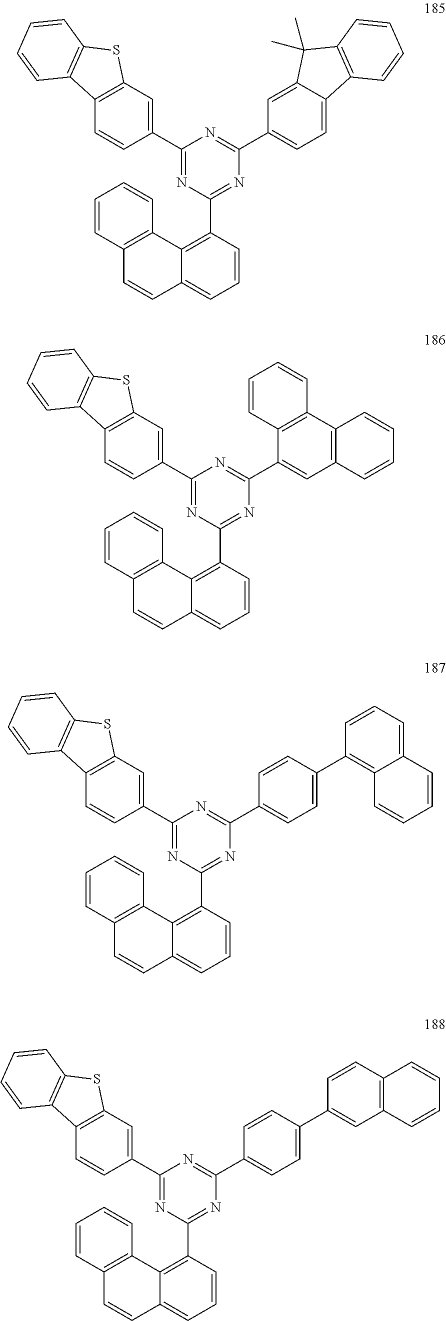 [SMILES: CC1(C)c2ccccc2-c2ccc(-c3nc(-c4ccc5c(c4)sc4ccccc45)nc(-c4cccc5ccc6ccccc6c45)n3)cc21 ; c1ccc2c(c1)cc(-c1nc(-c3ccc4c(c3)sc3ccccc34)nc(-c3cccc4ccc5ccccc5c34)n1)c1ccccc12 ; c1ccc2c(-c3ccc(-c4nc(-c5ccc6c(c5)sc5ccccc56)nc(-c5cccc6ccc7ccccc7c56)n4)cc3)cccc2c1 ; c1ccc2cc(-c3ccc(-c4nc(-c5ccc6c(c5)sc5ccccc56)nc(-c5cccc6ccc7ccccc7c56)n4)cc3)ccc2c1]

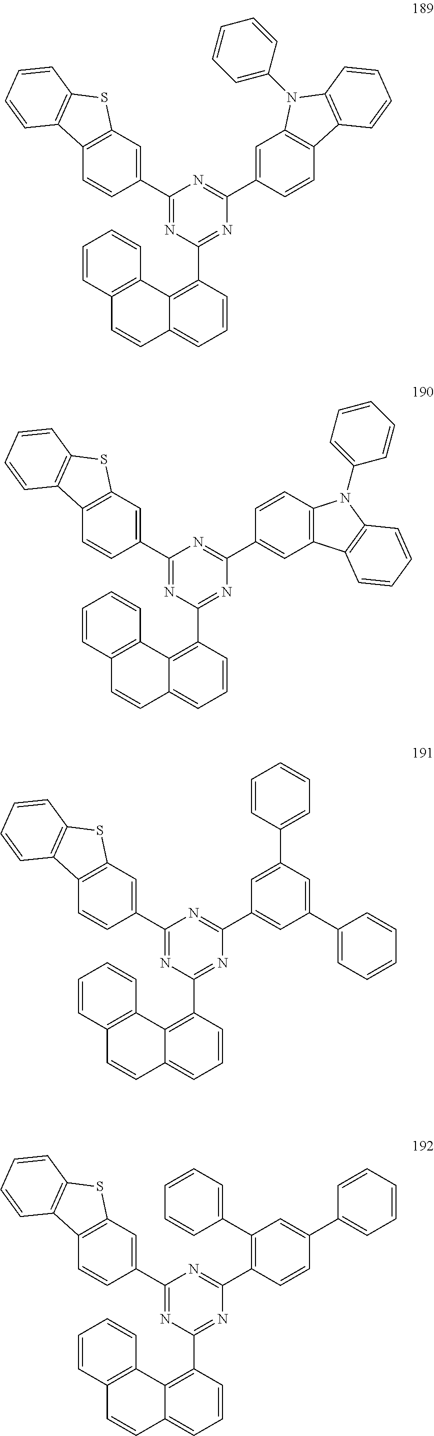 [SMILES: c1ccc(-n2c3ccccc3c3ccc(-c4nc(-c5ccc6c(c5)sc5ccccc56)nc(-c5cccc6ccc7ccccc7c56)n4)cc32)cc1 ; c1ccc(-n2c3ccccc3c3cc(-c4nc(-c5ccc6c(c5)sc5ccccc56)nc(-c5cccc6ccc7ccccc7c56)n4)ccc32)cc1 ; c1ccc(-c2cc(-c3ccccc3)cc(-c3nc(-c4ccc5c(c4)sc4ccccc45)nc(-c4cccc5ccc6ccccc6c45)n3)c2)cc1 ; c1ccc(-c2ccc(-c3nc(-c4ccc5c(c4)sc4ccccc45)nc(-c4cccc5ccc6ccccc6c45)n3)c(-c3ccccc3)c2)cc1]

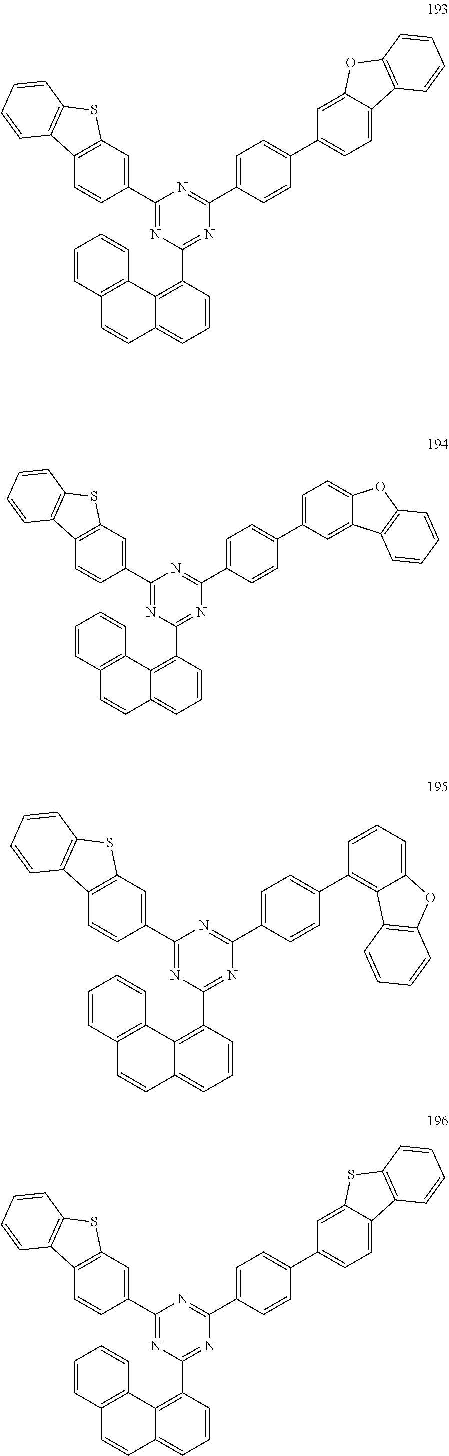 [SMILES: c1ccc2c(c1)ccc1cccc(-c3nc(-c4ccc(-c5ccc6c(c5)oc5ccccc56)cc4)nc(-c4ccc5c(c4)sc4ccccc45)n3)c12 ; c1ccc2c(c1)ccc1cccc(-c3nc(-c4ccc(-c5ccc6oc7ccccc7c6c5)cc4)nc(-c4ccc5c(c4)sc4ccccc45)n3)c12 ; c1ccc2c(c1)ccc1cccc(-c3nc(-c4ccc(-c5cccc6oc7ccccc7c56)cc4)nc(-c4ccc5c(c4)sc4ccccc45)n3)c12 ; c1ccc2c(c1)ccc1cccc(-c3nc(-c4ccc(-c5ccc6c(c5)sc5ccccc56)cc4)nc(-c4ccc5c(c4)sc4ccccc45)n3)c12]

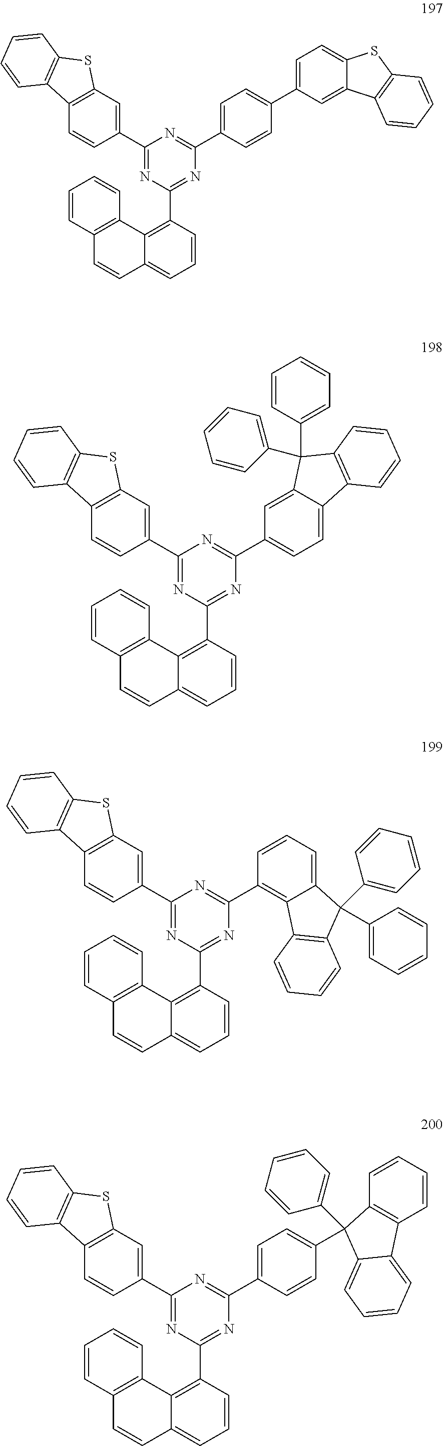 [SMILES: c1ccc2c(c1)ccc1cccc(-c3nc(-c4ccc(-c5ccc6sc7ccccc7c6c5)cc4)nc(-c4ccc5c(c4)sc4ccccc45)n3)c12 ; c1ccc(C2(c3ccccc3)c3ccccc3-c3ccc(-c4nc(-c5ccc6c(c5)sc5ccccc56)nc(-c5cccc6ccc7ccccc7c56)n4)cc32)cc1 ; c1ccc(C2(c3ccccc3)c3ccccc3-c3c(-c4nc(-c5ccc6c(c5)sc5ccccc56)nc(-c5cccc6ccc7ccccc7c56)n4)cccc32)cc1 ; c1ccc(C2(c3ccc(-c4nc(-c5ccc6c(c5)sc5ccccc56)nc(-c5cccc6ccc7ccccc7c56)n4)cc3)c3ccccc3-c3ccccc32)cc1]

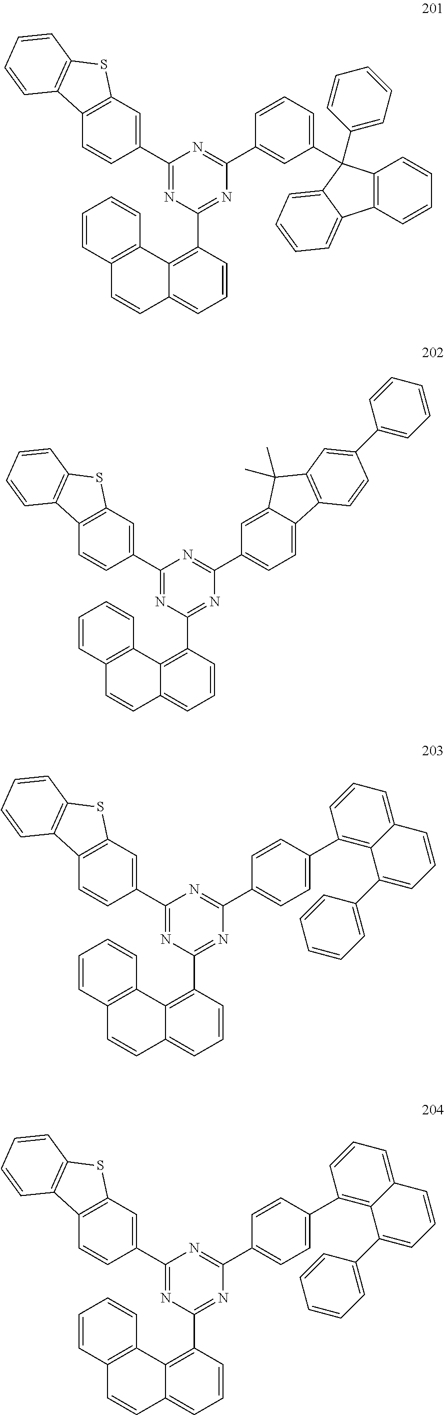 [SMILES: c1ccc(C2(c3cccc(-c4nc(-c5ccc6c(c5)sc5ccccc56)nc(-c5cccc6ccc7ccccc7c56)n4)c3)c3ccccc3-c3ccccc32)cc1 ; CC1(C)c2cc(-c3ccccc3)ccc2-c2ccc(-c3nc(-c4ccc5c(c4)sc4ccccc45)nc(-c4cccc5ccc6ccccc6c45)n3)cc21 ; c1ccc(-c2cccc3cccc(-c4ccc(-c5nc(-c6ccc7c(c6)sc6ccccc67)nc(-c6cccc7ccc8ccccc8c67)n5)cc4)c23)cc1 ; c1ccc(-c2cccc3cccc(-c4ccc(-c5nc(-c6ccc7c(c6)sc6ccccc67)nc(-c6cccc7ccc8ccccc8c67)n5)cc4)c23)cc1]

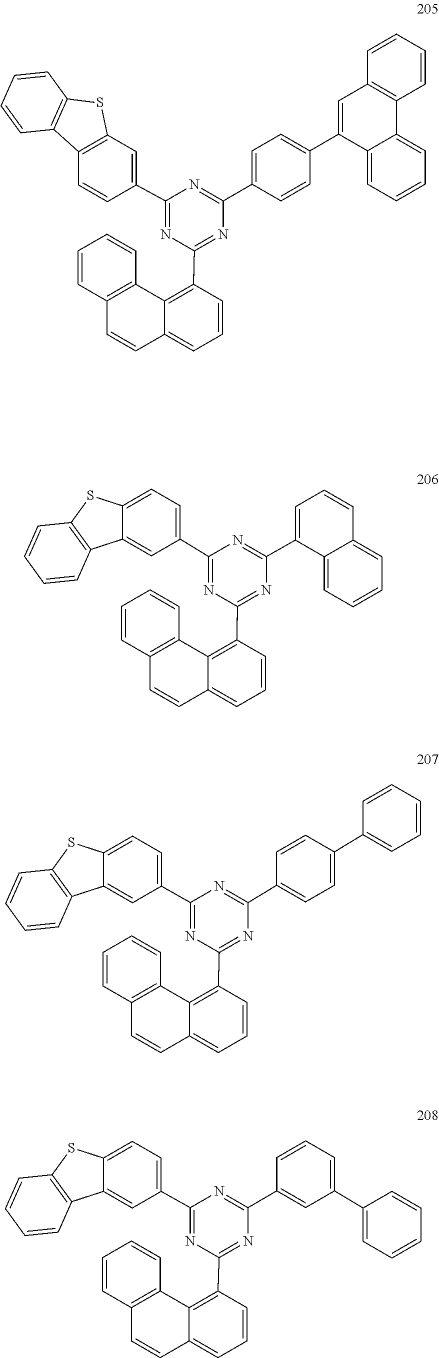 [SMILES: c1ccc2c(c1)cc(-c1ccc(-c3nc(-c4ccc5c(c4)sc4ccccc45)nc(-c4cccc5ccc6ccccc6c45)n3)cc1)c1ccccc12 ; c1ccc2c(-c3nc(-c4ccc5sc6ccccc6c5c4)nc(-c4cccc5ccc6ccccc6c45)n3)cccc2c1 ; c1ccc(-c2ccc(-c3nc(-c4ccc5sc6ccccc6c5c4)nc(-c4cccc5ccc6ccccc6c45)n3)cc2)cc1 ; c1ccc(-c2cccc(-c3nc(-c4ccc5sc6ccccc6c5c4)nc(-c4cccc5ccc6ccccc6c45)n3)c2)cc1]

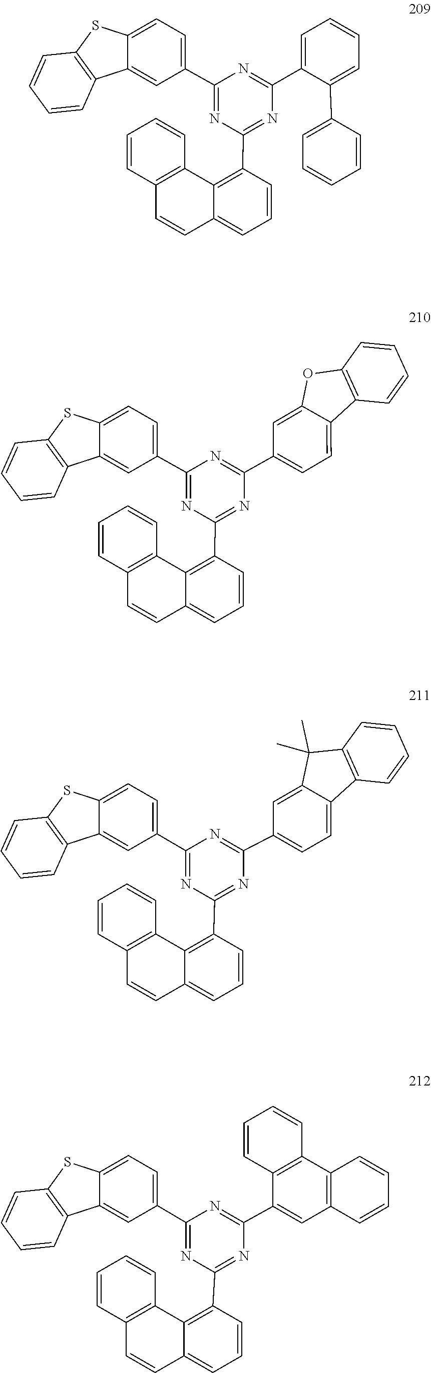 [SMILES: c1ccc(-c2ccccc2-c2nc(-c3ccc4sc5ccccc5c4c3)nc(-c3cccc4ccc5ccccc5c34)n2)cc1 ; c1ccc2c(c1)ccc1cccc(-c3nc(-c4ccc5c(c4)oc4ccccc45)nc(-c4ccc5sc6ccccc6c5c4)n3)c12 ; CC1(C)c2ccccc2-c2ccc(-c3nc(-c4ccc5sc6ccccc6c5c4)nc(-c4cccc5ccc6ccccc6c45)n3)cc21 ; c1ccc2c(c1)cc(-c1nc(-c3ccc4sc5ccccc5c4c3)nc(-c3cccc4ccc5ccccc5c34)n1)c1ccccc12]

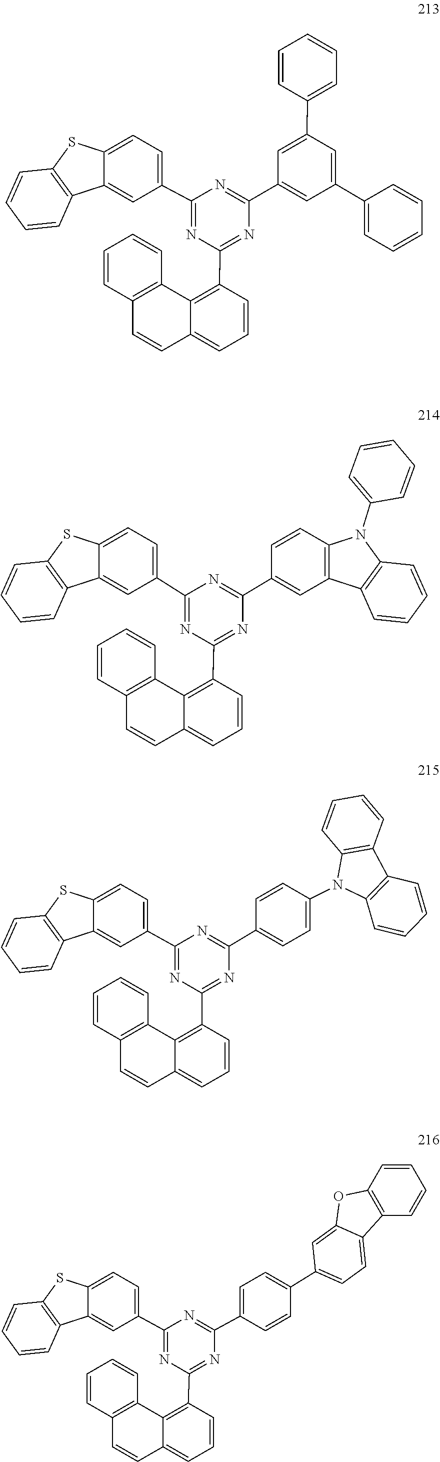 [SMILES: c1ccc(-c2cc(-c3ccccc3)cc(-c3nc(-c4ccc5sc6ccccc6c5c4)nc(-c4cccc5ccc6ccccc6c45)n3)c2)cc1 ; c1ccc(-n2c3ccccc3c3cc(-c4nc(-c5ccc6sc7ccccc7c6c5)nc(-c5cccc6ccc7ccccc7c56)n4)ccc32)cc1 ; c1ccc2c(c1)ccc1cccc(-c3nc(-c4ccc(-n5c6ccccc6c6ccccc65)cc4)nc(-c4ccc5sc6ccccc6c5c4)n3)c12 ; c1ccc2c(c1)ccc1cccc(-c3nc(-c4ccc(-c5ccc6c(c5)oc5ccccc56)cc4)nc(-c4ccc5sc6ccccc6c5c4)n3)c12]

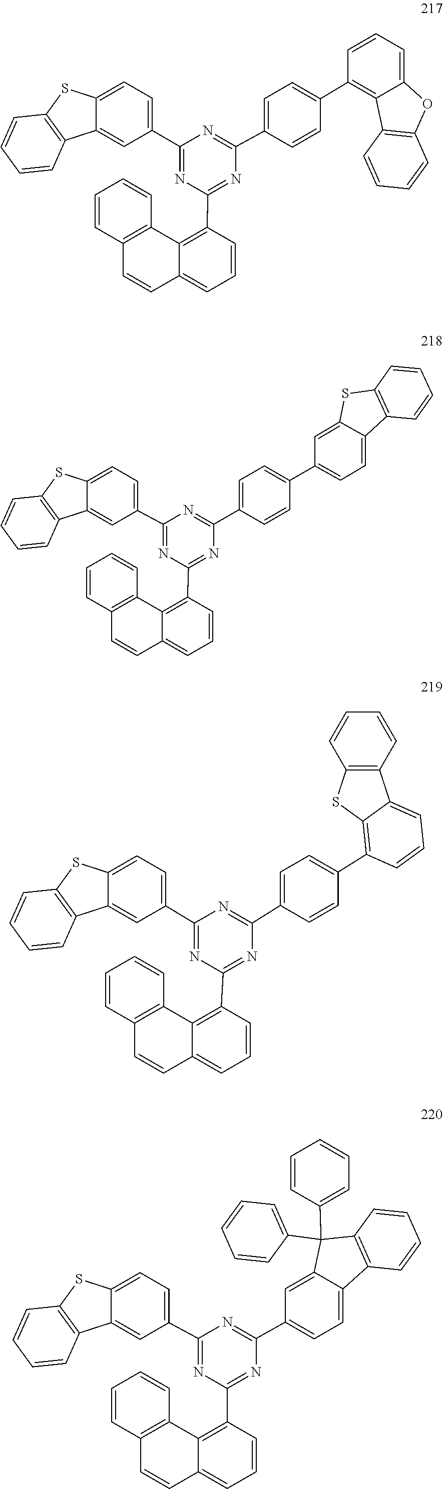 [SMILES: c1ccc2c(c1)ccc1cccc(-c3nc(-c4ccc(-c5cccc6oc7ccccc7c56)cc4)nc(-c4ccc5sc6ccccc6c5c4)n3)c12 ; c1ccc2c(c1)ccc1cccc(-c3nc(-c4ccc(-c5ccc6c(c5)sc5ccccc56)cc4)nc(-c4ccc5sc6ccccc6c5c4)n3)c12 ; c1ccc2c(c1)ccc1cccc(-c3nc(-c4ccc(-c5cccc6c5sc5ccccc56)cc4)nc(-c4ccc5sc6ccccc6c5c4)n3)c12 ; c1ccc(C2(c3ccccc3)c3ccccc3-c3ccc(-c4nc(-c5ccc6sc7ccccc7c6c5)nc(-c5cccc6ccc7ccccc7c56)n4)cc32)cc1]

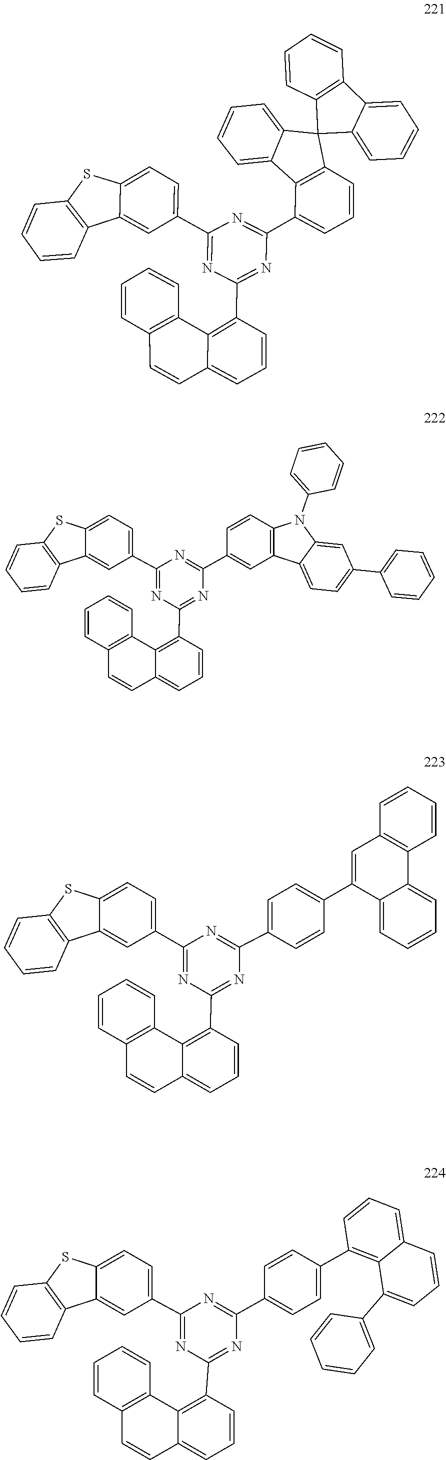 [SMILES: c1ccc2c(c1)-c1ccccc1C21c2ccccc2-c2c(-c3nc(-c4ccc5sc6ccccc6c5c4)nc(-c4cccc5ccc6ccccc6c45)n3)cccc21 ; c1ccc(-c2ccc3c4cc(-c5nc(-c6ccc7sc8ccccc8c7c6)nc(-c6cccc7ccc8ccccc8c67)n5)ccc4n(-c4ccccc4)c3c2)cc1 ; c1ccc2c(c1)cc(-c1ccc(-c3nc(-c4ccc5sc6ccccc6c5c4)nc(-c4cccc5ccc6ccccc6c45)n3)cc1)c1ccccc12 ; c1ccc(-c2cccc3cccc(-c4ccc(-c5nc(-c6ccc7sc8ccccc8c7c6)nc(-c6cccc7ccc8ccccc8c67)n5)cc4)c23)cc1]

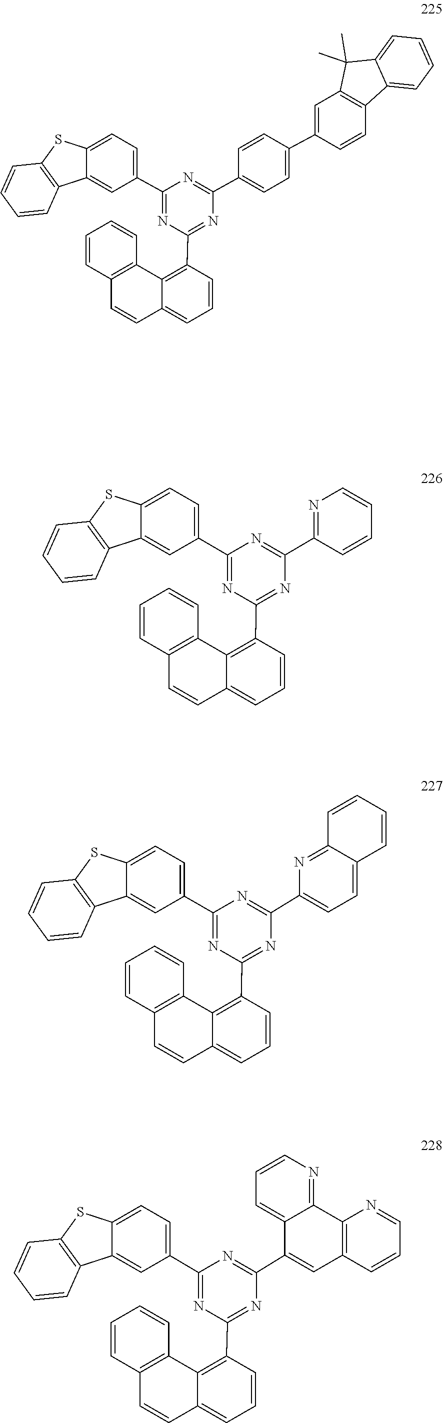 [SMILES: CC1(C)c2ccccc2-c2ccc(-c3ccc(-c4nc(-c5ccc6sc7ccccc7c6c5)nc(-c5cccc6ccc7ccccc7c56)n4)cc3)cc21 ; c1ccc(-c2nc(-c3ccc4sc5ccccc5c4c3)nc(-c3cccc4ccc5ccccc5c34)n2)nc1 ; c1ccc2nc(-c3nc(-c4ccc5sc6ccccc6c5c4)nc(-c4cccc5ccc6ccccc6c45)n3)ccc2c1 ; c1ccc2c(c1)ccc1cccc(-c3nc(-c4ccc5sc6ccccc6c5c4)nc(-c4cc5cccnc5c5ncccc45)n3)c12]

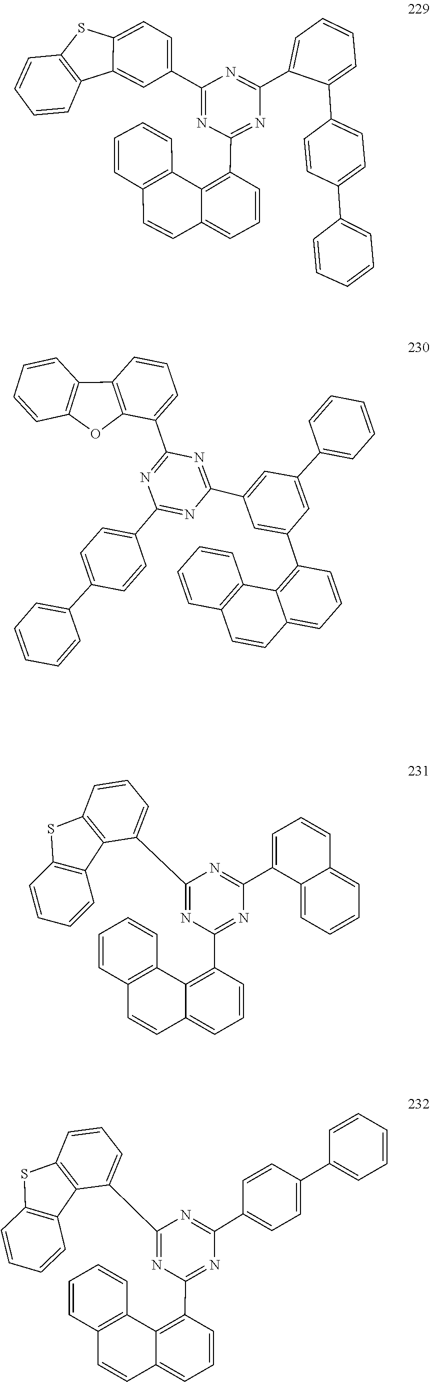 [SMILES: c1ccc(-c2ccc(-c3ccccc3-c3nc(-c4ccc5sc6ccccc6c5c4)nc(-c4cccc5ccc6ccccc6c45)n3)cc2)cc1 ; c1ccc(-c2ccc(-c3nc(-c4cc(-c5ccccc5)cc(-c5cccc6ccc7ccccc7c56)c4)nc(-c4cccc5c4oc4ccccc45)n3)cc2)cc1 ; c1ccc2c(-c3nc(-c4cccc5ccc6ccccc6c45)nc(-c4cccc5sc6ccccc6c45)n3)cccc2c1 ; c1ccc(-c2ccc(-c3nc(-c4cccc5ccc6ccccc6c45)nc(-c4cccc5sc6ccccc6c45)n3)cc2)cc1]

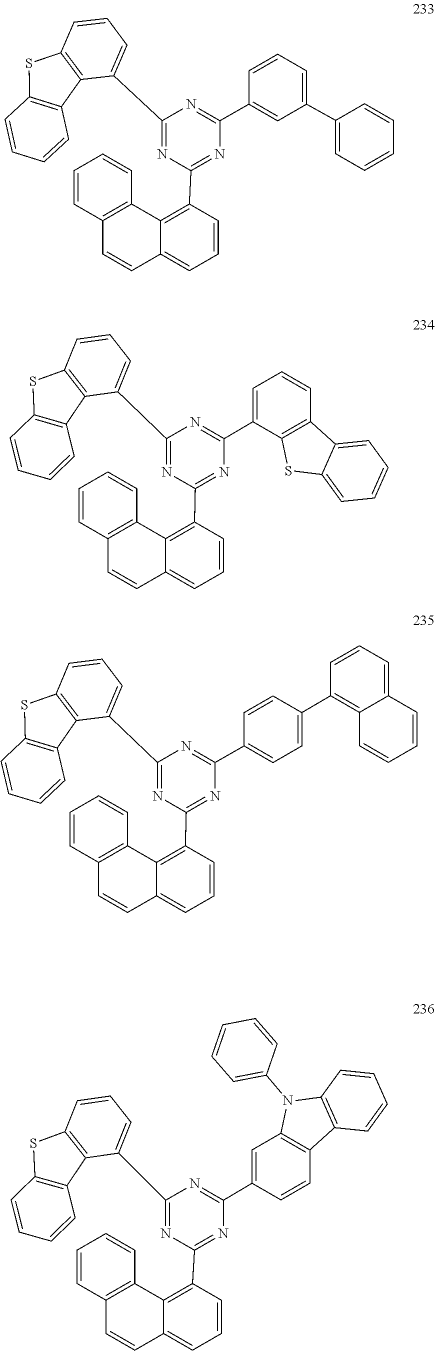 [SMILES: c1ccc(-c2cccc(-c3nc(-c4cccc5ccc6ccccc6c45)nc(-c4cccc5sc6ccccc6c45)n3)c2)cc1 ; c1ccc2c(c1)ccc1cccc(-c3nc(-c4cccc5c4sc4ccccc45)nc(-c4cccc5sc6ccccc6c45)n3)c12 ; c1ccc2c(-c3ccc(-c4nc(-c5cccc6ccc7ccccc7c56)nc(-c5cccc6sc7ccccc7c56)n4)cc3)cccc2c1 ; c1ccc(-n2c3ccccc3c3ccc(-c4nc(-c5cccc6ccc7ccccc7c56)nc(-c5cccc6sc7ccccc7c56)n4)cc32)cc1]

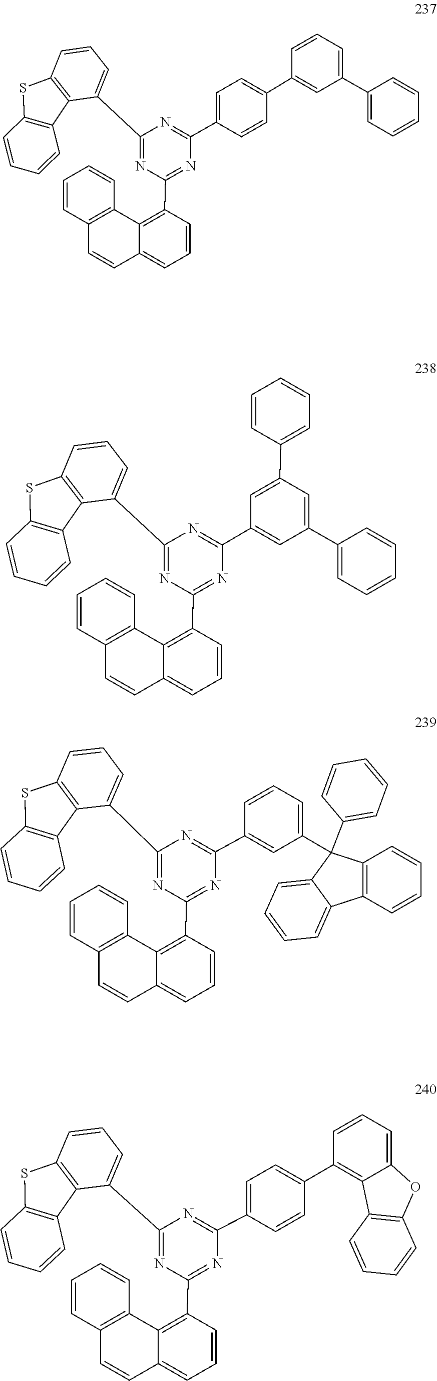 [SMILES: c1ccc(-c2cccc(-c3ccc(-c4nc(-c5cccc6ccc7ccccc7c56)nc(-c5cccc6sc7ccccc7c56)n4)cc3)c2)cc1 ; c1ccc(-c2cc(-c3ccccc3)cc(-c3nc(-c4cccc5ccc6ccccc6c45)nc(-c4cccc5sc6ccccc6c45)n3)c2)cc1 ; c1ccc(C2(c3cccc(-c4nc(-c5cccc6ccc7ccccc7c56)nc(-c5cccc6sc7ccccc7c56)n4)c3)c3ccccc3-c3ccccc32)cc1 ; c1ccc2c(c1)ccc1cccc(-c3nc(-c4ccc(-c5cccc6oc7ccccc7c56)cc4)nc(-c4cccc5sc6ccccc6c45)n3)c12]

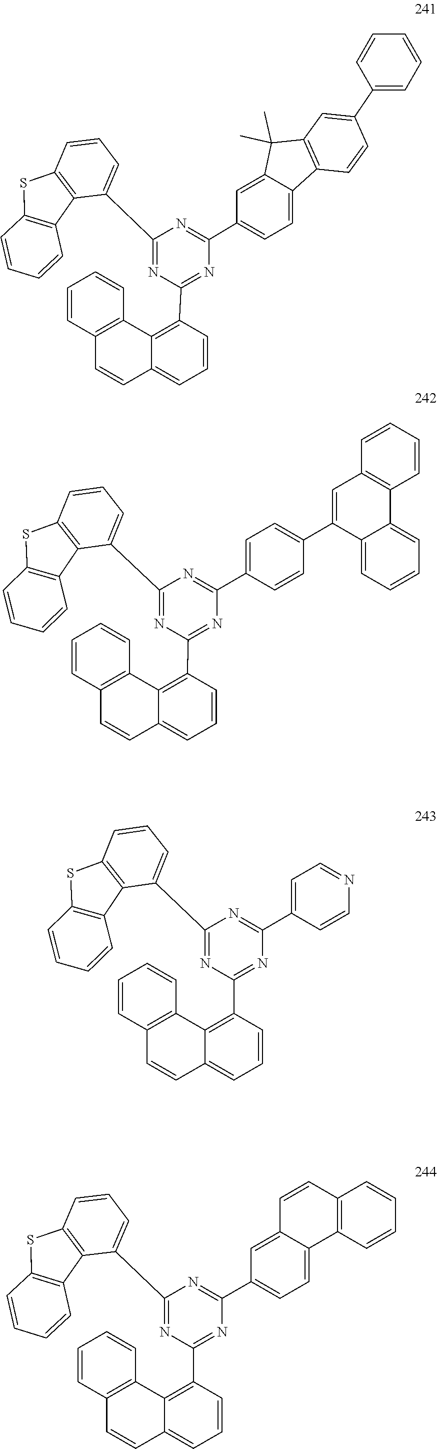 [SMILES: CC1(C)c2cc(-c3ccccc3)ccc2-c2ccc(-c3nc(-c4cccc5ccc6ccccc6c45)nc(-c4cccc5sc6ccccc6c45)n3)cc21 ; c1ccc2c(c1)cc(-c1ccc(-c3nc(-c4cccc5ccc6ccccc6c45)nc(-c4cccc5sc6ccccc6c45)n3)cc1)c1ccccc12 ; c1ccc2c(c1)ccc1cccc(-c3nc(-c4ccncc4)nc(-c4cccc5sc6ccccc6c45)n3)c12 ; c1ccc2c(c1)ccc1cc(-c3nc(-c4cccc5ccc6ccccc6c45)nc(-c4cccc5sc6ccccc6c45)n3)ccc12]

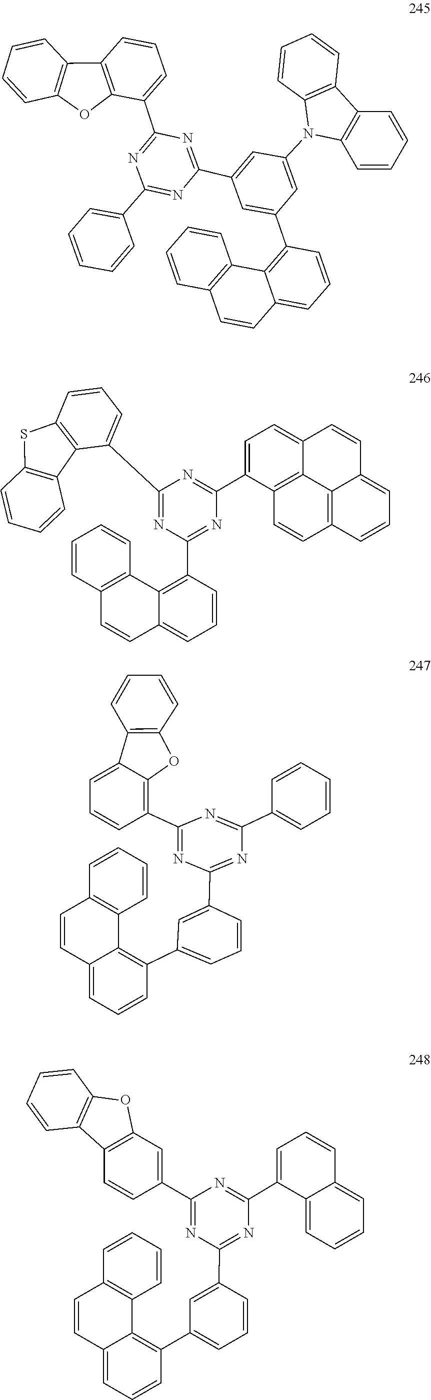

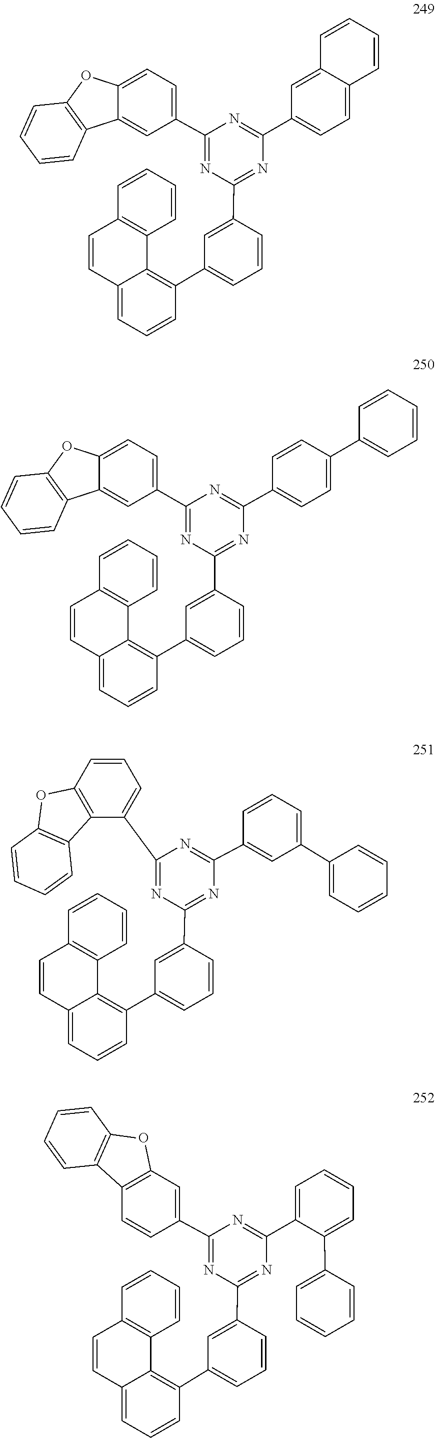 [SMILES: c1cc(-c2nc(-c3ccc4ccccc4c3)nc(-c3ccc4oc5ccccc5c4c3)n2)cc(-c2cccc3ccc4ccccc4c23)c1 ; c1ccc(-c2ccc(-c3nc(-c4cccc(-c5cccc6ccc7ccccc7c56)c4)nc(-c4ccc5oc6ccccc6c5c4)n3)cc2)cc1 ; c1ccc(-c2cccc(-c3nc(-c4cccc(-c5cccc6ccc7ccccc7c56)c4)nc(-c4cccc5oc6ccccc6c45)n3)c2)cc1 ; c1ccc(-c2ccccc2-c2nc(-c3cccc(-c4cccc5ccc6ccccc6c45)c3)nc(-c3ccc4c(c3)oc3ccccc34)n2)cc1]

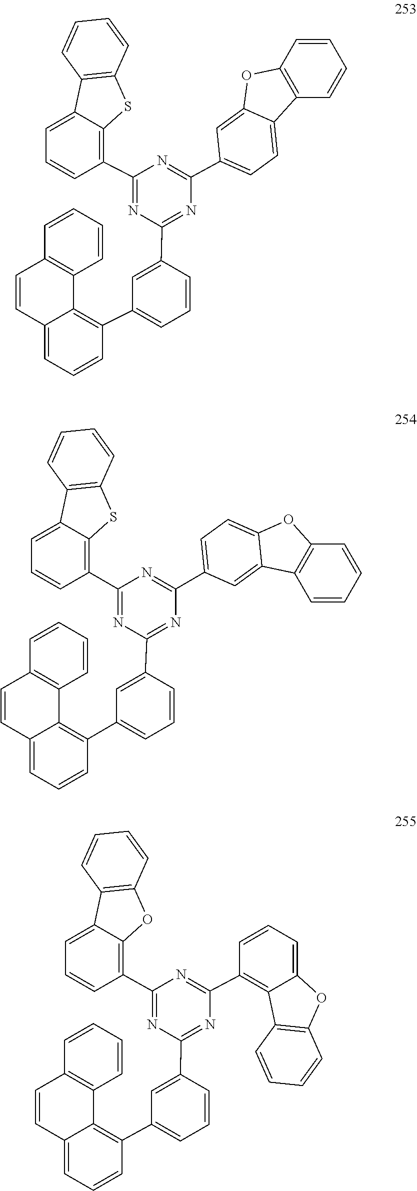 [SMILES: c1cc(-c2nc(-c3ccc4c(c3)oc3ccccc34)nc(-c3cccc4c3sc3ccccc34)n2)cc(-c2cccc3ccc4ccccc4c23)c1 ; c1cc(-c2nc(-c3ccc4oc5ccccc5c4c3)nc(-c3cccc4c3sc3ccccc34)n2)cc(-c2cccc3ccc4ccccc4c23)c1 ; c1cc(-c2nc(-c3cccc4c3oc3ccccc34)nc(-c3cccc4oc5ccccc5c34)n2)cc(-c2cccc3ccc4ccccc4c23)c1]

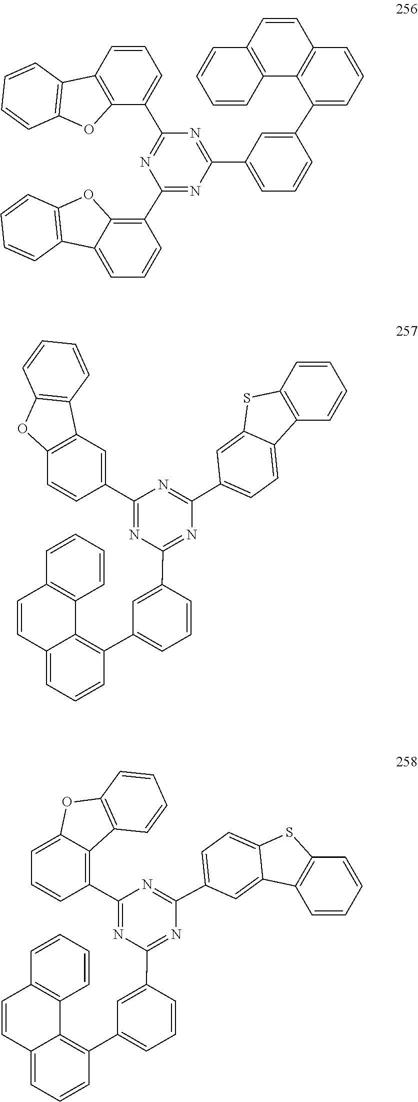 [SMILES: c1cc(-c2nc(-c3cccc4c3oc3ccccc34)nc(-c3cccc4c3oc3ccccc34)n2)cc(-c2cccc3ccc4ccccc4c23)c1 ; c1cc(-c2nc(-c3ccc4c(c3)sc3ccccc34)nc(-c3ccc4oc5ccccc5c4c3)n2)cc(-c2cccc3ccc4ccccc4c23)c1 ; c1cc(-c2nc(-c3ccc4sc5ccccc5c4c3)nc(-c3cccc4oc5ccccc5c34)n2)cc(-c2cccc3ccc4ccccc4c23)c1]

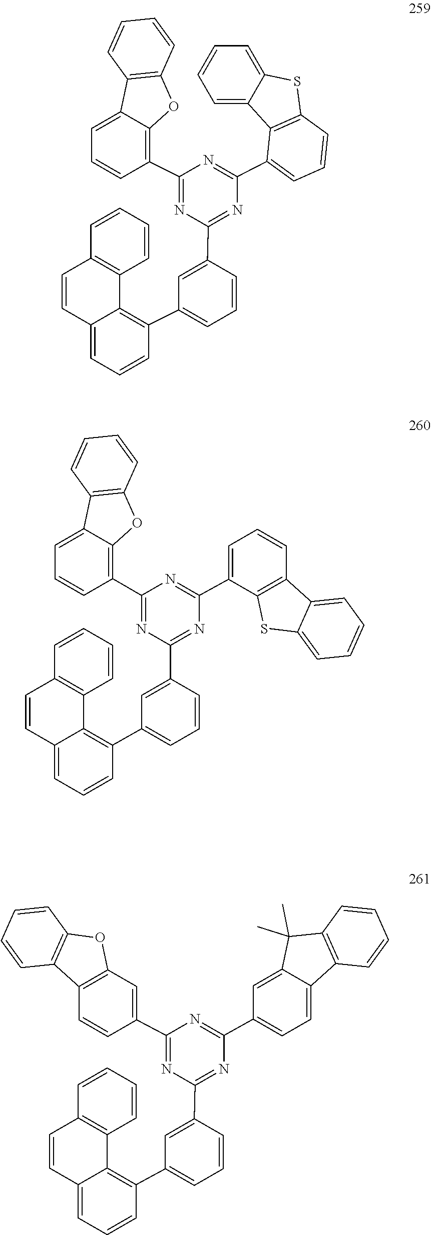 [SMILES: c1cc(-c2nc(-c3cccc4c3oc3ccccc34)nc(-c3cccc4sc5ccccc5c34)n2)cc(-c2cccc3ccc4ccccc4c23)c1 ; c1cc(-c2nc(-c3cccc4c3oc3ccccc34)nc(-c3cccc4c3sc3ccccc34)n2)cc(-c2cccc3ccc4ccccc4c23)c1 ; CC1(C)c2ccccc2-c2ccc(-c3nc(-c4cccc(-c5cccc6ccc7ccccc7c56)c4)nc(-c4ccc5c(c4)oc4ccccc45)n3)cc21]

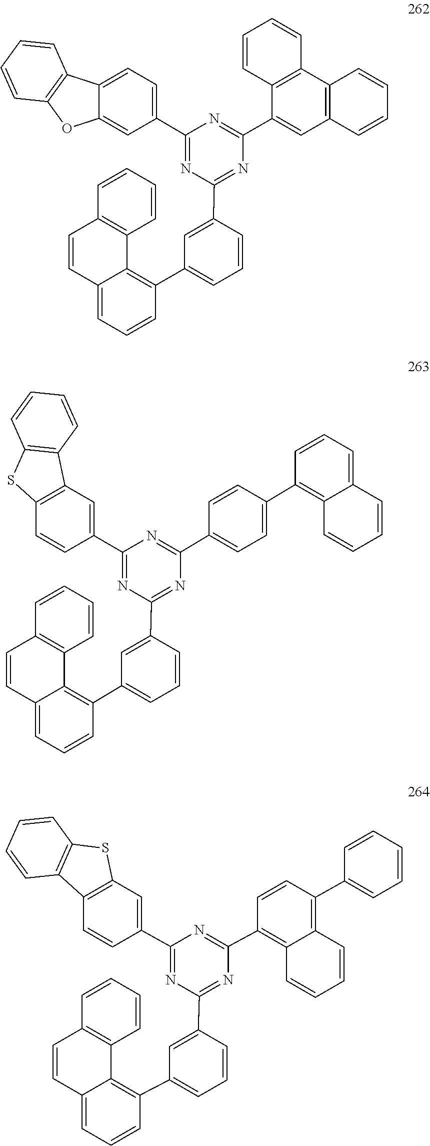 [SMILES: c1cc(-c2nc(-c3ccc4c(c3)oc3ccccc34)nc(-c3cc4ccccc4c4ccccc34)n2)cc(-c2cccc3ccc4ccccc4c23)c1 ; c1cc(-c2nc(-c3ccc(-c4cccc5ccccc45)cc3)nc(-c3ccc4sc5ccccc5c4c3)n2)cc(-c2cccc3ccc4ccccc4c23)c1 ; c1ccc(-c2ccc(-c3nc(-c4cccc(-c5cccc6ccc7ccccc7c56)c4)nc(-c4ccc5c(c4)sc4ccccc45)n3)c3ccccc23)cc1]

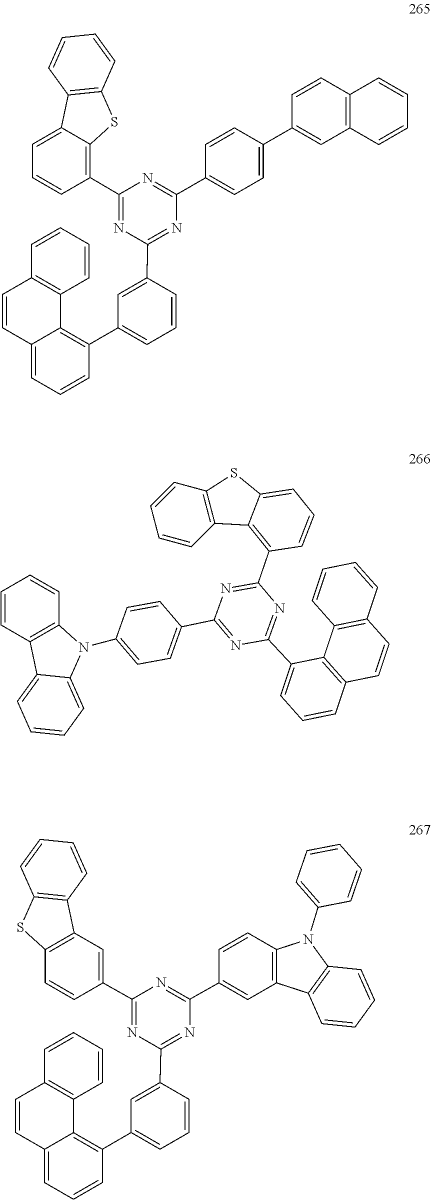 [SMILES: c1cc(-c2nc(-c3ccc(-c4ccc5ccccc5c4)cc3)nc(-c3cccc4c3sc3ccccc34)n2)cc(-c2cccc3ccc4ccccc4c23)c1 ; c1ccc2c(c1)ccc1cccc(-c3nc(-c4ccc(-n5c6ccccc6c6ccccc65)cc4)nc(-c4cccc5sc6ccccc6c45)n3)c12 ; c1ccc(-n2c3ccccc3c3cc(-c4nc(-c5cccc(-c6cccc7ccc8ccccc8c67)c5)nc(-c5ccc6sc7ccccc7c6c5)n4)ccc32)cc1]

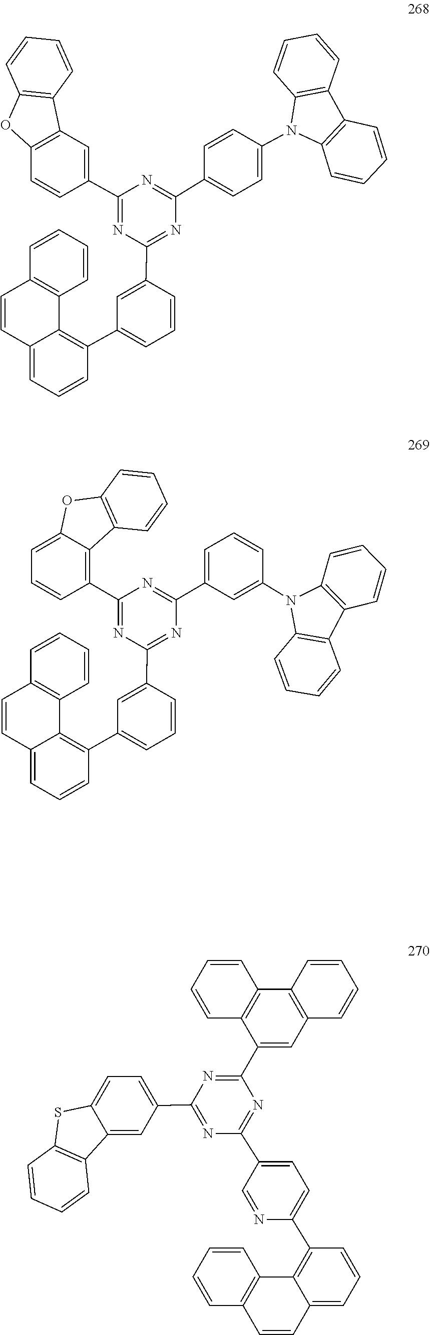 [SMILES: c1cc(-c2nc(-c3ccc(-n4c5ccccc5c5ccccc54)cc3)nc(-c3ccc4oc5ccccc5c4c3)n2)cc(-c2cccc3ccc4ccccc4c23)c1 ; c1cc(-c2nc(-c3cccc(-n4c5ccccc5c5ccccc54)c3)nc(-c3cccc4oc5ccccc5c34)n2)cc(-c2cccc3ccc4ccccc4c23)c1 ; c1ccc2c(c1)cc(-c1nc(-c3ccc(-c4cccc5ccc6ccccc6c45)nc3)nc(-c3ccc4sc5ccccc5c4c3)n1)c1ccccc12]

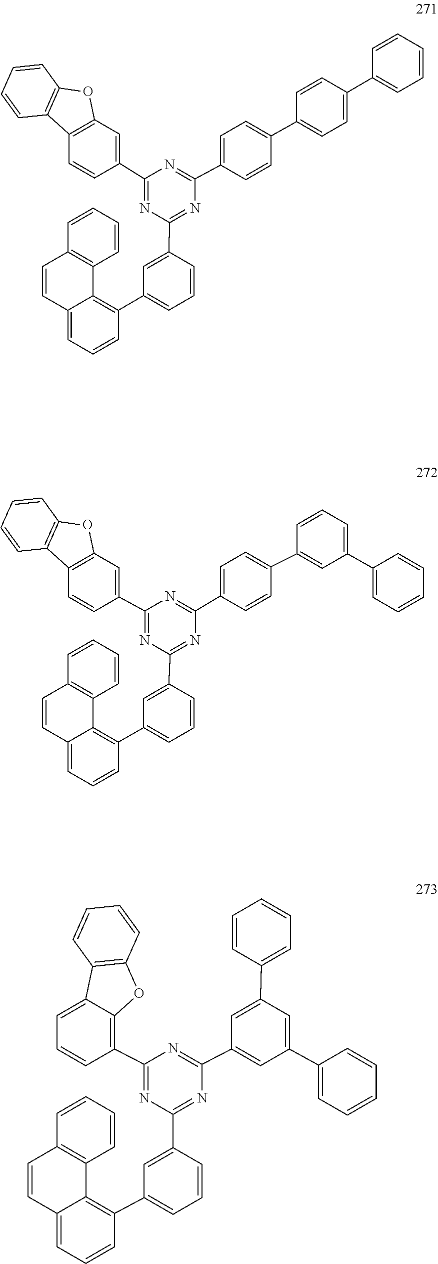 [SMILES: c1ccc(-c2ccc(-c3ccc(-c4nc(-c5cccc(-c6cccc7ccc8ccccc8c67)c5)nc(-c5ccc6c(c5)oc5ccccc56)n4)cc3)cc2)cc1 ; c1ccc(-c2cccc(-c3ccc(-c4nc(-c5cccc(-c6cccc7ccc8ccccc8c67)c5)nc(-c5ccc6c(c5)oc5ccccc56)n4)cc3)c2)cc1 ; c1ccc(-c2cc(-c3ccccc3)cc(-c3nc(-c4cccc(-c5cccc6ccc7ccccc7c56)c4)nc(-c4cccc5c4oc4ccccc45)n3)c2)cc1]

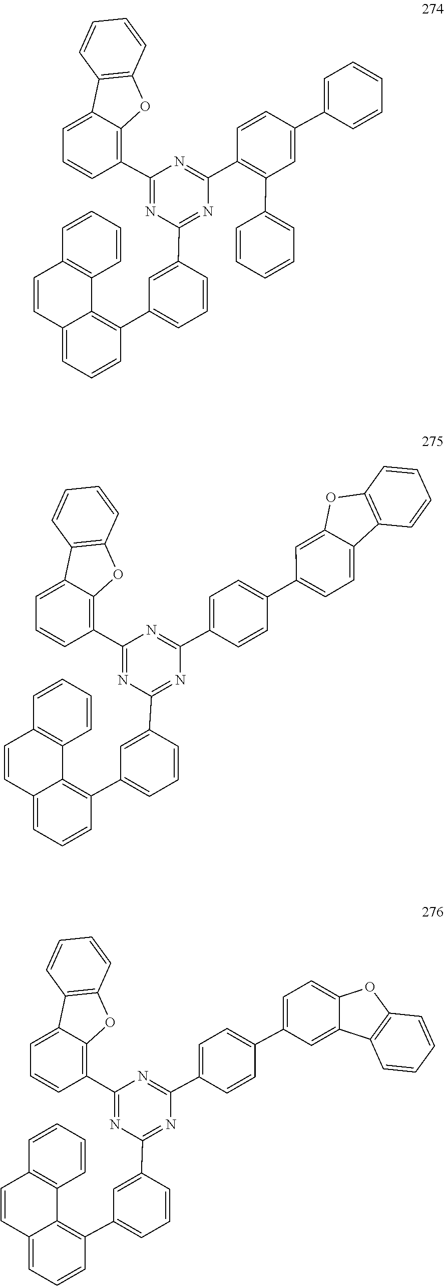 [SMILES: c1ccc(-c2ccc(-c3nc(-c4cccc(-c5cccc6ccc7ccccc7c56)c4)nc(-c4cccc5c4oc4ccccc45)n3)c(-c3ccccc3)c2)cc1 ; c1cc(-c2nc(-c3ccc(-c4ccc5c(c4)oc4ccccc45)cc3)nc(-c3cccc4c3oc3ccccc34)n2)cc(-c2cccc3ccc4ccccc4c23)c1 ; c1cc(-c2nc(-c3ccc(-c4ccc5oc6ccccc6c5c4)cc3)nc(-c3cccc4c3oc3ccccc34)n2)cc(-c2cccc3ccc4ccccc4c23)c1]

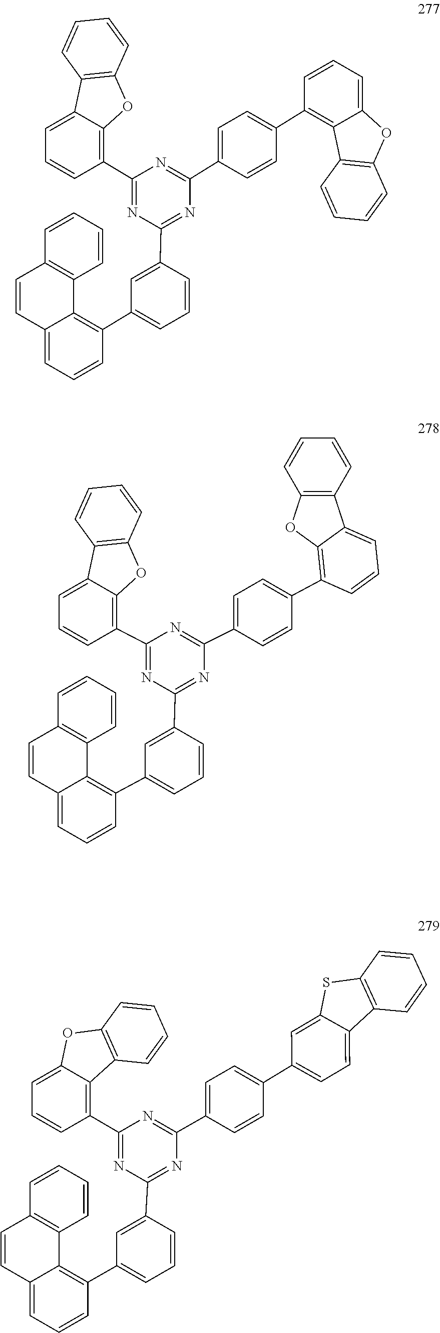 [SMILES: c1cc(-c2nc(-c3ccc(-c4cccc5oc6ccccc6c45)cc3)nc(-c3cccc4c3oc3ccccc34)n2)cc(-c2cccc3ccc4ccccc4c23)c1 ; c1cc(-c2nc(-c3ccc(-c4cccc5c4oc4ccccc45)cc3)nc(-c3cccc4c3oc3ccccc34)n2)cc(-c2cccc3ccc4ccccc4c23)c1 ; c1cc(-c2nc(-c3ccc(-c4ccc5c(c4)sc4ccccc45)cc3)nc(-c3cccc4oc5ccccc5c34)n2)cc(-c2cccc3ccc4ccccc4c23)c1]

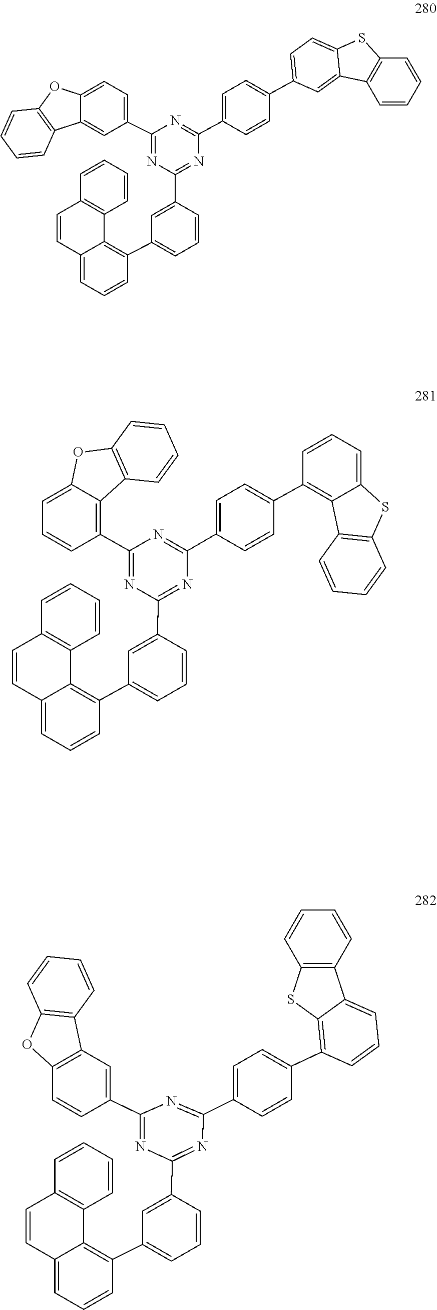 [SMILES: c1cc(-c2nc(-c3ccc(-c4ccc5sc6ccccc6c5c4)cc3)nc(-c3ccc4oc5ccccc5c4c3)n2)cc(-c2cccc3ccc4ccccc4c23)c1 ; c1cc(-c2nc(-c3ccc(-c4cccc5sc6ccccc6c45)cc3)nc(-c3cccc4oc5ccccc5c34)n2)cc(-c2cccc3ccc4ccccc4c23)c1 ; c1cc(-c2nc(-c3ccc(-c4cccc5c4sc4ccccc45)cc3)nc(-c3ccc4oc5ccccc5c4c3)n2)cc(-c2cccc3ccc4ccccc4c23)c1]

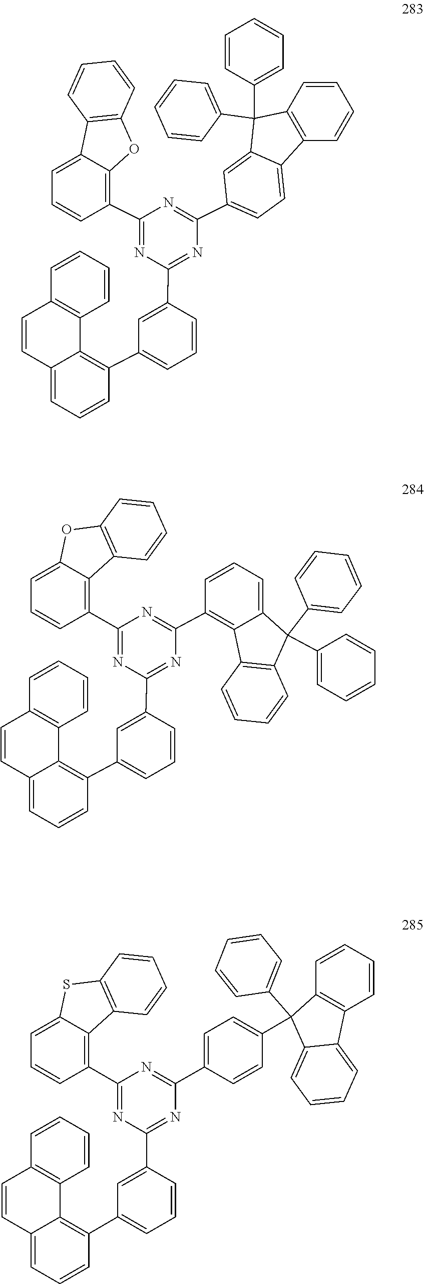 [SMILES: c1ccc(C2(c3ccccc3)c3ccccc3-c3ccc(-c4nc(-c5cccc(-c6cccc7ccc8ccccc8c67)c5)nc(-c5cccc6c5oc5ccccc56)n4)cc32)cc1 ; c1ccc(C2(c3ccccc3)c3ccccc3-c3c(-c4nc(-c5cccc(-c6cccc7ccc8ccccc8c67)c5)nc(-c5cccc6oc7ccccc7c56)n4)cccc32)cc1 ; c1ccc(C2(c3ccc(-c4nc(-c5cccc(-c6cccc7ccc8ccccc8c67)c5)nc(-c5cccc6sc7ccccc7c56)n4)cc3)c3ccccc3-c3ccccc32)cc1]

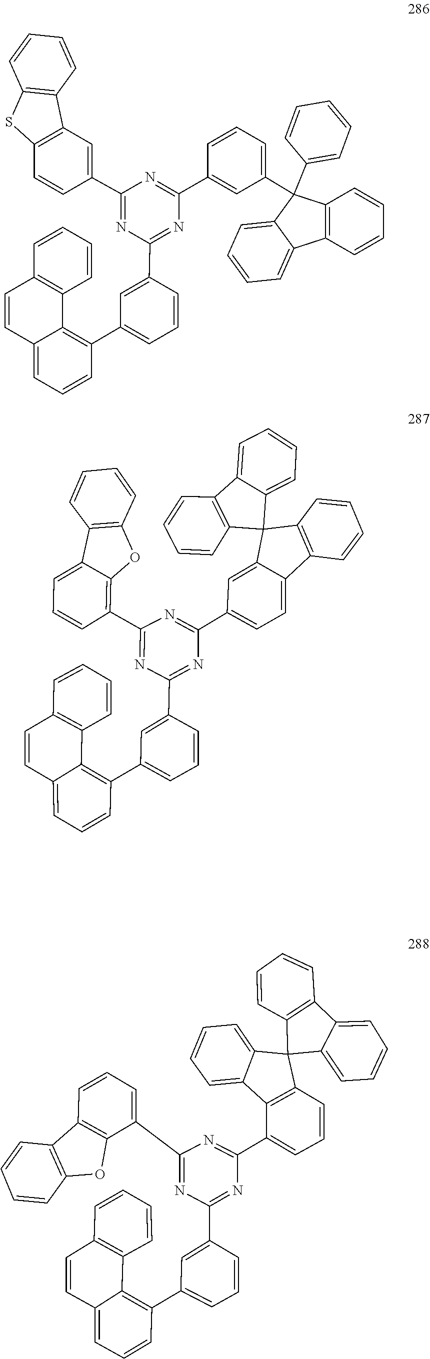 [SMILES: c1ccc(C2(c3cccc(-c4nc(-c5cccc(-c6cccc7ccc8ccccc8c67)c5)nc(-c5ccc6sc7ccccc7c6c5)n4)c3)c3ccccc3-c3ccccc32)cc1 ; c1cc(-c2nc(-c3ccc4c(c3)C3(c5ccccc5-c5ccccc53)c3ccccc3-4)nc(-c3cccc4c3oc3ccccc34)n2)cc(-c2cccc3ccc4ccccc4c23)c1 ; c1cc(-c2nc(-c3cccc4c3-c3ccccc3C43c4ccccc4-c4ccccc43)nc(-c3cccc4c3oc3ccccc34)n2)cc(-c2cccc3ccc4ccccc4c23)c1]

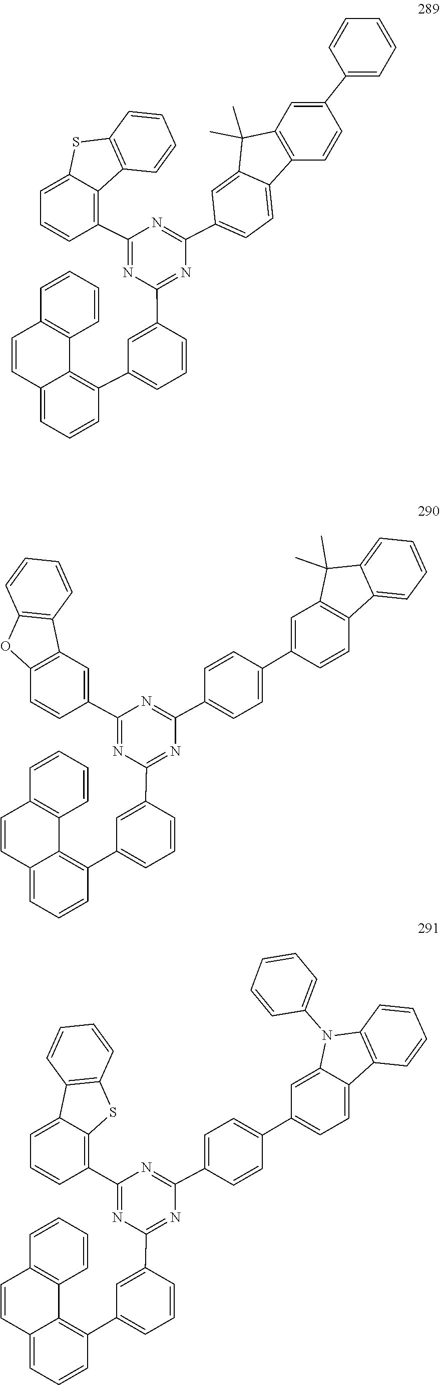 [SMILES: CC1(C)c2cc(-c3ccccc3)ccc2-c2ccc(-c3nc(-c4cccc(-c5cccc6ccc7ccccc7c56)c4)nc(-c4cccc5sc6ccccc6c45)n3)cc21 ; CC1(C)c2ccccc2-c2ccc(-c3ccc(-c4nc(-c5cccc(-c6cccc7ccc8ccccc8c67)c5)nc(-c5ccc6oc7ccccc7c6c5)n4)cc3)cc21 ; c1ccc(-n2c3ccccc3c3ccc(-c4ccc(-c5nc(-c6cccc(-c7cccc8ccc9ccccc9c78)c6)nc(-c6cccc7c6sc6ccccc67)n5)cc4)cc32)cc1]

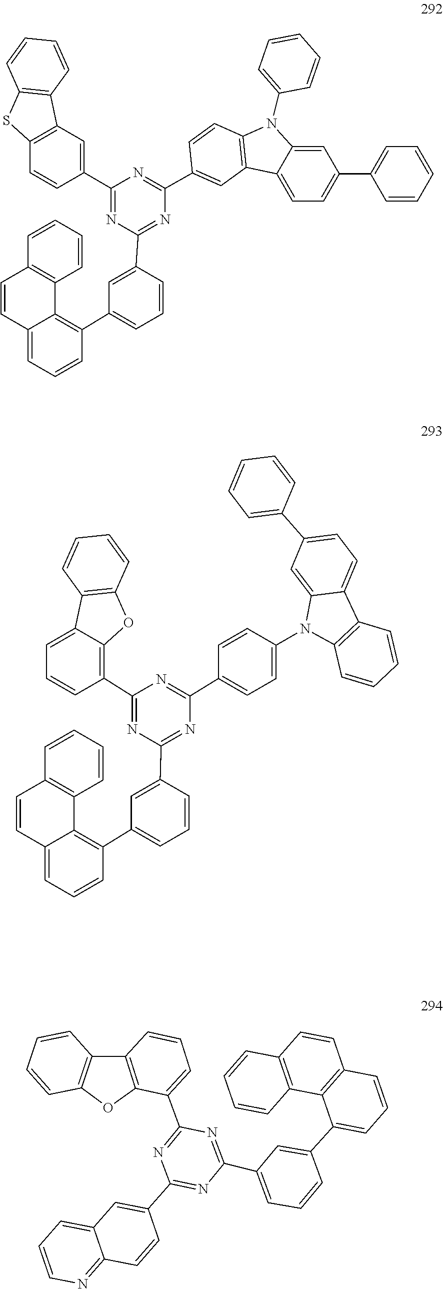 [SMILES: c1ccc(-c2ccc3c4cc(-c5nc(-c6cccc(-c7cccc8ccc9ccccc9c78)c6)nc(-c6ccc7sc8ccccc8c7c6)n5)ccc4n(-c4ccccc4)c3c2)cc1 ; c1ccc(-c2ccc3c4ccccc4n(-c4ccc(-c5nc(-c6cccc(-c7cccc8ccc9ccccc9c78)c6)nc(-c6cccc7c6oc6ccccc67)n5)cc4)c3c2)cc1 ; c1cc(-c2nc(-c3ccc4ncccc4c3)nc(-c3cccc4c3oc3ccccc34)n2)cc(-c2cccc3ccc4ccccc4c23)c1]

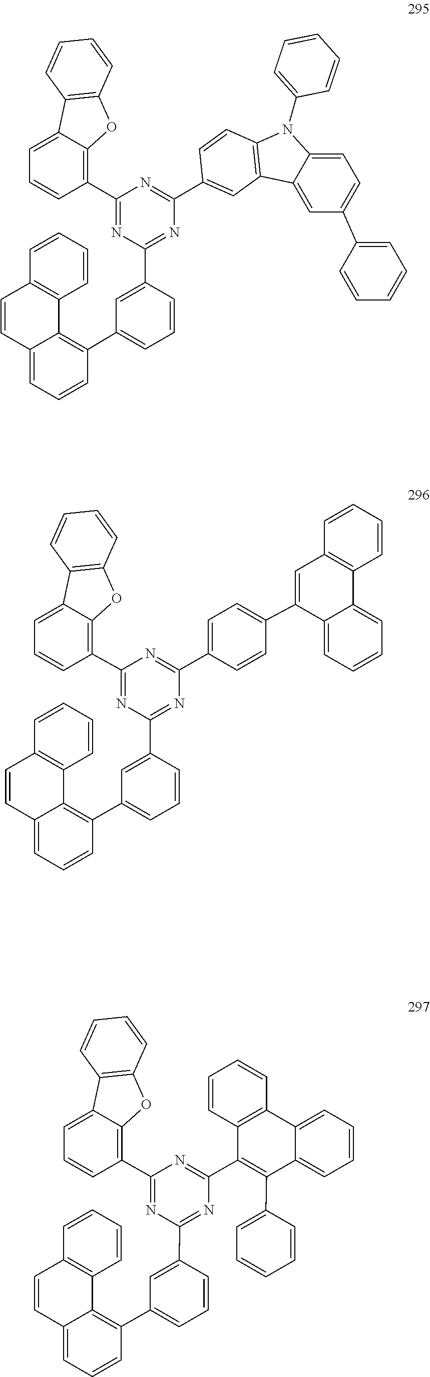 [SMILES: c1ccc(-c2ccc3c(c2)c2cc(-c4nc(-c5cccc(-c6cccc7ccc8ccccc8c67)c5)nc(-c5cccc6c5oc5ccccc56)n4)ccc2n3-c2ccccc2)cc1 ; c1cc(-c2nc(-c3ccc(-c4cc5ccccc5c5ccccc45)cc3)nc(-c3cccc4c3oc3ccccc34)n2)cc(-c2cccc3ccc4ccccc4c23)c1 ; c1ccc(-c2c(-c3nc(-c4cccc(-c5cccc6ccc7ccccc7c56)c4)nc(-c4cccc5c4oc4ccccc45)n3)c3ccccc3c3ccccc23)cc1]

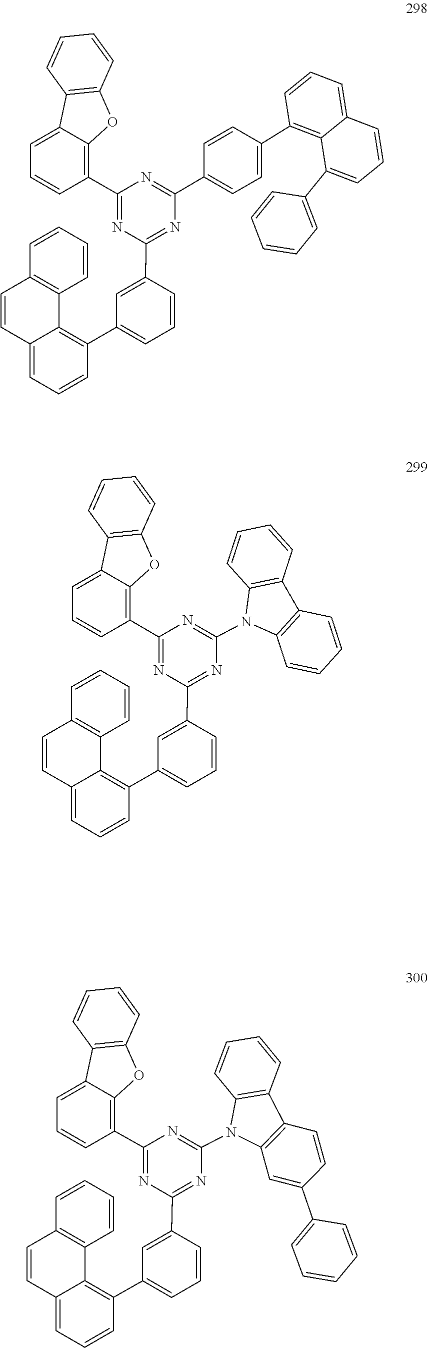 [SMILES: c1ccc(-c2cccc3cccc(-c4ccc(-c5nc(-c6cccc(-c7cccc8ccc9ccccc9c78)c6)nc(-c6cccc7c6oc6ccccc67)n5)cc4)c23)cc1 ; c1cc(-c2nc(-c3cccc4c3oc3ccccc34)nc(-n3c4ccccc4c4ccccc43)n2)cc(-c2cccc3ccc4ccccc4c23)c1 ; c1ccc(-c2ccc3c4ccccc4n(-c4nc(-c5cccc(-c6cccc7ccc8ccccc8c67)c5)nc(-c5cccc6c5oc5ccccc56)n4)c3c2)cc1]

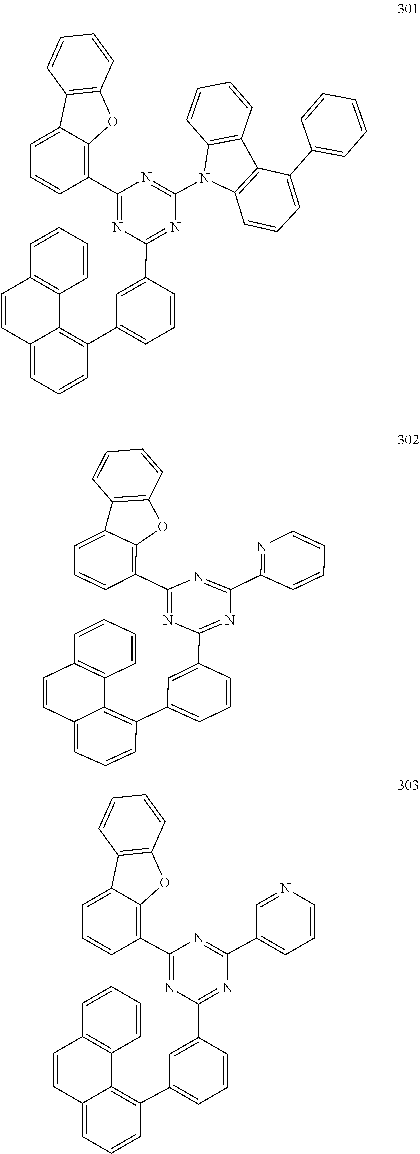

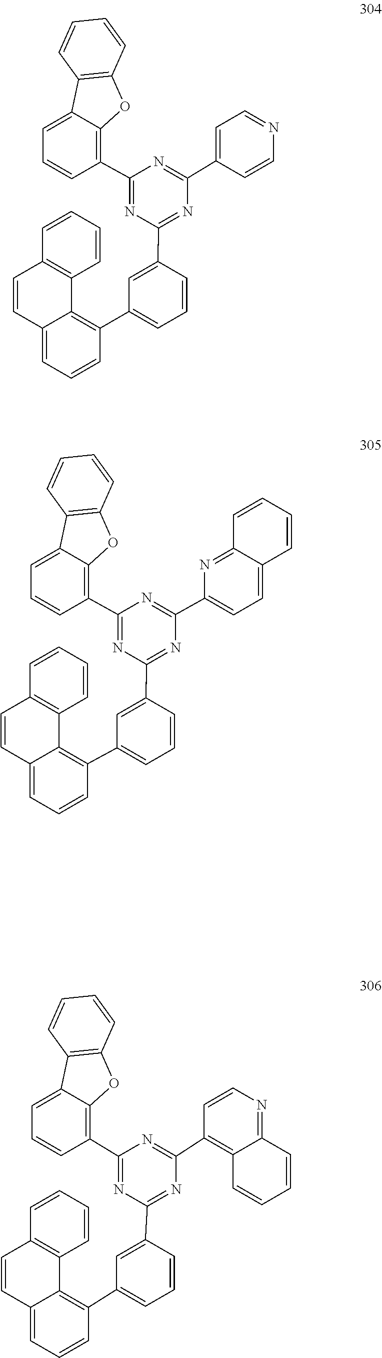 [SMILES: c1cc(-c2nc(-c3ccncc3)nc(-c3cccc4c3oc3ccccc34)n2)cc(-c2cccc3ccc4ccccc4c23)c1 ; c1cc(-c2nc(-c3ccc4ccccc4n3)nc(-c3cccc4c3oc3ccccc34)n2)cc(-c2cccc3ccc4ccccc4c23)c1 ; c1cc(-c2nc(-c3ccnc4ccccc34)nc(-c3cccc4c3oc3ccccc34)n2)cc(-c2cccc3ccc4ccccc4c23)c1]

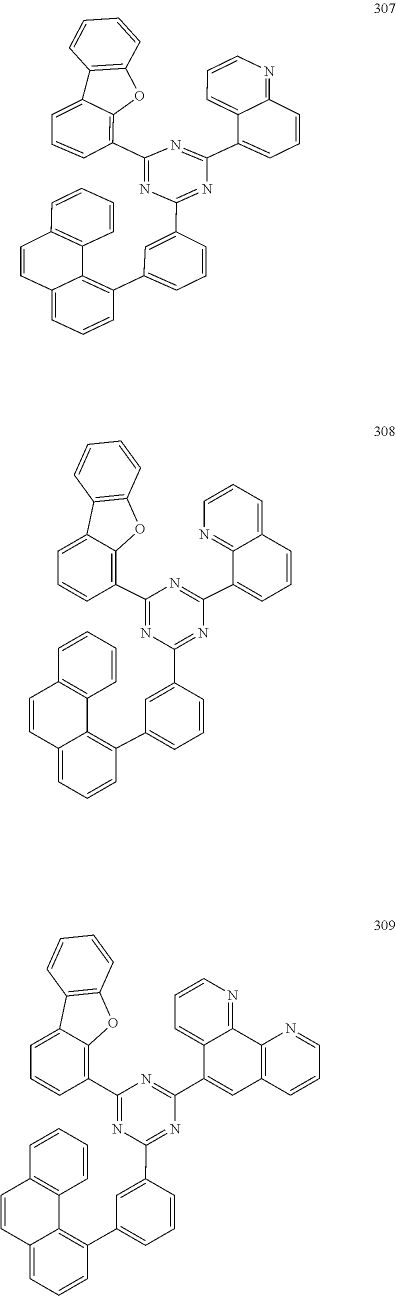 [SMILES: c1cc(-c2nc(-c3cccc4ncccc34)nc(-c3cccc4c3oc3ccccc34)n2)cc(-c2cccc3ccc4ccccc4c23)c1 ; c1cc(-c2nc(-c3cccc4cccnc34)nc(-c3cccc4c3oc3ccccc34)n2)cc(-c2cccc3ccc4ccccc4c23)c1 ; c1cc(-c2nc(-c3cc4cccnc4c4ncccc34)nc(-c3cccc4c3oc3ccccc34)n2)cc(-c2cccc3ccc4ccccc4c23)c1]

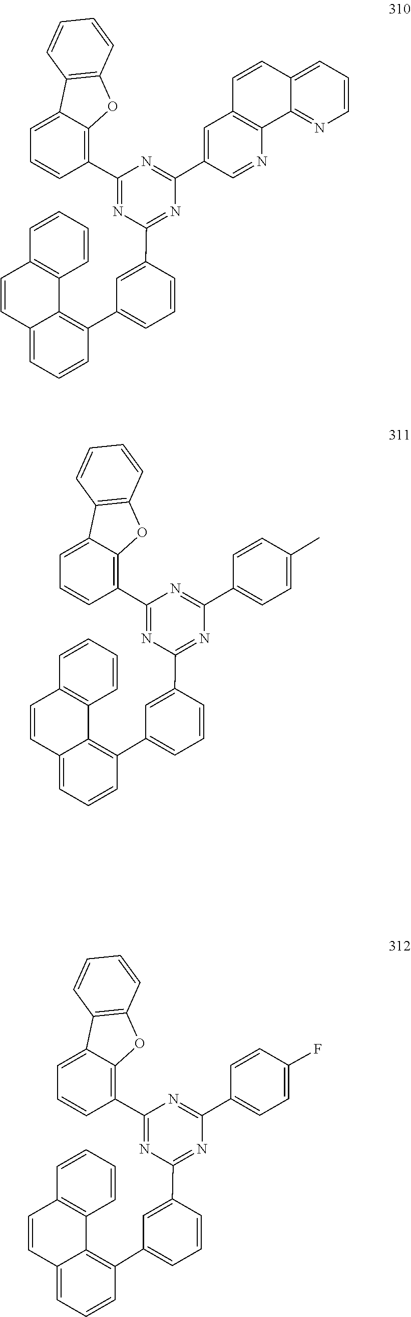 [SMILES: c1cc(-c2nc(-c3cnc4c(ccc5cccnc54)c3)nc(-c3cccc4c3oc3ccccc34)n2)cc(-c2cccc3ccc4ccccc4c23)c1 ; Cc1ccc(-c2nc(-c3cccc(-c4cccc5ccc6ccccc6c45)c3)nc(-c3cccc4c3oc3ccccc34)n2)cc1 ; Fc1ccc(-c2nc(-c3cccc(-c4cccc5ccc6ccccc6c45)c3)nc(-c3cccc4c3oc3ccccc34)n2)cc1]

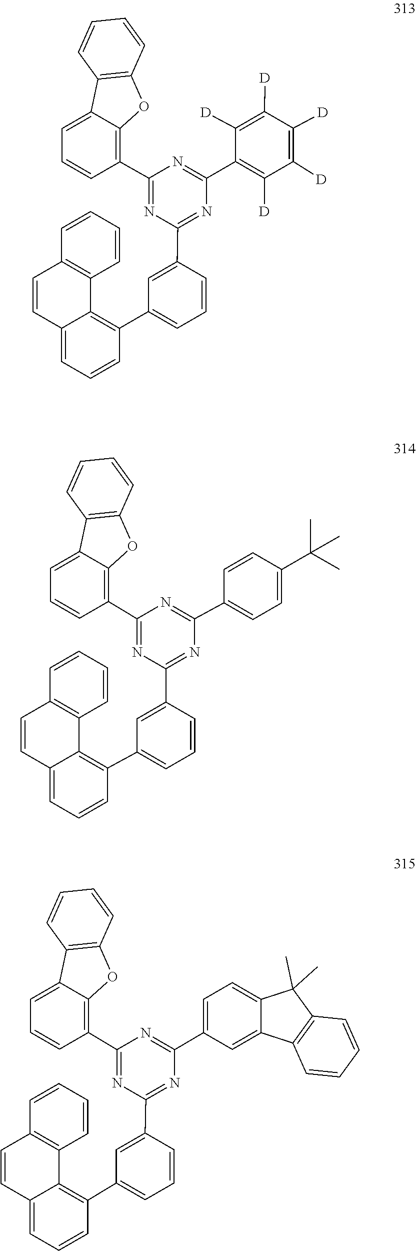 [SMILES: [2H]c1c([2H])c([2H])c(-c2nc(-c3cccc(-c4cccc5ccc6ccccc6c45)c3)nc(-c3cccc4c3oc3ccccc34)n2)c([2H])c1[2H] ; CC(C)(C)c1ccc(-c2nc(-c3cccc(-c4cccc5ccc6ccccc6c45)c3)nc(-c3cccc4c3oc3ccccc34)n2)cc1 ; CC1(C)c2ccccc2-c2cc(-c3nc(-c4cccc(-c5cccc6ccc7ccccc7c56)c4)nc(-c4cccc5c4oc4ccccc45)n3)ccc21]

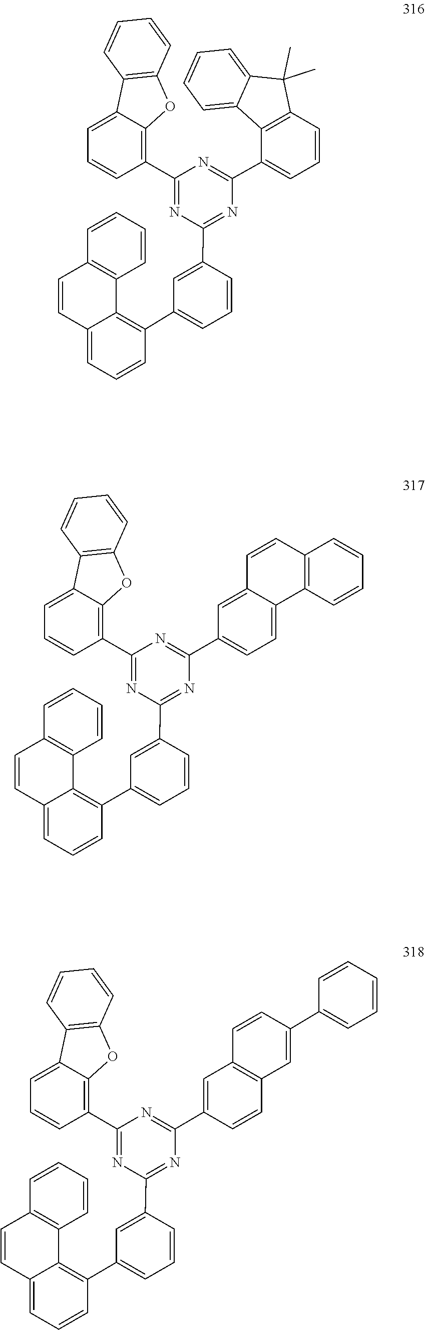 [SMILES: CC1(C)c2ccccc2-c2c(-c3nc(-c4cccc(-c5cccc6ccc7ccccc7c56)c4)nc(-c4cccc5c4oc4ccccc45)n3)cccc21 ; c1cc(-c2nc(-c3ccc4c(ccc5ccccc54)c3)nc(-c3cccc4c3oc3ccccc34)n2)cc(-c2cccc3ccc4ccccc4c23)c1 ; c1ccc(-c2ccc3cc(-c4nc(-c5cccc(-c6cccc7ccc8ccccc8c67)c5)nc(-c5cccc6c5oc5ccccc56)n4)ccc3c2)cc1]

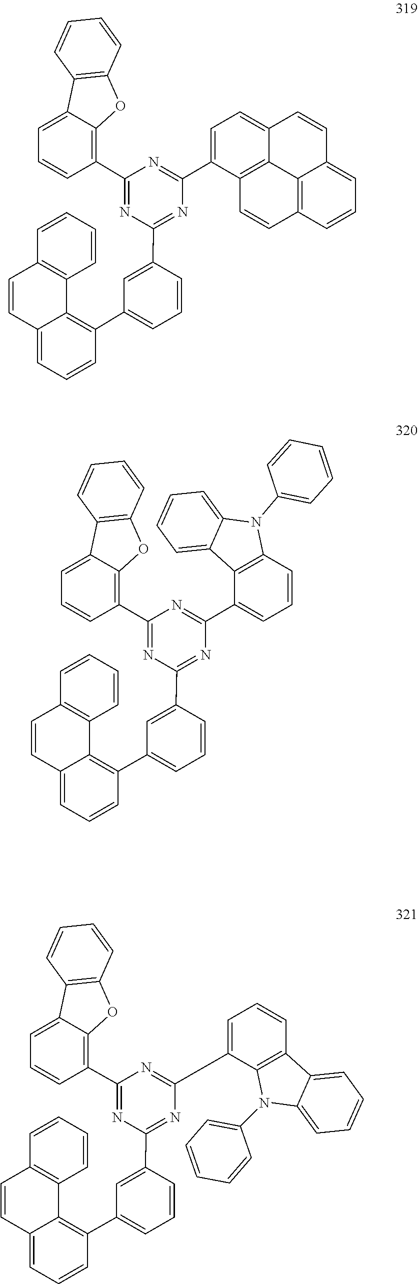 [SMILES: c1cc(-c2nc(-c3ccc4ccc5cccc6ccc3c4c56)nc(-c3cccc4c3oc3ccccc34)n2)cc(-c2cccc3ccc4ccccc4c23)c1 ; c1ccc(-n2c3ccccc3c3c(-c4nc(-c5cccc(-c6cccc7ccc8ccccc8c67)c5)nc(-c5cccc6c5oc5ccccc56)n4)cccc32)cc1 ; c1ccc(-n2c3ccccc3c3cccc(-c4nc(-c5cccc(-c6cccc7ccc8ccccc8c67)c5)nc(-c5cccc6c5oc5ccccc56)n4)c32)cc1]

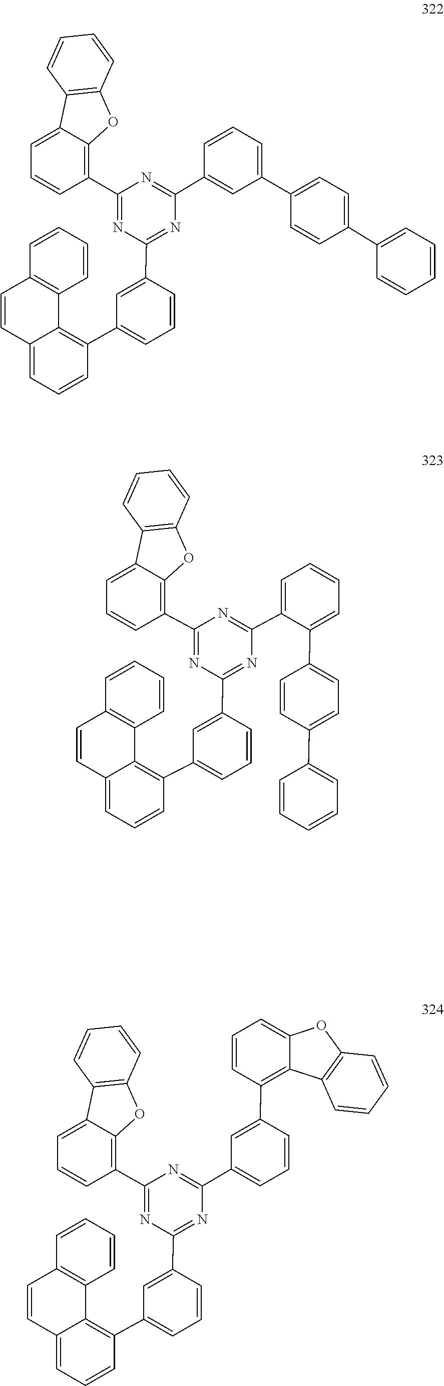 [SMILES: c1ccc(-c2ccc(-c3cccc(-c4nc(-c5cccc(-c6cccc7ccc8ccccc8c67)c5)nc(-c5cccc6c5oc5ccccc56)n4)c3)cc2)cc1 ; c1ccc(-c2ccc(-c3ccccc3-c3nc(-c4cccc(-c5cccc6ccc7ccccc7c56)c4)nc(-c4cccc5c4oc4ccccc45)n3)cc2)cc1 ; c1cc(-c2nc(-c3cccc(-c4cccc5oc6ccccc6c45)c3)nc(-c3cccc4c3oc3ccccc34)n2)cc(-c2cccc3ccc4ccccc4c23)c1]

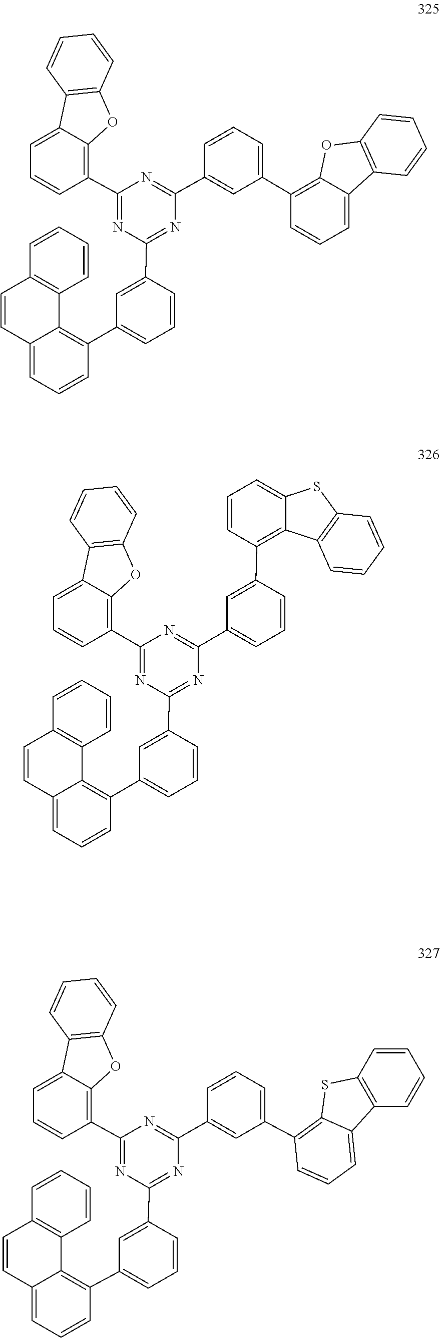 [SMILES: c1cc(-c2nc(-c3cccc(-c4cccc5ccc6ccccc6c45)c3)nc(-c3cccc4c3oc3ccccc34)n2)cc(-c2cccc3c2oc2ccccc23)c1 ; c1cc(-c2nc(-c3cccc(-c4cccc5sc6ccccc6c45)c3)nc(-c3cccc4c3oc3ccccc34)n2)cc(-c2cccc3ccc4ccccc4c23)c1 ; c1cc(-c2nc(-c3cccc(-c4cccc5ccc6ccccc6c45)c3)nc(-c3cccc4c3oc3ccccc34)n2)cc(-c2cccc3c2sc2ccccc23)c1]

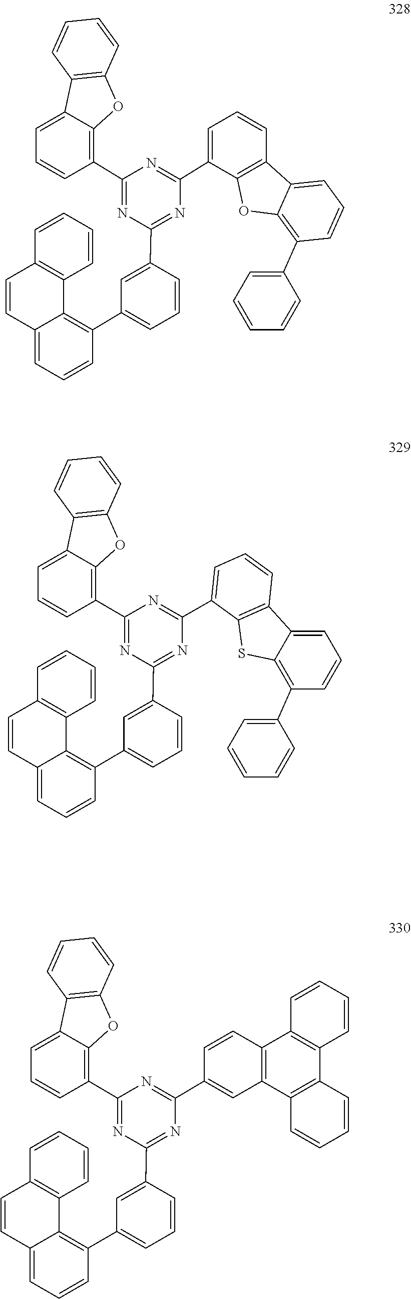 [SMILES: c1ccc(-c2cccc3c2oc2c(-c4nc(-c5cccc(-c6cccc7ccc8ccccc8c67)c5)nc(-c5cccc6c5oc5ccccc56)n4)cccc23)cc1 ; c1ccc(-c2cccc3c2sc2c(-c4nc(-c5cccc(-c6cccc7ccc8ccccc8c67)c5)nc(-c5cccc6c5oc5ccccc56)n4)cccc23)cc1 ; c1cc(-c2nc(-c3ccc4c5ccccc5c5ccccc5c4c3)nc(-c3cccc4c3oc3ccccc34)n2)cc(-c2cccc3ccc4ccccc4c23)c1]

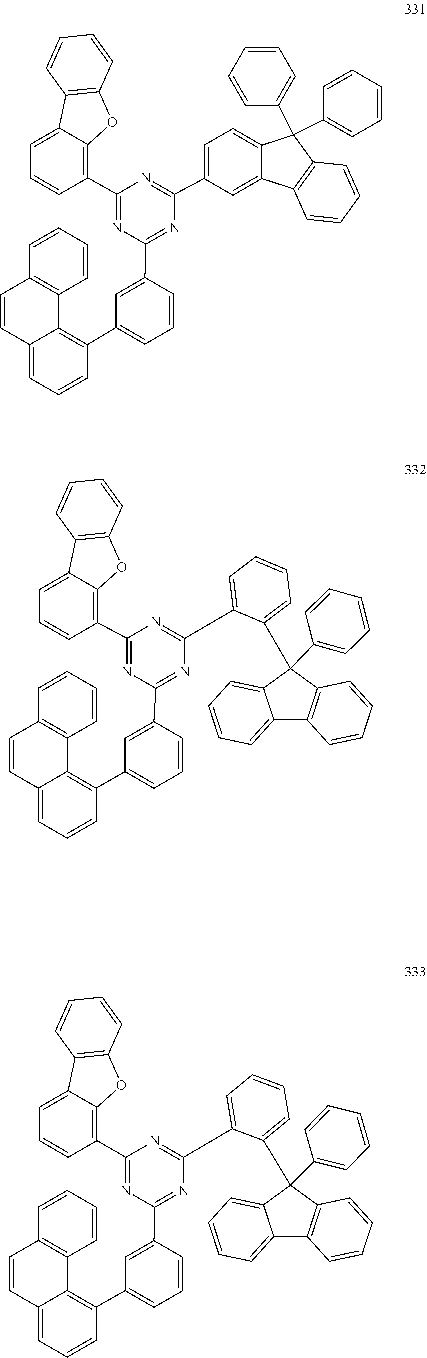 [SMILES: c1ccc(C2(c3ccccc3)c3ccccc3-c3cc(-c4nc(-c5cccc(-c6cccc7ccc8ccccc8c67)c5)nc(-c5cccc6c5oc5ccccc56)n4)ccc32)cc1 ; c1ccc(C2(c3ccccc3-c3nc(-c4cccc(-c5cccc6ccc7ccccc7c56)c4)nc(-c4cccc5c4oc4ccccc45)n3)c3ccccc3-c3ccccc32)cc1 ; c1ccc(C2(c3ccccc3-c3nc(-c4cccc(-c5cccc6ccc7ccccc7c56)c4)nc(-c4cccc5c4oc4ccccc45)n3)c3ccccc3-c3ccccc32)cc1]

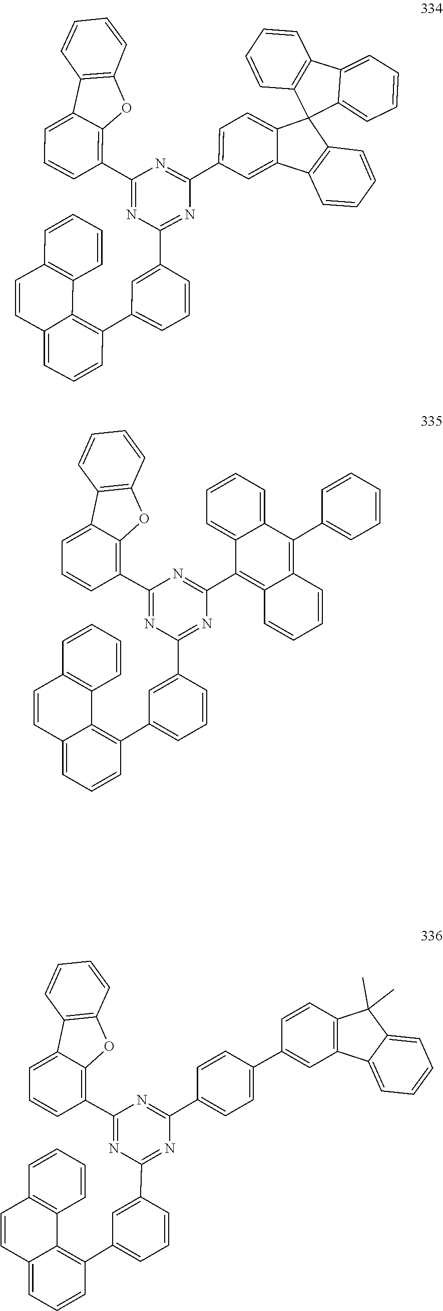 [SMILES: c1cc(-c2nc(-c3ccc4c(c3)-c3ccccc3C43c4ccccc4-c4ccccc43)nc(-c3cccc4c3oc3ccccc34)n2)cc(-c2cccc3ccc4ccccc4c23)c1 ; c1ccc(-c2c3ccccc3c(-c3nc(-c4cccc(-c5cccc6ccc7ccccc7c56)c4)nc(-c4cccc5c4oc4ccccc45)n3)c3ccccc23)cc1 ; CC1(C)c2ccccc2-c2cc(-c3ccc(-c4nc(-c5cccc(-c6cccc7ccc8ccccc8c67)c5)nc(-c5cccc6c5oc5ccccc56)n4)cc3)ccc21]

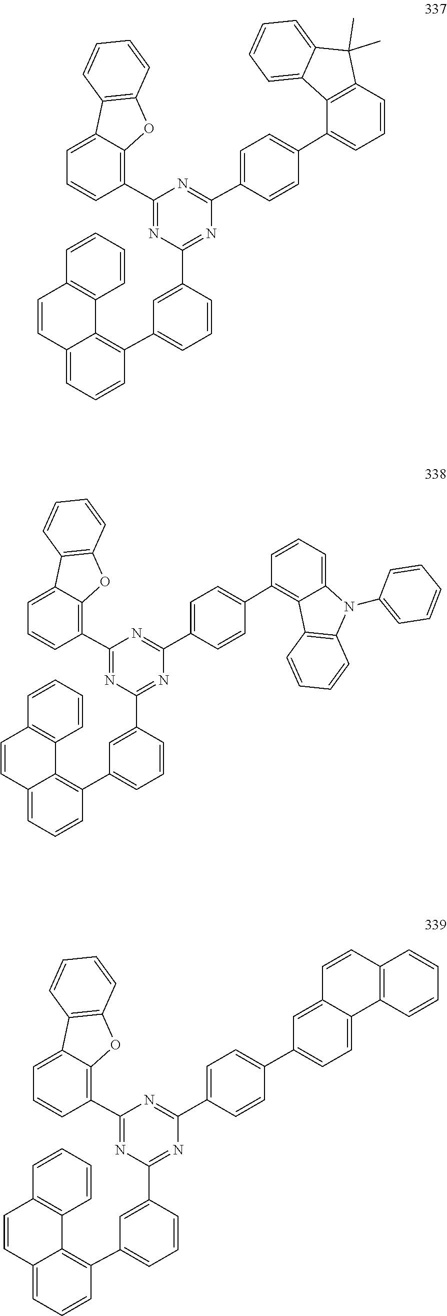 [SMILES: CC1(C)c2ccccc2-c2c(-c3ccc(-c4nc(-c5cccc(-c6cccc7ccc8ccccc8c67)c5)nc(-c5cccc6c5oc5ccccc56)n4)cc3)cccc21 ; c1ccc(-n2c3ccccc3c3c(-c4ccc(-c5nc(-c6cccc(-c7cccc8ccc9ccccc9c78)c6)nc(-c6cccc7c6oc6ccccc67)n5)cc4)cccc32)cc1 ; c1cc(-c2nc(-c3ccc(-c4ccc5c(ccc6ccccc65)c4)cc3)nc(-c3cccc4c3oc3ccccc34)n2)cc(-c2cccc3ccc4ccccc4c23)c1]

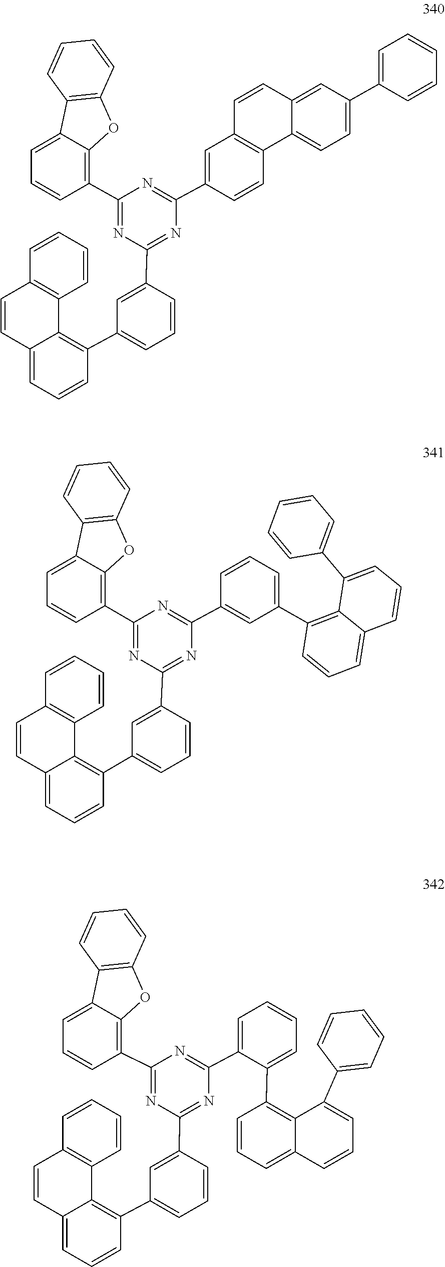 [SMILES: c1ccc(-c2ccc3c(ccc4cc(-c5nc(-c6cccc(-c7cccc8ccc9ccccc9c78)c6)nc(-c6cccc7c6oc6ccccc67)n5)ccc43)c2)cc1 ; c1ccc(-c2cccc3cccc(-c4cccc(-c5nc(-c6cccc(-c7cccc8ccc9ccccc9c78)c6)nc(-c6cccc7c6oc6ccccc67)n5)c4)c23)cc1 ; c1ccc(-c2cccc3cccc(-c4ccccc4-c4nc(-c5cccc(-c6cccc7ccc8ccccc8c67)c5)nc(-c5cccc6c5oc5ccccc56)n4)c23)cc1]

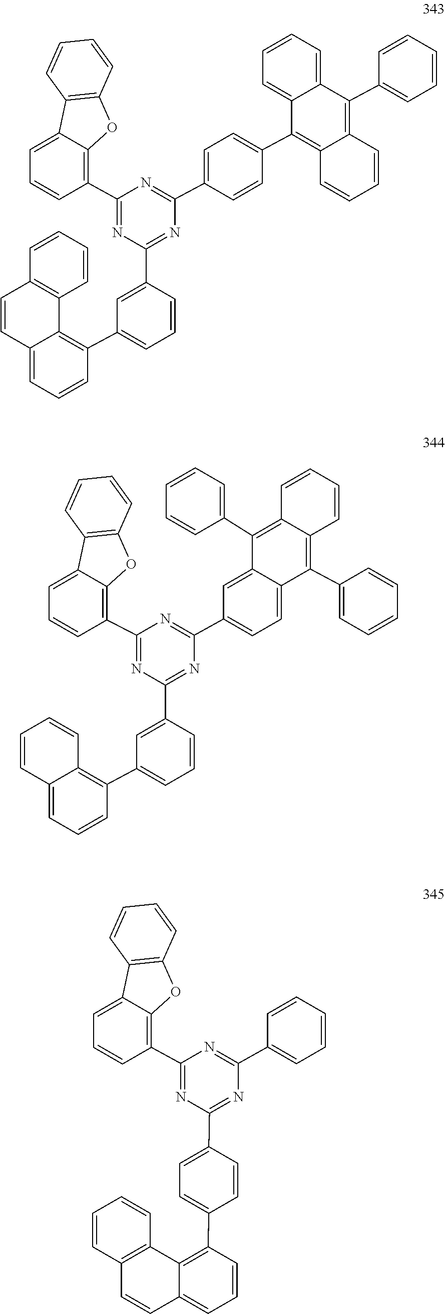 [SMILES: c1ccc(-c2c3ccccc3c(-c3ccc(-c4nc(-c5cccc(-c6cccc7ccc8ccccc8c67)c5)nc(-c5cccc6c5oc5ccccc56)n4)cc3)c3ccccc23)cc1 ; c1ccc(-c2c3ccccc3c(-c3ccccc3)c3cc(-c4nc(-c5cccc(-c6cccc7ccccc67)c5)nc(-c5cccc6c5oc5ccccc56)n4)ccc23)cc1 ; c1ccc(-c2nc(-c3ccc(-c4cccc5ccc6ccccc6c45)cc3)nc(-c3cccc4c3oc3ccccc34)n2)cc1]

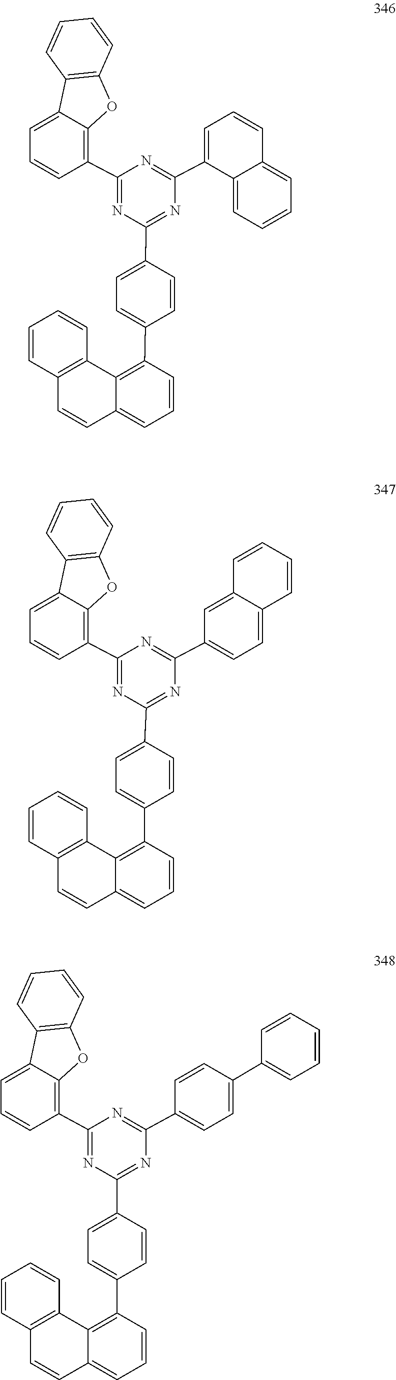 [SMILES: c1ccc2c(-c3nc(-c4ccc(-c5cccc6ccc7ccccc7c56)cc4)nc(-c4cccc5c4oc4ccccc45)n3)cccc2c1 ; c1ccc2cc(-c3nc(-c4ccc(-c5cccc6ccc7ccccc7c56)cc4)nc(-c4cccc5c4oc4ccccc45)n3)ccc2c1 ; c1ccc(-c2ccc(-c3nc(-c4ccc(-c5cccc6ccc7ccccc7c56)cc4)nc(-c4cccc5c4oc4ccccc45)n3)cc2)cc1]

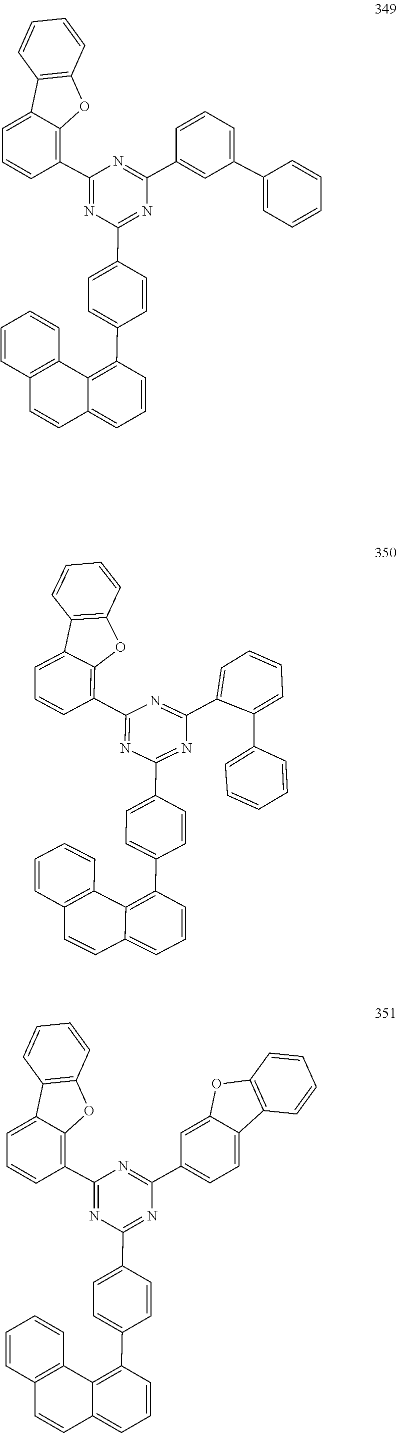 [SMILES: c1ccc(-c2cccc(-c3nc(-c4ccc(-c5cccc6ccc7ccccc7c56)cc4)nc(-c4cccc5c4oc4ccccc45)n3)c2)cc1 ; c1ccc(-c2ccccc2-c2nc(-c3ccc(-c4cccc5ccc6ccccc6c45)cc3)nc(-c3cccc4c3oc3ccccc34)n2)cc1 ; c1ccc2c(c1)ccc1cccc(-c3ccc(-c4nc(-c5ccc6c(c5)oc5ccccc56)nc(-c5cccc6c5oc5ccccc56)n4)cc3)c12]

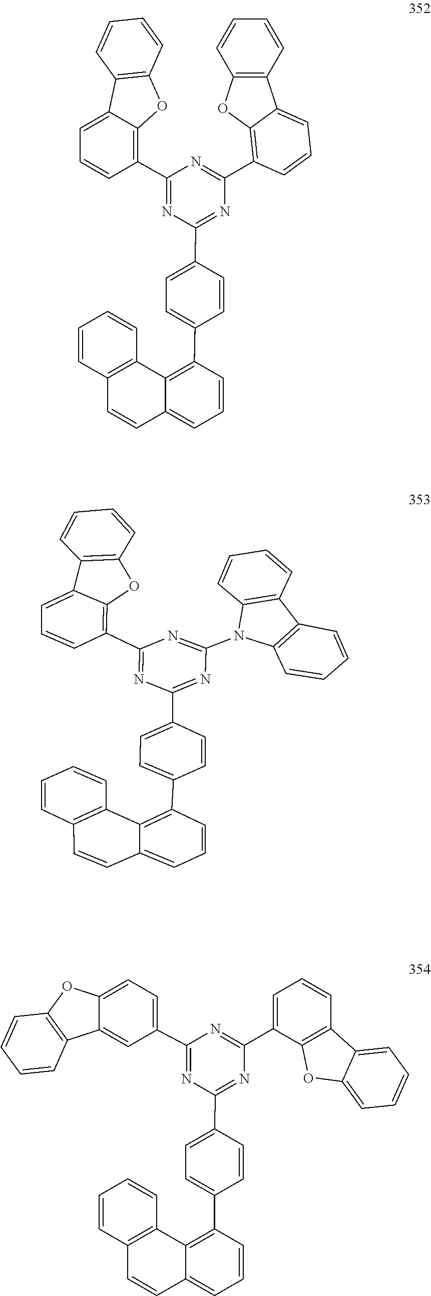 [SMILES: c1ccc2c(c1)ccc1cccc(-c3ccc(-c4nc(-c5cccc6c5oc5ccccc56)nc(-c5cccc6c5oc5ccccc56)n4)cc3)c12 ; c1ccc2c(c1)ccc1cccc(-c3ccc(-c4nc(-c5cccc6c5oc5ccccc56)nc(-n5c6ccccc6c6ccccc65)n4)cc3)c12 ; c1ccc2c(c1)ccc1cccc(-c3ccc(-c4nc(-c5ccc6oc7ccccc7c6c5)nc(-c5cccc6c5oc5ccccc56)n4)cc3)c12]

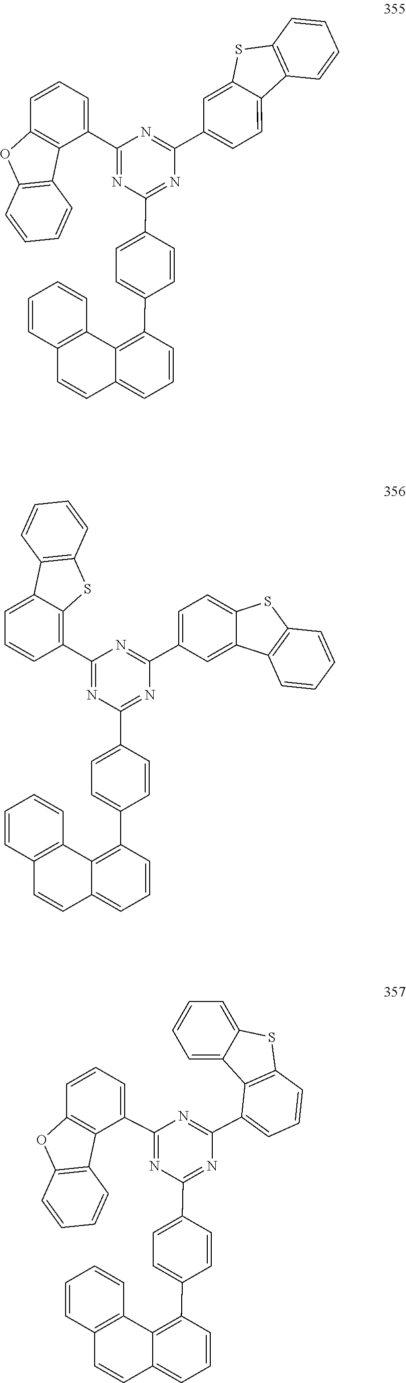 [SMILES: c1ccc2c(c1)ccc1cccc(-c3ccc(-c4nc(-c5ccc6c(c5)sc5ccccc56)nc(-c5cccc6oc7ccccc7c56)n4)cc3)c12 ; c1ccc2c(c1)ccc1cccc(-c3ccc(-c4nc(-c5ccc6sc7ccccc7c6c5)nc(-c5cccc6c5sc5ccccc56)n4)cc3)c12 ; c1ccc2c(c1)ccc1cccc(-c3ccc(-c4nc(-c5cccc6oc7ccccc7c56)nc(-c5cccc6sc7ccccc7c56)n4)cc3)c12]

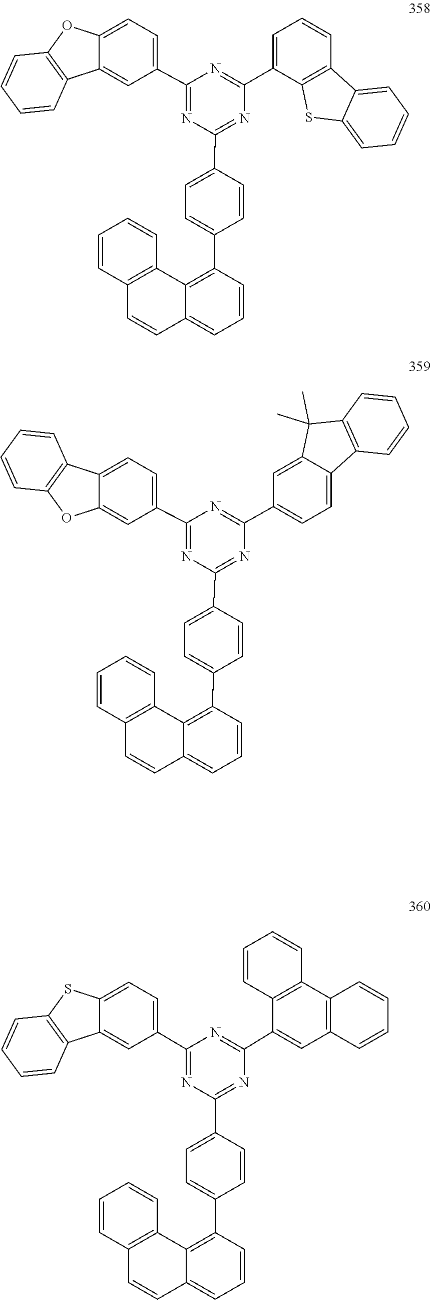 [SMILES: c1ccc2c(c1)ccc1cccc(-c3ccc(-c4nc(-c5ccc6oc7ccccc7c6c5)nc(-c5cccc6c5sc5ccccc56)n4)cc3)c12 ; CC1(C)c2ccccc2-c2ccc(-c3nc(-c4ccc(-c5cccc6ccc7ccccc7c56)cc4)nc(-c4ccc5c(c4)oc4ccccc45)n3)cc21 ; c1ccc2c(c1)cc(-c1nc(-c3ccc(-c4cccc5ccc6ccccc6c45)cc3)nc(-c3ccc4sc5ccccc5c4c3)n1)c1ccccc12]

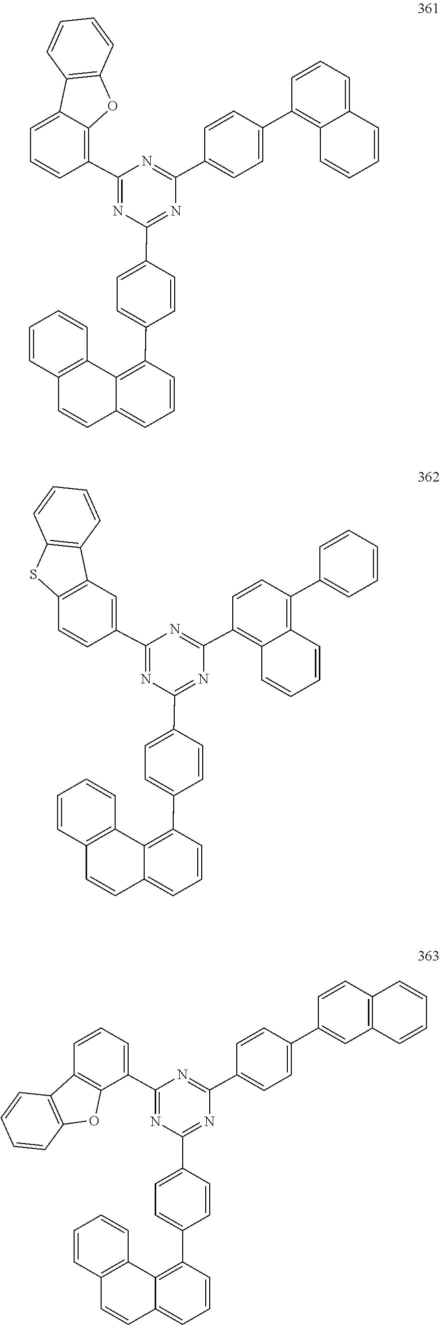 [SMILES: c1ccc2c(-c3ccc(-c4nc(-c5ccc(-c6cccc7ccc8ccccc8c67)cc5)nc(-c5cccc6c5oc5ccccc56)n4)cc3)cccc2c1 ; c1ccc(-c2ccc(-c3nc(-c4ccc(-c5cccc6ccc7ccccc7c56)cc4)nc(-c4ccc5sc6ccccc6c5c4)n3)c3ccccc23)cc1 ; c1ccc2cc(-c3ccc(-c4nc(-c5ccc(-c6cccc7ccc8ccccc8c67)cc5)nc(-c5cccc6c5oc5ccccc56)n4)cc3)ccc2c1]

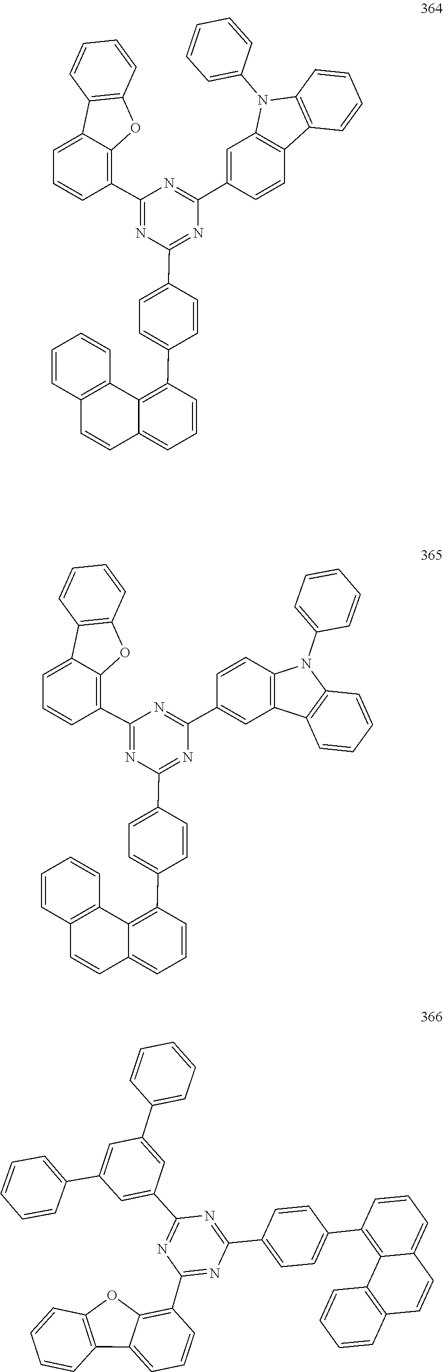 [SMILES: c1ccc(-n2c3ccccc3c3ccc(-c4nc(-c5ccc(-c6cccc7ccc8ccccc8c67)cc5)nc(-c5cccc6c5oc5ccccc56)n4)cc32)cc1 ; c1ccc(-n2c3ccccc3c3cc(-c4nc(-c5ccc(-c6cccc7ccc8ccccc8c67)cc5)nc(-c5cccc6c5oc5ccccc56)n4)ccc32)cc1 ; c1ccc(-c2cc(-c3ccccc3)cc(-c3nc(-c4ccc(-c5cccc6ccc7ccccc7c56)cc4)nc(-c4cccc5c4oc4ccccc45)n3)c2)cc1]

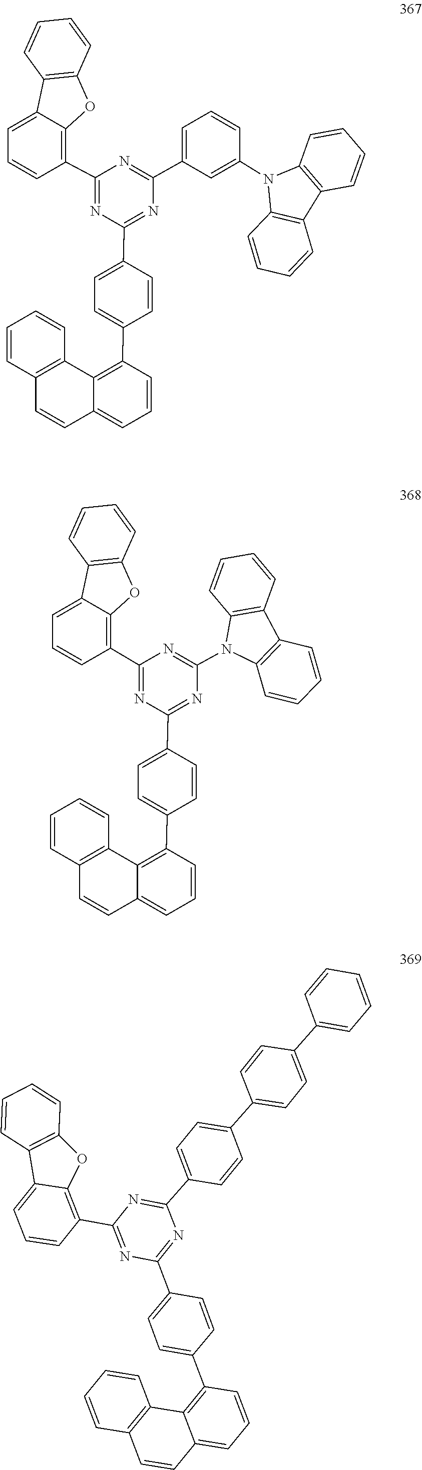 [SMILES: c1cc(-c2nc(-c3ccc(-c4cccc5ccc6ccccc6c45)cc3)nc(-c3cccc4c3oc3ccccc34)n2)cc(-n2c3ccccc3c3ccccc32)c1 ; c1ccc2c(c1)ccc1cccc(-c3ccc(-c4nc(-c5cccc6c5oc5ccccc56)nc(-n5c6ccccc6c6ccccc65)n4)cc3)c12 ; c1ccc(-c2ccc(-c3ccc(-c4nc(-c5ccc(-c6cccc7ccc8ccccc8c67)cc5)nc(-c5cccc6c5oc5ccccc56)n4)cc3)cc2)cc1]

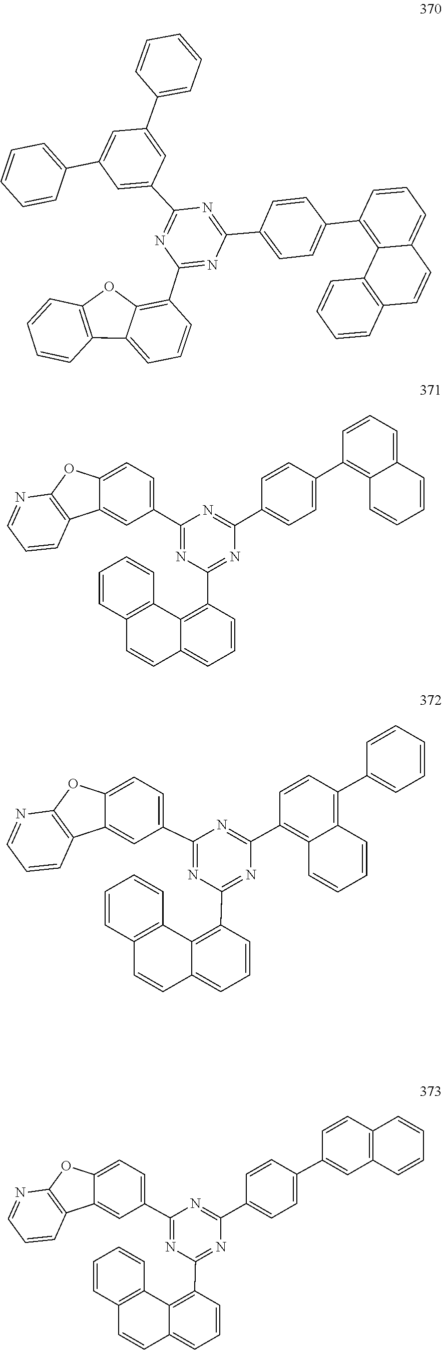 [SMILES: c1ccc(-c2cc(-c3ccccc3)cc(-c3nc(-c4ccc(-c5cccc6ccc7ccccc7c56)cc4)nc(-c4cccc5c4oc4ccccc45)n3)c2)cc1 ; c1ccc2c(-c3ccc(-c4nc(-c5ccc6oc7ncccc7c6c5)nc(-c5cccc6ccc7ccccc7c56)n4)cc3)cccc2c1 ; c1ccc(-c2ccc(-c3nc(-c4ccc5oc6ncccc6c5c4)nc(-c4cccc5ccc6ccccc6c45)n3)c3ccccc23)cc1 ; c1ccc2cc(-c3ccc(-c4nc(-c5ccc6oc7ncccc7c6c5)nc(-c5cccc6ccc7ccccc7c56)n4)cc3)ccc2c1]

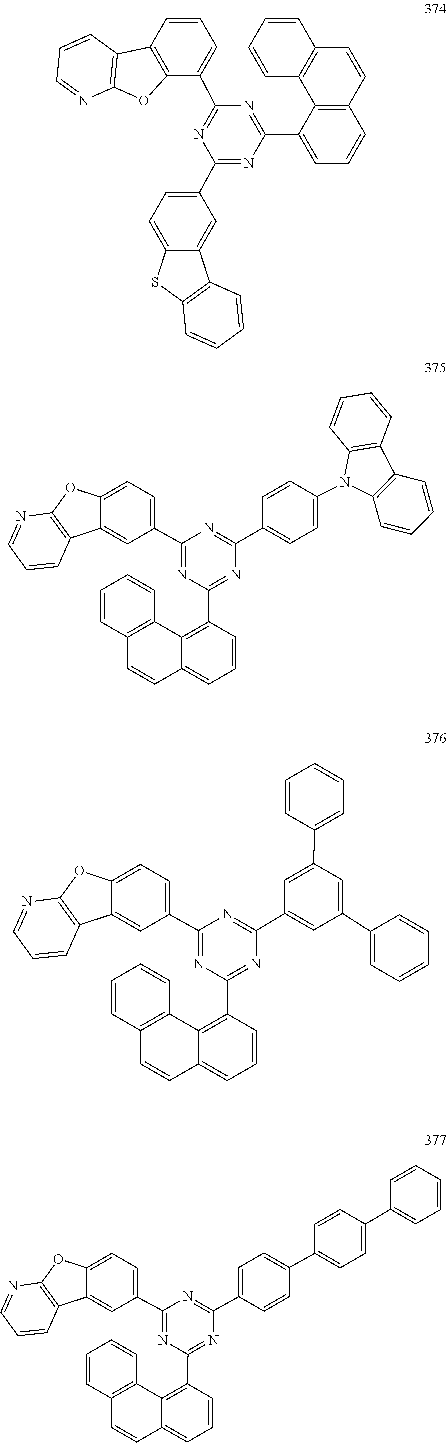 [SMILES: c1ccc2c(c1)ccc1cccc(-c3nc(-c4ccc5sc6ccccc6c5c4)nc(-c4cccc5c4oc4ncccc45)n3)c12 ; c1ccc2c(c1)ccc1cccc(-c3nc(-c4ccc(-n5c6ccccc6c6ccccc65)cc4)nc(-c4ccc5oc6ncccc6c5c4)n3)c12 ; c1ccc(-c2cc(-c3ccccc3)cc(-c3nc(-c4ccc5oc6ncccc6c5c4)nc(-c4cccc5ccc6ccccc6c45)n3)c2)cc1 ; c1ccc(-c2ccc(-c3ccc(-c4nc(-c5ccc6oc7ncccc7c6c5)nc(-c5cccc6ccc7ccccc7c56)n4)cc3)cc2)cc1]

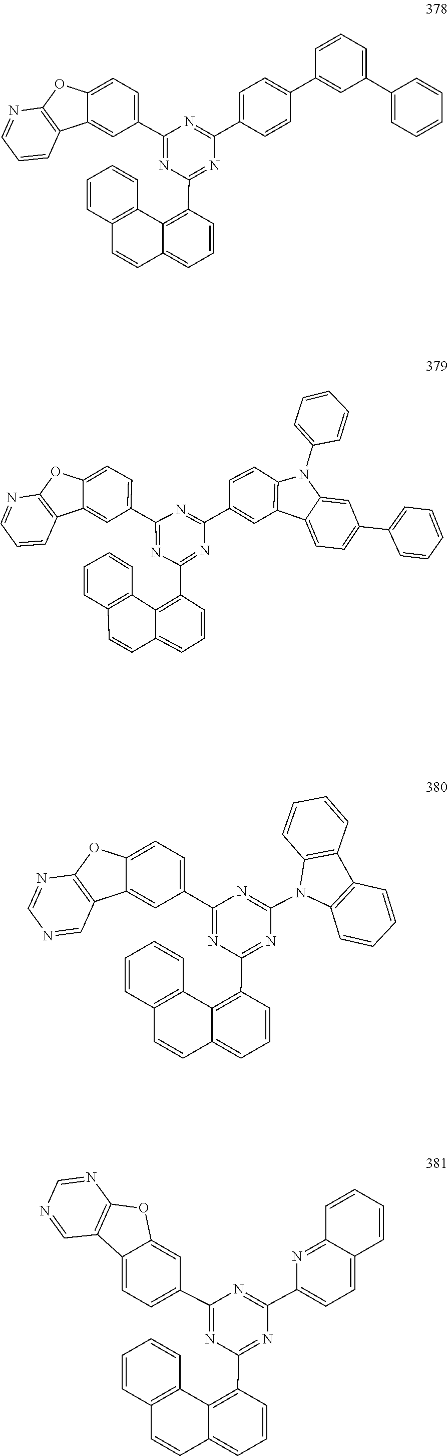 [SMILES: c1ccc(-c2cccc(-c3ccc(-c4nc(-c5ccc6oc7ncccc7c6c5)nc(-c5cccc6ccc7ccccc7c56)n4)cc3)c2)cc1 ; c1ccc(-c2ccc3c4cc(-c5nc(-c6ccc7oc8ncccc8c7c6)nc(-c6cccc7ccc8ccccc8c67)n5)ccc4n(-c4ccccc4)c3c2)cc1 ; c1ccc2c(c1)ccc1cccc(-c3nc(-c4ccc5oc6ncncc6c5c4)nc(-n4c5ccccc5c5ccccc54)n3)c12 ; c1ccc2nc(-c3nc(-c4ccc5c(c4)oc4ncncc45)nc(-c4cccc5ccc6ccccc6c45)n3)ccc2c1]

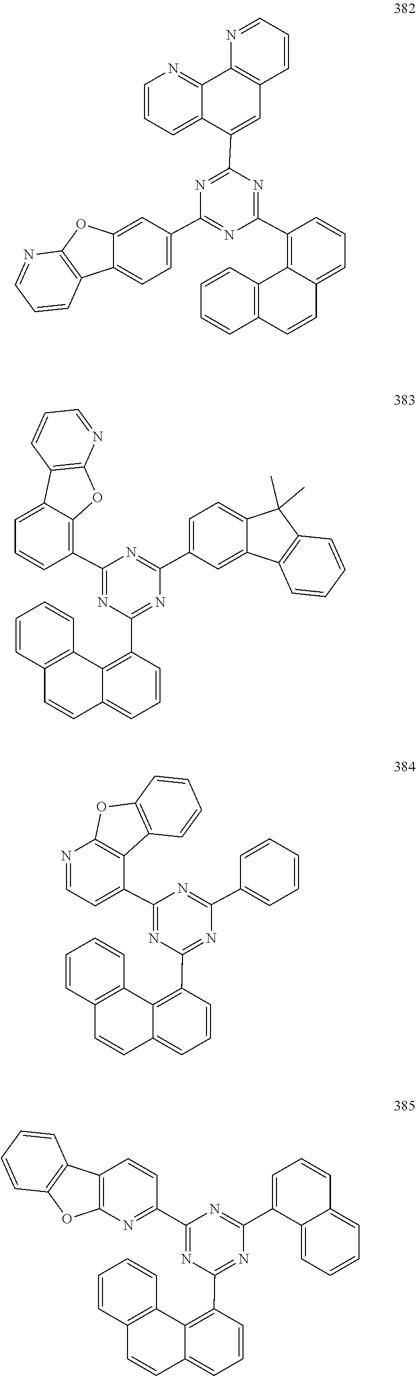 [SMILES: c1ccc2c(c1)ccc1cccc(-c3nc(-c4ccc5c(c4)oc4ncccc45)nc(-c4cc5cccnc5c5ncccc45)n3)c12 ; CC1(C)c2ccccc2-c2cc(-c3nc(-c4cccc5c4oc4ncccc45)nc(-c4cccc5ccc6ccccc6c45)n3)ccc21 ; c1ccc(-c2nc(-c3cccc4ccc5ccccc5c34)nc(-c3ccnc4oc5ccccc5c34)n2)cc1 ; c1ccc2c(-c3nc(-c4ccc5c(n4)oc4ccccc45)nc(-c4cccc5ccc6ccccc6c45)n3)cccc2c1]

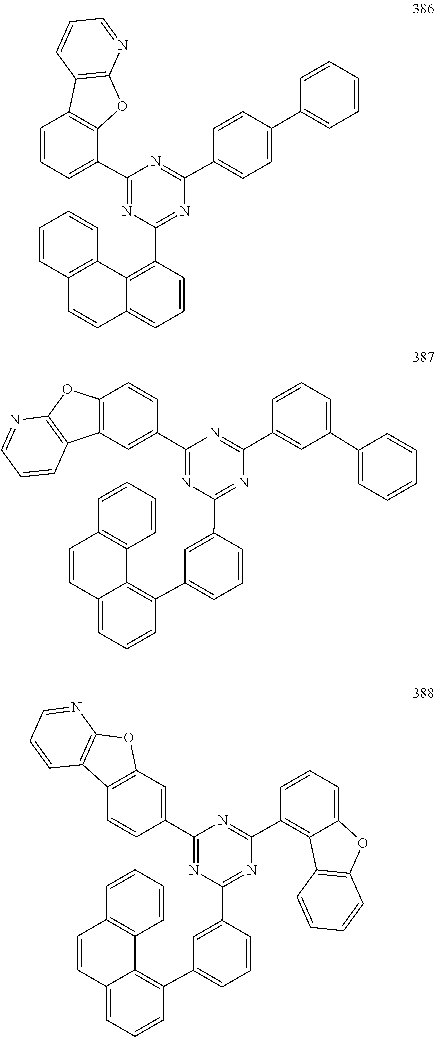 [SMILES: c1ccc(-c2ccc(-c3nc(-c4cccc5c4oc4ncccc45)nc(-c4cccc5ccc6ccccc6c45)n3)cc2)cc1 ; c1ccc(-c2cccc(-c3nc(-c4cccc(-c5cccc6ccc7ccccc7c56)c4)nc(-c4ccc5oc6ncccc6c5c4)n3)c2)cc1 ; c1cc(-c2nc(-c3ccc4c(c3)oc3ncccc34)nc(-c3cccc4oc5ccccc5c34)n2)cc(-c2cccc3ccc4ccccc4c23)c1]

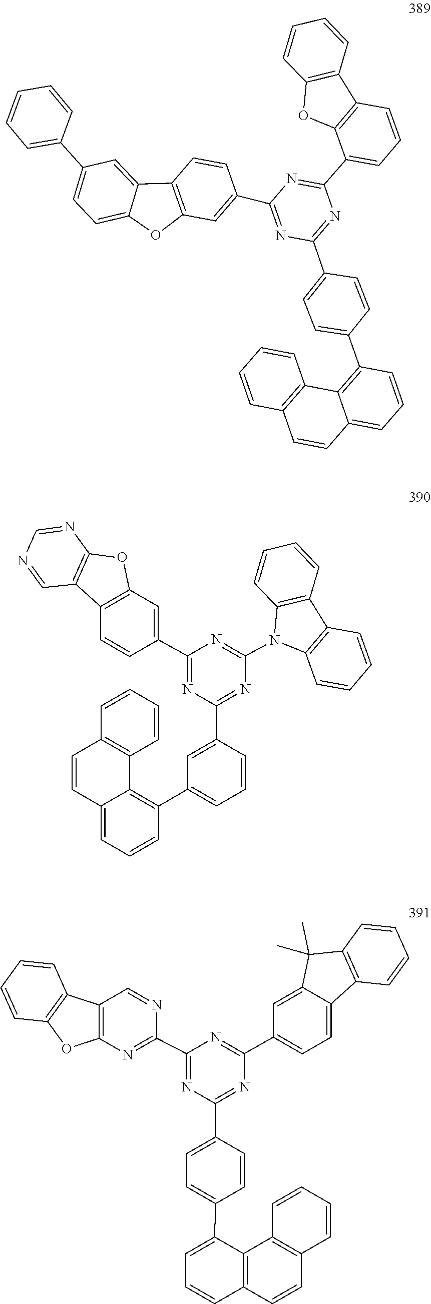 [SMILES: c1ccc(-c2ccc3oc4cc(-c5nc(-c6ccc(-c7cccc8ccc9ccccc9c78)cc6)nc(-c6cccc7c6oc6ccccc67)n5)ccc4c3c2)cc1 ; c1cc(-c2nc(-c3ccc4c(c3)oc3ncncc34)nc(-n3c4ccccc4c4ccccc43)n2)cc(-c2cccc3ccc4ccccc4c23)c1 ; CC1(C)c2ccccc2-c2ccc(-c3nc(-c4ccc(-c5cccc6ccc7ccccc7c56)cc4)nc(-c4ncc5c(n4)oc4ccccc45)n3)cc21]

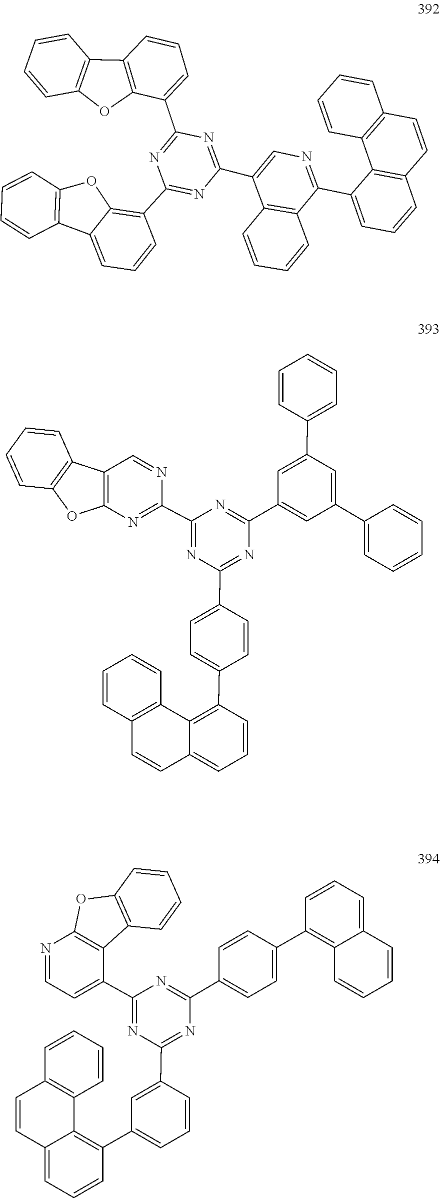 [SMILES: c1ccc2c(c1)ccc1cccc(-c3ncc(-c4nc(-c5cccc6c5oc5ccccc56)nc(-c5cccc6c5oc5ccccc56)n4)c4ccccc34)c12 ; c1ccc(-c2cc(-c3ccccc3)cc(-c3nc(-c4ccc(-c5cccc6ccc7ccccc7c56)cc4)nc(-c4ncc5c(n4)oc4ccccc45)n3)c2)cc1 ; c1cc(-c2nc(-c3ccc(-c4cccc5ccccc45)cc3)nc(-c3ccnc4oc5ccccc5c34)n2)cc(-c2cccc3ccc4ccccc4c23)c1]

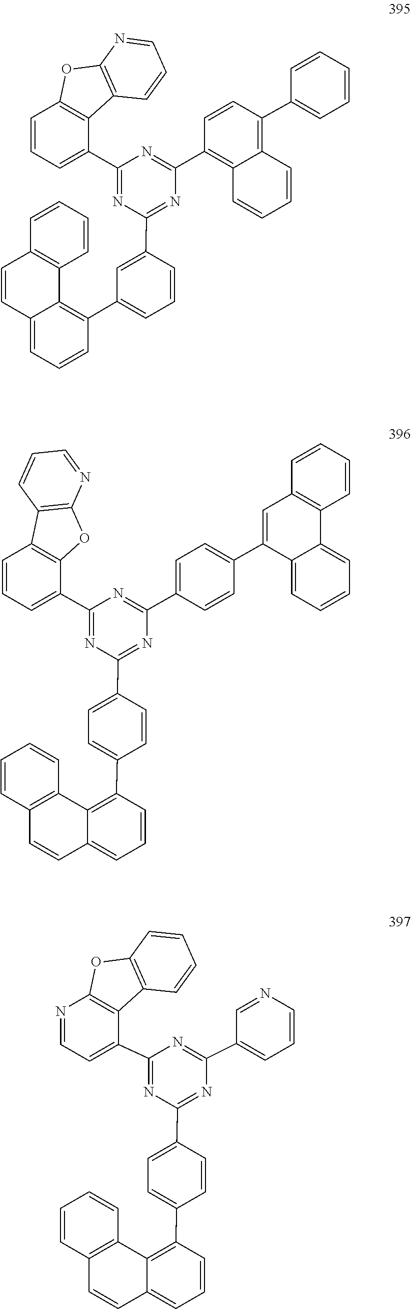 [SMILES: c1ccc(-c2ccc(-c3nc(-c4cccc(-c5cccc6ccc7ccccc7c56)c4)nc(-c4cccc5oc6ncccc6c45)n3)c3ccccc23)cc1 ; c1ccc2c(c1)cc(-c1ccc(-c3nc(-c4ccc(-c5cccc6ccc7ccccc7c56)cc4)nc(-c4cccc5c4oc4ncccc45)n3)cc1)c1ccccc12 ; c1cncc(-c2nc(-c3ccc(-c4cccc5ccc6ccccc6c45)cc3)nc(-c3ccnc4oc5ccccc5c34)n2)c1]

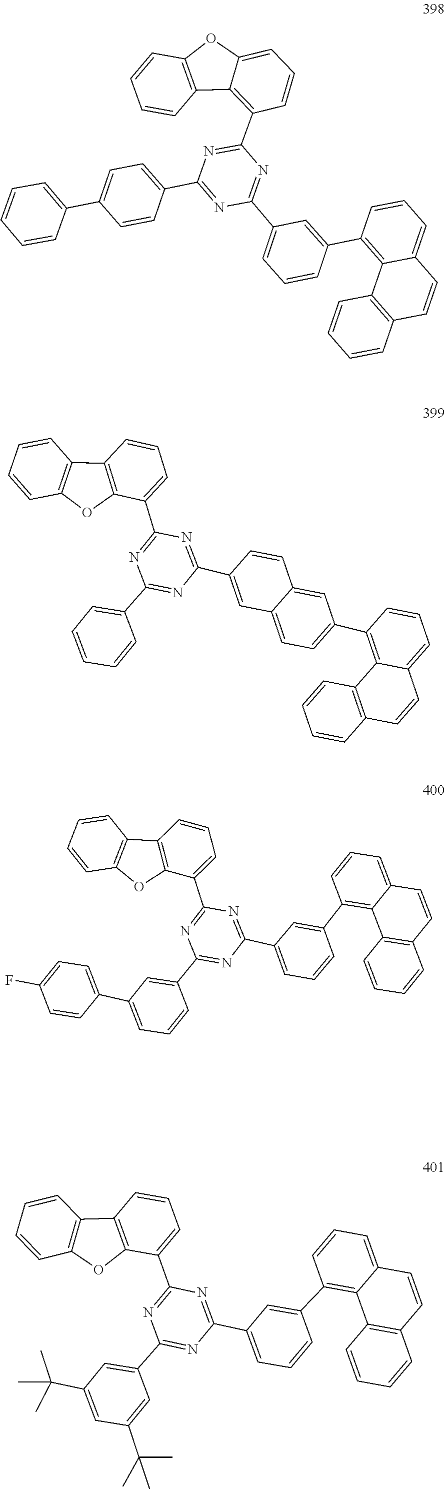 [SMILES: c1ccc(-c2ccc(-c3nc(-c4cccc(-c5cccc6ccc7ccccc7c56)c4)nc(-c4cccc5oc6ccccc6c45)n3)cc2)cc1 ; c1ccc(-c2nc(-c3ccc4cc(-c5cccc6ccc7ccccc7c56)ccc4c3)nc(-c3cccc4c3oc3ccccc34)n2)cc1 ; Fc1ccc(-c2cccc(-c3nc(-c4cccc(-c5cccc6ccc7ccccc7c56)c4)nc(-c4cccc5c4oc4ccccc45)n3)c2)cc1 ; CC(C)(C)c1cc(-c2nc(-c3cccc(-c4cccc5ccc6ccccc6c45)c3)nc(-c3cccc4c3oc3ccccc34)n2)cc(C(C)(C)C)c1]

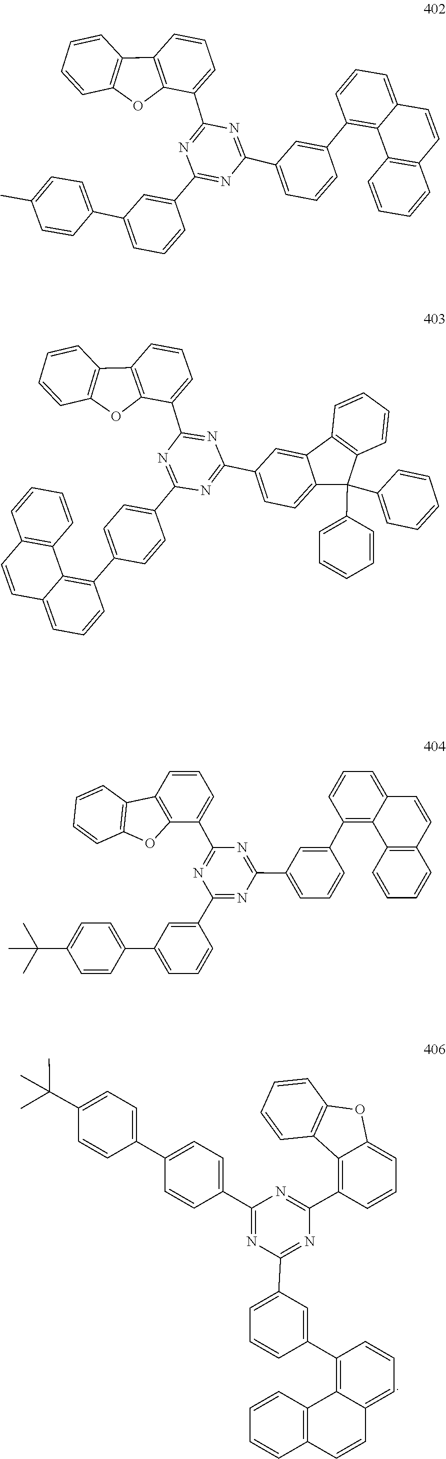 [SMILES: Cc1ccc(-c2cccc(-c3nc(-c4cccc(-c5cccc6ccc7ccccc7c56)c4)nc(-c4cccc5c4oc4ccccc45)n3)c2)cc1 ; c1ccc(C2(c3ccccc3)c3ccccc3-c3cc(-c4nc(-c5ccc(-c6cccc7ccc8ccccc8c67)cc5)nc(-c5cccc6c5oc5ccccc56)n4)ccc32)cc1 ; CC(C)(C)c1ccc(-c2cccc(-c3nc(-c4cccc(-c5cccc6ccc7ccccc7c56)c4)nc(-c4cccc5c4oc4ccccc45)n3)c2)cc1 ; CC(C)(C)c1ccc(-c2ccc(-c3nc(-c4cccc(-c5cccc6ccc7ccccc7c56)c4)nc(-c4cccc5oc6ccccc6c45)n3)cc2)cc1]

The present disclosure also provides an organic electroluminescent device including an anode and a cathode which are oppositely disposed, and a functional layer disposed between the anode and the cathode; and the functional layer includes the nitrogen-containing compound of the present disclosure.

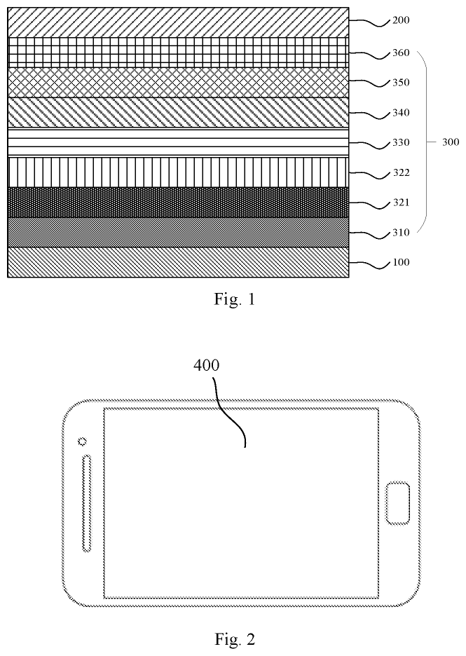

For example, as shown in FIG. 1, the organic electroluminescent device includes an anode 100 and a cathode 200 which are disposed oppositely, and a functional layer 300 disposed between the anode 100 and the cathode 200; and the functional layer 300 includes the nitrogen-containing compound provided by the present disclosure.

According to one embodiment, the organic electroluminescent device can be, for example, a green organic electroluminescent device.

Optionally, the functional layer 300 includes an organic electroluminescent layer 330, and the organic electroluminescent layer 330 includes the nitrogen-containing compound of the present disclosure.

Optionally, the organic electroluminescent layer 330 may be composed of a single light-emitting material and may also include a host material and a guest material. Optionally, the organic electroluminescent layer 330 is composed of the host material and the guest material, holes injected into the organic electroluminescent layer 330 and electrons injected into the organic electroluminescent layer 330 may be recombined in the organic electroluminescent layer 330 to form excitons, the excitons transfer energy to the host material, and the host material transfers energy to the guest material, so that the guest material can emit light.

The host material of the organic electroluminescent layer 330 may be a metal chelate compound, a bis-styryl derivative, an aromatic amine derivative, a dibenzofuran derivative, or other types of materials, which is not specially limited in the present disclosure. In one embodiment of the present disclosure, the host material of the organic electroluminescent layer 330 is a mixture of the compound of the present disclosure with other compounds, such as GH-P1.

The guest material of the organic electroluminescent layer 330 may be a compound having a condensed aryl ring or its derivative, a compound having a heteroaryl ring or its derivative, an aromatic amine derivative, or other materials, and in one embodiment of the present disclosure, the guest material of the organic electroluminescent layer 330 is Ir(npy)₂acac.

In one example of the present disclosure, the organic electroluminescent device may include an anode 100, a hole transport layer 321, an electron blocking layer 322, an organic electroluminescent layer 330 as an energy conversion layer, an electron transport layer 350, and a cathode 200 which are stacked in sequence. The nitrogen-containing compound provided by the present disclosure can be applied to the organic electroluminescent layer 330 of the organic electroluminescent device, which can effectively improve the luminous efficiency and service life of the organic electroluminescent device.

Optionally, the anode 100 includes the following anode materials, which are preferably materials having a large work function that facilitate hole injection into the functional layer. Specific examples of the anode materials include metals such as nickel, platinum, vanadium, chromium, copper, zinc, and gold or their alloys; metal oxides such as zinc oxide, indium oxide, indium tin oxide (ITO), and indium zinc oxide (IZO); combination of metals and oxides such as ZnO:Al or SnO₂:Sb; or conductive polymers such as poly(3-methylthiophene), poly[3,4-(ethylene-1,2-dioxy)thiophene] (PEDT), polypyrrole, and polyaniline, but are not limited to thereto. It preferably includes a transparent electrode containing indium tin oxide (ITO) as the anode.

Optionally, the hole transport layer 321 can include one or more hole transport materials, and the hole transport materials can be selected from a carbazole polymer, carbazole-connected triarylamine compounds or other types of compounds, which are not specially limited in the present disclosure. For example, in one embodiment of the present disclosure, the hole transport layer 321 is composed of a compound NPB.

Optionally, the electron blocking layer 322 includes one or more electron blocking materials, the electron blocking layer is also referred to as a second hole transport property, and the electron blocking materials may be selected from a carbazole polymer or other types of compounds, which are not specially limited in the present disclosure. For example, in some examples of the present disclosure, the electron blocking layer 322 is composed of TCBPA.

Optionally, the electron transport layer 350 may be of a single-layer structure or a multi-layer structure, which may include one or more electron transport materials, and the electron transport materials are selected from a benzimidazole derivative, an oxadiazole derivative, a quinoxaline derivative, or other electron transport materials, which are not specially limited in the present disclosure. For example, in one embodiment of the present disclosure, the electron transport layer 350 may be composed of TPyQB and LiQ.

Optionally, the cathode 200 includes the following cathode materials, which are materials with a small work function that facilitate electron injection into the functional layer. Specific examples of the cathode materials include metals such as magnesium, calcium, sodium, potassium, titanium, indium, yttrium, lithium, gadolinium, aluminum, silver, tin, and lead or their alloys; or multilayer materials such as LiF/Al, Liq/Al, LiO₂/Al, LiF/Ca, LiF/Al, and BaF₂/Ca, but are not limited to this. A metal electrode containing magnesium and silver as the cathode is preferably included.

Optionally, as shown in FIG. 1, a hole injection layer 310 may also be arranged between the anode 100 and the hole transport layer 321 to enhance the ability to inject holes into the hole transport layer 321. The hole injection layer 310 can be made of a benzidine derivative, a starburst arylamine compound, a phthalocyanine derivative or other materials, which is not specially limited in the present disclosure. In one embodiment of the present disclosure, the hole injection layer 310 may be composed of HAT-CN.

Optionally, as shown in FIG. 1, an electron injection layer 360 may also be arranged between the cathode 200 and the electron transport layer 350 to enhance the ability to inject electrons into the electron transport layer 350. The electron injection layer 360 may include an inorganic material such as an alkali metal sulfide and an alkali metal halide, or may include a complex of an alkali metal and an organic substance. In one embodiment of the present disclosure, the electron injection layer 360 may include Yb (ytterbium).

Optionally, a hole blocking layer 340 may also be arranged between the organic electroluminescent layer 330 and the electron transport layer 350.

Examples of the present disclosure also provide an electronic apparatus, including the organic electroluminescent device described above. Since the electronic apparatus has the above-described organic electroluminescent device, the electronic apparatus has the same beneficial effects, which is not repeated here.

For example, as shown in FIG. 2, the present disclosure provides an electronic apparatus 400, including the organic electroluminescent device described above. The electronic apparatus 400 may be a display device, a lighting device, an optical communication device, or other types of electronic devices, and may include, for example, but is not limited to, a computer screen, a mobile phone screen, a television, electronic paper, an emergency lighting lamp, an optical module, and the like. Since the electronic apparatus 400 has the above-described organic electroluminescent device, the electronic apparatus 400 has the same beneficial effects, which will not be repeated here.

In the following, the present disclosure will be further described in detail by way of examples. However, the following examples are merely illustrative of the present disclosure and are not intended to limit the present disclosure.

### Synthetic Example: Synthesis of Compounds

### Preparation Example 1: Synthesis of Compound 3

1) Synthesis of Intermediate Sub A-1

An intermediate sub A-1 was synthesized by the following synthetic route:

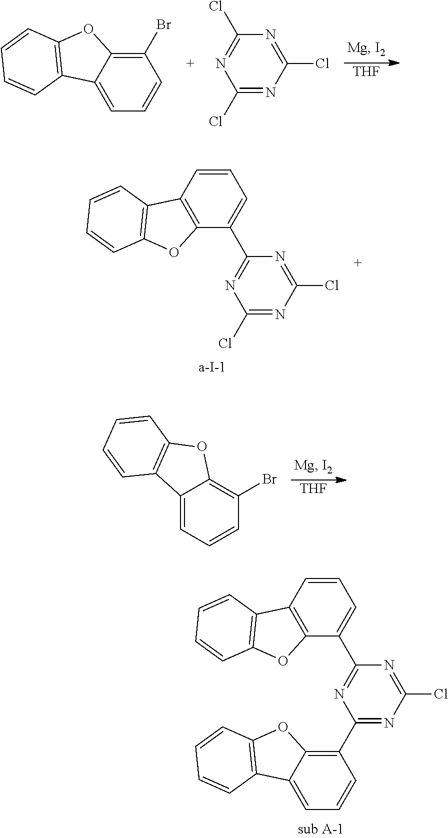

Synthesis of Intermediate a-I-1

Under the protection of N₂, magnesium sheets (2.9 g, 120 mmol) and 30 mL of tetrahydrofuran were added into a three-necked flask, the temperature of the system was raised to 80° C., and a mixture of iodine (0.6 g, 2.4 mmol) and 4-bromodibenzofuran (30.0 g, 120 mmol) dissolved completely in 30 mL of THF was slowly added dropwise to the system where magnesium sheets and tetrahydrofuran reacted within 30 min, and the temperature was controlled at 80° C. during the dropping progress. After the dropwise addition was complete, a reaction was carried out under stirring at 80° C. for 2 h. After the reaction solution was cooled at room temperature, 2,4,6-trichloro-1,3,5-triazine (22.3 g, 120 mmol) dissolved in 80 mL of THE was added dropwise to the mixed solution, and the reaction was completed after stirring for 3 h. After the reaction was completed, the reaction solution was extracted with toluene and water, the organic phases were combined, an organic layer was dried over anhydrous magnesium sulfate, filtered, and concentrated by distillation under reduced pressure; and a crude product was purified by silica gel column chromatography, recrystallized by using methanol, and filtered to obtain an intermediate a-I-1 (24.2 g, 63%) as a solid.

Synthesis of Intermediate Sub A-1

Under the protection of N₂, magnesium sheets (1.52 g, 63.7 mmol) and 30 mL of tetrahydrofuran were added into a three-necked flask, the temperature of the system was raised to 80° C., a mixture of iodine (0.32 g, 1.26 mmol) and 4-bromodibenzofuran (15.73 g, 63.7 mmol) dissolved completely in 30 mL of THF was slowly added dropwise to the system where magnesium sheets and tetrahydrofuran reacted within 30 min, and the temperature was controlled at 80° C. during the dropping progress. After the dropwise addition was complete, a reaction was carried out under stirring at 80° C. for 2 h. After the reaction solution was cooled at room temperature, the intermediate a-I-1 (20.13 g, 63.7 mmol) dissolved in 40 mL of THF was added dropwise to the mixed solution, and the reaction was completed after stirring for 3 h. After the reaction was completed, the reaction solution was extracted with toluene and water, the organic phases were combined, an organic layer was dried over anhydrous magnesium sulfate, filtered, and concentrated by distillation under reduced pressure; and a crude product was purified by silica gel column chromatography, recrystallized by using methanol, and filtered to obtain the intermediate sub A-1 (22.5 g, 79%) as a solid.

2) Synthesis of Intermediate Sub B-1

An intermediate sub B-1 was synthesized by the following synthetic route:

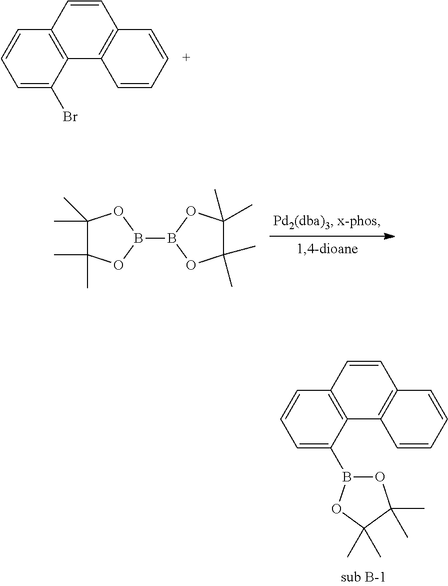

4-Bromophenanthrene (50.0 g, 194.4 mmol), bis(pinacolato)diboron (74.1 g, 291.6 mmol), tris(dibenzylideneacetone)dipalladium (1.7 g, 1.9 mmol), 2-dicyclohexylphosphino-2′,4′,6′-triisopropylbiphenyl (1.8 g, 3.8 mmol), and 1,4-dioxane (500 mL) were added into a round bottom flask, heated to 100° C. under nitrogen protection, then heated for reflux and stirred for 12 h. After the reaction was completed, the solution was cooled to room temperature, the reaction solution was extracted with toluene and water, the organic phases were combined, an organic layer was dried over anhydrous magnesium sulfate, filtered, concentrated, and pulped with n-heptane to obtain the intermediate sub B-1 (23.6 g, 40%) as a solid compound.

3) Preparation of Compound 3

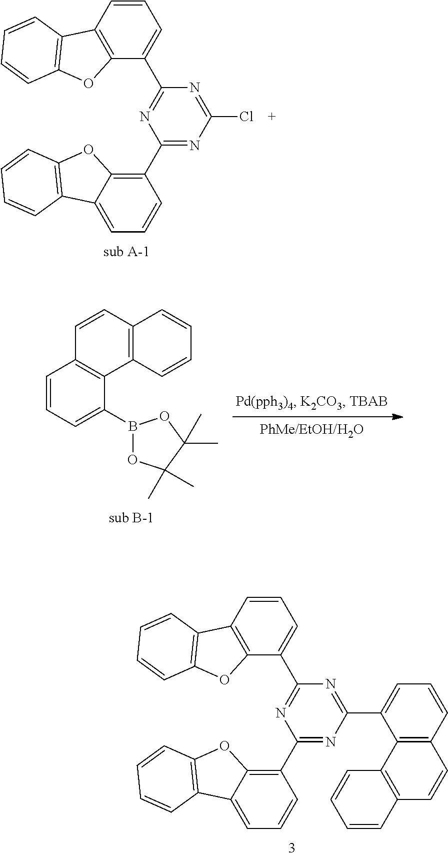 [SMILES: Clc1nc(-c2cccc3c2oc2ccccc23)nc(-c2cccc3c2oc2ccccc23)n1 ; CC1(C)OB(c2cccc3ccc4ccccc4c23)OC1(C)C ; c1ccc2c(c1)ccc1cccc(-c3nc(-c4cccc5c4oc4ccccc45)nc(-c4cccc5c4oc4ccccc45)n3)c12]

The intermediate sub A-1 (10.0 g, 22.3 mmol), the intermediate sub B-1 (7.1 g, 23.4 mmol), tetrakis(triphenylphosphine)palladium (0.5 g, 0.4 mmol), potassium carbonate (6.2 g, 44.6 mmol), tetrabutylammonium bromide (0.1 g, 0.4 mmol), toluene (80 mL), ethanol (20 mL) and deionized water (20 mL) were added into a three-necked flask, heated to 76° C. under nitrogen protection, then heated for reflux and stirred for 8 h. After the reaction was completed, the solution was cooled to room temperature, the reaction solution was extracted with toluene and water, the organic phases were combined, and an organic layer was dried over anhydrous magnesium sulfate, filtered, and concentrated; and a crude product was purified by silica gel column chromatography to obtain a compound 3 (7.7 g, 59%) as a solid. m/z=590.18 [M+H]⁺.

### Preparation Examples 2 to 10: Synthesis of Compounds 1, 27, 149, 124, 374, 266, 17, 65, and 31

Intermediates sub A-2 to sub A-17 shown in Table 1 were prepared with reference to the synthesis method for the intermediate sub A-1, except that a raw material A in Table 1 was used instead of the raw material 4-bromodibenzofuran in the preparation of the intermediate a-I-1, and a raw material B in Table 1 was used instead of the raw material 4-bromodibenzofuran in the preparation of the intermediate sub A-1.

TABLE 1

| Intermediate | Raw material | Raw material | Intermediate | |
|---|---|---|---|---|
| No. | A | B | structure | Yield (%) |
| Sub A-2 | 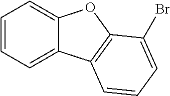 | 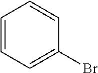 | 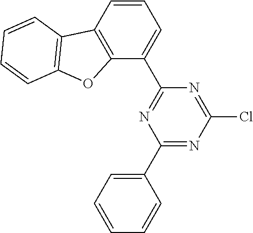 [SMILES: Clc1nc(-c2ccccc2)nc(-c2cccc3c2oc2ccccc23)n1] | 54 |
| Sub A-3 | 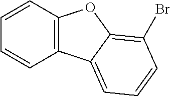 | 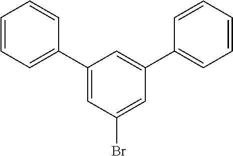 | 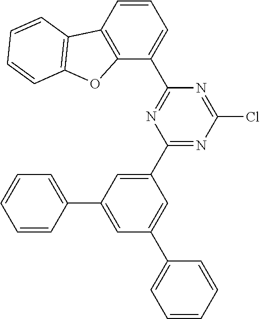 [SMILES: Clc1nc(-c2cc(-c3ccccc3)cc(-c3ccccc3)c2)nc(-c2cccc3c2oc2ccccc23)n1] | 51 |
| Sub A-4 | 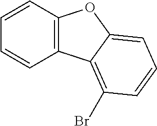 | 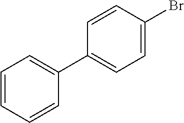 | 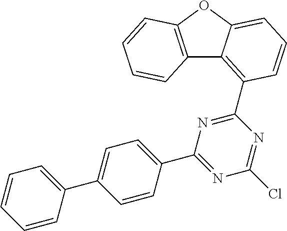 [SMILES: Clc1nc(-c2ccc(-c3ccccc3)cc2)nc(-c2cccc3oc4ccccc4c23)n1] | 60 |
| Sub A-5 | 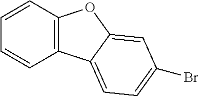 | 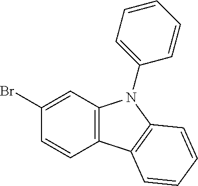 | 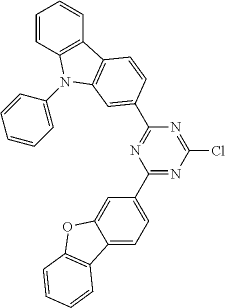 [SMILES: Clc1nc(-c2ccc3c(c2)oc2ccccc23)nc(-c2ccc3c4ccccc4n(-c4ccccc4)c3c2)n1] | 50 |
| Sub A-6 | 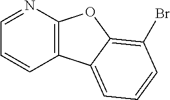 | 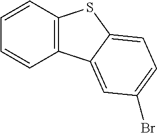 | 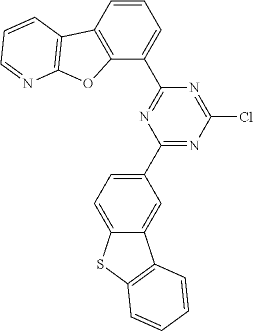 [SMILES: Clc1nc(-c2ccc3sc4ccccc4c3c2)nc(-c2cccc3c2oc2ncccc23)n1] | 48 |
| Sub A-7 | 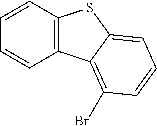 | 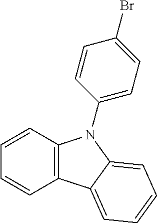 | 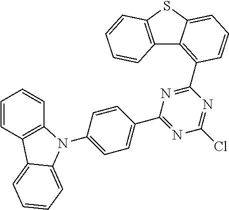 [SMILES: Clc1nc(-c2ccc(-n3c4ccccc4c4ccccc43)cc2)nc(-c2cccc3sc4ccccc4c23)n1] | 56 |
| Sub A-8 | 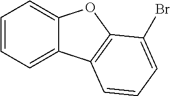 | 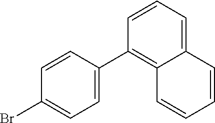 | 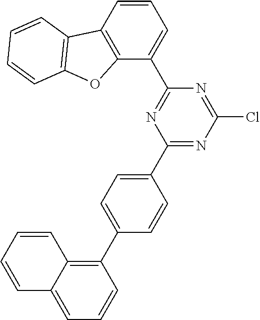 [SMILES: Clc1nc(-c2ccc(-c3cccc4ccccc34)cc2)nc(-c2cccc3c2oc2ccccc23)n1] | 58 |
| Sub A-9 | 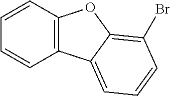 | 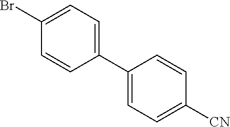 | 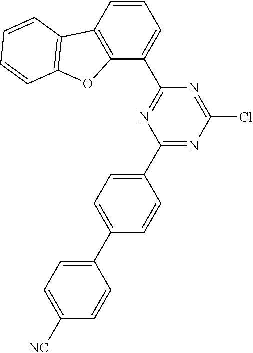 [SMILES: N#Cc1ccc(-c2ccc(-c3nc(Cl)nc(-c4cccc5c4oc4ccccc45)n3)cc2)cc1] | 53 |
| Sub A-10 | 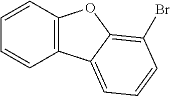 | 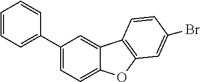 | 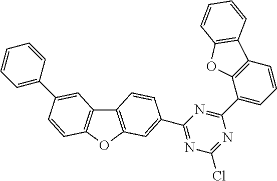 [SMILES: Clc1nc(-c2ccc3c(c2)oc2ccc(-c4ccccc4)cc23)nc(-c2cccc3c2oc2ccccc23)n1] | 51 |
| Sub A-11 | 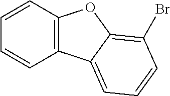 | 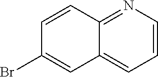 | 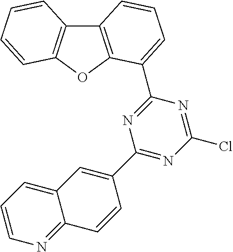 [SMILES: Clc1nc(-c2ccc3ncccc3c2)nc(-c2cccc3c2oc2ccccc23)n1] | 50 |
| Sub A-12 | 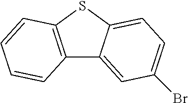 | 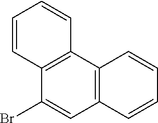 | 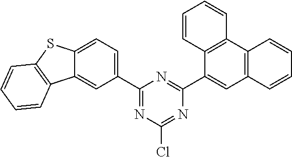 [SMILES: Clc1nc(-c2ccc3sc4ccccc4c3c2)nc(-c2cc3ccccc3c3ccccc23)n1] | 48 |
| Sub A-13 | 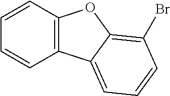 | 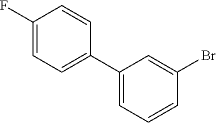 | 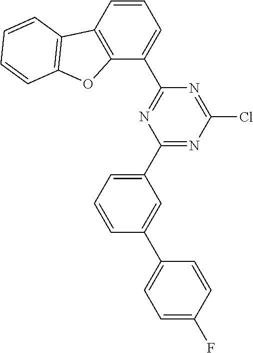 [SMILES: Fc1ccc(-c2cccc(-c3nc(Cl)nc(-c4cccc5c4oc4ccccc45)n3)c2)cc1] | 46 |
| Sub A-14 | 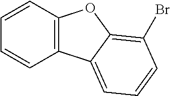 | 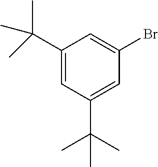 | 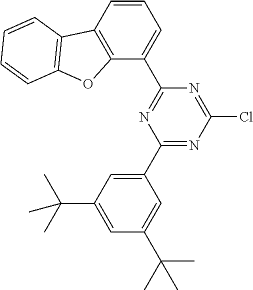 [SMILES: CC(C)(C)c1cc(-c2nc(Cl)nc(-c3cccc4c3oc3ccccc34)n2)cc(C(C)(C)C)c1] | 49 |
| Sub A-15 | 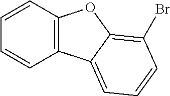 | 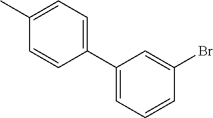 | 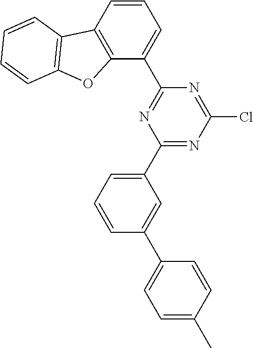 [SMILES: Cc1ccc(-c2cccc(-c3nc(Cl)nc(-c4cccc5c4oc4ccccc45)n3)c2)cc1] | 51 |
| Sub A-16 | 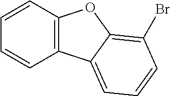 | 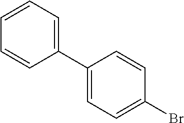 | 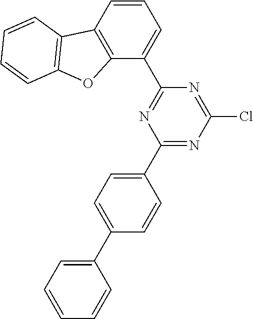 [SMILES: Clc1nc(-c2ccc(-c3ccccc3)cc2)nc(-c2cccc3c2oc2ccccc23)n1] | 65 |
| Sub A-17 | 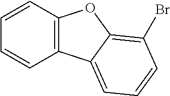 | 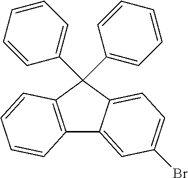 | 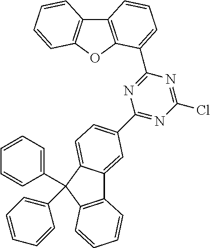 [SMILES: Clc1nc(-c2ccc3c(c2)-c2ccccc2C3(c2ccccc2)c2ccccc2)nc(-c2cccc3c2oc2ccccc23)n1] | 59 |

Compounds shown in Table 2 below were synthesized in a similar manner to that in Preparation example 1 except that intermediates sub A-2 to sub A-10 in Table 2 were used instead of the intermediate sub A-1.

TABLE 2

| | | | | Mass |
|---|---|---|---|---|
| Preparation | | | | spectrum |
| example | Sub A | Compound | Yield (%) | (m/z) |
| 2 | 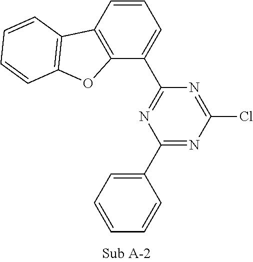 [SMILES: Clc1nc(-c2ccccc2)nc(-c2cccc3c2oc2ccccc23)n1] | 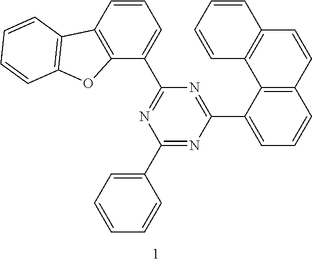 [SMILES: c1ccc(-c2nc(-c3cccc4c3oc3ccccc34)nc(-c3cccc4ccc5ccccc5c34)n2)cc1] | 54 | 500.17 |
| 3 | 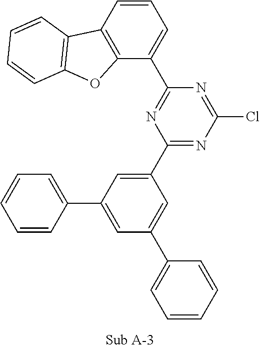 [SMILES: Clc1nc(-c2cc(-c3ccccc3)cc(-c3ccccc3)c2)nc(-c2cccc3c2oc2ccccc23)n1] | 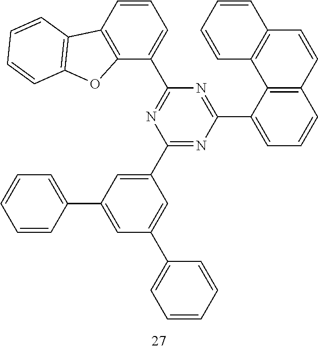 [SMILES: c1ccc(-c2cc(-c3ccccc3)cc(-c3nc(-c4cccc5c4oc4ccccc45)nc(-c4cccc5ccc6ccccc6c45)n3)c2)cc1] | 51 | 652.23 |
| 4 | 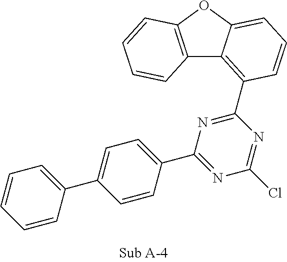 [SMILES: Clc1nc(-c2ccc(-c3ccccc3)cc2)nc(-c2cccc3oc4ccccc4c23)n1] | 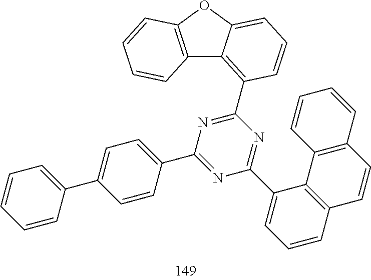 [SMILES: c1ccc(-c2ccc(-c3nc(-c4cccc5ccc6ccccc6c45)nc(-c4cccc5oc6ccccc6c45)n3)cc2)cc1] | 60 | 576.20 |
| 5 | 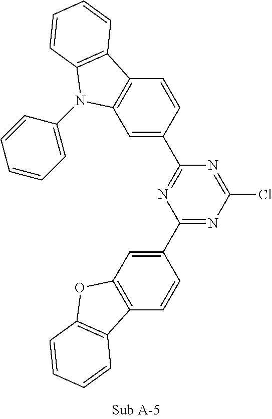 [SMILES: Clc1nc(-c2ccc3c(c2)oc2ccccc23)nc(-c2ccc3c4ccccc4n(-c4ccccc4)c3c2)n1] | 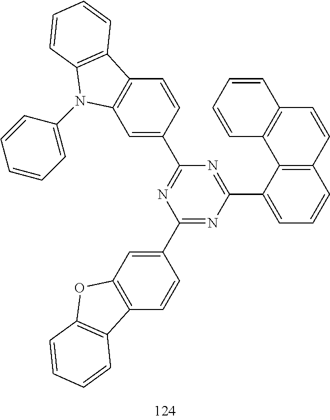 [SMILES: c1ccc(-n2c3ccccc3c3ccc(-c4nc(-c5ccc6c(c5)oc5ccccc56)nc(-c5cccc6ccc7ccccc7c56)n4)cc32)cc1] | 50 | 665.23 |
| 6 | 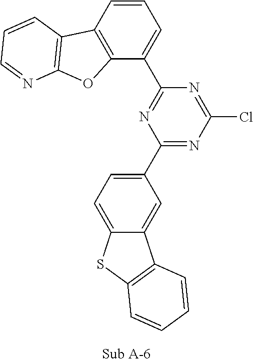 [SMILES: Clc1nc(-c2ccc3sc4ccccc4c3c2)nc(-c2cccc3c2oc2ncccc23)n1] | 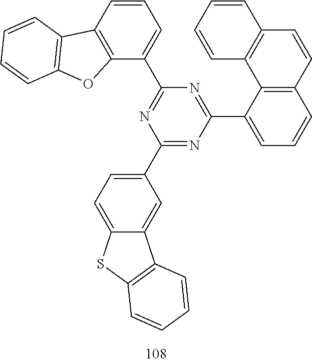 [SMILES: c1ccc2c(c1)ccc1cccc(-c3nc(-c4ccc5sc6ccccc6c5c4)nc(-c4cccc5c4oc4ccccc45)n3)c12] | 48 | 607.15 |
| 7 | 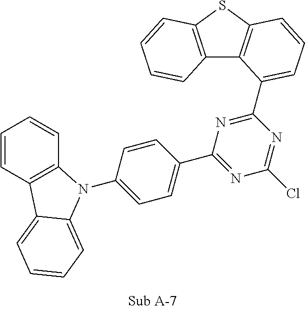 [SMILES: Clc1nc(-c2ccc(-n3c4ccccc4c4ccccc43)cc2)nc(-c2cccc3sc4ccccc4c23)n1] | 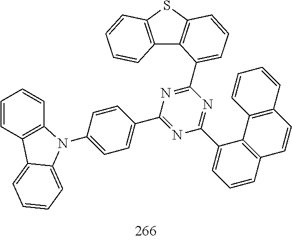 [SMILES: c1ccc2c(c1)ccc1cccc(-c3nc(-c4ccc(-n5c6ccccc6c6ccccc65)cc4)nc(-c4cccc5sc6ccccc6c45)n3)c12] | 56 | 681.20 |
| 8 | 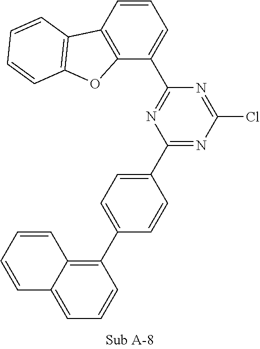 [SMILES: Clc1nc(-c2ccc(-c3cccc4ccccc34)cc2)nc(-c2cccc3c2oc2ccccc23)n1] | 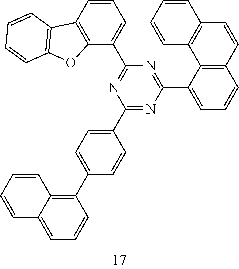 [SMILES: c1ccc2c(-c3ccc(-c4nc(-c5cccc6c5oc5ccccc56)nc(-c5cccc6ccc7ccccc7c56)n4)cc3)cccc2c1] | 58 | 626.22 |
| 9 | 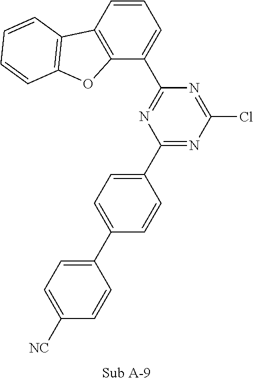 [SMILES: N#Cc1ccc(-c2ccc(-c3nc(Cl)nc(-c4cccc5c4oc4ccccc45)n3)cc2)cc1] | 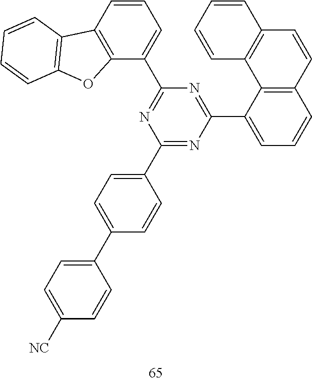 [SMILES: N#Cc1ccc(-c2ccc(-c3nc(-c4cccc5c4oc4ccccc45)nc(-c4cccc5ccc6ccccc6c45)n3)cc2)cc1] | 53 | 601.20 |
| 10 | 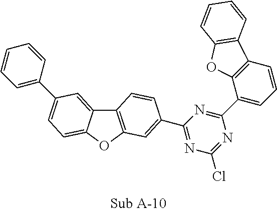 [SMILES: Clc1nc(-c2ccc3c(c2)oc2ccc(-c4ccccc4)cc23)nc(-c2cccc3c2oc2ccccc23)n1] | 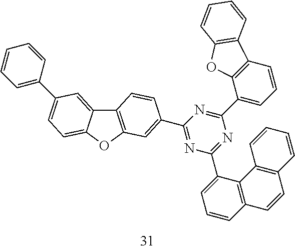 [SMILES: c1ccc(-c2ccc3oc4cc(-c5nc(-c6cccc7c6oc6ccccc67)nc(-c6cccc7ccc8ccccc8c67)n5)ccc4c3c2)cc1] | 51 | 666.21 |

### Preparation Example 11: Synthesis of Compound 256

A compound 256 was synthesized by the following synthetic route:

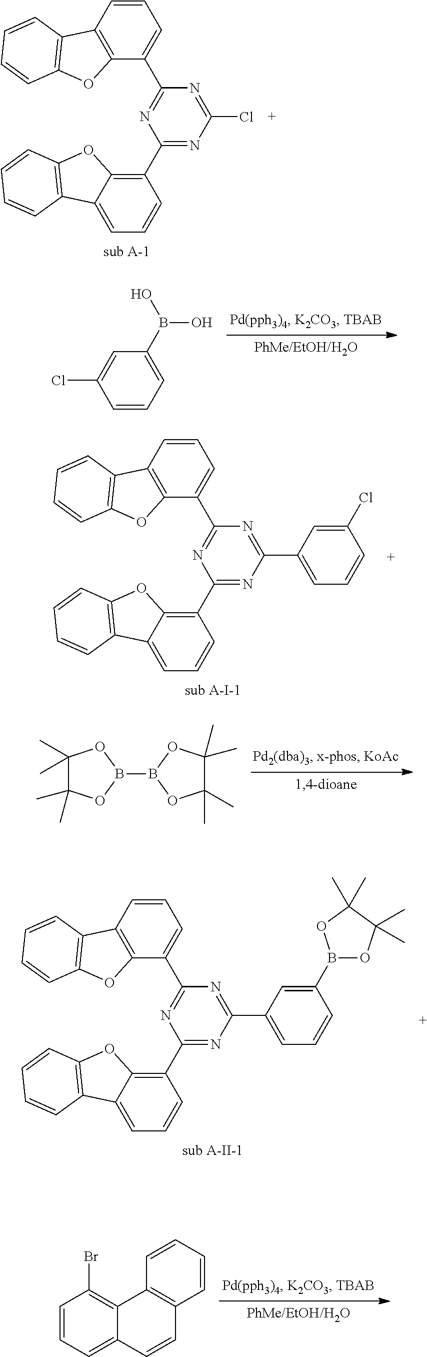

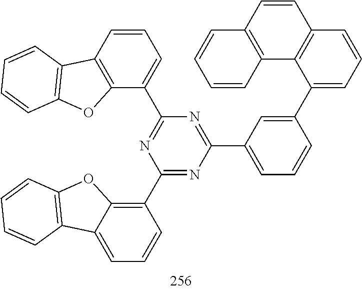 [SMILES: c1cc(-c2nc(-c3cccc4c3oc3ccccc34)nc(-c3cccc4c3oc3ccccc34)n2)cc(-c2cccc3ccc4ccccc4c23)c1]

1) Synthesis of Intermediate Sub A-I-1

The intermediate sub A-1 (30.0 g, 66.9 mmol), 3-chlorophenylboronic acid (11.5 g, 73.6 mmol), tetrakis(triphenylphosphine)palladium (1.5 g, 1.3 mmol), potassium carbonate (18.5 g, 133.9 mmol), tetrabutylammonium bromide (0.2 g, 0.6 mmol), toluene (240 mL), ethanol (60 mL) and deionized water (60 mL) were added into a three-necked flask, heated to 76° C. under nitrogen protection, then heated for reflux and stirred for 8 h. After the reaction was completed, the solution was cooled to room temperature, the reaction solution was extracted with toluene and water, the organic phases were combined, and an organic layer was dried over anhydrous magnesium sulfate, filtered, and concentrated; and a crude product was purified by silica gel column chromatography to obtain an intermediate sub A-I-1 (22.8 g, 65%) as a solid compound.

2) Synthesis of Intermediate Sub A-II-1

The intermediate sub A-I-1 (20 g, 38.1 mmol), bis(pinacolato)diboron (14.5 g, 57.2 mmol), tris(dibenzylideneacetone)dipalladium (0.3 g, 0.4 mmol), 2-dicyclohexylphosphino-2′,4′,6′-triisopropylbiphenyl (0.4 g, 0.7 mmol), and potassium acetate (7.5 g, 76.3 mmol) were added into 1,4-dioxane (200 mL), and a reaction was carried out under reflux at 100° C. for 12 h. When the reaction was completed, the reaction was extracted with dichloromethane and water. An organic layer was dried over magnesium sulfate, and concentrated, and the resulting compound was pulped with ethanol twice to obtain an intermediate sub A-II-1 (16.2 g, 69%).

3) Synthesis of Compound 256

The intermediate sub A-II-1 (15.8 g, 25.6 mmol), 4-bromophenanthrene (6.0 g, 23.3 mmol), tetrakis(triphenylphosphine)palladium (0.5 g, 0.5 mmol), potassium carbonate (6.4 g, 46.6 mmol), tetrabutylammonium bromide (0.07 g, 0.2 mmol), toluene (120 mL), ethanol (30 mL) and deionized water (30 mL) were added into a three-necked flask, heated to 76° C. under nitrogen protection, then heated for reflux and stirred for 13 h. After the reaction was completed, the solution was cooled to room temperature, the reaction solution was extracted with toluene and water, the organic phases were combined, and an organic layer was dried over anhydrous magnesium sulfate, filtered, and concentrated; and a crude product was purified by silica gel column chromatography to obtain the compound 256 (9.2 g, 59%) as a solid. MS [M+H]⁺=666.21.

### Preparation Examples 12 to 24: Synthesis of Compounds 294, 366, 389, 398, 399, 245, 230, 270, 392, 400, 401, 402, and 403

Compounds shown in Table 3 below were synthesized in a similar manner to that in Preparation example 11 except that a raw material C in Table 3 was used instead of 3-chlorophenylboronic acid in the preparation of the intermediate sub A-1, and an intermediate sub Ain Table 3 was used instead of the intermediate sub A-1.

TABLE 3

| Prep- | | | | | | |
|---|---|---|---|---|---|---|
| ara- | | | | | | |
| tion | | | | | | Mass |
| exam- | | Raw material | | | Yield | spec- |
| ple | Sub A | C | Intermediate sub A-II | Compound | % | trum |
| 12 | 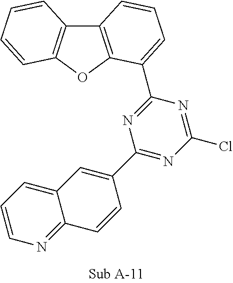 [SMILES: Clc1nc(-c2ccc3ncccc3c2)nc(-c2cccc3c2oc2ccccc23)n1] | 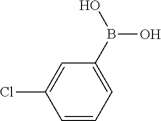 | 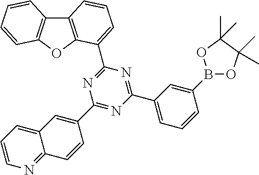 [SMILES: CC1(C)OB(c2cccc(-c3nc(-c4ccc5ncccc5c4)nc(-c4cccc5c4oc4ccccc45)n3)c2)OC1(C)C] | 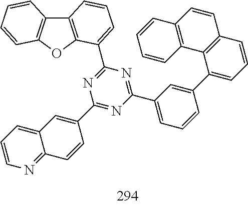 [SMILES: c1cc(-c2nc(-c3ccc4ncccc4c3)nc(-c3cccc4c3oc3ccccc34)n2)cc(-c2cccc3ccc4ccccc4c23)c1] | 52 | 627.21 |
| 13 | 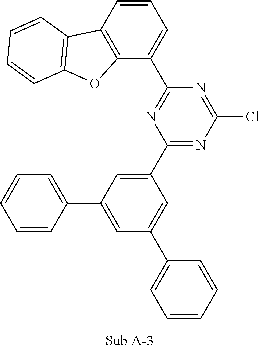 [SMILES: Clc1nc(-c2cc(-c3ccccc3)cc(-c3ccccc3)c2)nc(-c2cccc3c2oc2ccccc23)n1] | 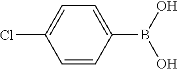 | 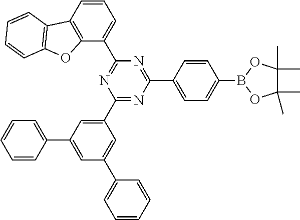 [SMILES: CC1(C)OB(c2ccc(-c3nc(-c4cc(-c5ccccc5)cc(-c5ccccc5)c4)nc(-c4cccc5c4oc4ccccc45)n3)cc2)OC1(C)C] | 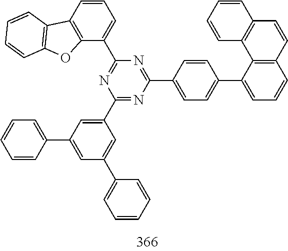 [SMILES: c1ccc(-c2cc(-c3ccccc3)cc(-c3nc(-c4ccc(-c5cccc6ccc7ccccc7c56)cc4)nc(-c4cccc5c4oc4ccccc45)n3)c2)cc1] | 56 | 728.26 |
| 14 | 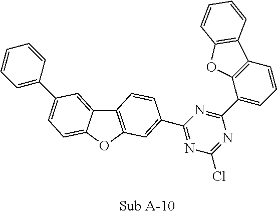 [SMILES: Clc1nc(-c2ccc3c(c2)oc2ccc(-c4ccccc4)cc23)nc(-c2cccc3c2oc2ccccc23)n1] | 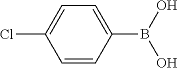 | 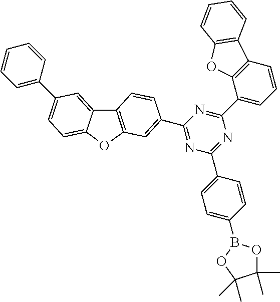 [SMILES: CC1(C)OB(c2ccc(-c3nc(-c4ccc5c(c4)oc4ccc(-c6ccccc6)cc45)nc(-c4cccc5c4oc4ccccc45)n3)cc2)OC1(C)C] | 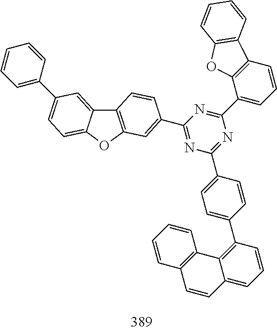 [SMILES: c1ccc(-c2ccc3oc4cc(-c5nc(-c6ccc(-c7cccc8ccc9ccccc9c78)cc6)nc(-c6cccc7c6oc6ccccc67)n5)ccc4c3c2)cc1] | 54 | 742.24 |
| 15 | 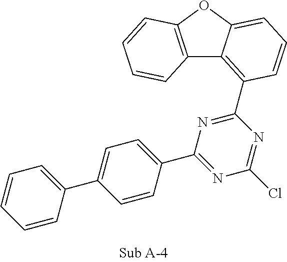 [SMILES: Clc1nc(-c2ccc(-c3ccccc3)cc2)nc(-c2cccc3oc4ccccc4c23)n1] | 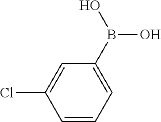 | 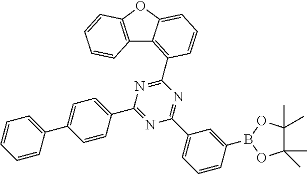 [SMILES: CC1(C)OB(c2cccc(-c3nc(-c4ccc(-c5ccccc5)cc4)nc(-c4cccc5oc6ccccc6c45)n3)c2)OC1(C)C] | 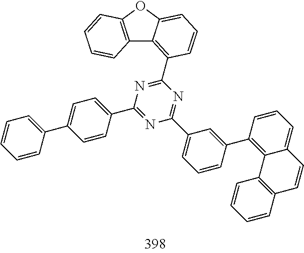 [SMILES: c1ccc(-c2ccc(-c3nc(-c4cccc(-c5cccc6ccc7ccccc7c56)c4)nc(-c4cccc5oc6ccccc6c45)n3)cc2)cc1] | 55 | 652.23 |
| 16 | 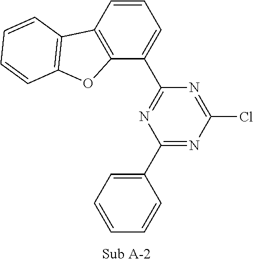 [SMILES: Clc1nc(-c2ccccc2)nc(-c2cccc3c2oc2ccccc23)n1] | 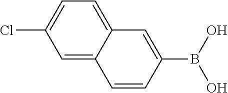 [SMILES: OB(O)c1ccc2cc(Cl)ccc2c1] | 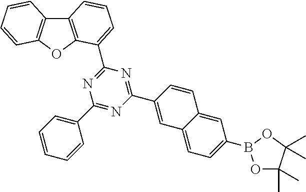 [SMILES: CC1(C)OB(c2ccc3cc(-c4nc(-c5ccccc5)nc(-c5cccc6c5oc5ccccc56)n4)ccc3c2)OC1(C)C] | 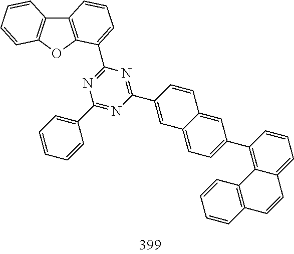 [SMILES: c1ccc(-c2nc(-c3ccc4cc(-c5cccc6ccc7ccccc7c56)ccc4c3)nc(-c3cccc4c3oc3ccccc34)n2)cc1] | 52 | 625.22 |
| 17 | 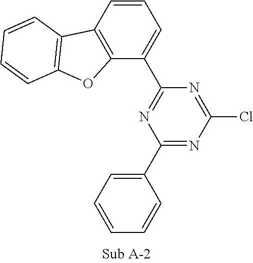 [SMILES: Clc1nc(-c2ccccc2)nc(-c2cccc3c2oc2ccccc23)n1] | 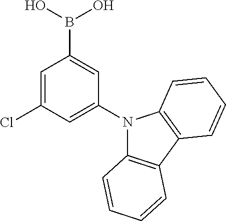 [SMILES: OB(O)c1cc(Cl)cc(-n2c3ccccc3c3ccccc32)c1] | 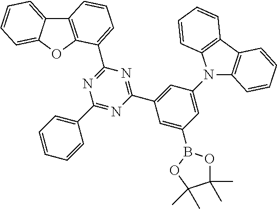 [SMILES: CC1(C)OB(c2cc(-c3nc(-c4ccccc4)nc(-c4cccc5c4oc4ccccc45)n3)cc(-n3c4ccccc4c4ccccc43)c2)OC1(C)C] | 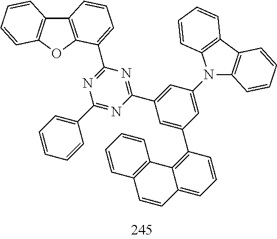 [SMILES: c1ccc(-c2nc(-c3cc(-c4cccc5ccc6ccccc6c45)cc(-n4c5ccccc5c5ccccc54)c3)nc(-c3cccc4c3oc3ccccc34)n2)cc1] | 48 | 741.26 |
| 18 | 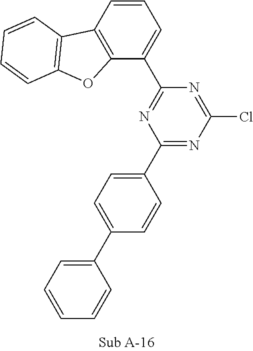 [SMILES: Clc1nc(-c2ccc(-c3ccccc3)cc2)nc(-c2cccc3c2oc2ccccc23)n1] | 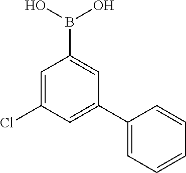 [SMILES: OB(O)c1cc(Cl)cc(-c2ccccc2)c1] | 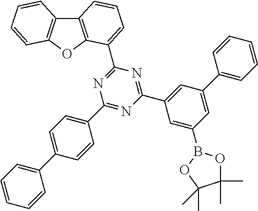 [SMILES: CC1(C)OB(c2cc(-c3ccccc3)cc(-c3nc(-c4ccc(-c5ccccc5)cc4)nc(-c4cccc5c4oc4ccccc45)n3)c2)OC1(C)C] | 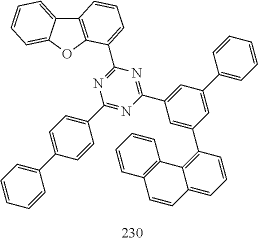 [SMILES: c1ccc(-c2ccc(-c3nc(-c4cc(-c5ccccc5)cc(-c5cccc6ccc7ccccc7c56)c4)nc(-c4cccc5c4oc4ccccc45)n3)cc2)cc1] | 57 | 728.26 |
| 19 | 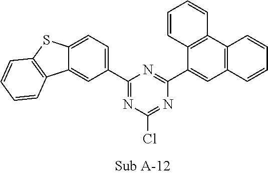 [SMILES: Clc1nc(-c2ccc3sc4ccccc4c3c2)nc(-c2cc3ccccc3c3ccccc23)n1] | 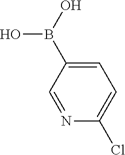 | 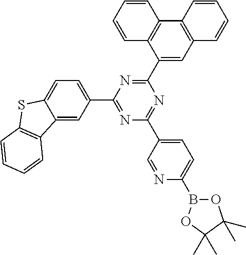 [SMILES: CC1(C)OB(c2ccc(-c3nc(-c4ccc5sc6ccccc6c5c4)nc(-c4cc5ccccc5c5ccccc45)n3)cn2)OC1(C)C] | 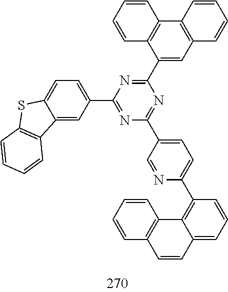 [SMILES: c1ccc2c(c1)cc(-c1nc(-c3ccc(-c4cccc5ccc6ccccc6c45)nc3)nc(-c3ccc4sc5ccccc5c4c3)n1)c1ccccc12] | 45 | 693.20 |
| 20 | 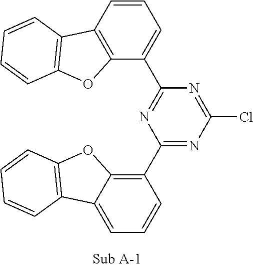 [SMILES: Clc1nc(-c2cccc3c2oc2ccccc23)nc(-c2cccc3c2oc2ccccc23)n1] | 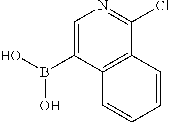 [SMILES: OB(O)c1cnc(Cl)c2ccccc12] | 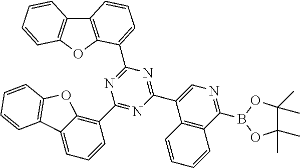 [SMILES: CC1(C)OB(c2ncc(-c3nc(-c4cccc5c4oc4ccccc45)nc(-c4cccc5c4oc4ccccc45)n3)c3ccccc23)OC1(C)C] | 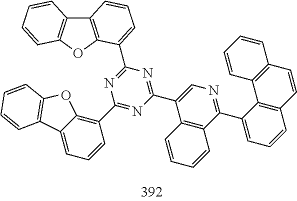 [SMILES: c1ccc2c(c1)ccc1cccc(-c3ncc(-c4nc(-c5cccc6c5oc5ccccc56)nc(-c5cccc6c5oc5ccccc56)n4)c4ccccc34)c12] | 53 | 717.22 |
| 21 | 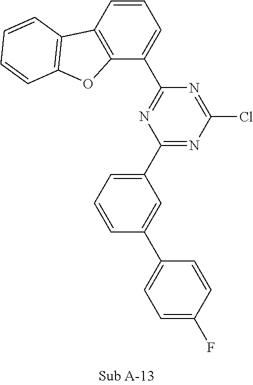 [SMILES: Fc1ccc(-c2cccc(-c3nc(Cl)nc(-c4cccc5c4oc4ccccc45)n3)c2)cc1] | 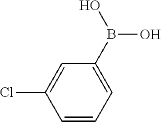 | 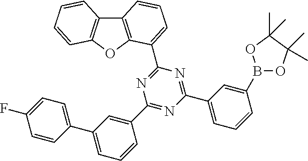 [SMILES: CC1(C)OB(c2cccc(-c3nc(-c4cccc(-c5ccc(F)cc5)c4)nc(-c4cccc5c4oc4ccccc45)n3)c2)OC1(C)C] | 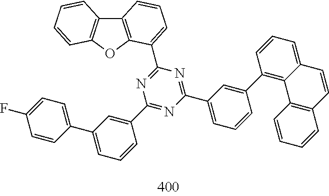 [SMILES: Fc1ccc(-c2cccc(-c3nc(-c4cccc(-c5cccc6ccc7ccccc7c56)c4)nc(-c4cccc5c4oc4ccccc45)n3)c2)cc1] | 57 | 670.22 |
| 22 | 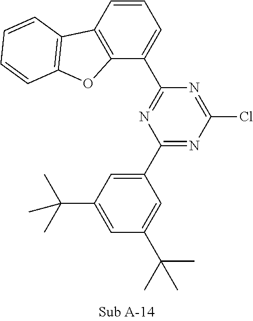 [SMILES: CC(C)(C)c1cc(-c2nc(Cl)nc(-c3cccc4c3oc3ccccc34)n2)cc(C(C)(C)C)c1] | 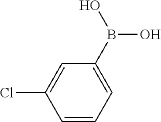 | 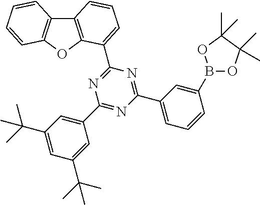 [SMILES: CC(C)(C)c1cc(-c2nc(-c3cccc(B4OC(C)(C)C(C)(C)O4)c3)nc(-c3cccc4c3oc3ccccc34)n2)cc(C(C)(C)C)c1] | 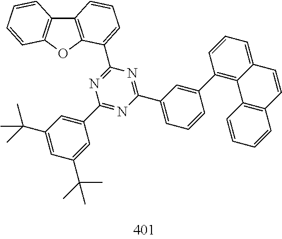 [SMILES: CC(C)(C)c1cc(-c2nc(-c3cccc(-c4cccc5ccc6ccccc6c45)c3)nc(-c3cccc4c3oc3ccccc34)n2)cc(C(C)(C)C)c1] | 52 | 688.32 |
| 23 | 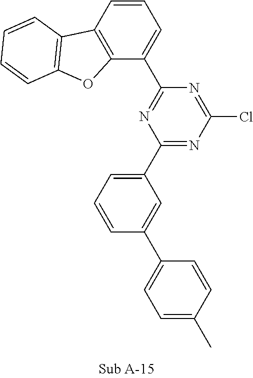 [SMILES: Cc1ccc(-c2cccc(-c3nc(Cl)nc(-c4cccc5c4oc4ccccc45)n3)c2)cc1] | 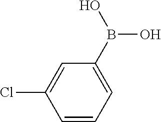 | 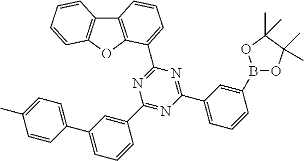 | 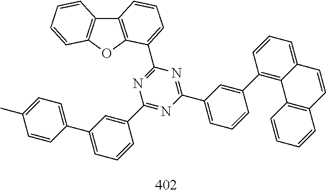 [SMILES: Cc1ccc(-c2cccc(-c3nc(-c4cccc(-c5cccc6ccc7ccccc7c56)c4)nc(-c4cccc5c4oc4ccccc45)n3)c2)cc1] | 55 | 666.25 |
| 24 | 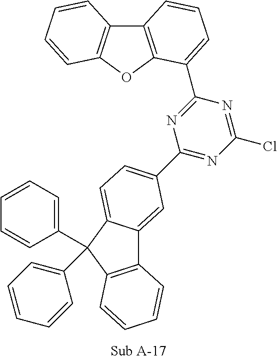 [SMILES: Clc1nc(-c2ccc3c(c2)-c2ccccc2C3(c2ccccc2)c2ccccc2)nc(-c2cccc3c2oc2ccccc23)n1] | 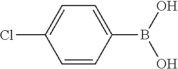 | 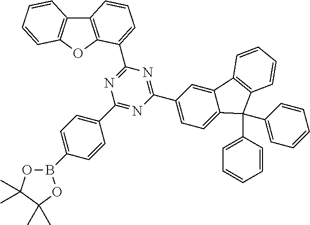 [SMILES: CC1(C)OB(c2ccc(-c3nc(-c4ccc5c(c4)-c4ccccc4C5(c4ccccc4)c4ccccc4)nc(-c4cccc5c4oc4ccccc45)n3)cc2)OC1(C)C] | 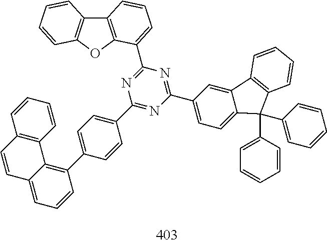 [SMILES: c1ccc(C2(c3ccccc3)c3ccccc3-c3cc(-c4nc(-c5ccc(-c6cccc7ccc8ccccc8c67)cc5)nc(-c5cccc6c5oc5ccccc56)n4)ccc32)cc1] | 6 | 816.29 |

NMR Data for Some Compounds are Shown in Table 4 Below:

TABLE 4

| Compound | NMR data |
|---|---|
| Compound 1 | ¹H NMR (400 MHz, CD₂Cl₂): δ8.87-8.85 (d, 2H), δ8.72-8.68 (d, 1H), δ8.61-8.59 |
| | (d, 1H), δ8.43-8.40 (d, 1H), δ8.30-8.27 (d, 2H), δ8.14-8.12 (d, 2H), δ8.05-8.03 |
| | (d, 1H), δ7.88-7.85 (d, 1H), δ7.72-7.51 (m, 9H), δ7.33-7.31 (m, 1H). |
| Compound 256 | ¹H NMR (400 MHz, CD₂Cl₂): δ9.04 (s, 1H), δ8.63-8.57 (d, 3H), δ8.45-8.40 (d, |
| | 2H), δ8.33-8.31 (d, 1H), δ8.06-8.04 (d, 2H), δ7.95-7.93 (d, 1H), δ7.87-7.51 (m, |
| | 15H), δ7.34-7.31 (t, 2H). |

Manufacture and Evaluation of Organic Electroluminescent Device

### Example 1

Green Organic Electroluminescent Device

An anode was prepared by the following process: an ITO substrate having an ITO thickness of 110 nm was cut into a dimension of 40 mm (length)×40 mm (width)×0.7 mm (thickness) to be prepared into an experimental substrate with an anode, a cathode overlap region and an insulating layer pattern by adopting a photoetching process, and surface treatment was performed on the substrate by utilizing plasma such as ultraviolet ozone so as to increase the work function of the anode. The surface of the ITO substrate may also be cleaned with an organic solvent to clean impurities and oil on the surface.

HAT-CN was vacuum evaporated on the ITO substrate to form a hole injection layer (HIL) having a thickness of 10 nm, and NPB was vacuum evaporated on the hole injection layer to form a hole transport layer having a thickness of 110 nm.

TCBPA was vacuum evaporated on the hole transport layer to form an electron blocking layer having a thickness of 35 nm.

A compound 3 and GH-P1 which were used as a host, and Ir(npy)₂acac which was used as a dopant were co-evaporated on the electron blocking layer in a mass ratio of 50%:45%:5% to form a green organic electroluminescent layer (EML) having a thickness of 38 nm.

TPyQB and LiQ were mixed at a weight ratio of 1:1 and evaporated to form an electron transport layer (ETL) having a thickness of 28 nm.

Yb was evaporated on the electron transport layer to form an electron injection layer (EIL) having a thickness of 1.5 nm.

Magnesium (Mg) and silver (Ag) were mixed and vacuum evaporated at an evaporation rate of 1:9 on the electron injection layer to form a cathode having a thickness of 14 nm.

In addition, CP-1 having a thickness of 66 nm was vacuum evaporated on the cathode, thus completing the manufacture of the organic electroluminescent device.

### Examples 2 to 24

An organic electroluminescent device was manufactured by the same method as that in Example 1 except that compounds shown in Table 5 were used instead of the compound 3 in Example 1 when the organic electroluminescent layer was formed.

### Comparative Examples 1 to 6

An organic electroluminescent device was manufactured by the same method as that in Example 1, except that compounds A, B, C, D, E, and F were used instead of the compound 3 in Example 1 when the organic electroluminescent layer was formed.

When the organic electroluminescent device was manufactured, the structures of materials used in Comparative examples 1 to 6 and Examples 1 to 24 are as follows:

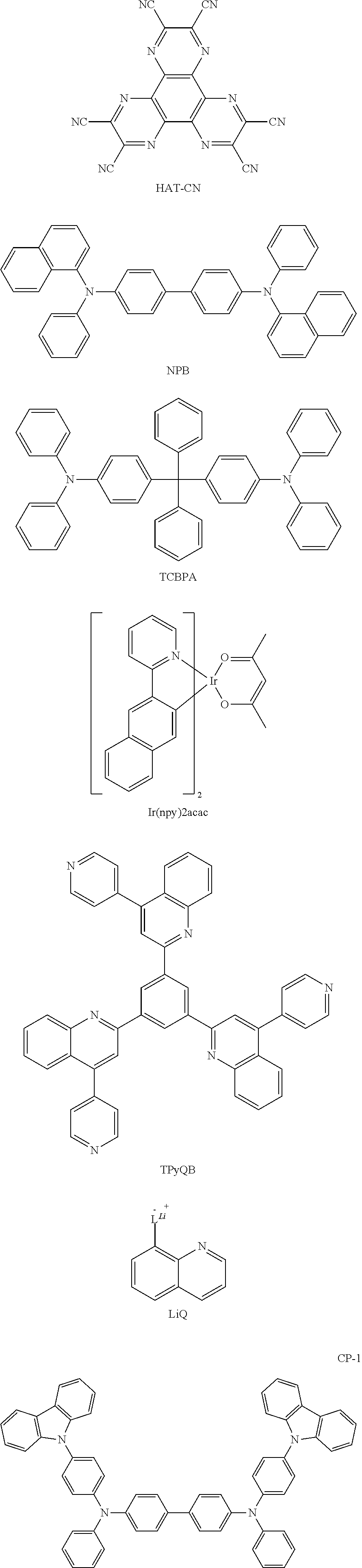

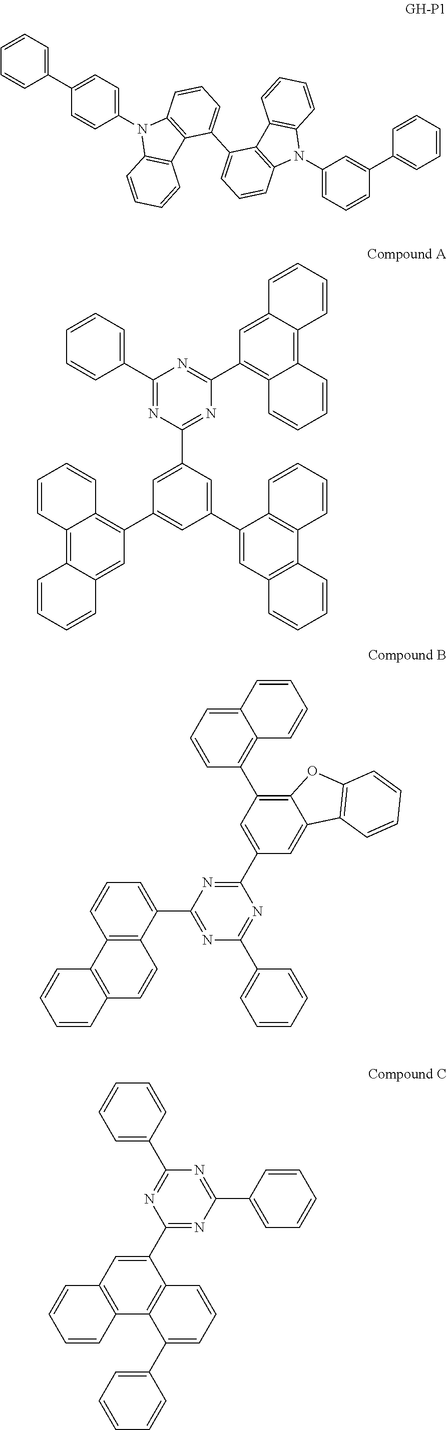 [SMILES: c1ccc(-c2ccc(-n3c4ccccc4c4c(-c5cccc6c5c5ccccc5n6-c5cccc(-c6ccccc6)c5)cccc43)cc2)cc1 ; c1ccc(-c2nc(-c3cc(-c4cc5ccccc5c5ccccc45)cc(-c4cc5ccccc5c5ccccc45)c3)nc(-c3cc4ccccc4c4ccccc34)n2)cc1 ; c1ccc(-c2nc(-c3cc(-c4cccc5ccccc45)c4oc5ccccc5c4c3)nc(-c3cccc4c3ccc3ccccc34)n2)cc1 ; c1ccc(-c2nc(-c3ccccc3)nc(-c3cc4ccccc4c4c(-c5ccccc5)cccc34)n2)cc1]

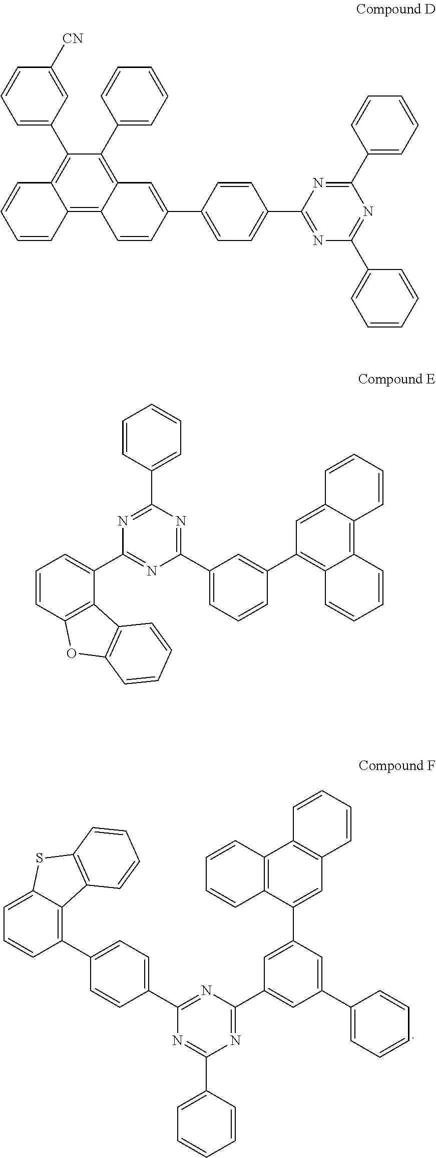 [SMILES: N#Cc1cccc(-c2c(-c3ccccc3)c3cc(-c4ccc(-c5nc(-c6ccccc6)nc(-c6ccccc6)n5)cc4)ccc3c3ccccc23)c1 ; c1ccc(-c2nc(-c3cccc(-c4cc5ccccc5c5ccccc45)c3)nc(-c3cccc4oc5ccccc5c34)n2)cc1 ; c1ccc(-c2cc(-c3nc(-c4ccccc4)nc(-c4ccc(-c5cccc6sc7ccccc7c56)cc4)n3)cc(-c3cc4ccccc4c4ccccc34)c2)cc1]

The green organic electroluminescent devices manufactured in Examples 1 to 24 and Comparative examples 1 to 6 were subjected to performance tests, specifically the IVL performance of the devices was tested under a condition of 10 mA/cm², and the T95 device service life was tested under a condition of 20 mA/cm², and the test results are shown in Table 5.

TABLE 5

| | | | | | External | T95 |
|---|---|---|---|---|---|---|
| | | Driving | Current | Chromaticity | quantum | service |
| | | Voltage | efficiency | coordinate | efficiency | life |
| Example No. | Compound X | (V) | (Cd/A) | CIEx, CIEy | EQE (%) | (h) |
| Example 1 | Compound 3 | 4.04 | 82.02 | 0.266, 0.700 | 31.2 | 201 |
| Example 2 | Compound 1 | 4.04 | 81.64 | 0.264, 0.702 | 31.0 | 199 |
| Example 3 | Compound 27 | 4.05 | 81.78 | 0.265, 0.700 | 31.1 | 198 |
| Example 4 | Compound 149 | 4.00 | 82.91 | 0.263, 0.703 | 31.5 | 230 |
| Example 5 | Compound 124 | 4.02 | 81.03 | 0.264, 0.702 | 30.8 | 202 |
| Example 6 | Compound 374 | 4.02 | 82.07 | 0.263, 0.702 | 31.2 | 218 |
| Example 7 | Compound 266 | 3.99 | 82.79 | 0.264, 0.702 | 31.5 | 198 |
| Example 8 | Compound 17 | 3.99 | 81.94 | 0.264, 0.702 | 31.1 | 220 |
| Example 9 | Compound 65 | 4.01 | 80.77 | 0.264, 0.703 | 30.7 | 195 |
| Example 10 | Compound 31 | 4.05 | 81.15 | 0.261, 0.705 | 30.8 | 215 |
| Example 11 | Compound 256 | 4.02 | 80.97 | 0.261, 0.705 | 30.7 | 211 |
| Example 12 | Compound 294 | 3.99 | 80.37 | 0.263, 0.702 | 30.5 | 202 |
| Example 13 | Compound 366 | 3.97 | 81.06 | 0.263, 0.703 | 30.8 | 196 |
| Example 14 | Compound 389 | 4.01 | 82.02 | 0.262, 0.704 | 31.2 | 218 |
| Example 15 | Compound 398 | 4.03 | 80.97 | 0.263, 0.702 | 30.8 | 222 |
| Example 16 | Compound 399 | 4.05 | 81.76 | 0.266, 0.700 | 31.1 | 207 |
| Example 17 | Compound 245 | 3.95 | 82.48 | 0.263, 0.702 | 31.3 | 212 |
| Example 18 | Compound 230 | 4.03 | 80.62 | 0.262, 0.704 | 30.6 | 203 |
| Example 19 | Compound 270 | 3.95 | 81.38 | 0.263, 0.703 | 30.9 | 197 |
| Example 20 | Compound 392 | 4.02 | 82.33 | 0.262, 0.704 | 31.3 | 222 |
| Example 21 | Compound 400 | 4.02 | 82.99 | 0.263, 0.700 | 31.3 | 208 |
| Example 22 | Compound 401 | 4.01 | 82.34 | 0.262, 0.701 | 31.8 | 224 |
| Example 23 | Compound 402 | 4.03 | 82.37 | 0.262, 0.702 | 31.9 | 213 |
| Example 24 | Compound 403 | 4.02 | 82.38 | 0.261, 0.703 | 31.5 | 212 |
| Comparative | Compound A | 4.06 | 67.8 | 0.266, 0.700 | 21.4 | 138 |
| example 1 | | | | | | |
| Comparative | Compound B | 3.99 | 59.7 | 0.262, 0.704 | 16.9 | 156 |
| example 2 | | | | | | |
| Comparative | Compound C | 3.91 | 62.3 | 0.266, 0.700 | 20.1 | 150 |
| example 3 | | | | | | |
| Comparative | Compound D | 3.91 | 62.6 | 0.266, 0.700 | 20.2 | 150 |
| example 4 | | | | | | |
| Comparative | Compound E | 3.98 | 65.4 | 0.266, 0.700 | 20.3 | 173 |
| example 5 | | | | | | |
| Comparative | Compound F | 4.00 | 62.7 | 0.266, 0.700 | 20.0 | 162 |
| example 6 | | | | | | |

As can be seen from the device performance test results in Table 5, Examples 1 to 24 in which the compounds of the present disclosure were used as the host material of the N-type green organic electroluminescent layer have the advantages that the luminous efficiency Cd/A was improved by at least 18.5%, the external quantum efficiency was improved by at least 42.5%, and the T95 service life was improved by at least 12.7% in the case where the chromaticity coordinates did not differ much compared with Comparative examples 1 to 6.

Thus, when the nitrogen-containing compound of the present disclosure is used for manufacturing the green organic electroluminescent device, the service life of the organic electroluminescent device can be effectively prolonged, and the luminous efficiency of the organic electroluminescent device can be greatly improved.

Preferred examples of the present disclosure have been described in detail above, but the present disclosure is not limited to specific details in the above-described examples, various simple modifications may be made to the technical solutions of the present disclosure within the technical idea of the present disclosure, and these simple modifications are all within the protection scope of the present disclosure.

1. A nitrogen-containing compound, having a structure as shown in a formula 1: 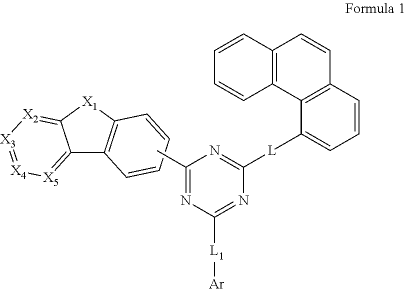
wherein X₁ is selected from O or S;
X₂, X₃, X₄, and X₅ are the same as or different from each other, and are each independently selected from C(H) or N;
L and L₁ are respectively and independently selected from a single bond, substituted or unsubstituted arylene with 6 to 30 carbon atoms, and substituted or unsubstituted heteroarylene with 3 to 30 carbon atoms;
Ar is selected from substituted or unsubstituted aryl with 6 to 30 carbon atoms, substituted or unsubstituted heteroaryl with 3 to 30 carbon atoms, substituted or unsubstituted alkyl with 1 to 20 carbon atoms, and substituted or unsubstituted cycloalkyl with 3 to 20 carbon atoms;
substituents in L, L₁ and Ar are the same as or different from each other, and are each independently selected from deuterium, a halogen group, cyano, heteroaryl with 3 to 20 carbon atoms, aryl with 6 to 20 carbon atoms which can be optionally substituted by 0, 1, 2, 3, 4, or 5 substituents independently selected from deuterium, fluorine, cyano, methyl, and tert-butyl, trialkylsilyl with 3 to 12 carbon atoms, triarylsilyl with 18 to 24 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 18 carbon atoms, arylthio with 6 to 18 carbon atoms, and phosphinyloxy with 6 to 18 carbon atoms; and
in L, L₁ and Ar, optionally, any two adjacent substituents form a ring.

2. The nitrogen-containing compound according to claim 1, wherein L and L₁ are respectively and independently selected from a single bond, substituted or unsubstituted arylene with 6 to 20 carbon atoms, and substituted or unsubstituted heteroarylene with 5 to 20 carbon atoms.

3. The nitrogen-containing compound according to claim 1, wherein L and L₁ are respectively and independently selected from a single bond, substituted or unsubstituted phenylene, substituted or unsubstituted naphthylene, substituted or unsubstituted biphenylene, substituted or unsubstituted pyridylidene, substituted or unsubstituted quinolylidene, substituted or unsubstituted fluorenylidene, substituted or unsubstituted carbazolylidene, substituted or unsubstituted dibenzofurylidene, substituted or unsubstituted dibenzothienylene, substituted or unsubstituted phenanthrylene, substituted or unsubstituted anthrylene, and substituted or unsubstituted N-phenylcarbazolylidene;
or, a group formed by connecting any two of the above groups by a single bond.

4. The nitrogen-containing compound according to claim 1, wherein L and L₁ are respectively and independently selected from a single bond, and a substituted or unsubstituted group V; wherein the unsubstituted group V is selected from the group consisting of: 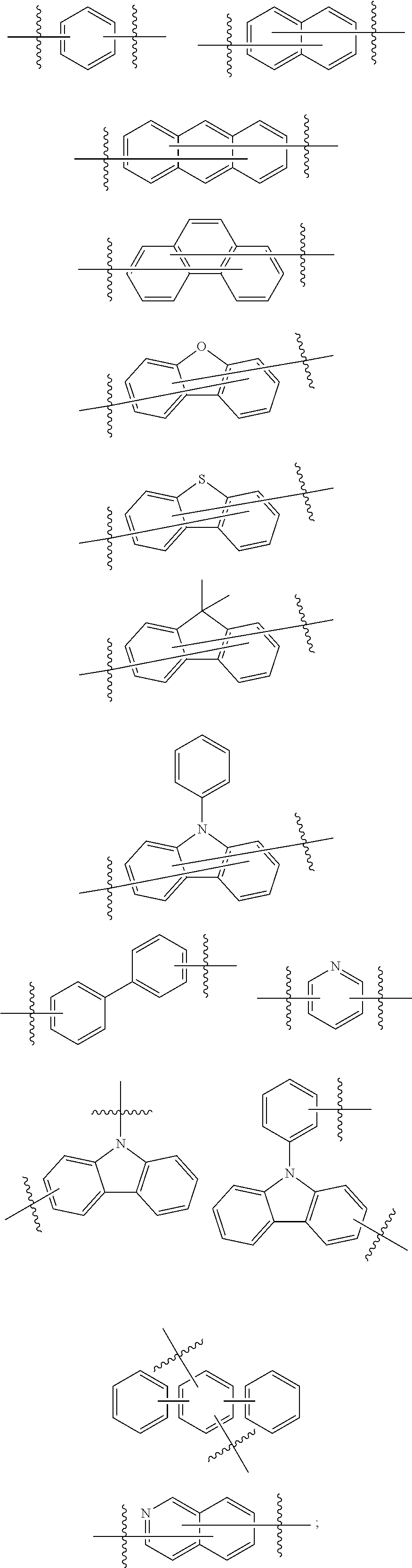
wherein 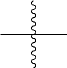
represents a chemical bond; and the substituted group V has one or more substituents, and the substituents are each independently selected from deuterium, fluorine, cyano, a halogen group, methyl, ethyl, n-propyl, isopropyl, tert-butyl, phenyl, naphthyl, biphenyl, and carbazolyl; when the group V has two or more substituents, the two or more substituents are the same or different.

5. The nitrogen-containing compound according to claim 1, wherein Ar is selected from substituted or unsubstituted aryl with 6 to 26 carbon atoms, and substituted or unsubstituted heteroaryl with 5 to 20 carbon atoms.

6. The nitrogen-containing compound according to claim 1, wherein Ar is selected from a substituted or unsubstituted group W, wherein the unsubstituted group W is selected from the group consisting of: 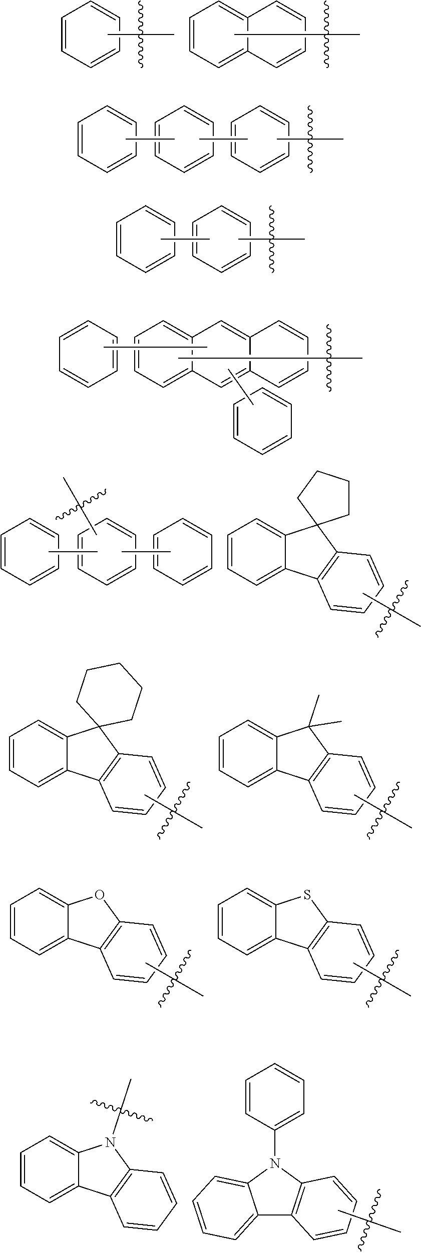 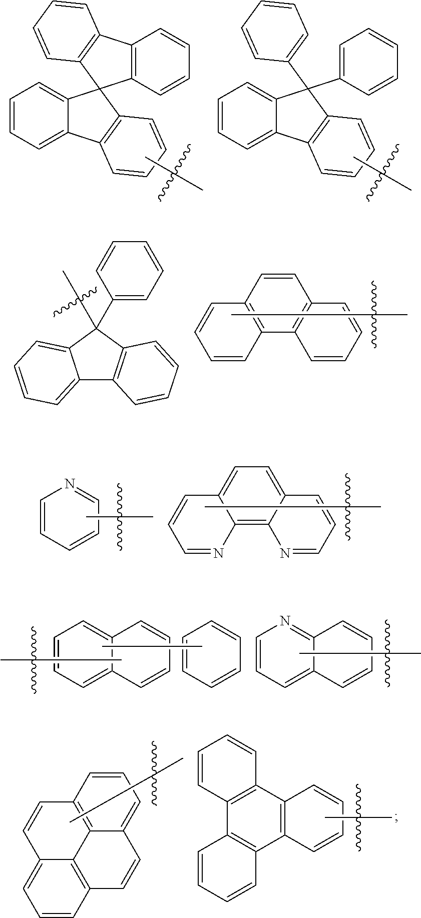
wherein 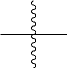
represents a chemical bond; and the substituted group W has one or more substituents, and the substituents are each independently selected from deuterium, fluorine, cyano, a halogen group, methyl, ethyl, n-propyl, isopropyl, tert-butyl, phenyl, naphthyl, biphenyl, cyclopentyl, and cyclohexyl; when the group W has two or more substituents, the two or more substituents are the same or different.

7. The nitrogen-containing compound according to claim 1, wherein the nitrogen-containing compound is selected from the group consisting of the following compounds:

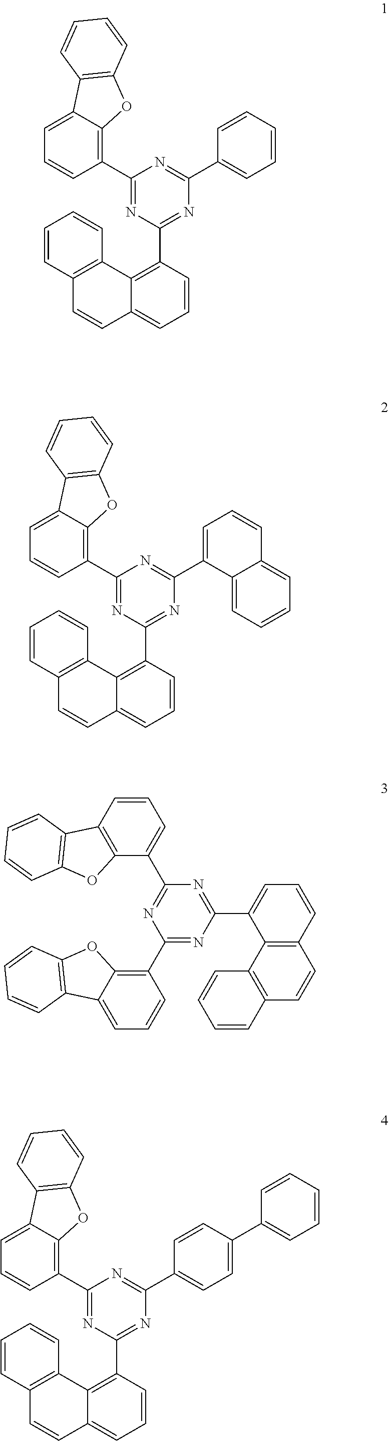 [SMILES: c1ccc(-c2nc(-c3cccc4c3oc3ccccc34)nc(-c3cccc4ccc5ccccc5c34)n2)cc1 ; c1ccc2c(-c3nc(-c4cccc5c4oc4ccccc45)nc(-c4cccc5ccc6ccccc6c45)n3)cccc2c1 ; c1ccc2c(c1)ccc1cccc(-c3nc(-c4cccc5c4oc4ccccc45)nc(-c4cccc5c4oc4ccccc45)n3)c12 ; c1ccc(-c2ccc(-c3nc(-c4cccc5c4oc4ccccc45)nc(-c4cccc5ccc6ccccc6c45)n3)cc2)cc1]

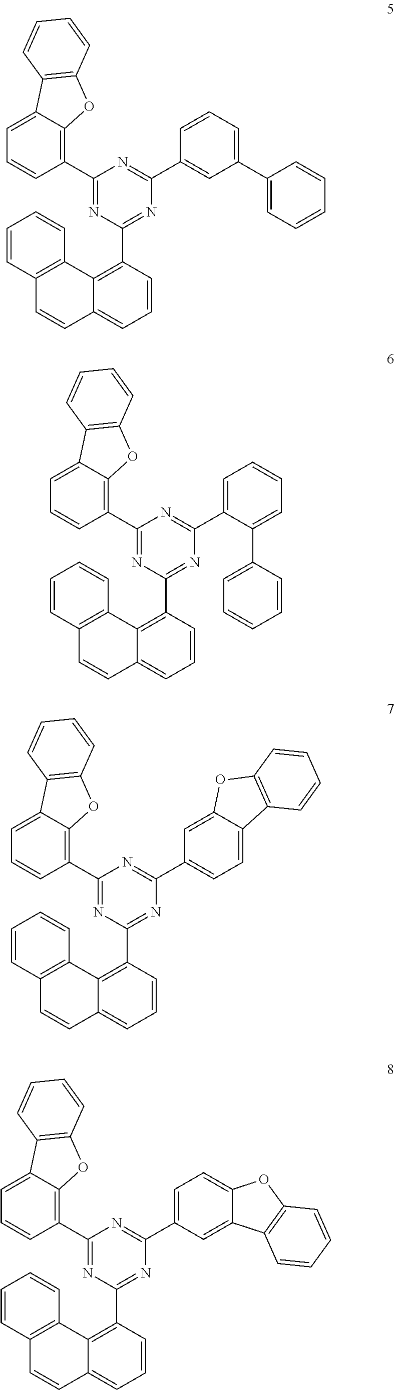

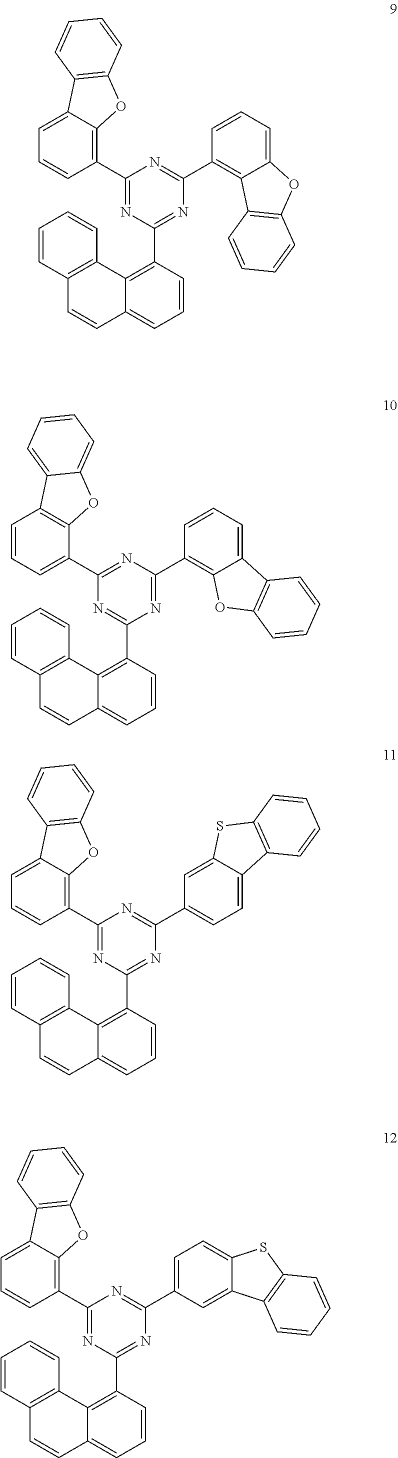

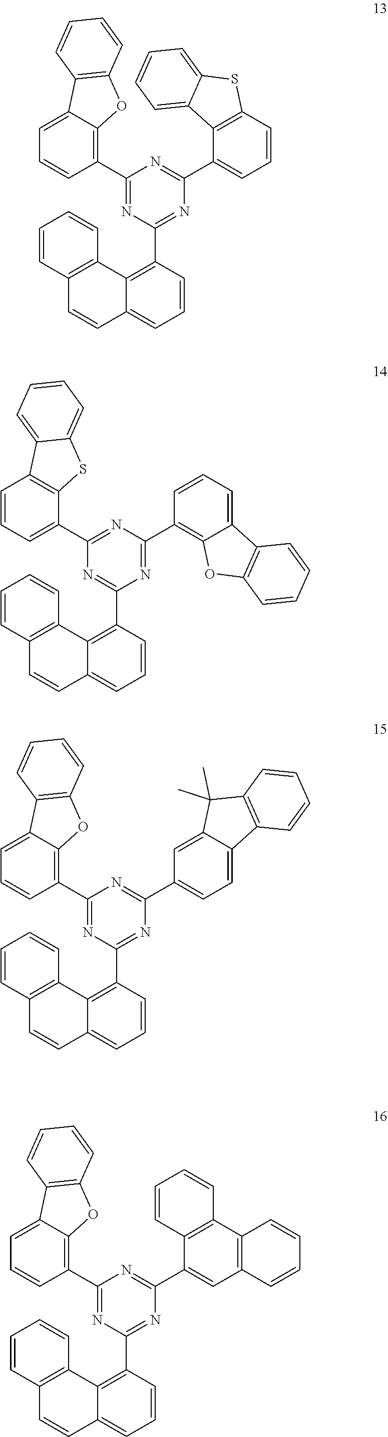 [SMILES: c1ccc2c(c1)ccc1cccc(-c3nc(-c4cccc5c4oc4ccccc45)nc(-c4cccc5sc6ccccc6c45)n3)c12 ; c1ccc2c(c1)ccc1cccc(-c3nc(-c4cccc5c4oc4ccccc45)nc(-c4cccc5c4sc4ccccc45)n3)c12 ; CC1(C)c2ccccc2-c2ccc(-c3nc(-c4cccc5c4oc4ccccc45)nc(-c4cccc5ccc6ccccc6c45)n3)cc21 ; c1ccc2c(c1)cc(-c1nc(-c3cccc4c3oc3ccccc34)nc(-c3cccc4ccc5ccccc5c34)n1)c1ccccc12]

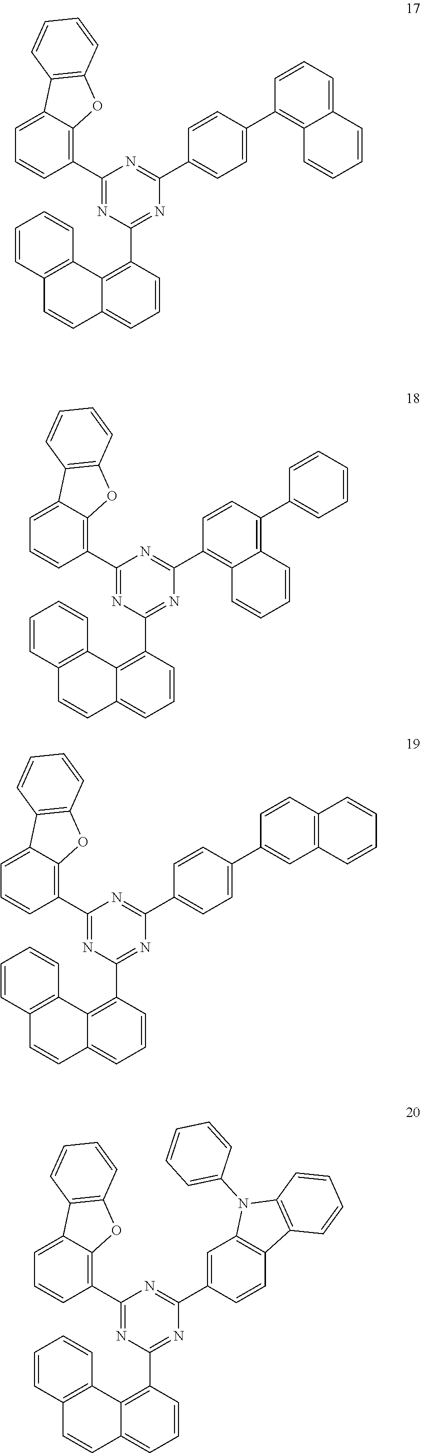

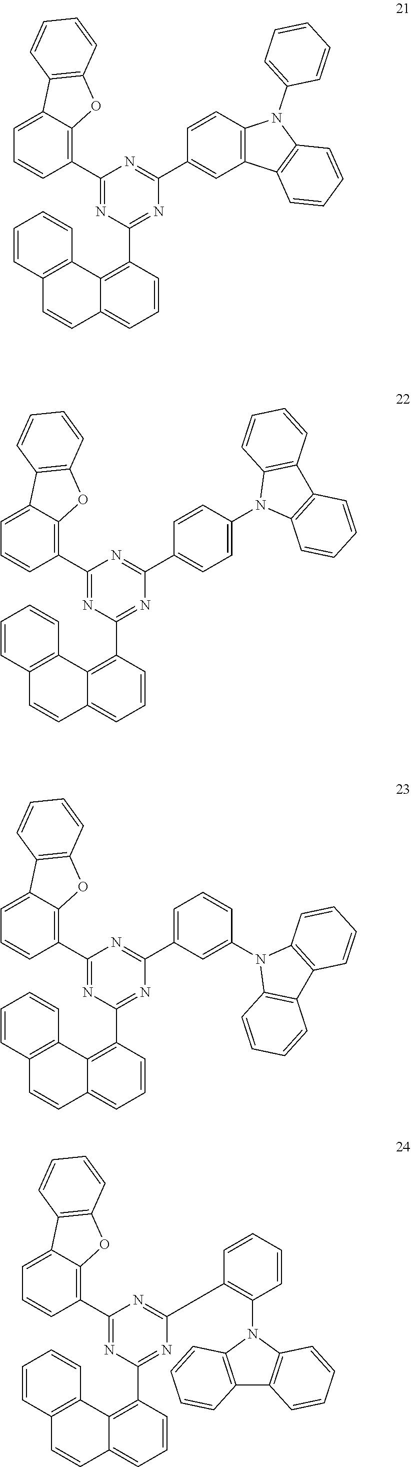 [SMILES: c1ccc(-n2c3ccccc3c3cc(-c4nc(-c5cccc6c5oc5ccccc56)nc(-c5cccc6ccc7ccccc7c56)n4)ccc32)cc1 ; c1ccc2c(c1)ccc1cccc(-c3nc(-c4ccc(-n5c6ccccc6c6ccccc65)cc4)nc(-c4cccc5c4oc4ccccc45)n3)c12 ; c1cc(-c2nc(-c3cccc4c3oc3ccccc34)nc(-c3cccc4ccc5ccccc5c34)n2)cc(-n2c3ccccc3c3ccccc32)c1 ; c1ccc(-n2c3ccccc3c3ccccc32)c(-c2nc(-c3cccc4c3oc3ccccc34)nc(-c3cccc4ccc5ccccc5c34)n2)c1]

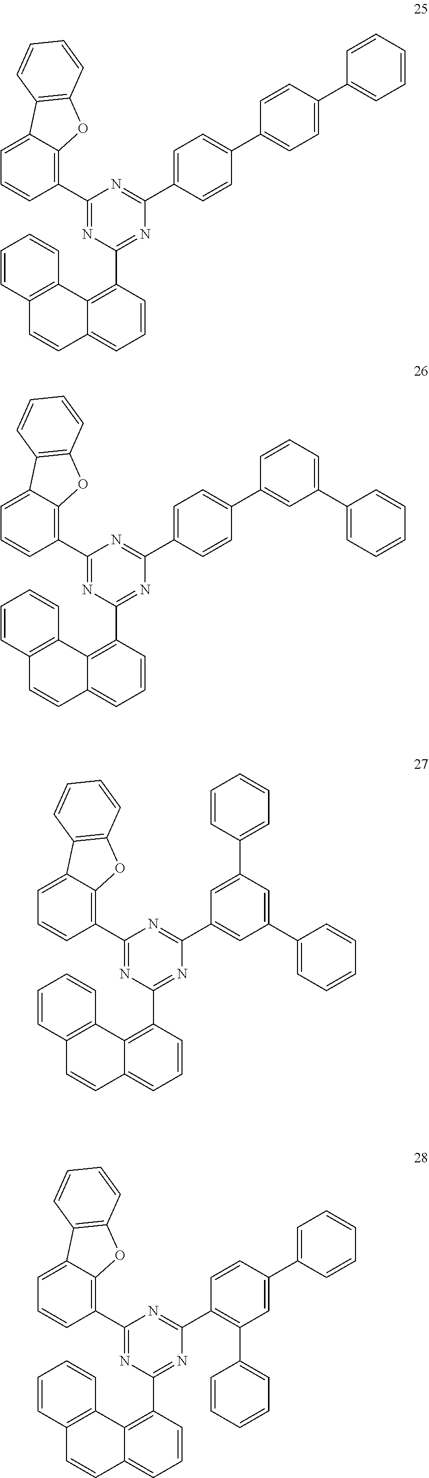

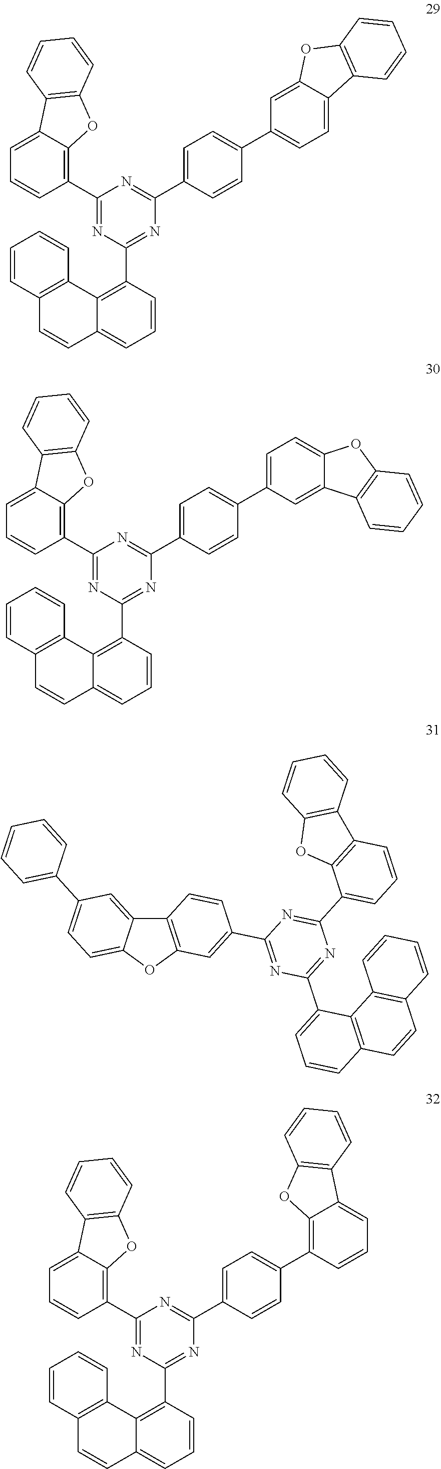

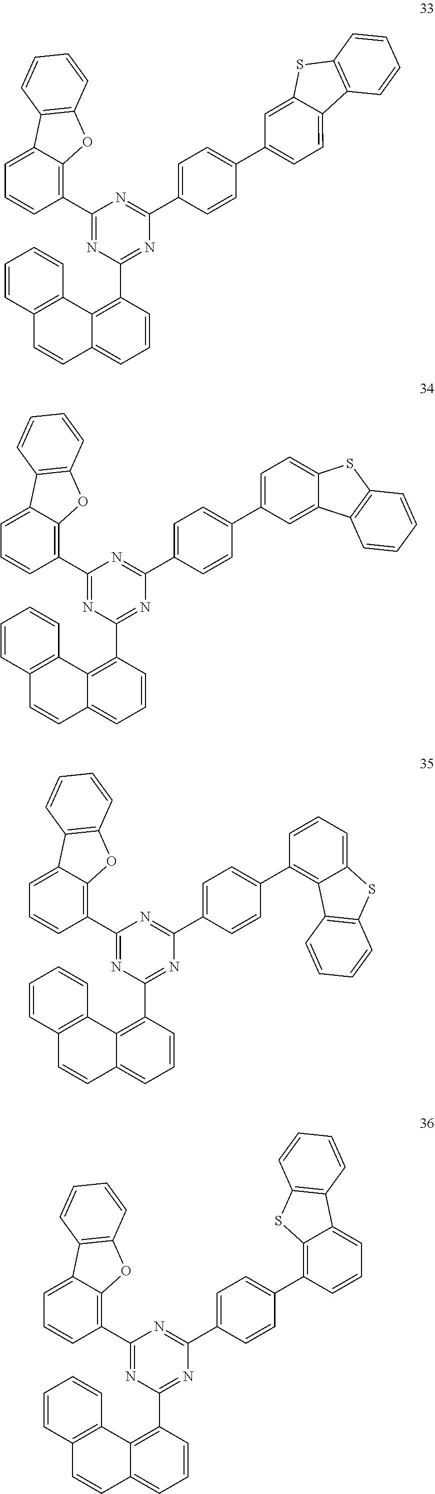 [SMILES: c1ccc2c(c1)ccc1cccc(-c3nc(-c4ccc(-c5ccc6c(c5)sc5ccccc56)cc4)nc(-c4cccc5c4oc4ccccc45)n3)c12 ; c1ccc2c(c1)ccc1cccc(-c3nc(-c4ccc(-c5ccc6sc7ccccc7c6c5)cc4)nc(-c4cccc5c4oc4ccccc45)n3)c12 ; c1ccc2c(c1)ccc1cccc(-c3nc(-c4ccc(-c5cccc6sc7ccccc7c56)cc4)nc(-c4cccc5c4oc4ccccc45)n3)c12 ; c1ccc2c(c1)ccc1cccc(-c3nc(-c4ccc(-c5cccc6c5sc5ccccc56)cc4)nc(-c4cccc5c4oc4ccccc45)n3)c12]

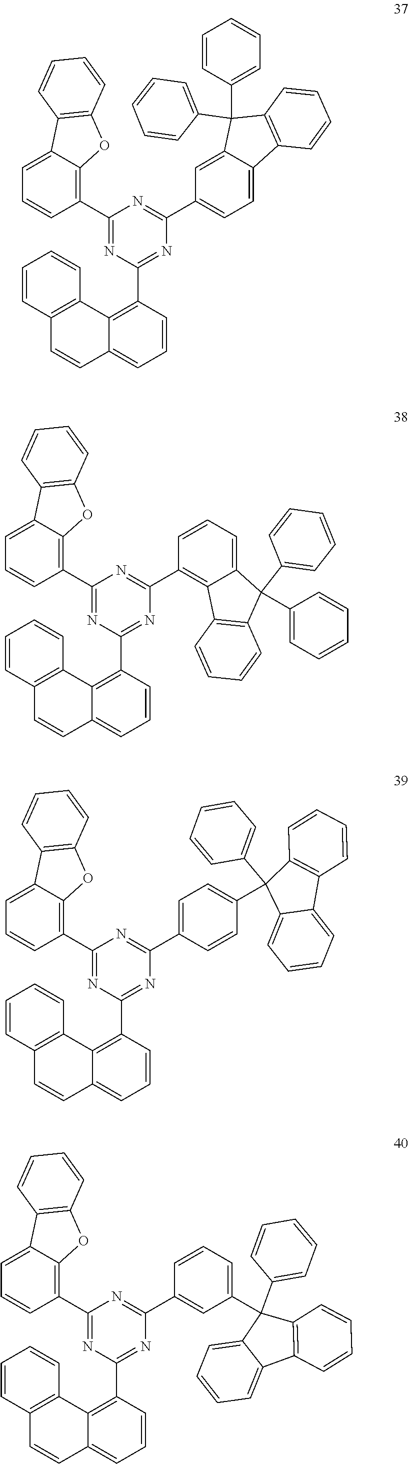 [SMILES: c1ccc(C2(c3ccccc3)c3ccccc3-c3ccc(-c4nc(-c5cccc6c5oc5ccccc56)nc(-c5cccc6ccc7ccccc7c56)n4)cc32)cc1 ; c1ccc(C2(c3ccccc3)c3ccccc3-c3c(-c4nc(-c5cccc6c5oc5ccccc56)nc(-c5cccc6ccc7ccccc7c56)n4)cccc32)cc1 ; c1ccc(C2(c3ccc(-c4nc(-c5cccc6c5oc5ccccc56)nc(-c5cccc6ccc7ccccc7c56)n4)cc3)c3ccccc3-c3ccccc32)cc1 ; c1ccc(C2(c3cccc(-c4nc(-c5cccc6c5oc5ccccc56)nc(-c5cccc6ccc7ccccc7c56)n4)c3)c3ccccc3-c3ccccc32)cc1]

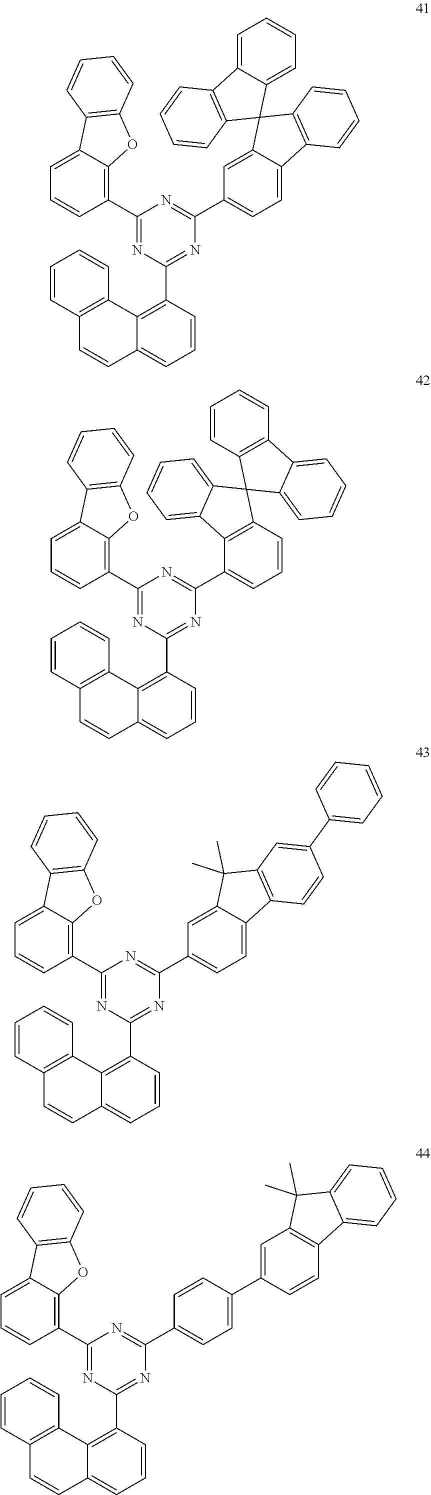 [SMILES: c1ccc2c(c1)-c1ccccc1C21c2ccccc2-c2ccc(-c3nc(-c4cccc5c4oc4ccccc45)nc(-c4cccc5ccc6ccccc6c45)n3)cc21 ; c1ccc2c(c1)-c1ccccc1C21c2ccccc2-c2c(-c3nc(-c4cccc5c4oc4ccccc45)nc(-c4cccc5ccc6ccccc6c45)n3)cccc21 ; CC1(C)c2cc(-c3ccccc3)ccc2-c2ccc(-c3nc(-c4cccc5c4oc4ccccc45)nc(-c4cccc5ccc6ccccc6c45)n3)cc21 ; CC1(C)c2ccccc2-c2ccc(-c3ccc(-c4nc(-c5cccc6c5oc5ccccc56)nc(-c5cccc6ccc7ccccc7c56)n4)cc3)cc21]

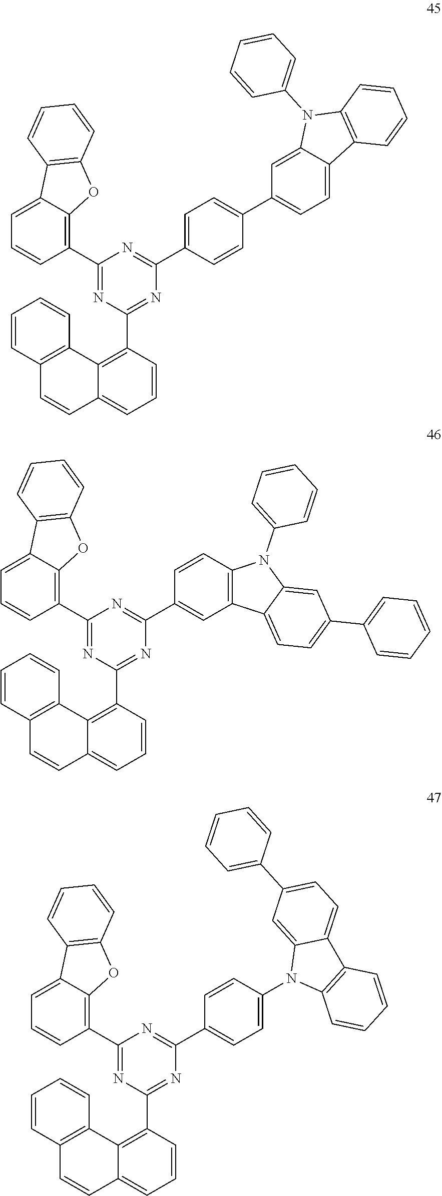

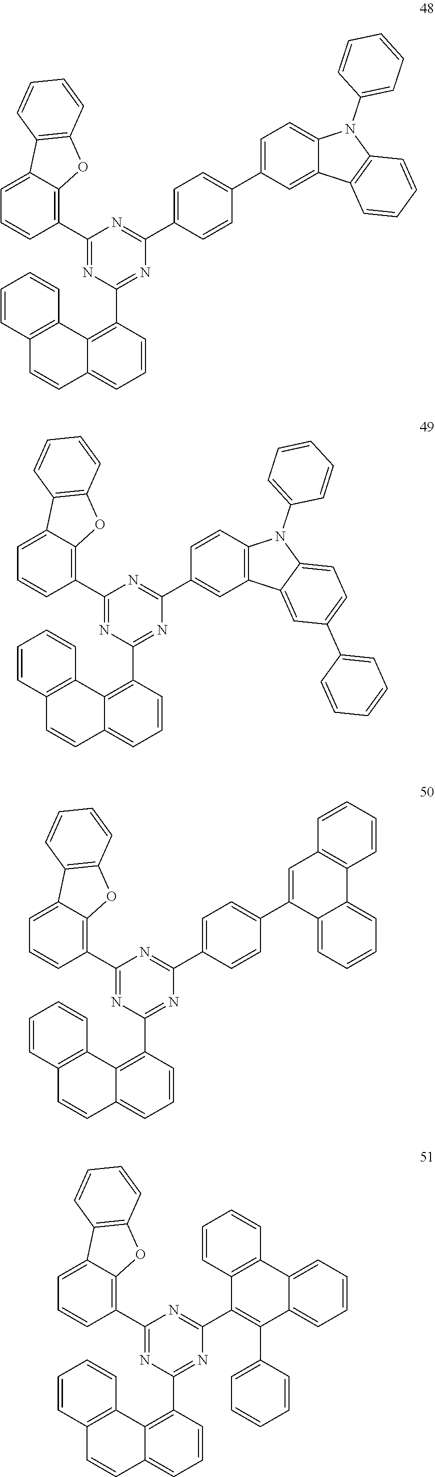 [SMILES: c1ccc(-n2c3ccccc3c3cc(-c4ccc(-c5nc(-c6cccc7c6oc6ccccc67)nc(-c6cccc7ccc8ccccc8c67)n5)cc4)ccc32)cc1 ; c1ccc(-c2ccc3c(c2)c2cc(-c4nc(-c5cccc6c5oc5ccccc56)nc(-c5cccc6ccc7ccccc7c56)n4)ccc2n3-c2ccccc2)cc1 ; c1ccc2c(c1)cc(-c1ccc(-c3nc(-c4cccc5c4oc4ccccc45)nc(-c4cccc5ccc6ccccc6c45)n3)cc1)c1ccccc12 ; c1ccc(-c2c(-c3nc(-c4cccc5c4oc4ccccc45)nc(-c4cccc5ccc6ccccc6c45)n3)c3ccccc3c3ccccc23)cc1]

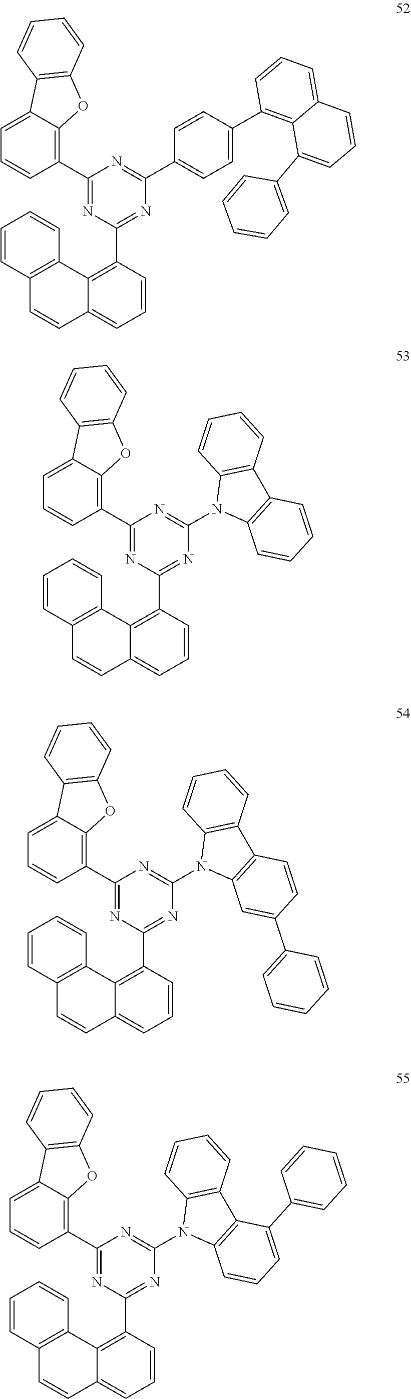

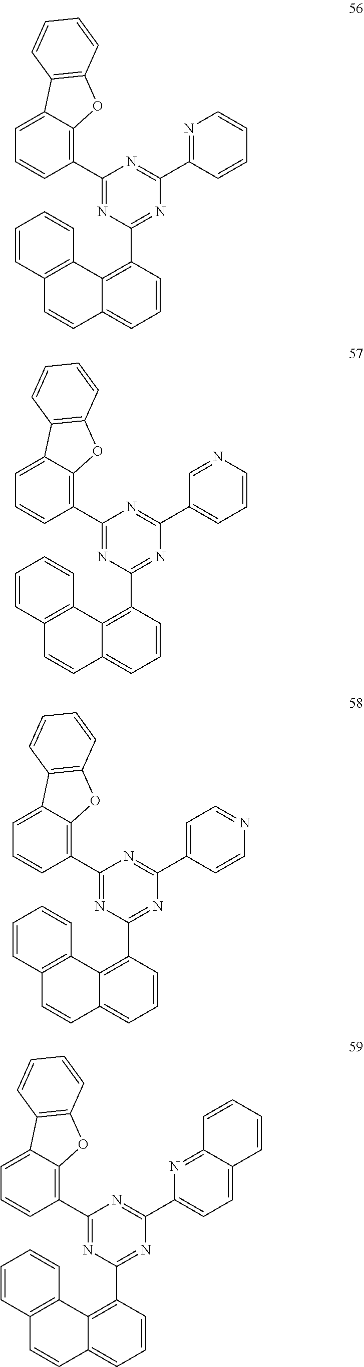

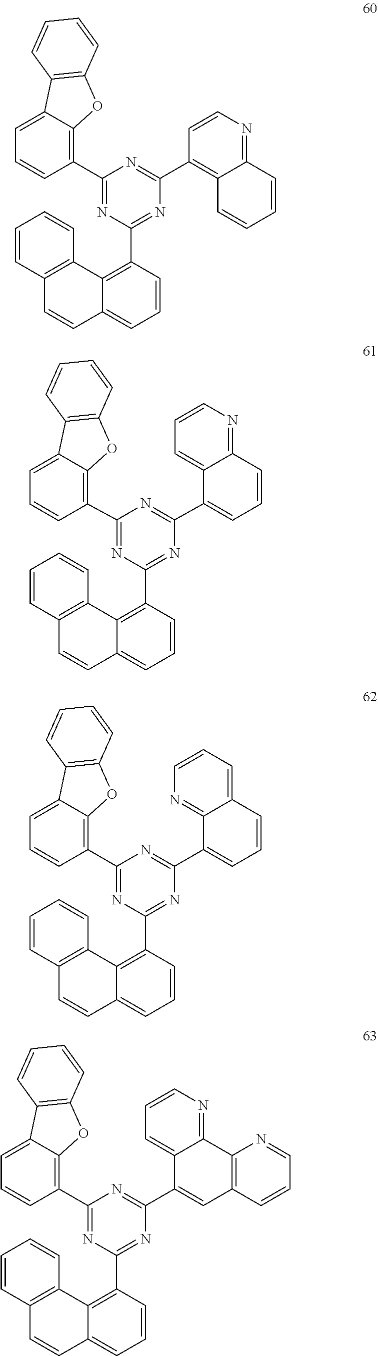

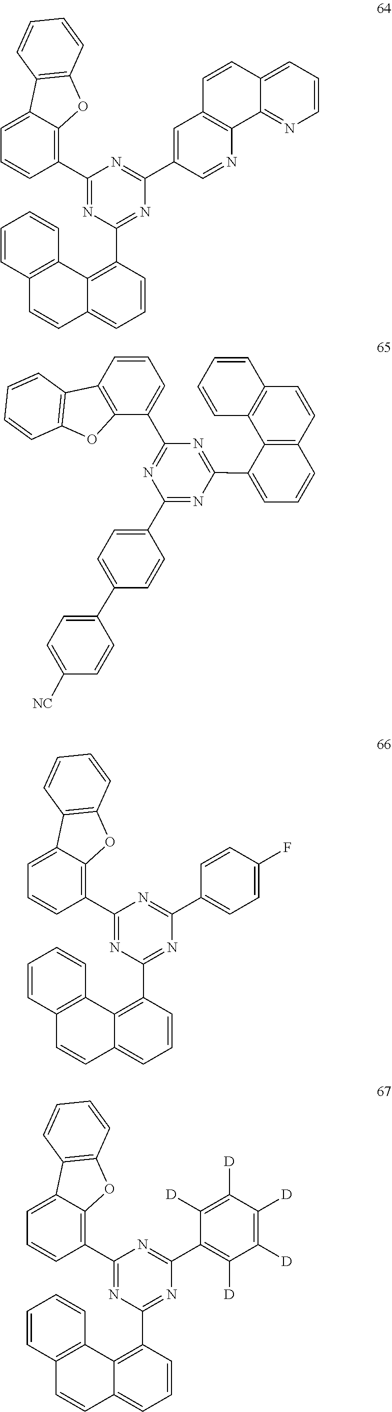

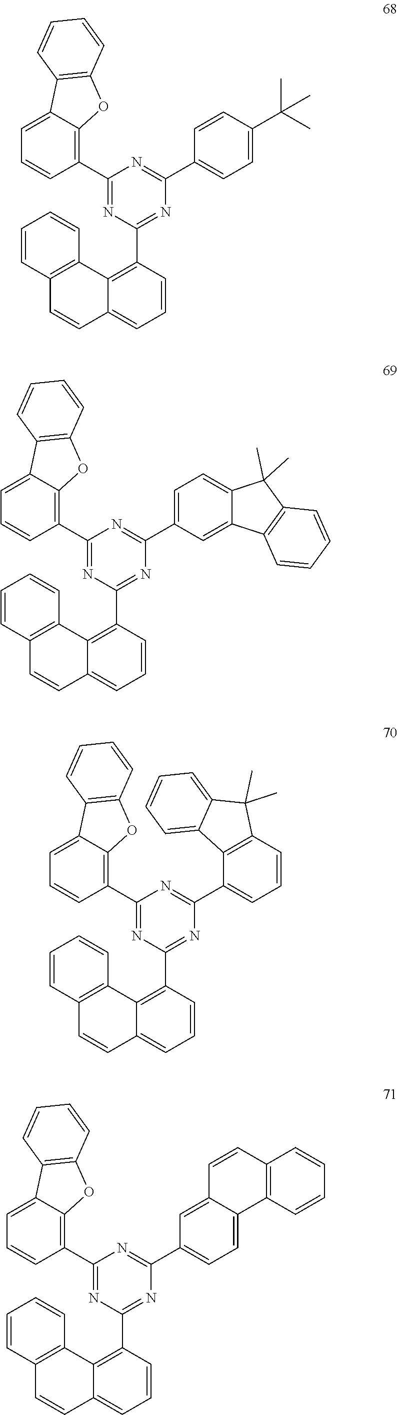 [SMILES: CC(C)(C)c1ccc(-c2nc(-c3cccc4c3oc3ccccc34)nc(-c3cccc4ccc5ccccc5c34)n2)cc1 ; CC1(C)c2ccccc2-c2cc(-c3nc(-c4cccc5c4oc4ccccc45)nc(-c4cccc5ccc6ccccc6c45)n3)ccc21 ; CC1(C)c2ccccc2-c2c(-c3nc(-c4cccc5c4oc4ccccc45)nc(-c4cccc5ccc6ccccc6c45)n3)cccc21 ; c1ccc2c(c1)ccc1cc(-c3nc(-c4cccc5c4oc4ccccc45)nc(-c4cccc5ccc6ccccc6c45)n3)ccc12]

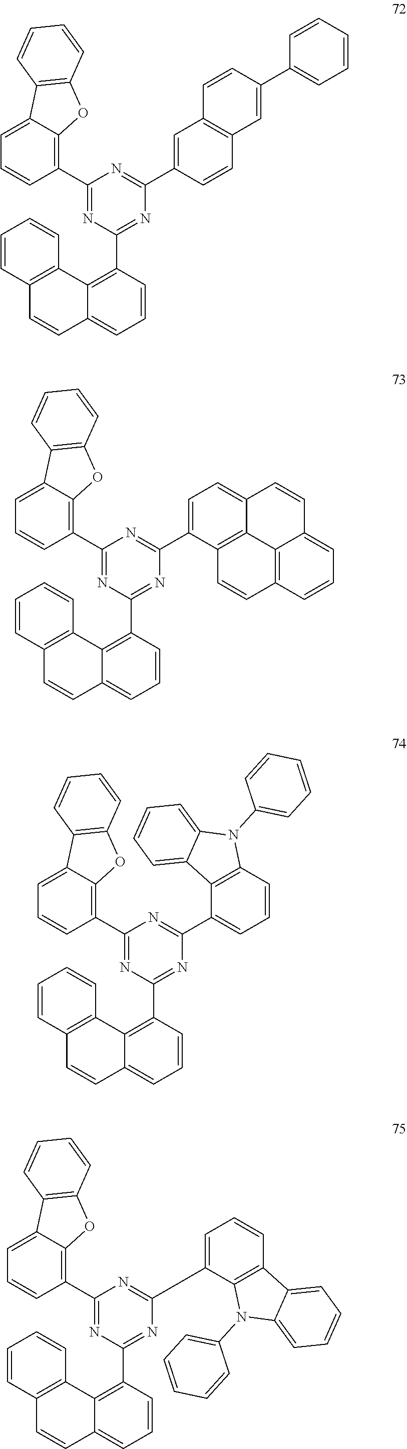 [SMILES: c1ccc(-c2ccc3cc(-c4nc(-c5cccc6c5oc5ccccc56)nc(-c5cccc6ccc7ccccc7c56)n4)ccc3c2)cc1 ; c1ccc2c(c1)ccc1cccc(-c3nc(-c4ccc5ccc6cccc7ccc4c5c67)nc(-c4cccc5c4oc4ccccc45)n3)c12 ; c1ccc(-n2c3ccccc3c3c(-c4nc(-c5cccc6c5oc5ccccc56)nc(-c5cccc6ccc7ccccc7c56)n4)cccc32)cc1 ; c1ccc(-n2c3ccccc3c3cccc(-c4nc(-c5cccc6c5oc5ccccc56)nc(-c5cccc6ccc7ccccc7c56)n4)c32)cc1]

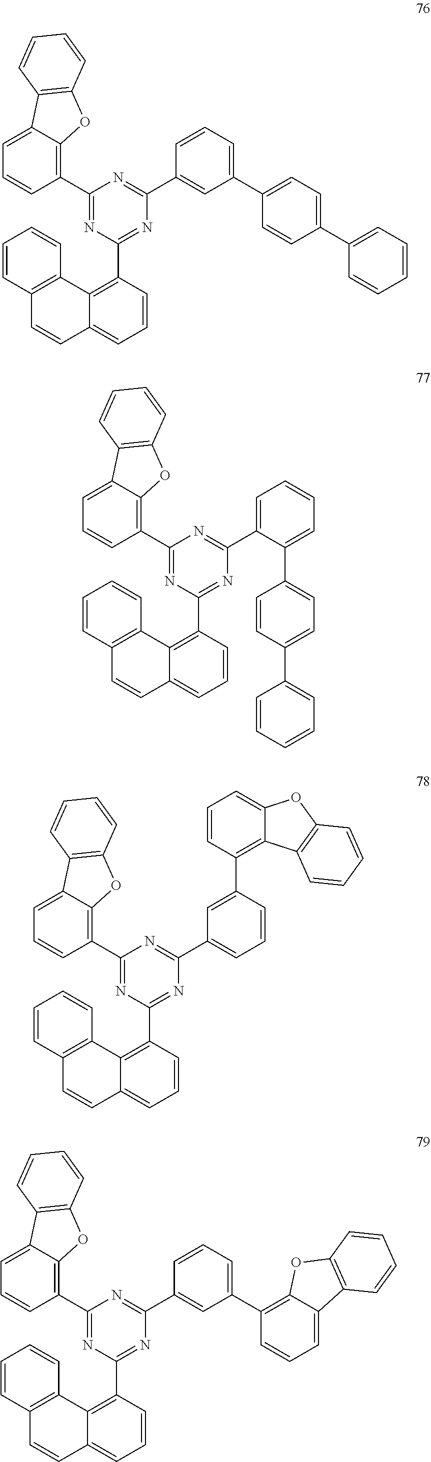 [SMILES: c1ccc(-c2ccc(-c3cccc(-c4nc(-c5cccc6c5oc5ccccc56)nc(-c5cccc6ccc7ccccc7c56)n4)c3)cc2)cc1 ; c1ccc(-c2ccc(-c3ccccc3-c3nc(-c4cccc5c4oc4ccccc45)nc(-c4cccc5ccc6ccccc6c45)n3)cc2)cc1 ; c1cc(-c2nc(-c3cccc4c3oc3ccccc34)nc(-c3cccc4ccc5ccccc5c34)n2)cc(-c2cccc3oc4ccccc4c23)c1 ; c1cc(-c2nc(-c3cccc4c3oc3ccccc34)nc(-c3cccc4ccc5ccccc5c34)n2)cc(-c2cccc3c2oc2ccccc23)c1]

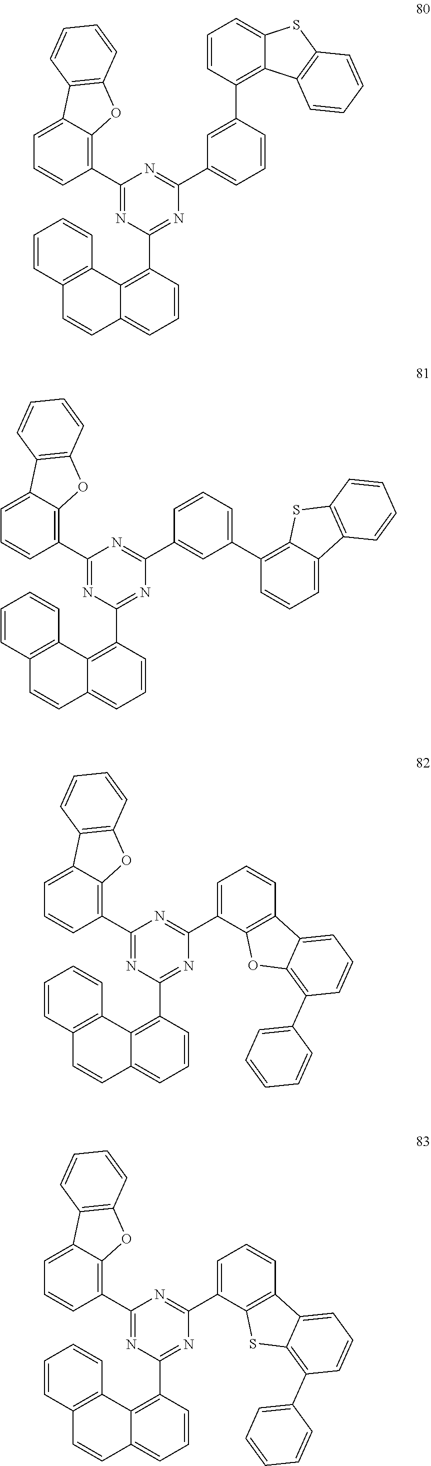 [SMILES: c1cc(-c2nc(-c3cccc4c3oc3ccccc34)nc(-c3cccc4ccc5ccccc5c34)n2)cc(-c2cccc3sc4ccccc4c23)c1 ; c1cc(-c2nc(-c3cccc4c3oc3ccccc34)nc(-c3cccc4ccc5ccccc5c34)n2)cc(-c2cccc3c2sc2ccccc23)c1 ; c1ccc(-c2cccc3c2oc2c(-c4nc(-c5cccc6c5oc5ccccc56)nc(-c5cccc6ccc7ccccc7c56)n4)cccc23)cc1 ; c1ccc(-c2cccc3c2sc2c(-c4nc(-c5cccc6c5oc5ccccc56)nc(-c5cccc6ccc7ccccc7c56)n4)cccc23)cc1]

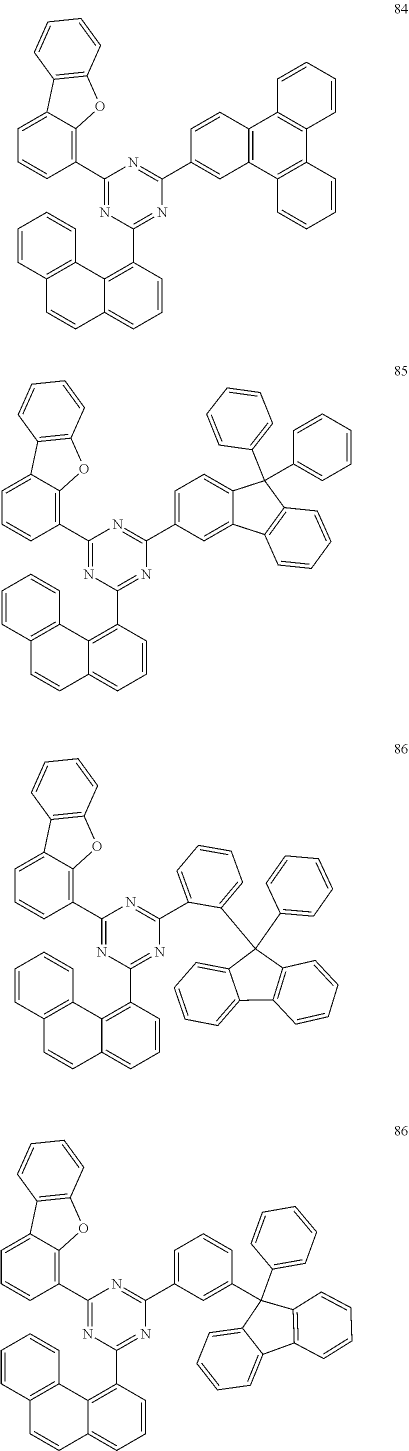 [SMILES: c1ccc2c(c1)ccc1cccc(-c3nc(-c4ccc5c6ccccc6c6ccccc6c5c4)nc(-c4cccc5c4oc4ccccc45)n3)c12 ; c1ccc(C2(c3ccccc3)c3ccccc3-c3cc(-c4nc(-c5cccc6c5oc5ccccc56)nc(-c5cccc6ccc7ccccc7c56)n4)ccc32)cc1 ; c1ccc(C2(c3ccccc3-c3nc(-c4cccc5c4oc4ccccc45)nc(-c4cccc5ccc6ccccc6c45)n3)c3ccccc3-c3ccccc32)cc1 ; c1ccc(C2(c3cccc(-c4nc(-c5cccc6c5oc5ccccc56)nc(-c5cccc6ccc7ccccc7c56)n4)c3)c3ccccc3-c3ccccc32)cc1]

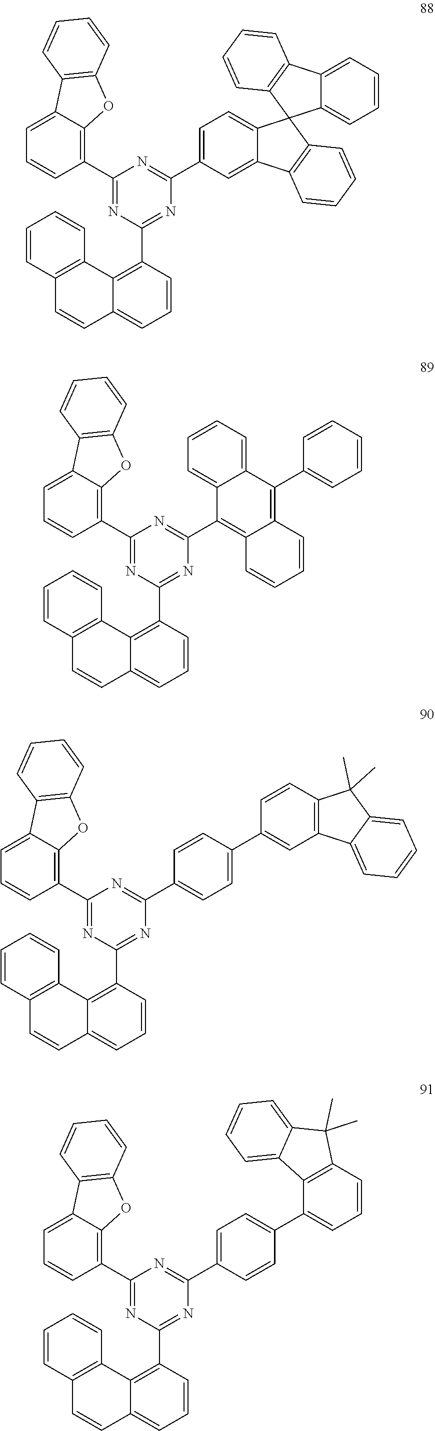 [SMILES: c1ccc2c(c1)-c1ccccc1C21c2ccccc2-c2cc(-c3nc(-c4cccc5c4oc4ccccc45)nc(-c4cccc5ccc6ccccc6c45)n3)ccc21 ; c1ccc(-c2c3ccccc3c(-c3nc(-c4cccc5c4oc4ccccc45)nc(-c4cccc5ccc6ccccc6c45)n3)c3ccccc23)cc1 ; CC1(C)c2ccccc2-c2cc(-c3ccc(-c4nc(-c5cccc6c5oc5ccccc56)nc(-c5cccc6ccc7ccccc7c56)n4)cc3)ccc21 ; CC1(C)c2ccccc2-c2c(-c3ccc(-c4nc(-c5cccc6c5oc5ccccc56)nc(-c5cccc6ccc7ccccc7c56)n4)cc3)cccc21]

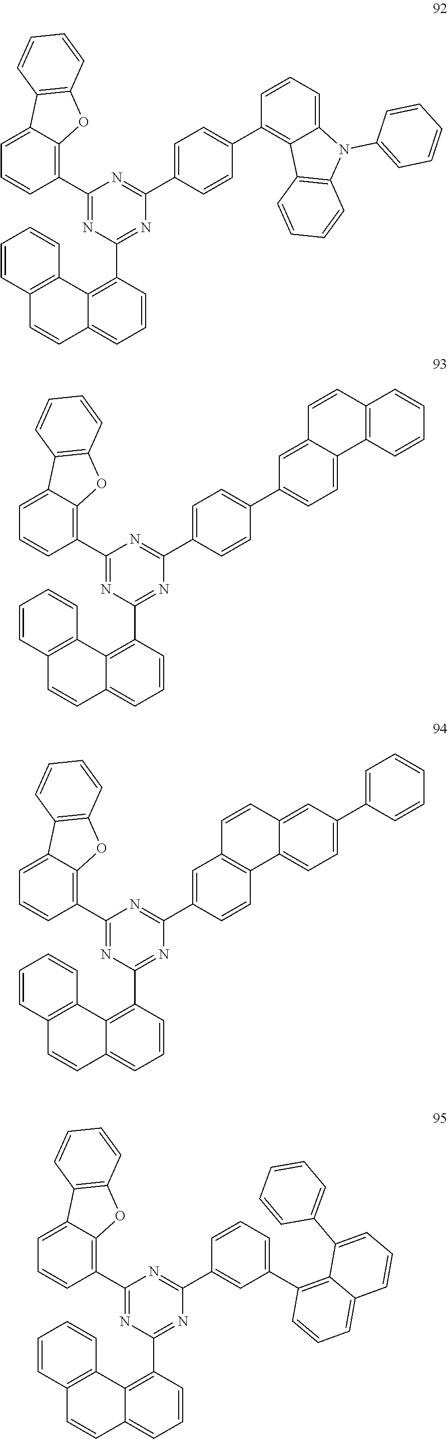 [SMILES: c1ccc(-n2c3ccccc3c3c(-c4ccc(-c5nc(-c6cccc7c6oc6ccccc67)nc(-c6cccc7ccc8ccccc8c67)n5)cc4)cccc32)cc1 ; c1ccc2c(c1)ccc1cc(-c3ccc(-c4nc(-c5cccc6c5oc5ccccc56)nc(-c5cccc6ccc7ccccc7c56)n4)cc3)ccc12 ; c1ccc(-c2ccc3c(ccc4cc(-c5nc(-c6cccc7c6oc6ccccc67)nc(-c6cccc7ccc8ccccc8c67)n5)ccc43)c2)cc1 ; c1ccc(-c2cccc3cccc(-c4cccc(-c5nc(-c6cccc7c6oc6ccccc67)nc(-c6cccc7ccc8ccccc8c67)n5)c4)c23)cc1]

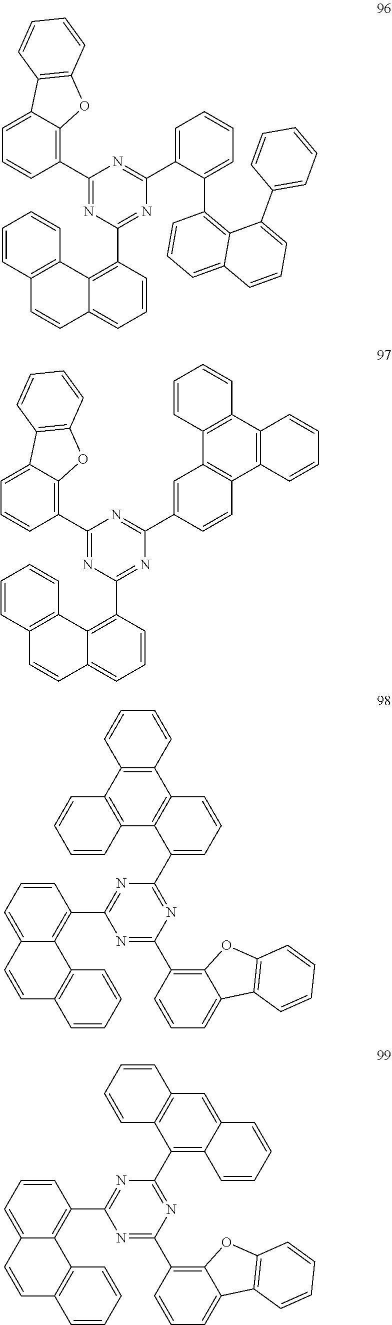

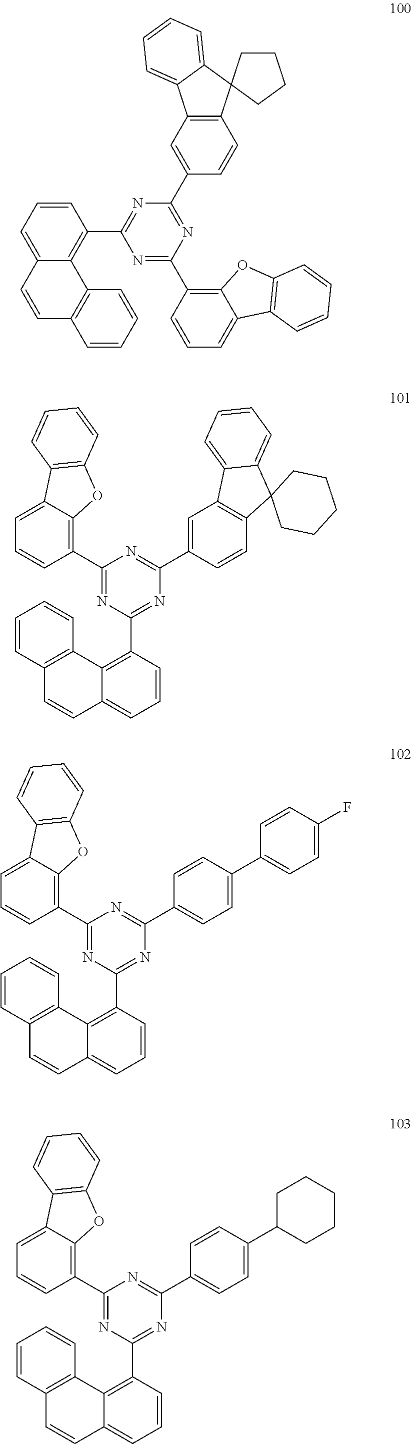 [SMILES: c1ccc2c(c1)-c1cc(-c3nc(-c4cccc5c4oc4ccccc45)nc(-c4cccc5ccc6ccccc6c45)n3)ccc1C21CCCC1 ; c1ccc2c(c1)-c1cc(-c3nc(-c4cccc5c4oc4ccccc45)nc(-c4cccc5ccc6ccccc6c45)n3)ccc1C21CCCCC1 ; Fc1ccc(-c2ccc(-c3nc(-c4cccc5c4oc4ccccc45)nc(-c4cccc5ccc6ccccc6c45)n3)cc2)cc1 ; c1ccc2c(c1)ccc1cccc(-c3nc(-c4ccc(C5CCCCC5)cc4)nc(-c4cccc5c4oc4ccccc45)n3)c12]

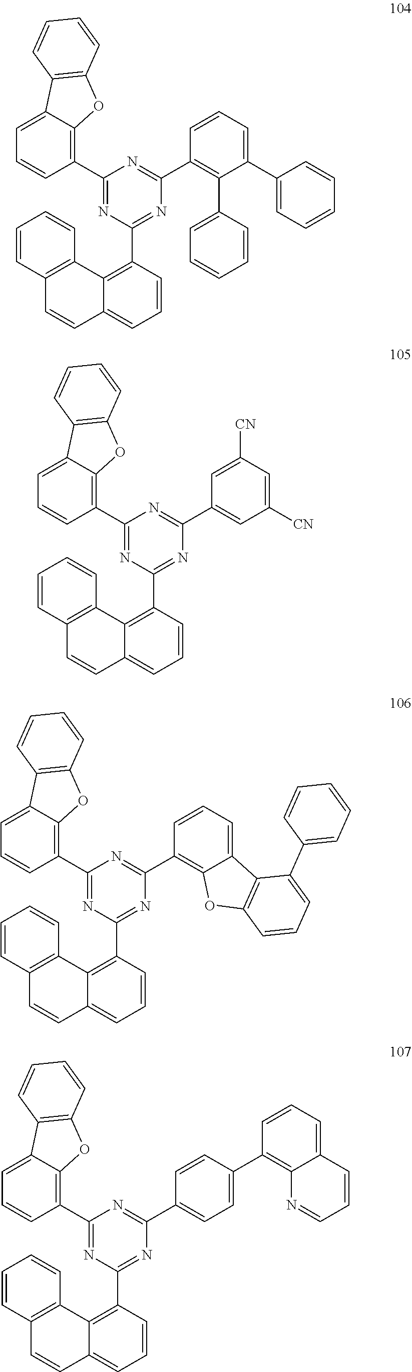

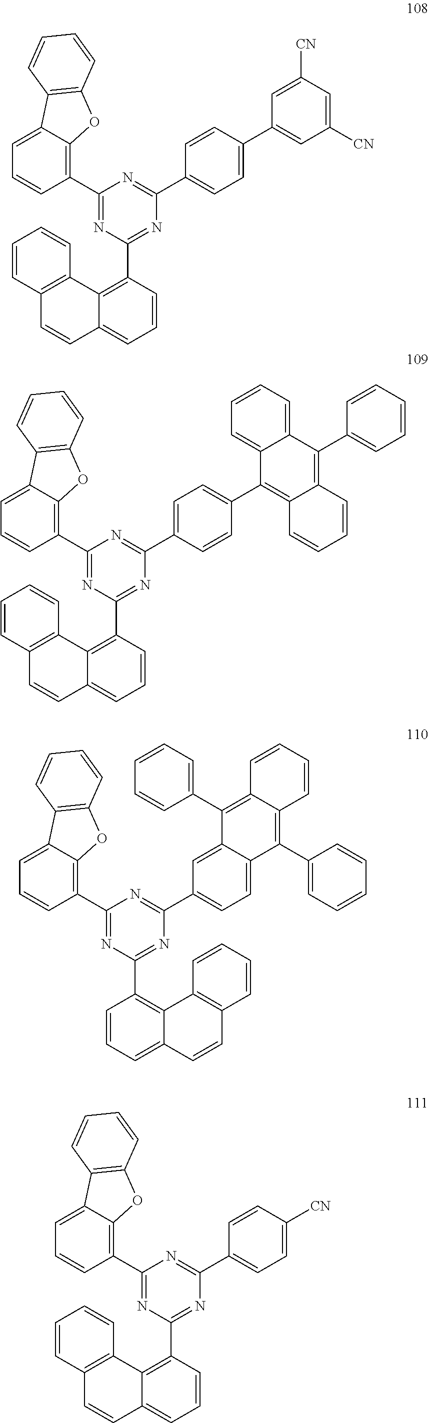 [SMILES: N#Cc1cc(C#N)cc(-c2ccc(-c3nc(-c4cccc5c4oc4ccccc45)nc(-c4cccc5ccc6ccccc6c45)n3)cc2)c1 ; c1ccc(-c2c3ccccc3c(-c3ccc(-c4nc(-c5cccc6c5oc5ccccc56)nc(-c5cccc6ccc7ccccc7c56)n4)cc3)c3ccccc23)cc1 ; c1ccc(-c2c3ccccc3c(-c3ccccc3)c3cc(-c4nc(-c5cccc6c5oc5ccccc56)nc(-c5cccc6ccc7ccccc7c56)n4)ccc23)cc1 ; N#Cc1ccc(-c2nc(-c3cccc4c3oc3ccccc34)nc(-c3cccc4ccc5ccccc5c34)n2)cc1]

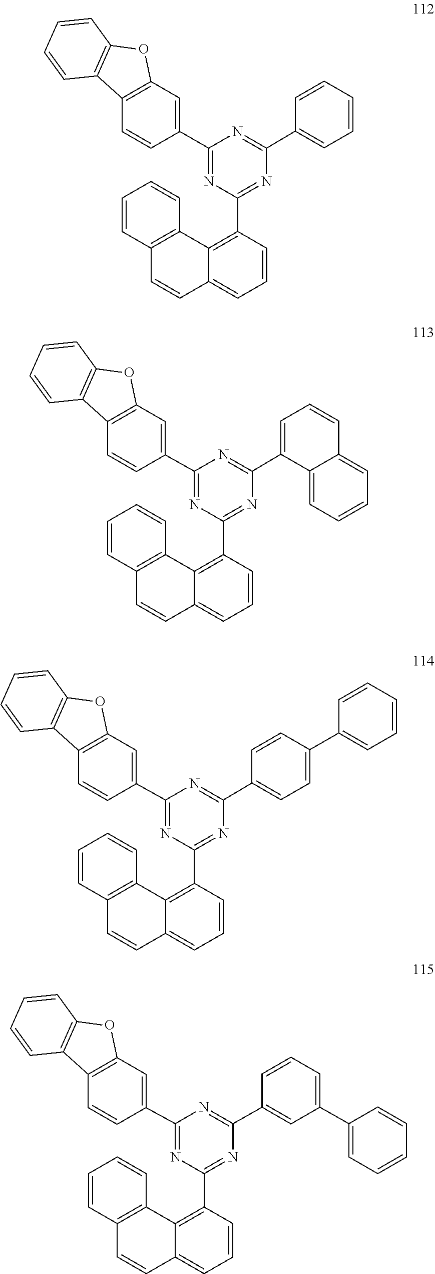 [SMILES: c1ccc(-c2nc(-c3ccc4c(c3)oc3ccccc34)nc(-c3cccc4ccc5ccccc5c34)n2)cc1 ; c1ccc2c(-c3nc(-c4ccc5c(c4)oc4ccccc45)nc(-c4cccc5ccc6ccccc6c45)n3)cccc2c1 ; c1ccc(-c2ccc(-c3nc(-c4ccc5c(c4)oc4ccccc45)nc(-c4cccc5ccc6ccccc6c45)n3)cc2)cc1 ; c1ccc(-c2cccc(-c3nc(-c4ccc5c(c4)oc4ccccc45)nc(-c4cccc5ccc6ccccc6c45)n3)c2)cc1]

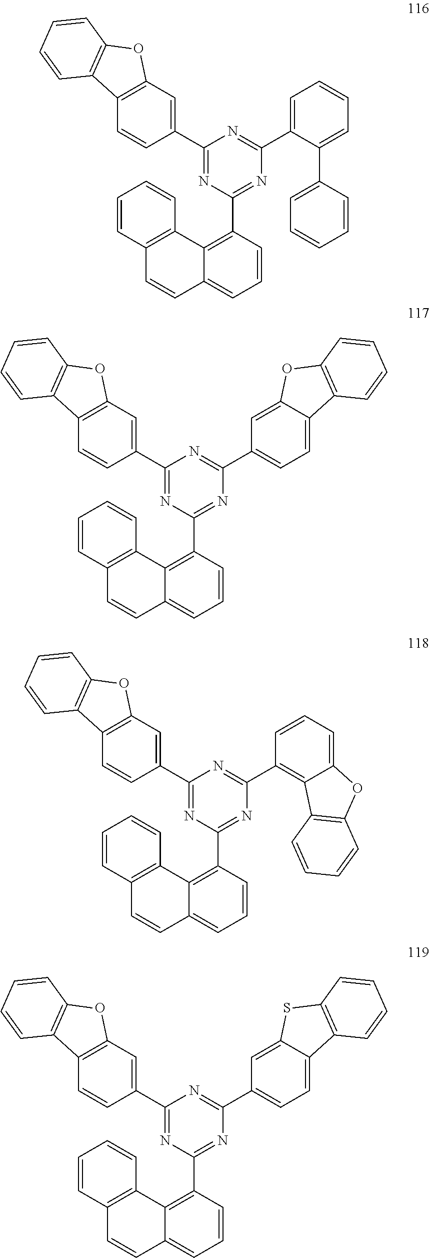 [SMILES: c1ccc(-c2ccccc2-c2nc(-c3ccc4c(c3)oc3ccccc34)nc(-c3cccc4ccc5ccccc5c34)n2)cc1 ; c1ccc2c(c1)ccc1cccc(-c3nc(-c4ccc5c(c4)oc4ccccc45)nc(-c4ccc5c(c4)oc4ccccc45)n3)c12 ; c1ccc2c(c1)ccc1cccc(-c3nc(-c4ccc5c(c4)oc4ccccc45)nc(-c4cccc5oc6ccccc6c45)n3)c12 ; c1ccc2c(c1)ccc1cccc(-c3nc(-c4ccc5c(c4)oc4ccccc45)nc(-c4ccc5c(c4)sc4ccccc45)n3)c12]

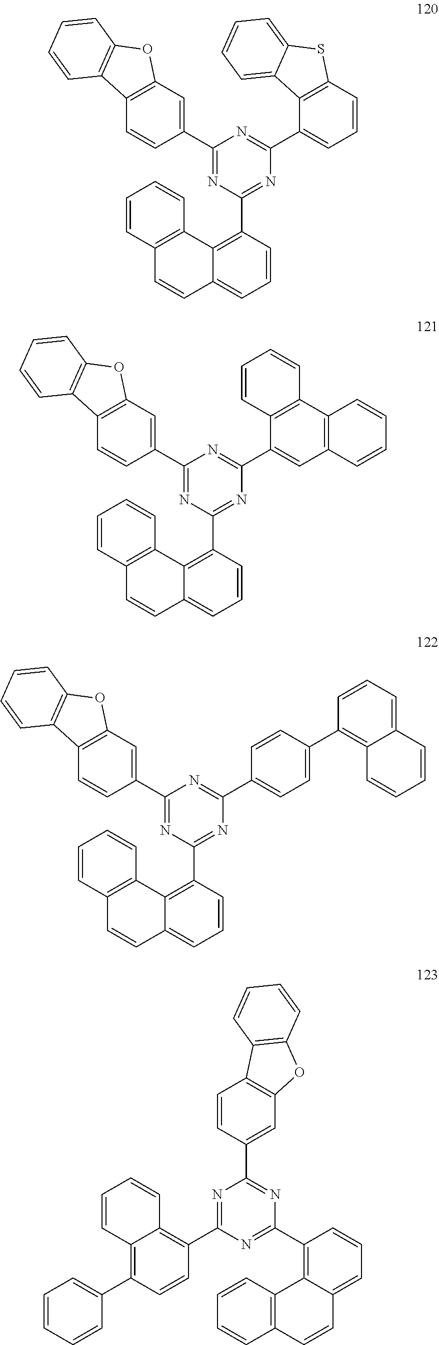 [SMILES: c1ccc2c(c1)ccc1cccc(-c3nc(-c4ccc5c(c4)oc4ccccc45)nc(-c4cccc5sc6ccccc6c45)n3)c12 ; c1ccc2c(c1)cc(-c1nc(-c3ccc4c(c3)oc3ccccc34)nc(-c3cccc4ccc5ccccc5c34)n1)c1ccccc12 ; c1ccc2c(-c3ccc(-c4nc(-c5ccc6c(c5)oc5ccccc56)nc(-c5cccc6ccc7ccccc7c56)n4)cc3)cccc2c1 ; c1ccc(-c2ccc(-c3nc(-c4ccc5c(c4)oc4ccccc45)nc(-c4cccc5ccc6ccccc6c45)n3)c3ccccc23)cc1]

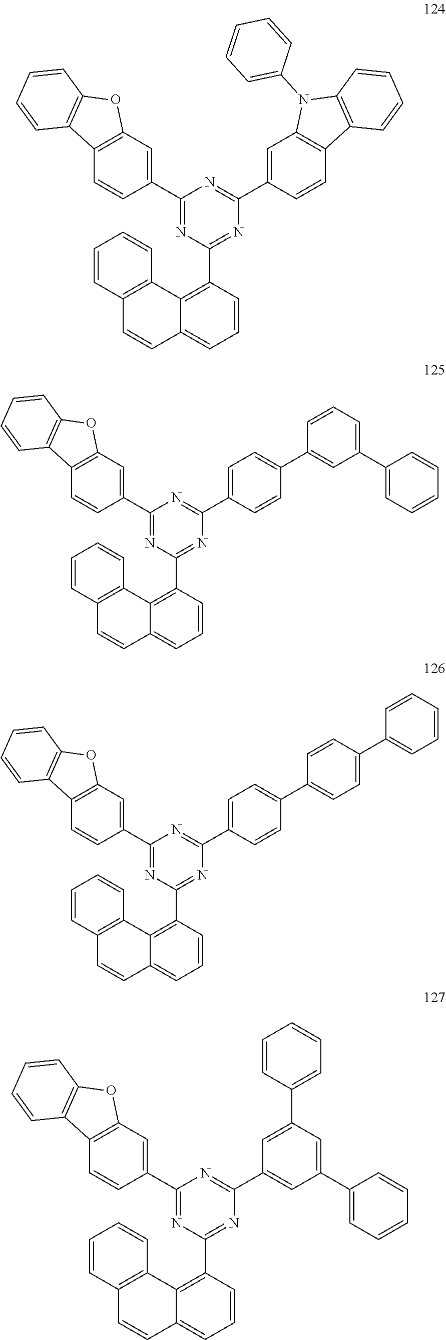 [SMILES: c1ccc(-n2c3ccccc3c3ccc(-c4nc(-c5ccc6c(c5)oc5ccccc56)nc(-c5cccc6ccc7ccccc7c56)n4)cc32)cc1 ; c1ccc(-c2cccc(-c3ccc(-c4nc(-c5ccc6c(c5)oc5ccccc56)nc(-c5cccc6ccc7ccccc7c56)n4)cc3)c2)cc1 ; c1ccc(-c2ccc(-c3ccc(-c4nc(-c5ccc6c(c5)oc5ccccc56)nc(-c5cccc6ccc7ccccc7c56)n4)cc3)cc2)cc1 ; c1ccc(-c2cc(-c3ccccc3)cc(-c3nc(-c4ccc5c(c4)oc4ccccc45)nc(-c4cccc5ccc6ccccc6c45)n3)c2)cc1]

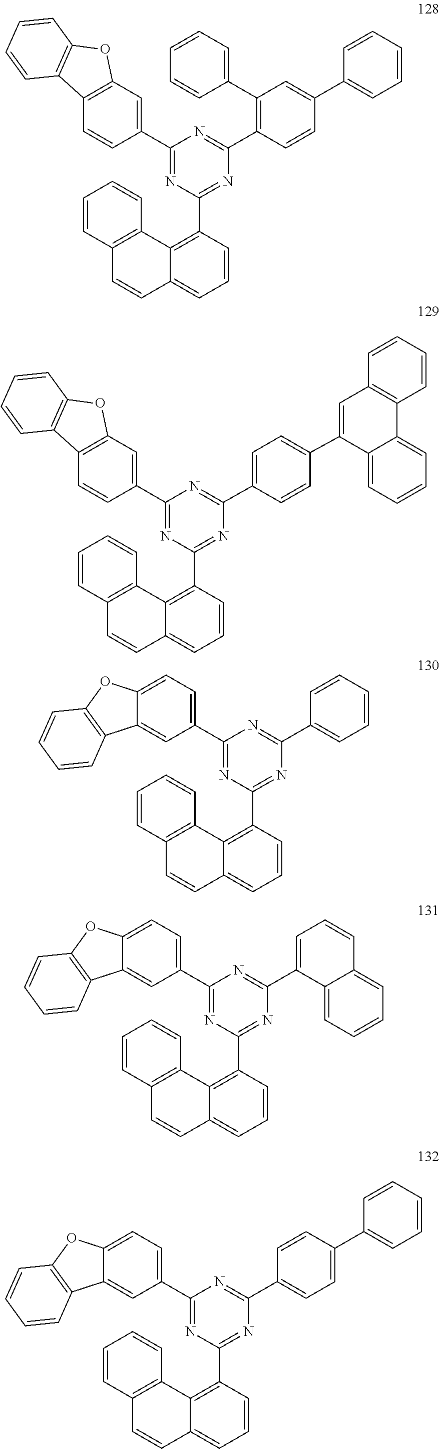

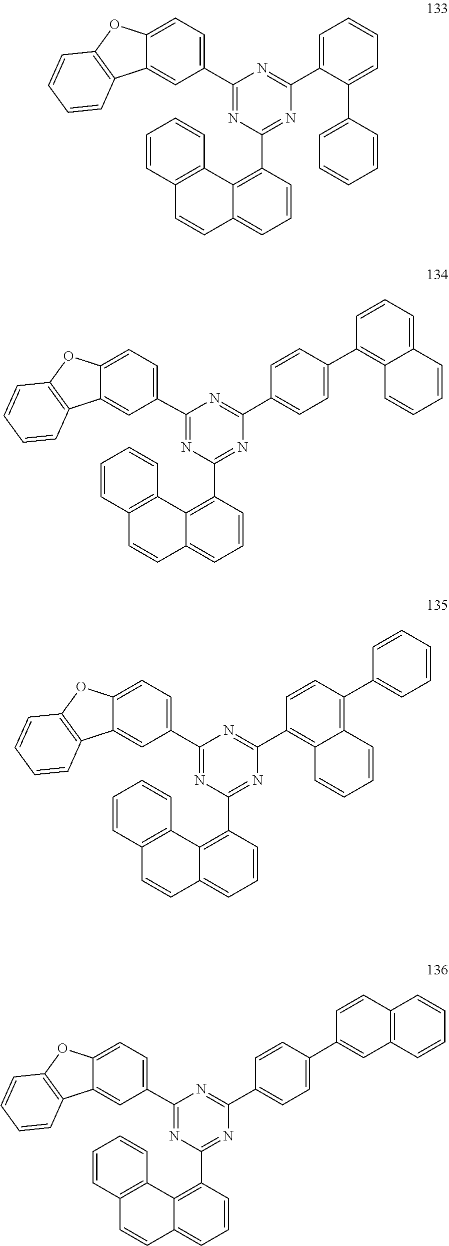 [SMILES: c1ccc(-c2ccccc2-c2nc(-c3ccc4oc5ccccc5c4c3)nc(-c3cccc4ccc5ccccc5c34)n2)cc1 ; c1ccc2c(-c3ccc(-c4nc(-c5ccc6oc7ccccc7c6c5)nc(-c5cccc6ccc7ccccc7c56)n4)cc3)cccc2c1 ; c1ccc(-c2ccc(-c3nc(-c4ccc5oc6ccccc6c5c4)nc(-c4cccc5ccc6ccccc6c45)n3)c3ccccc23)cc1 ; c1ccc2cc(-c3ccc(-c4nc(-c5ccc6oc7ccccc7c6c5)nc(-c5cccc6ccc7ccccc7c56)n4)cc3)ccc2c1]

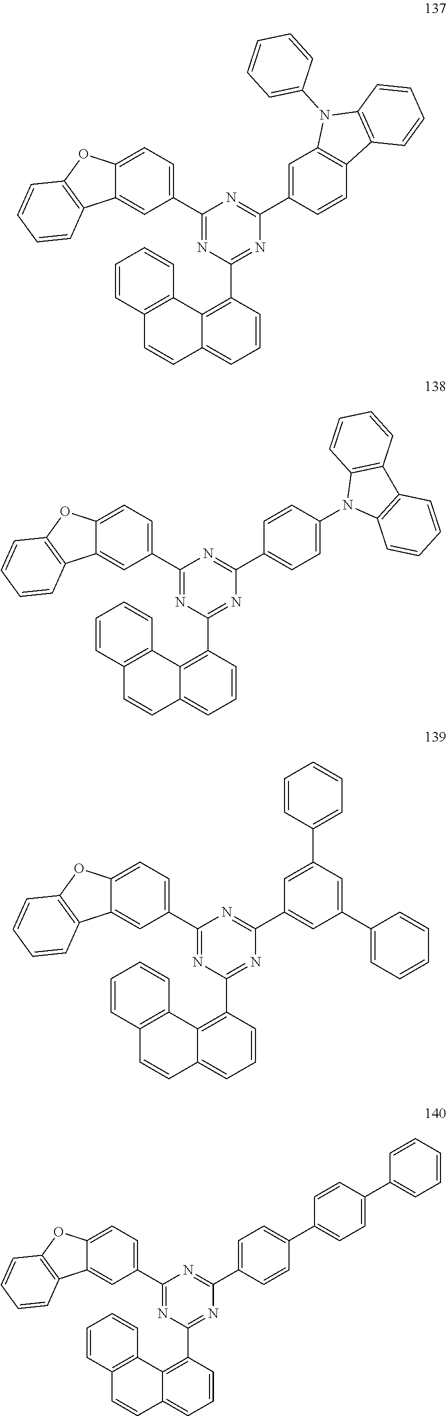 [SMILES: c1ccc(-n2c3ccccc3c3ccc(-c4nc(-c5ccc6oc7ccccc7c6c5)nc(-c5cccc6ccc7ccccc7c56)n4)cc32)cc1 ; c1ccc2c(c1)ccc1cccc(-c3nc(-c4ccc(-n5c6ccccc6c6ccccc65)cc4)nc(-c4ccc5oc6ccccc6c5c4)n3)c12 ; c1ccc(-c2cc(-c3ccccc3)cc(-c3nc(-c4ccc5oc6ccccc6c5c4)nc(-c4cccc5ccc6ccccc6c45)n3)c2)cc1 ; c1ccc(-c2ccc(-c3ccc(-c4nc(-c5ccc6oc7ccccc7c6c5)nc(-c5cccc6ccc7ccccc7c56)n4)cc3)cc2)cc1]

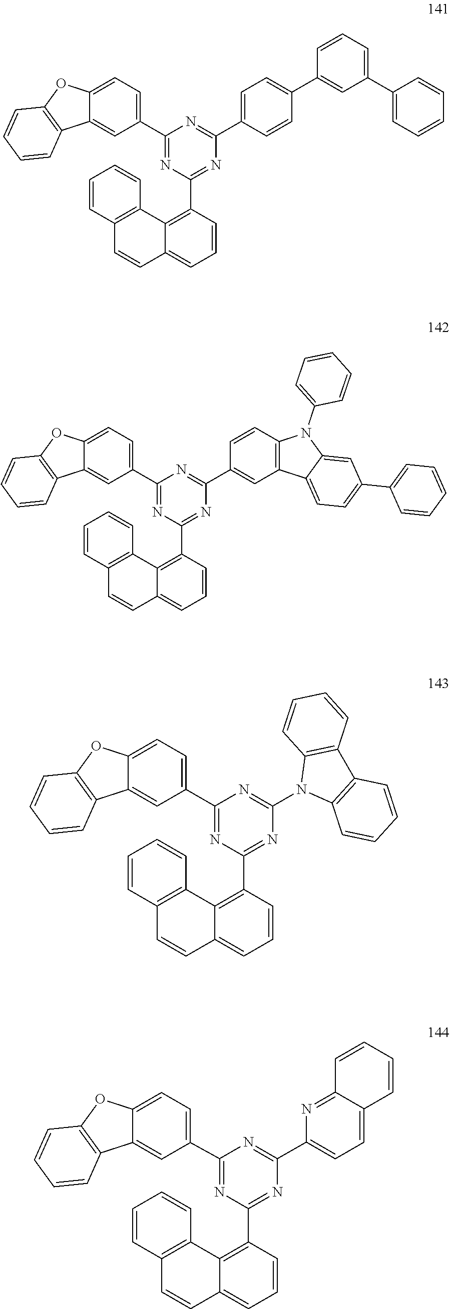 [SMILES: c1ccc(-c2cccc(-c3ccc(-c4nc(-c5ccc6oc7ccccc7c6c5)nc(-c5cccc6ccc7ccccc7c56)n4)cc3)c2)cc1 ; c1ccc(-c2ccc3c4cc(-c5nc(-c6ccc7oc8ccccc8c7c6)nc(-c6cccc7ccc8ccccc8c67)n5)ccc4n(-c4ccccc4)c3c2)cc1 ; c1ccc2c(c1)ccc1cccc(-c3nc(-c4ccc5oc6ccccc6c5c4)nc(-n4c5ccccc5c5ccccc54)n3)c12 ; c1ccc2nc(-c3nc(-c4ccc5oc6ccccc6c5c4)nc(-c4cccc5ccc6ccccc6c45)n3)ccc2c1]

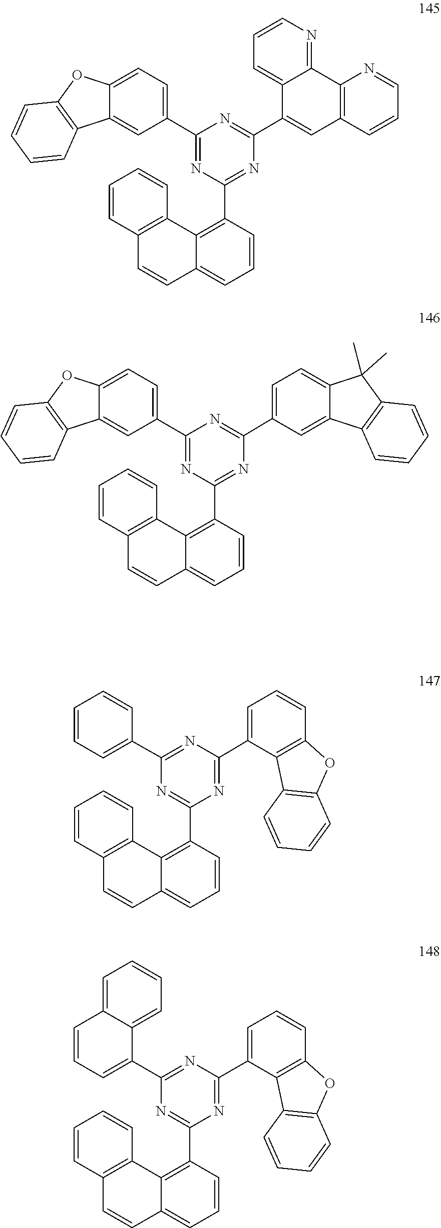 [SMILES: c1ccc2c(c1)ccc1cccc(-c3nc(-c4ccc5oc6ccccc6c5c4)nc(-c4cc5cccnc5c5ncccc45)n3)c12 ; CC1(C)c2ccccc2-c2cc(-c3nc(-c4ccc5oc6ccccc6c5c4)nc(-c4cccc5ccc6ccccc6c45)n3)ccc21 ; c1ccc(-c2nc(-c3cccc4ccc5ccccc5c34)nc(-c3cccc4oc5ccccc5c34)n2)cc1 ; c1ccc2c(-c3nc(-c4cccc5ccc6ccccc6c45)nc(-c4cccc5oc6ccccc6c45)n3)cccc2c1]

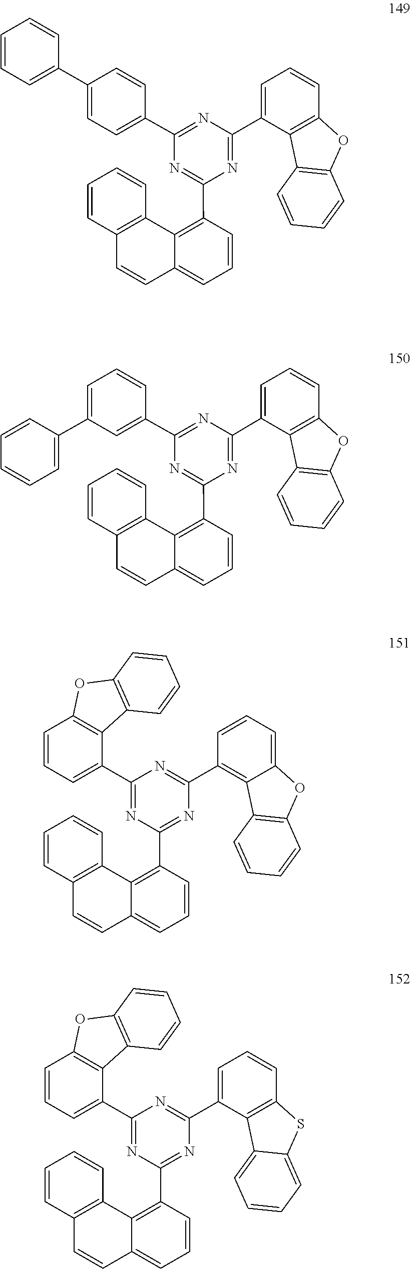 [SMILES: c1ccc(-c2ccc(-c3nc(-c4cccc5ccc6ccccc6c45)nc(-c4cccc5oc6ccccc6c45)n3)cc2)cc1 ; c1ccc(-c2cccc(-c3nc(-c4cccc5ccc6ccccc6c45)nc(-c4cccc5oc6ccccc6c45)n3)c2)cc1 ; c1ccc2c(c1)ccc1cccc(-c3nc(-c4cccc5oc6ccccc6c45)nc(-c4cccc5oc6ccccc6c45)n3)c12 ; c1ccc2c(c1)ccc1cccc(-c3nc(-c4cccc5oc6ccccc6c45)nc(-c4cccc5sc6ccccc6c45)n3)c12]

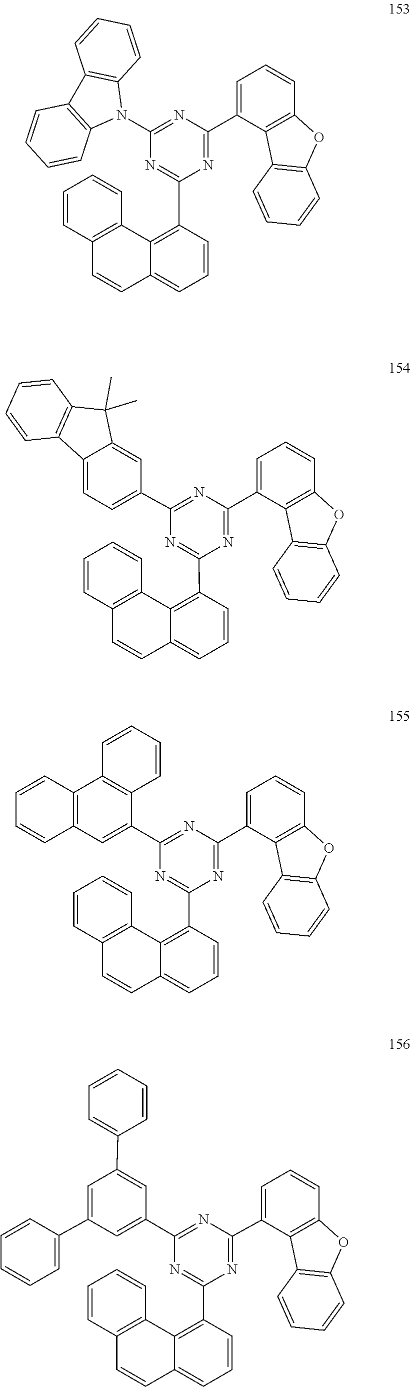 [SMILES: c1ccc2c(c1)ccc1cccc(-c3nc(-c4cccc5oc6ccccc6c45)nc(-n4c5ccccc5c5ccccc54)n3)c12 ; CC1(C)c2ccccc2-c2ccc(-c3nc(-c4cccc5ccc6ccccc6c45)nc(-c4cccc5oc6ccccc6c45)n3)cc21 ; c1ccc2c(c1)cc(-c1nc(-c3cccc4ccc5ccccc5c34)nc(-c3cccc4oc5ccccc5c34)n1)c1ccccc12 ; c1ccc(-c2cc(-c3ccccc3)cc(-c3nc(-c4cccc5ccc6ccccc6c45)nc(-c4cccc5oc6ccccc6c45)n3)c2)cc1]

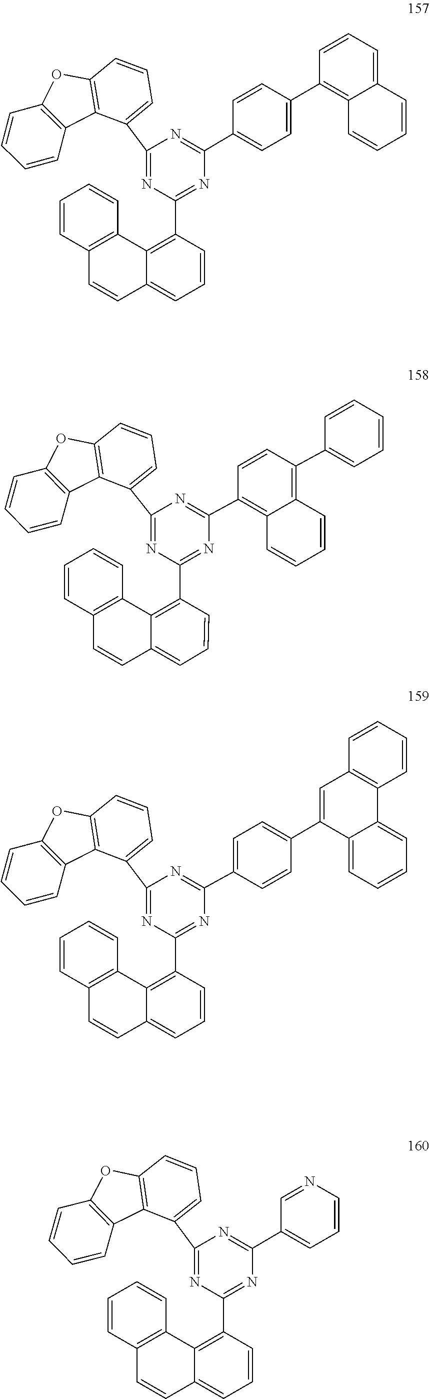 [SMILES: c1ccc2c(-c3ccc(-c4nc(-c5cccc6ccc7ccccc7c56)nc(-c5cccc6oc7ccccc7c56)n4)cc3)cccc2c1 ; c1ccc(-c2ccc(-c3nc(-c4cccc5ccc6ccccc6c45)nc(-c4cccc5oc6ccccc6c45)n3)c3ccccc23)cc1 ; c1ccc2c(c1)cc(-c1ccc(-c3nc(-c4cccc5ccc6ccccc6c45)nc(-c4cccc5oc6ccccc6c45)n3)cc1)c1ccccc12 ; c1cncc(-c2nc(-c3cccc4ccc5ccccc5c34)nc(-c3cccc4oc5ccccc5c34)n2)c1]

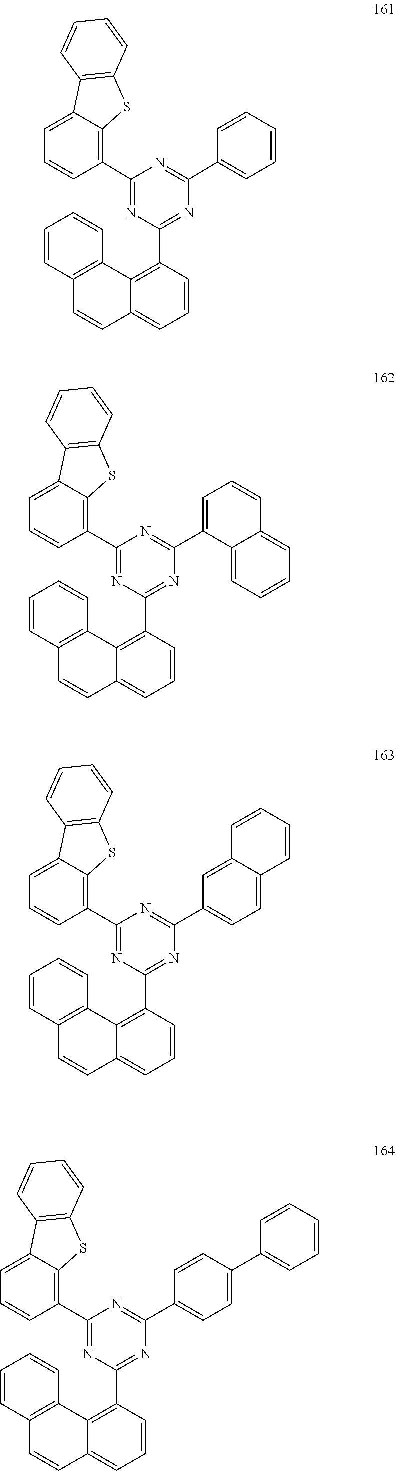 [SMILES: c1ccc(-c2nc(-c3cccc4c3sc3ccccc34)nc(-c3cccc4ccc5ccccc5c34)n2)cc1 ; c1ccc2c(-c3nc(-c4cccc5c4sc4ccccc45)nc(-c4cccc5ccc6ccccc6c45)n3)cccc2c1 ; c1ccc2cc(-c3nc(-c4cccc5c4sc4ccccc45)nc(-c4cccc5ccc6ccccc6c45)n3)ccc2c1 ; c1ccc(-c2ccc(-c3nc(-c4cccc5c4sc4ccccc45)nc(-c4cccc5ccc6ccccc6c45)n3)cc2)cc1]

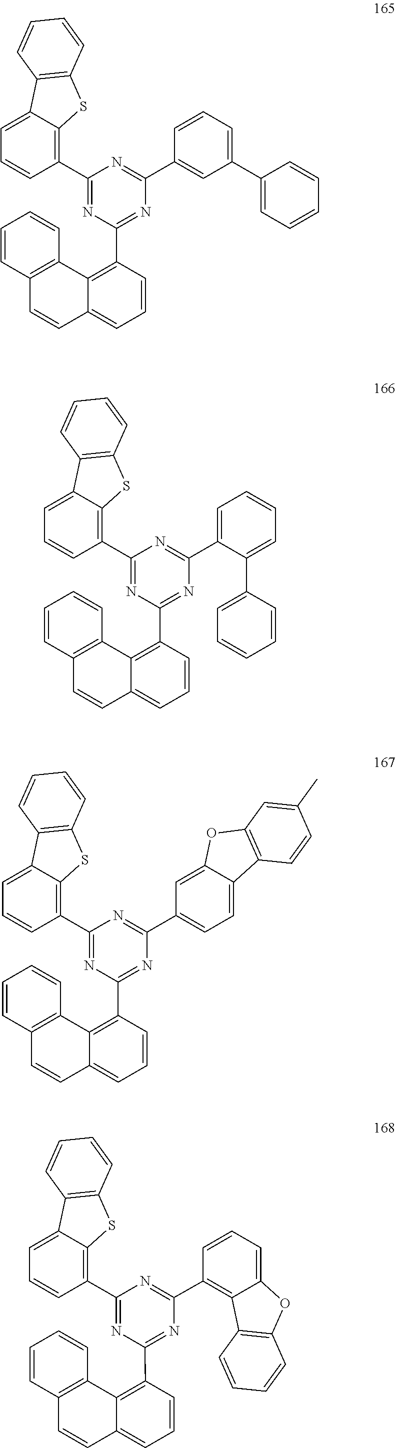 [SMILES: c1ccc(-c2cccc(-c3nc(-c4cccc5c4sc4ccccc45)nc(-c4cccc5ccc6ccccc6c45)n3)c2)cc1 ; c1ccc(-c2ccccc2-c2nc(-c3cccc4c3sc3ccccc34)nc(-c3cccc4ccc5ccccc5c34)n2)cc1 ; Cc1ccc2c(c1)oc1cc(-c3nc(-c4cccc5c4sc4ccccc45)nc(-c4cccc5ccc6ccccc6c45)n3)ccc12 ; c1ccc2c(c1)ccc1cccc(-c3nc(-c4cccc5c4sc4ccccc45)nc(-c4cccc5oc6ccccc6c45)n3)c12]

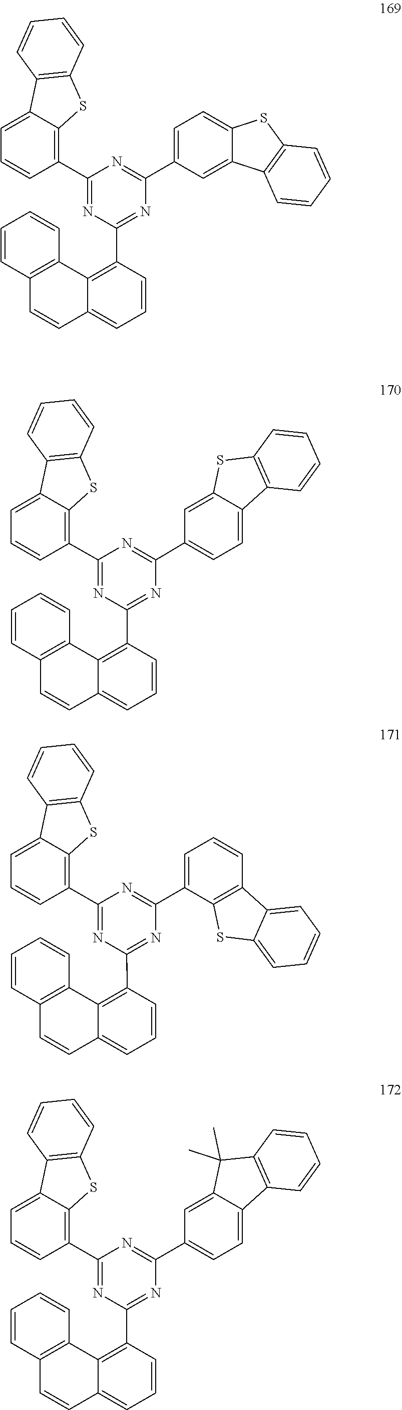

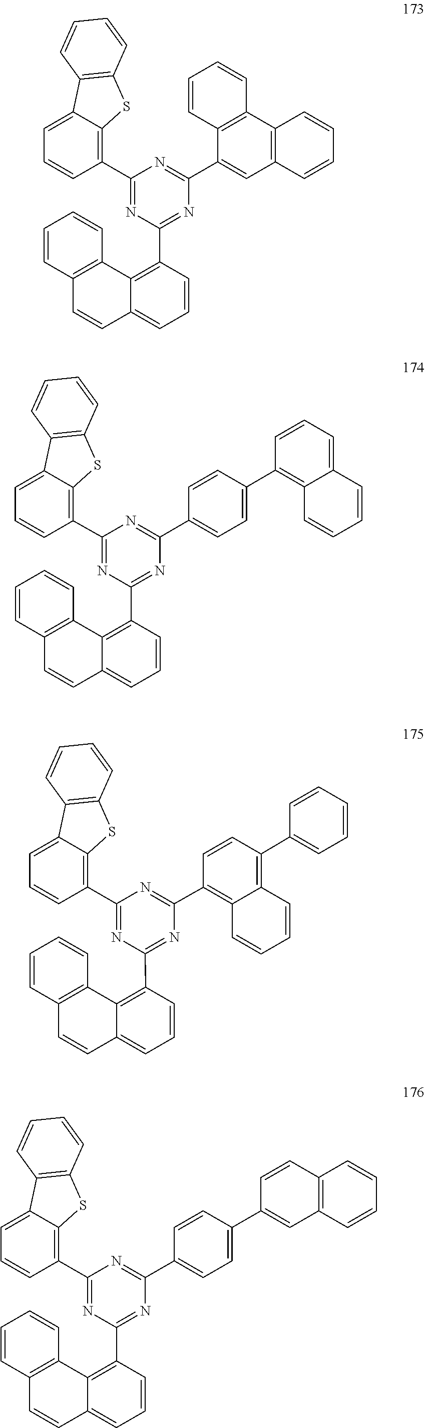 [SMILES: c1ccc2c(c1)cc(-c1nc(-c3cccc4c3sc3ccccc34)nc(-c3cccc4ccc5ccccc5c34)n1)c1ccccc12 ; c1ccc2c(-c3ccc(-c4nc(-c5cccc6c5sc5ccccc56)nc(-c5cccc6ccc7ccccc7c56)n4)cc3)cccc2c1 ; c1ccc(-c2ccc(-c3nc(-c4cccc5c4sc4ccccc45)nc(-c4cccc5ccc6ccccc6c45)n3)c3ccccc23)cc1 ; c1ccc2cc(-c3ccc(-c4nc(-c5cccc6c5sc5ccccc56)nc(-c5cccc6ccc7ccccc7c56)n4)cc3)ccc2c1]

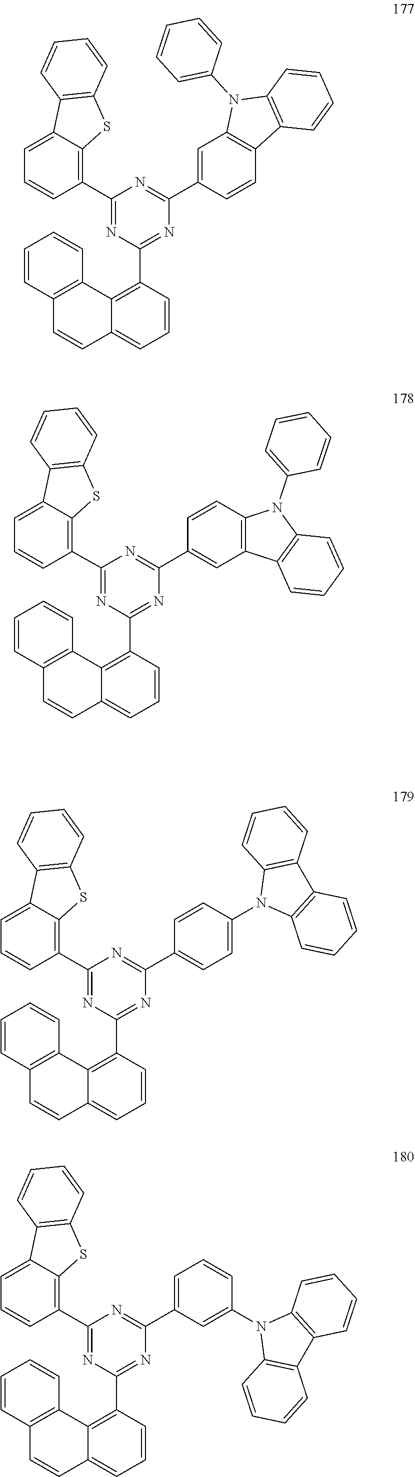 [SMILES: c1ccc(-n2c3ccccc3c3ccc(-c4nc(-c5cccc6c5sc5ccccc56)nc(-c5cccc6ccc7ccccc7c56)n4)cc32)cc1 ; c1ccc(-n2c3ccccc3c3cc(-c4nc(-c5cccc6c5sc5ccccc56)nc(-c5cccc6ccc7ccccc7c56)n4)ccc32)cc1 ; c1ccc2c(c1)ccc1cccc(-c3nc(-c4ccc(-n5c6ccccc6c6ccccc65)cc4)nc(-c4cccc5c4sc4ccccc45)n3)c12 ; c1cc(-c2nc(-c3cccc4c3sc3ccccc34)nc(-c3cccc4ccc5ccccc5c34)n2)cc(-n2c3ccccc3c3ccccc32)c1]

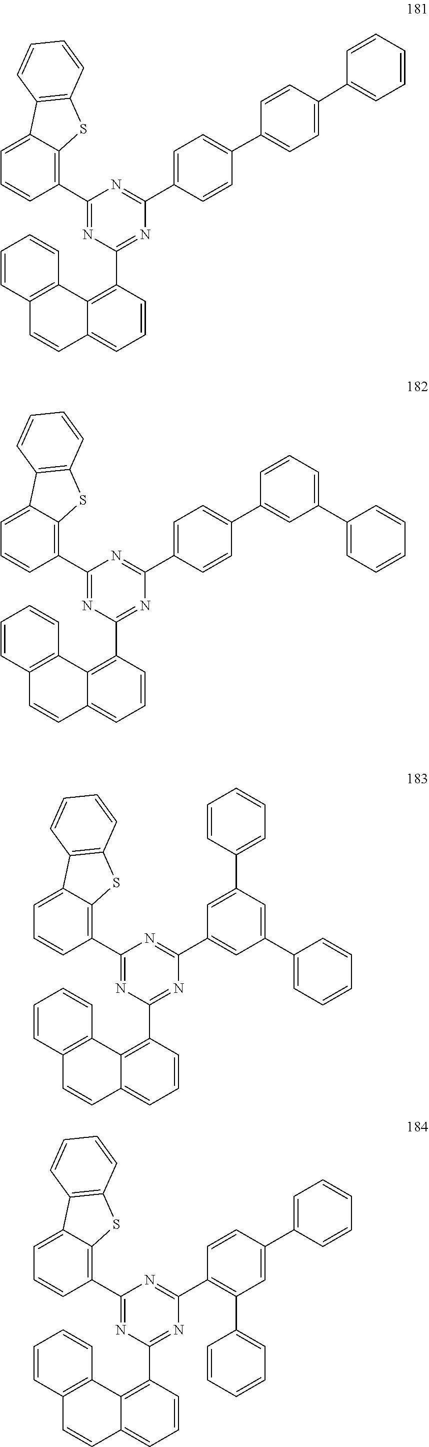

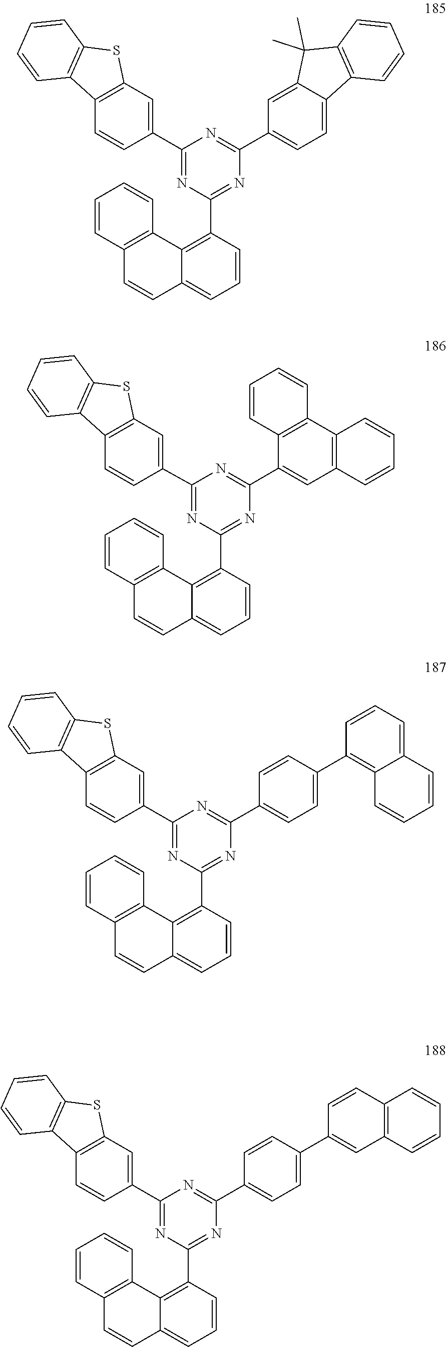 [SMILES: CC1(C)c2ccccc2-c2ccc(-c3nc(-c4ccc5c(c4)sc4ccccc45)nc(-c4cccc5ccc6ccccc6c45)n3)cc21 ; c1ccc2c(c1)cc(-c1nc(-c3ccc4c(c3)sc3ccccc34)nc(-c3cccc4ccc5ccccc5c34)n1)c1ccccc12 ; c1ccc2c(-c3ccc(-c4nc(-c5ccc6c(c5)sc5ccccc56)nc(-c5cccc6ccc7ccccc7c56)n4)cc3)cccc2c1 ; c1ccc2cc(-c3ccc(-c4nc(-c5ccc6c(c5)sc5ccccc56)nc(-c5cccc6ccc7ccccc7c56)n4)cc3)ccc2c1]

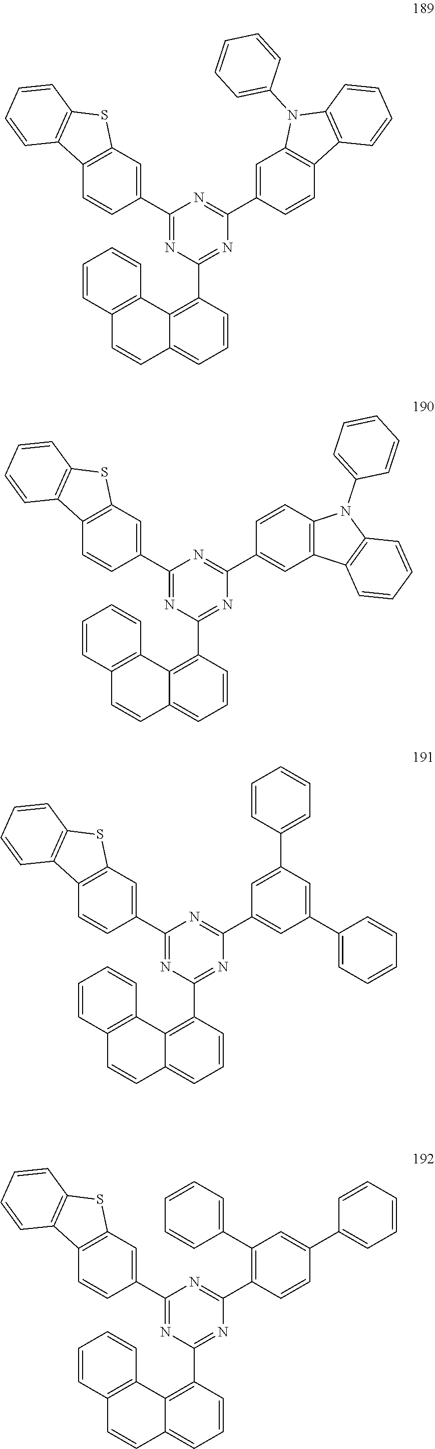 [SMILES: c1ccc(-n2c3ccccc3c3ccc(-c4nc(-c5ccc6c(c5)sc5ccccc56)nc(-c5cccc6ccc7ccccc7c56)n4)cc32)cc1 ; c1ccc(-n2c3ccccc3c3cc(-c4nc(-c5ccc6c(c5)sc5ccccc56)nc(-c5cccc6ccc7ccccc7c56)n4)ccc32)cc1 ; c1ccc(-c2cc(-c3ccccc3)cc(-c3nc(-c4ccc5c(c4)sc4ccccc45)nc(-c4cccc5ccc6ccccc6c45)n3)c2)cc1 ; c1ccc(-c2ccc(-c3nc(-c4ccc5c(c4)sc4ccccc45)nc(-c4cccc5ccc6ccccc6c45)n3)c(-c3ccccc3)c2)cc1]

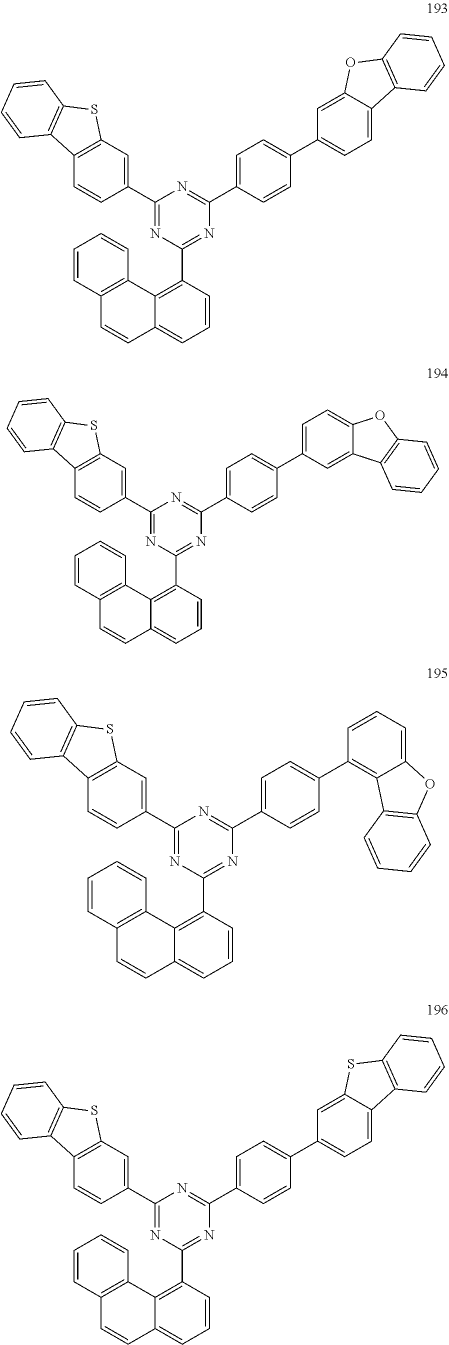 [SMILES: c1ccc2c(c1)ccc1cccc(-c3nc(-c4ccc(-c5ccc6c(c5)oc5ccccc56)cc4)nc(-c4ccc5c(c4)sc4ccccc45)n3)c12 ; c1ccc2c(c1)ccc1cccc(-c3nc(-c4ccc(-c5ccc6oc7ccccc7c6c5)cc4)nc(-c4ccc5c(c4)sc4ccccc45)n3)c12 ; c1ccc2c(c1)ccc1cccc(-c3nc(-c4ccc(-c5cccc6oc7ccccc7c56)cc4)nc(-c4ccc5c(c4)sc4ccccc45)n3)c12 ; c1ccc2c(c1)ccc1cccc(-c3nc(-c4ccc(-c5ccc6c(c5)sc5ccccc56)cc4)nc(-c4ccc5c(c4)sc4ccccc45)n3)c12]

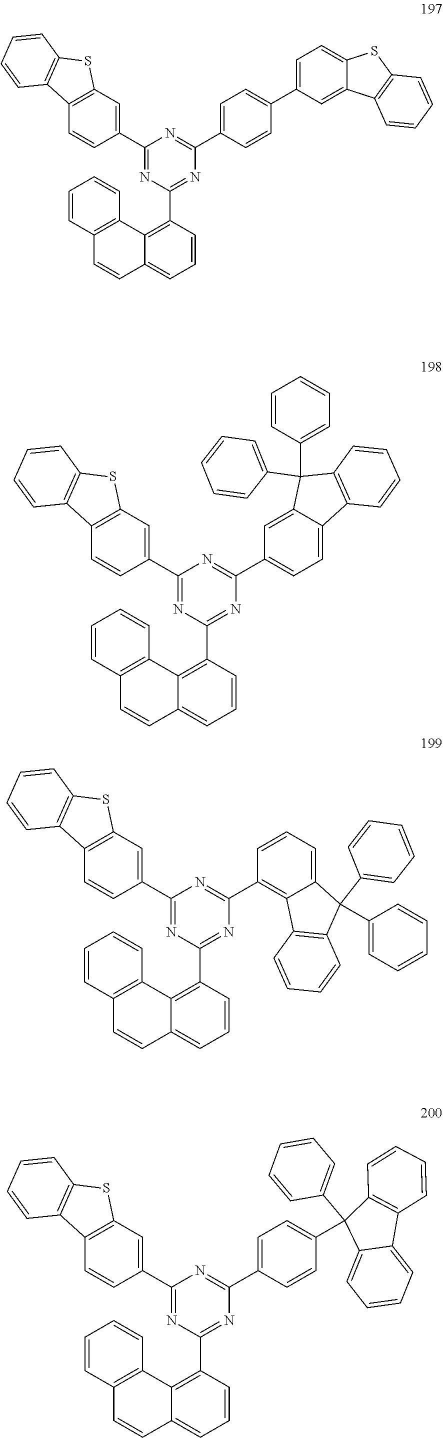 [SMILES: c1ccc2c(c1)ccc1cccc(-c3nc(-c4ccc(-c5ccc6sc7ccccc7c6c5)cc4)nc(-c4ccc5c(c4)sc4ccccc45)n3)c12 ; c1ccc(C2(c3ccccc3)c3ccccc3-c3ccc(-c4nc(-c5ccc6c(c5)sc5ccccc56)nc(-c5cccc6ccc7ccccc7c56)n4)cc32)cc1 ; c1ccc(C2(c3ccccc3)c3ccccc3-c3c(-c4nc(-c5ccc6c(c5)sc5ccccc56)nc(-c5cccc6ccc7ccccc7c56)n4)cccc32)cc1 ; c1ccc(C2(c3ccc(-c4nc(-c5ccc6c(c5)sc5ccccc56)nc(-c5cccc6ccc7ccccc7c56)n4)cc3)c3ccccc3-c3ccccc32)cc1]

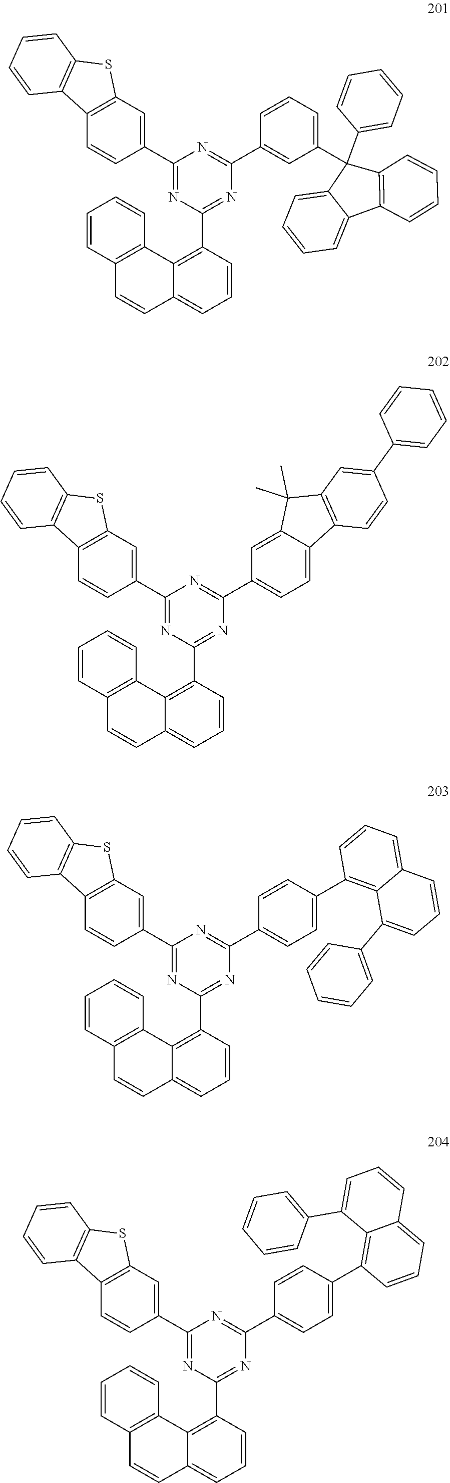 [SMILES: c1ccc(C2(c3cccc(-c4nc(-c5ccc6c(c5)sc5ccccc56)nc(-c5cccc6ccc7ccccc7c56)n4)c3)c3ccccc3-c3ccccc32)cc1 ; CC1(C)c2cc(-c3ccccc3)ccc2-c2ccc(-c3nc(-c4ccc5c(c4)sc4ccccc45)nc(-c4cccc5ccc6ccccc6c45)n3)cc21 ; c1ccc(-c2cccc3cccc(-c4ccc(-c5nc(-c6ccc7c(c6)sc6ccccc67)nc(-c6cccc7ccc8ccccc8c67)n5)cc4)c23)cc1 ; c1ccc(-c2cccc3cccc(-c4ccc(-c5nc(-c6ccc7c(c6)sc6ccccc67)nc(-c6cccc7ccc8ccccc8c67)n5)cc4)c23)cc1]

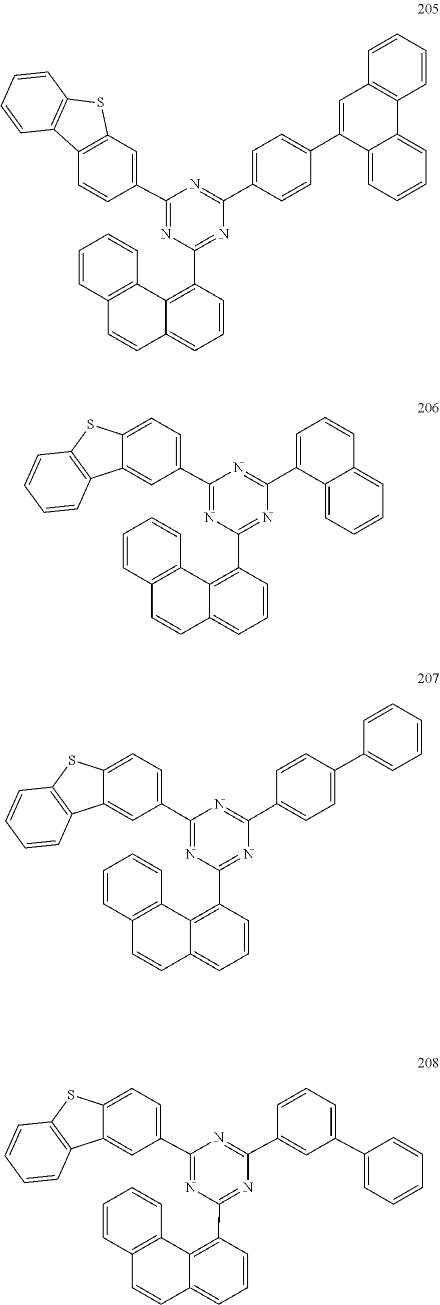 [SMILES: c1ccc2c(c1)cc(-c1ccc(-c3nc(-c4ccc5c(c4)sc4ccccc45)nc(-c4cccc5ccc6ccccc6c45)n3)cc1)c1ccccc12 ; c1ccc2c(-c3nc(-c4ccc5sc6ccccc6c5c4)nc(-c4cccc5ccc6ccccc6c45)n3)cccc2c1 ; c1ccc(-c2ccc(-c3nc(-c4ccc5sc6ccccc6c5c4)nc(-c4cccc5ccc6ccccc6c45)n3)cc2)cc1 ; c1ccc(-c2cccc(-c3nc(-c4ccc5sc6ccccc6c5c4)nc(-c4cccc5ccc6ccccc6c45)n3)c2)cc1]

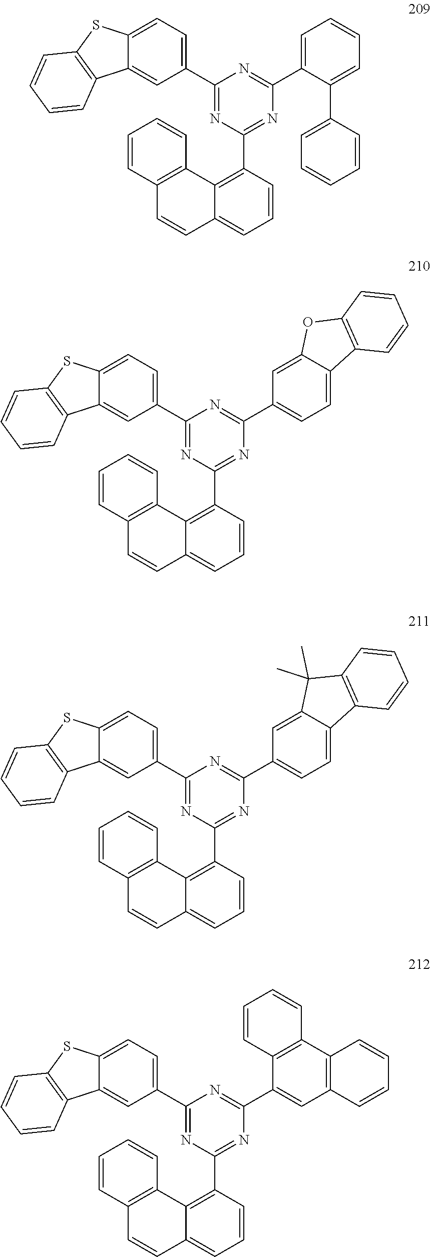 [SMILES: c1ccc(-c2ccccc2-c2nc(-c3ccc4sc5ccccc5c4c3)nc(-c3cccc4ccc5ccccc5c34)n2)cc1 ; c1ccc2c(c1)ccc1cccc(-c3nc(-c4ccc5c(c4)oc4ccccc45)nc(-c4ccc5sc6ccccc6c5c4)n3)c12 ; CC1(C)c2ccccc2-c2ccc(-c3nc(-c4ccc5sc6ccccc6c5c4)nc(-c4cccc5ccc6ccccc6c45)n3)cc21 ; c1ccc2c(c1)cc(-c1nc(-c3ccc4sc5ccccc5c4c3)nc(-c3cccc4ccc5ccccc5c34)n1)c1ccccc12]

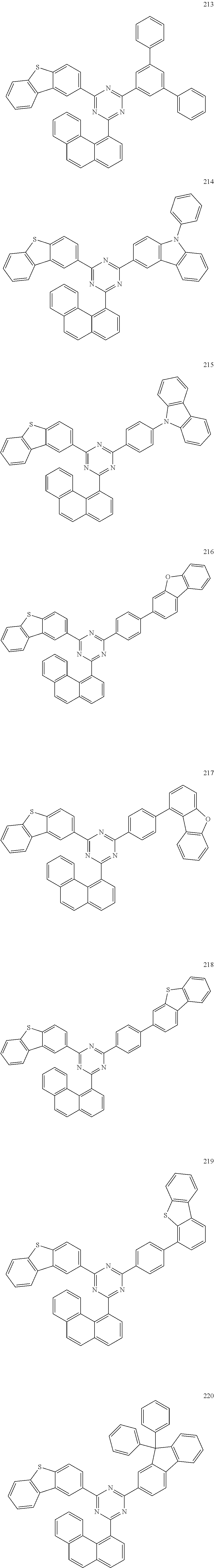

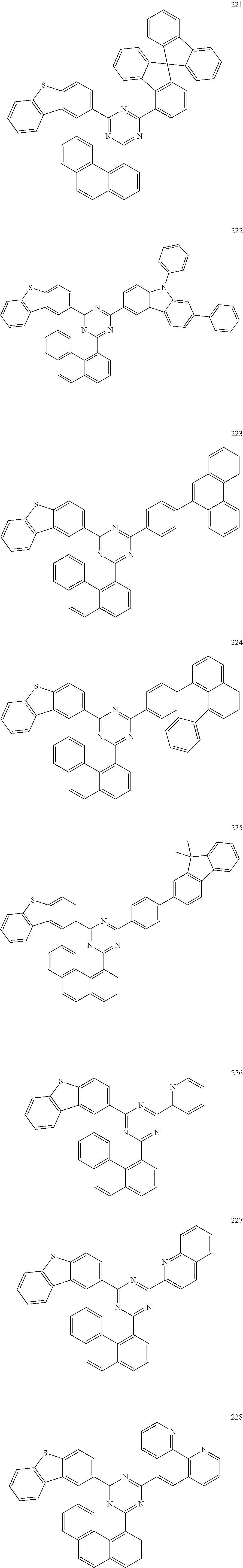

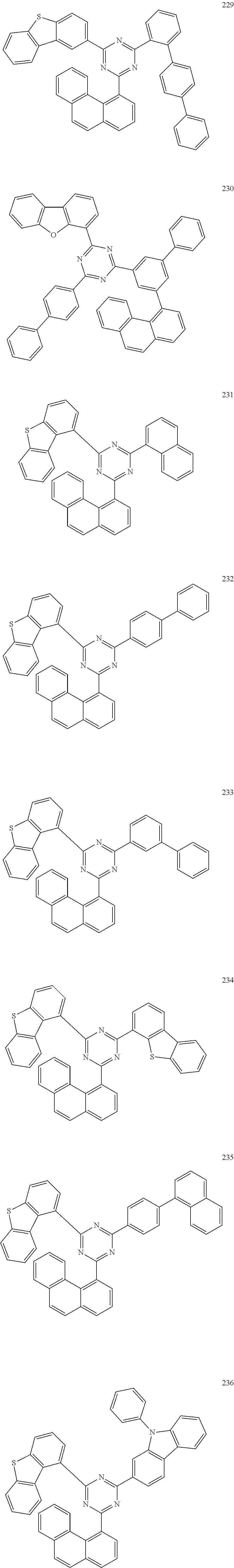

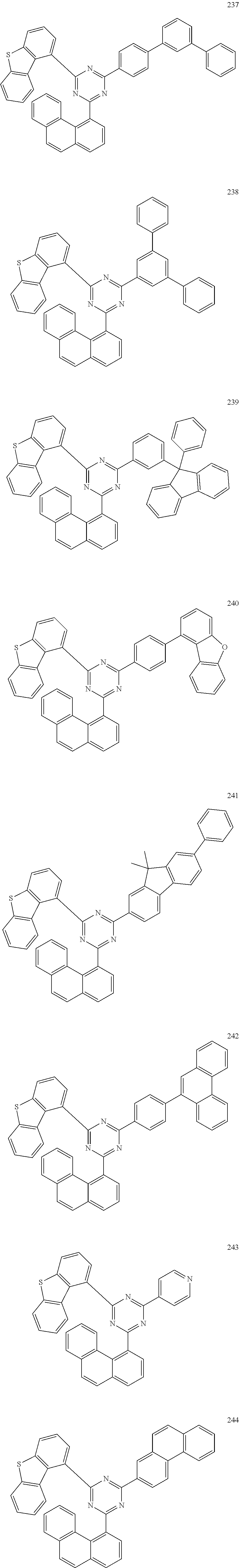

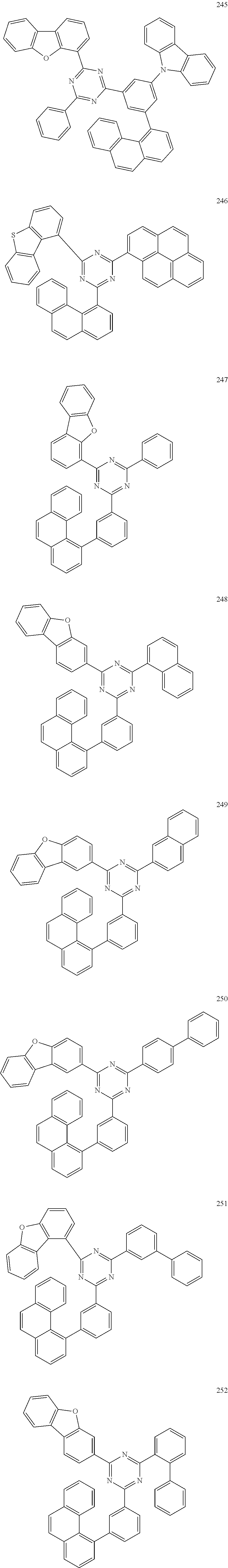

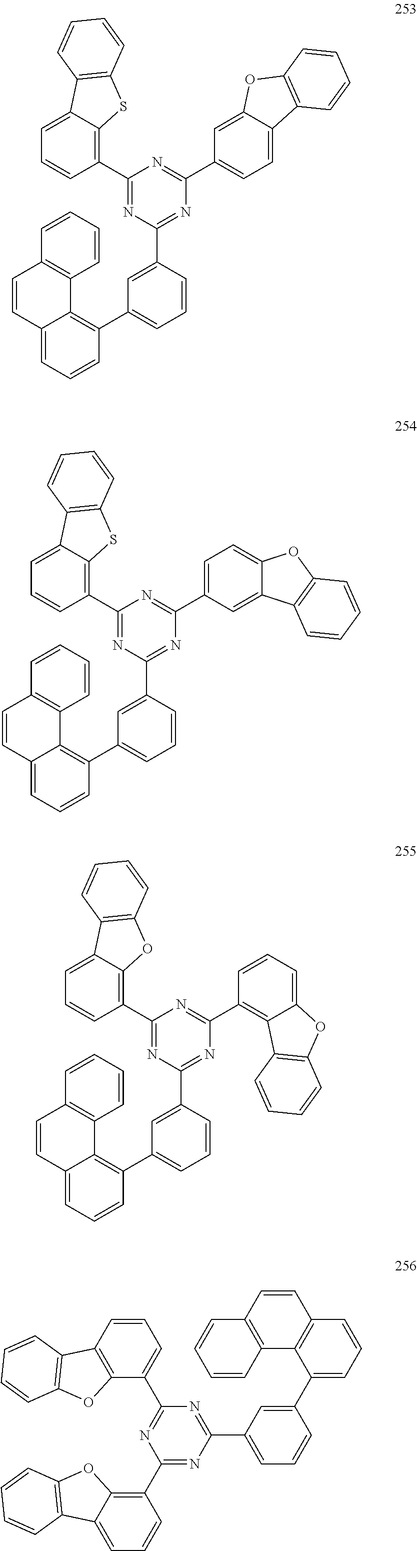 [SMILES: c1cc(-c2nc(-c3ccc4c(c3)oc3ccccc34)nc(-c3cccc4c3sc3ccccc34)n2)cc(-c2cccc3ccc4ccccc4c23)c1 ; c1cc(-c2nc(-c3ccc4oc5ccccc5c4c3)nc(-c3cccc4c3sc3ccccc34)n2)cc(-c2cccc3ccc4ccccc4c23)c1 ; c1cc(-c2nc(-c3cccc4c3oc3ccccc34)nc(-c3cccc4oc5ccccc5c34)n2)cc(-c2cccc3ccc4ccccc4c23)c1 ; c1cc(-c2nc(-c3cccc4c3oc3ccccc34)nc(-c3cccc4c3oc3ccccc34)n2)cc(-c2cccc3ccc4ccccc4c23)c1]

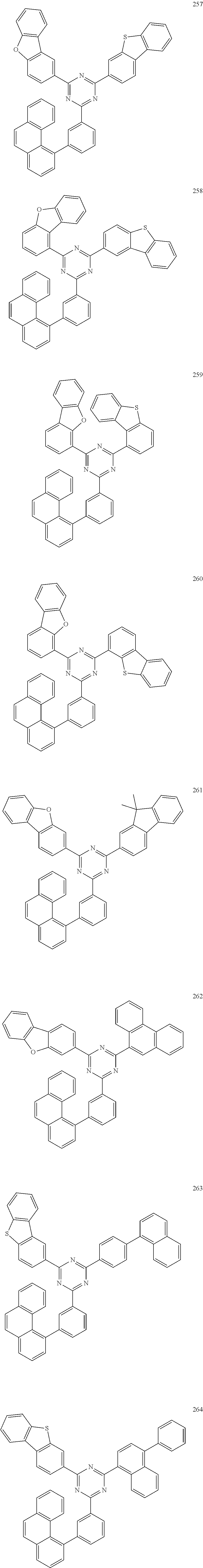

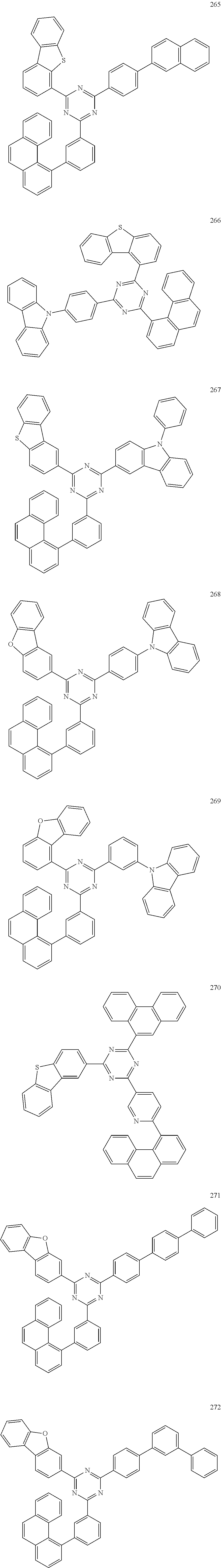

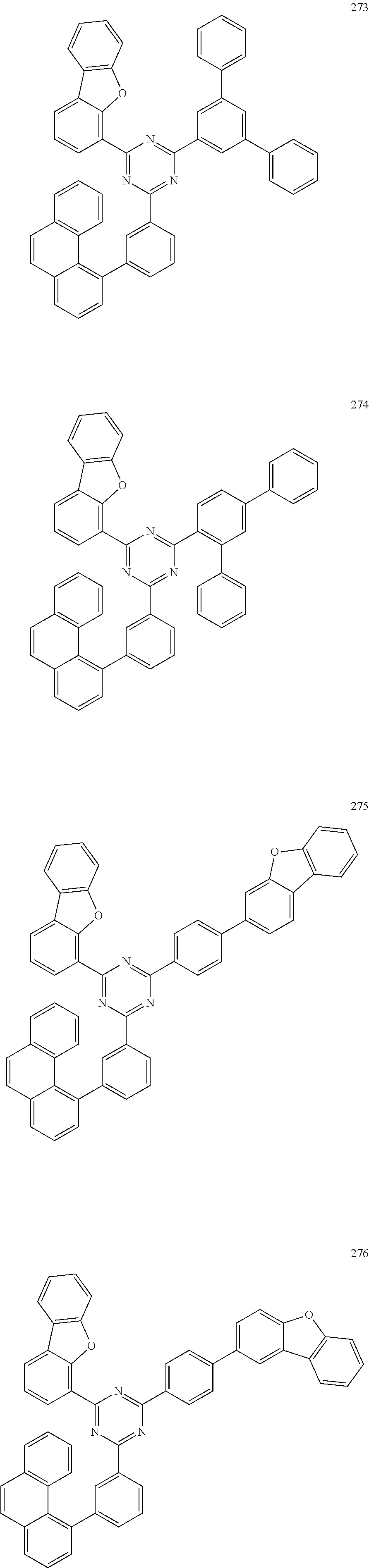 [SMILES: c1ccc(-c2cc(-c3ccccc3)cc(-c3nc(-c4cccc(-c5cccc6ccc7ccccc7c56)c4)nc(-c4cccc5c4oc4ccccc45)n3)c2)cc1 ; c1ccc(-c2ccc(-c3nc(-c4cccc(-c5cccc6ccc7ccccc7c56)c4)nc(-c4cccc5c4oc4ccccc45)n3)c(-c3ccccc3)c2)cc1 ; c1cc(-c2nc(-c3ccc(-c4ccc5c(c4)oc4ccccc45)cc3)nc(-c3cccc4c3oc3ccccc34)n2)cc(-c2cccc3ccc4ccccc4c23)c1 ; c1cc(-c2nc(-c3ccc(-c4ccc5oc6ccccc6c5c4)cc3)nc(-c3cccc4c3oc3ccccc34)n2)cc(-c2cccc3ccc4ccccc4c23)c1]

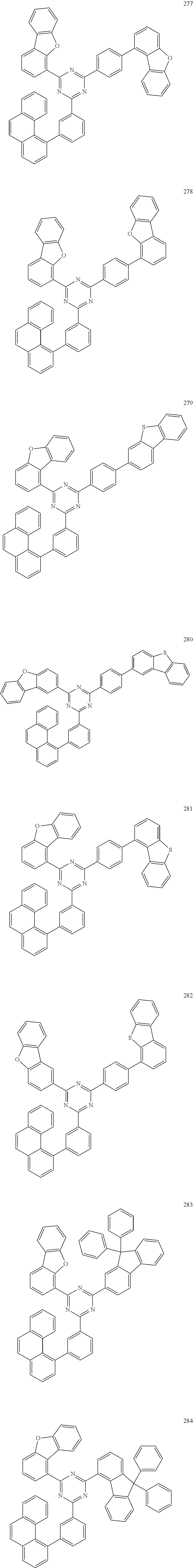

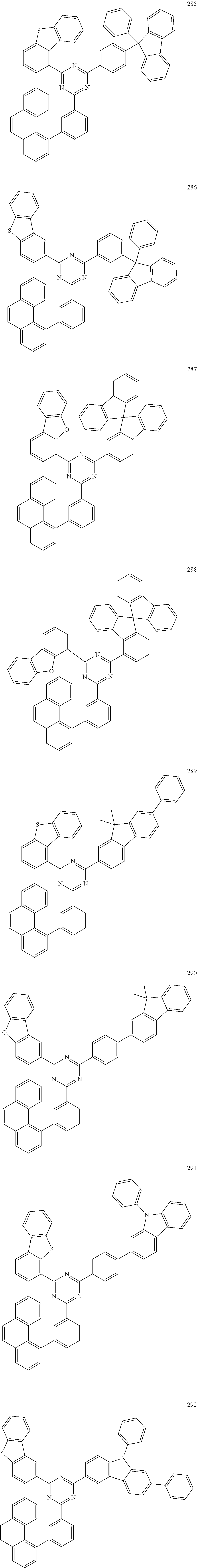

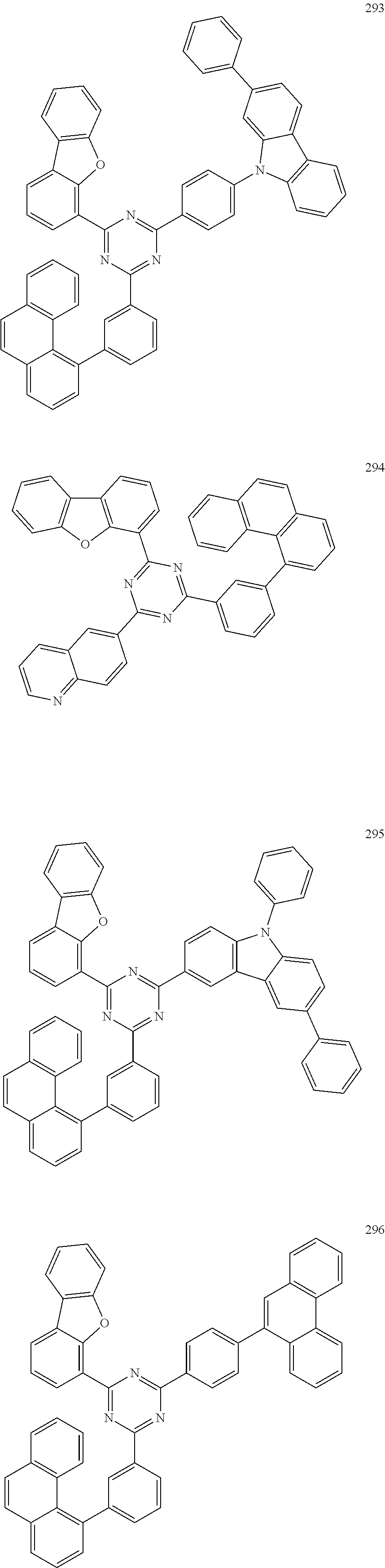 [SMILES: c1ccc(-c2ccc3c4ccccc4n(-c4ccc(-c5nc(-c6cccc(-c7cccc8ccc9ccccc9c78)c6)nc(-c6cccc7c6oc6ccccc67)n5)cc4)c3c2)cc1 ; c1cc(-c2nc(-c3ccc4ncccc4c3)nc(-c3cccc4c3oc3ccccc34)n2)cc(-c2cccc3ccc4ccccc4c23)c1 ; c1ccc(-c2ccc3c(c2)c2cc(-c4nc(-c5cccc(-c6cccc7ccc8ccccc8c67)c5)nc(-c5cccc6c5oc5ccccc56)n4)ccc2n3-c2ccccc2)cc1 ; c1cc(-c2nc(-c3ccc(-c4cc5ccccc5c5ccccc45)cc3)nc(-c3cccc4c3oc3ccccc34)n2)cc(-c2cccc3ccc4ccccc4c23)c1]

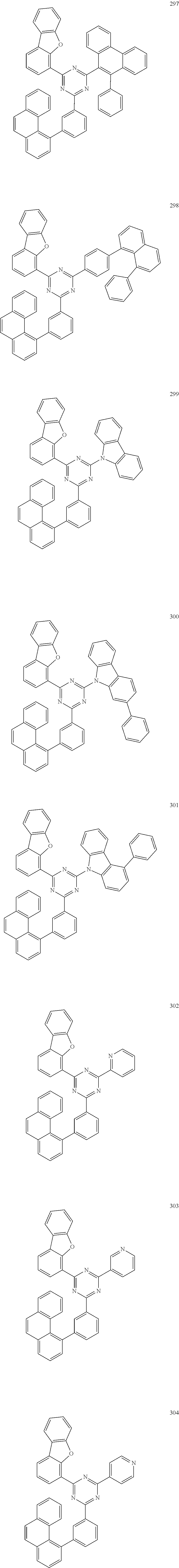

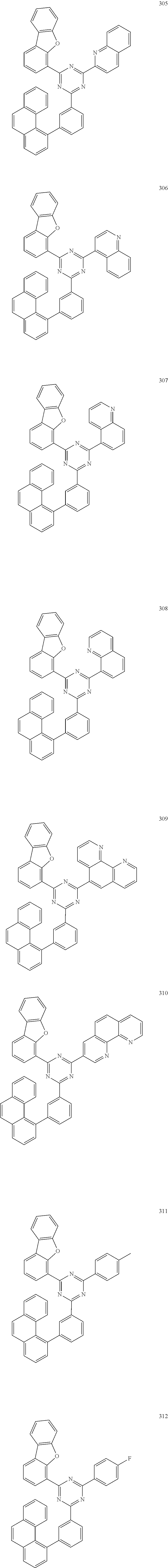

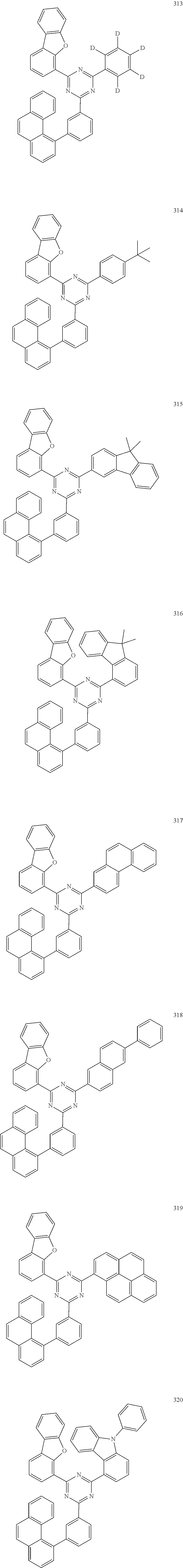

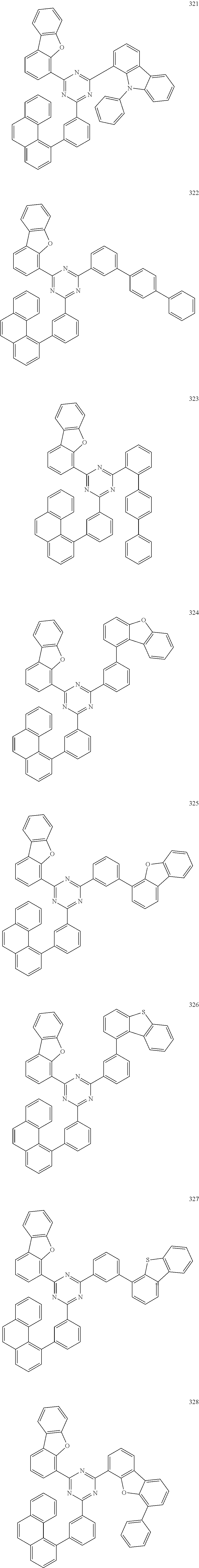

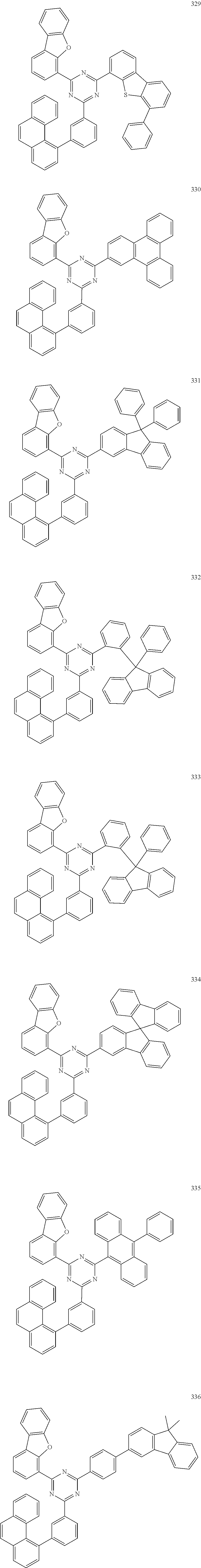

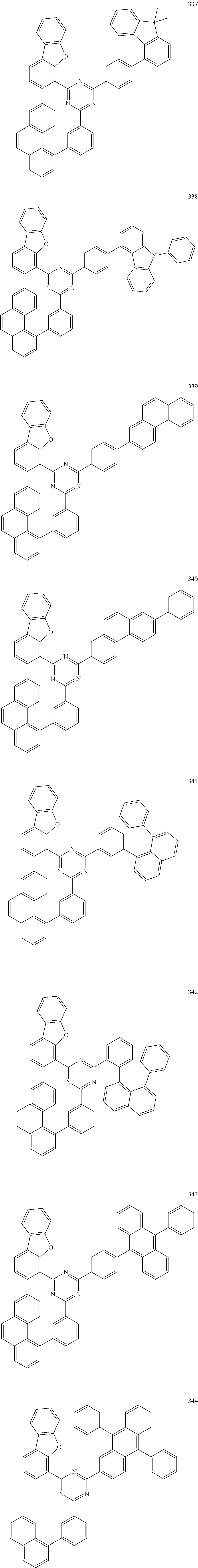

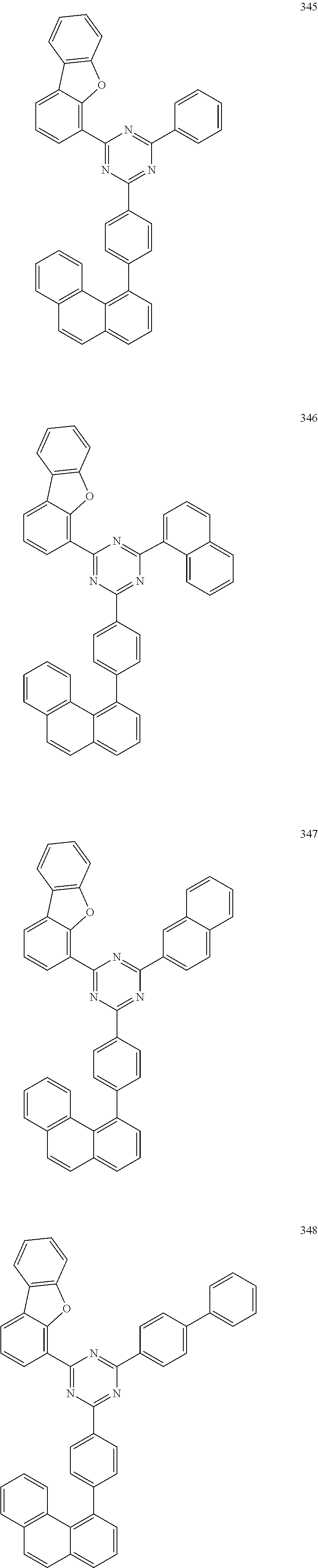 [SMILES: c1ccc(-c2nc(-c3ccc(-c4cccc5ccc6ccccc6c45)cc3)nc(-c3cccc4c3oc3ccccc34)n2)cc1 ; c1ccc2c(-c3nc(-c4ccc(-c5cccc6ccc7ccccc7c56)cc4)nc(-c4cccc5c4oc4ccccc45)n3)cccc2c1 ; c1ccc2cc(-c3nc(-c4ccc(-c5cccc6ccc7ccccc7c56)cc4)nc(-c4cccc5c4oc4ccccc45)n3)ccc2c1 ; c1ccc(-c2ccc(-c3nc(-c4ccc(-c5cccc6ccc7ccccc7c56)cc4)nc(-c4cccc5c4oc4ccccc45)n3)cc2)cc1]

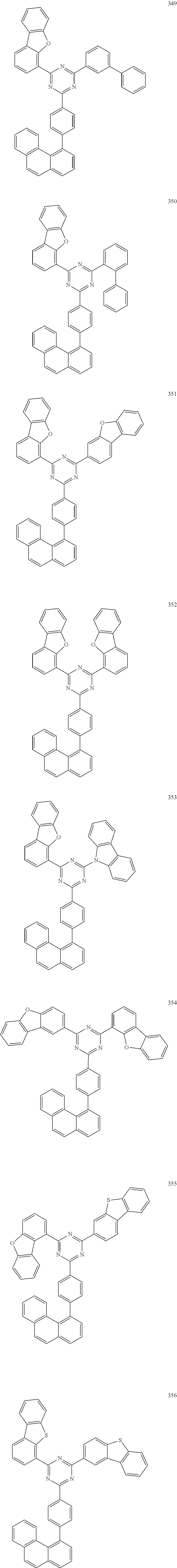

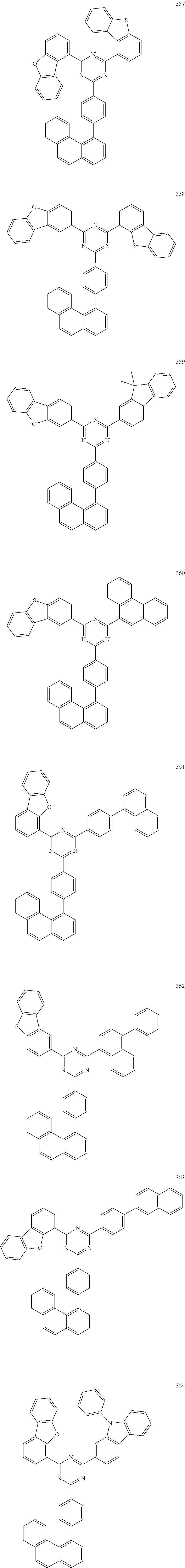

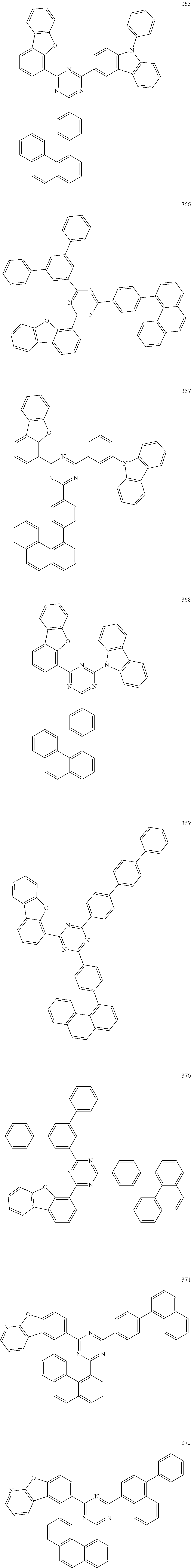

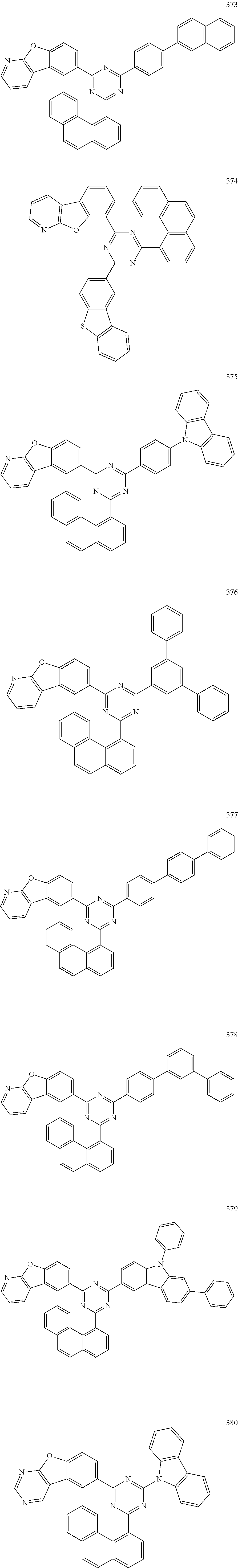

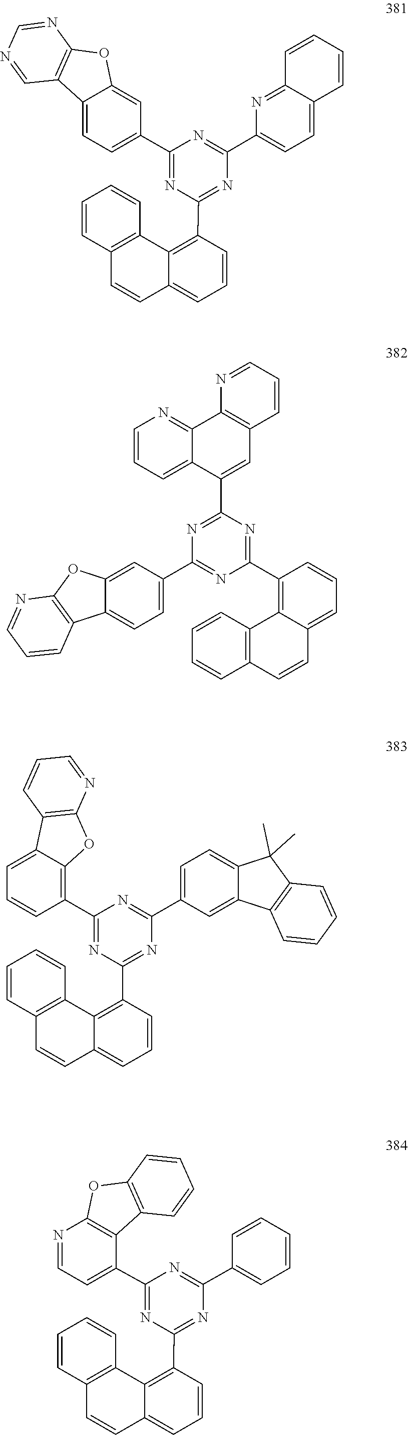 [SMILES: c1ccc2nc(-c3nc(-c4ccc5c(c4)oc4ncncc45)nc(-c4cccc5ccc6ccccc6c45)n3)ccc2c1 ; c1ccc2c(c1)ccc1cccc(-c3nc(-c4ccc5c(c4)oc4ncccc45)nc(-c4cc5cccnc5c5ncccc45)n3)c12 ; CC1(C)c2ccccc2-c2cc(-c3nc(-c4cccc5c4oc4ncccc45)nc(-c4cccc5ccc6ccccc6c45)n3)ccc21 ; c1ccc(-c2nc(-c3cccc4ccc5ccccc5c34)nc(-c3ccnc4oc5ccccc5c34)n2)cc1]

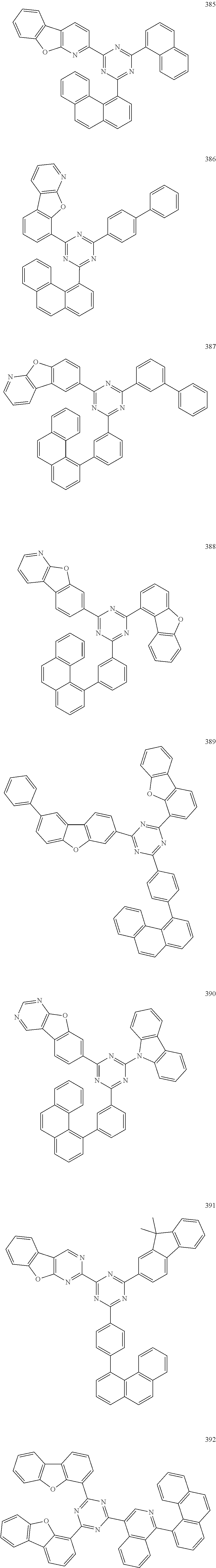

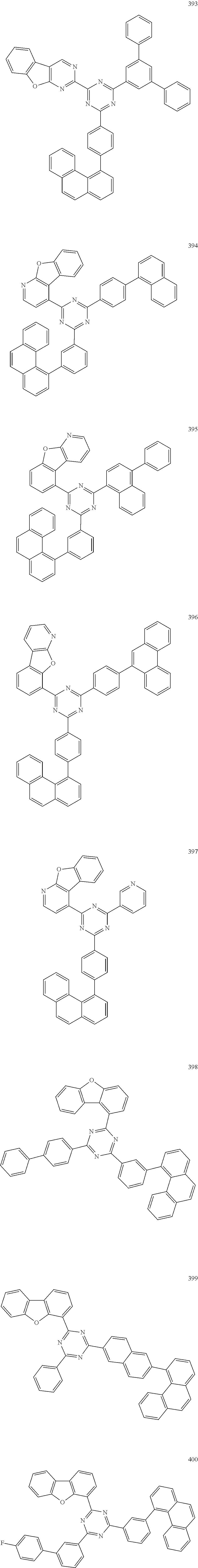

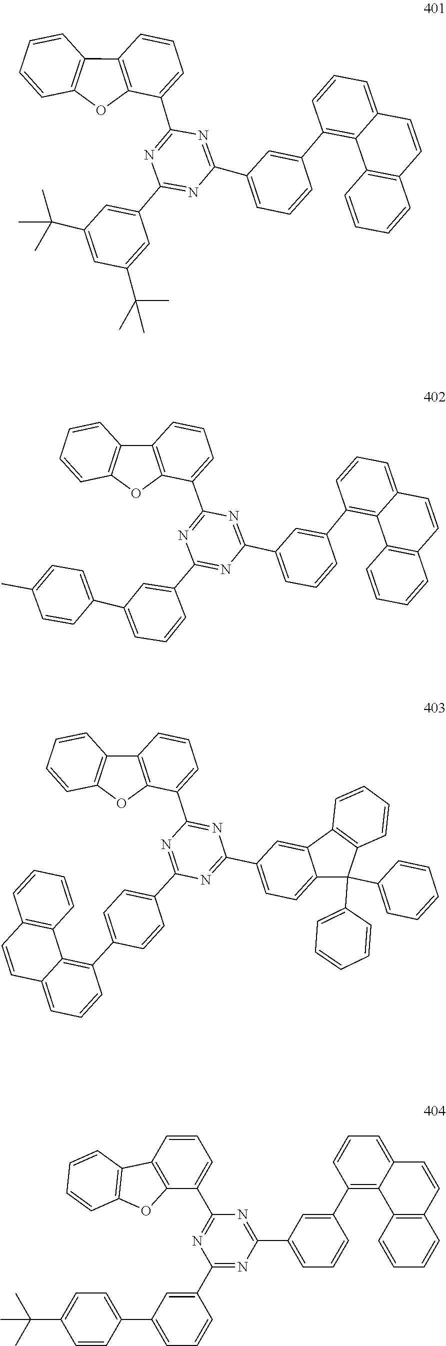 [SMILES: CC(C)(C)c1cc(-c2nc(-c3cccc(-c4cccc5ccc6ccccc6c45)c3)nc(-c3cccc4c3oc3ccccc34)n2)cc(C(C)(C)C)c1 ; Cc1ccc(-c2cccc(-c3nc(-c4cccc(-c5cccc6ccc7ccccc7c56)c4)nc(-c4cccc5c4oc4ccccc45)n3)c2)cc1 ; c1ccc(C2(c3ccccc3)c3ccccc3-c3cc(-c4nc(-c5ccc(-c6cccc7ccc8ccccc8c67)cc5)nc(-c5cccc6c5oc5ccccc56)n4)ccc32)cc1 ; CC(C)(C)c1ccc(-c2cccc(-c3nc(-c4cccc(-c5cccc6ccc7ccccc7c56)c4)nc(-c4cccc5c4oc4ccccc45)n3)c2)cc1]

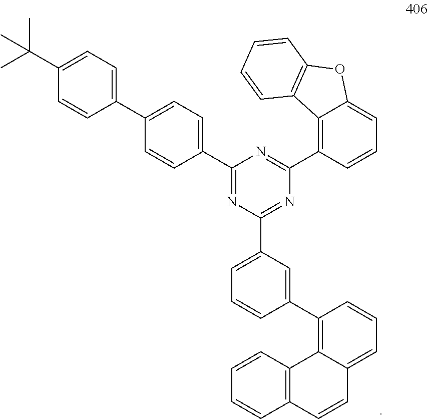 [SMILES: CC(C)(C)c1ccc(-c2ccc(-c3nc(-c4cccc(-c5cccc6ccc7ccccc7c56)c4)nc(-c4cccc5oc6ccccc6c45)n3)cc2)cc1]

8. An organic electroluminescent device, comprising an anode and a cathode which are disposed oppositely, and a functional layer disposed between the anode and the cathode; wherein
the functional layer comprises the nitrogen-containing compound according to claim 1.

9. The organic electroluminescent device according to claim 8, wherein the organic electroluminescent layer comprises a host material, and the host material contains the nitrogen-containing compound.

10. An electronic apparatus, comprising the organic electroluminescent device according to claim 8.

11. The nitrogen-containing compound according to claim 2, wherein substituents in the L and the L₁ are respectively and independently selected from deuterium, a halogen group, cyano, alkyl with 1 to 5 carbon atoms, aryl with 6 to 12 carbon atoms, and heteroaryl with 5 to 12 carbon atoms.

12. The nitrogen-containing compound according to claim 3, wherein substituents in L and L₁ are respectively and independently selected from deuterium, fluorine, cyano, methyl, ethyl, n-propyl, isopropyl, tert-butyl, phenyl, naphthyl, biphenyl, and carbazolyl.

13. The nitrogen-containing compound according to claim 5, wherein substituents in the Ar are selected from deuterium, fluorine, cyano, alkyl with 1 to 5 carbon atoms, aryl with 6 to 20 carbon atoms, heteroaryl with 12 to 18 carbon atoms, and cycloalkyl with 5 to 10 carbon atoms; or, any two adjacent substituents form a saturated or unsaturated ring having 5 to 8 carbon atoms.

14. The organic electroluminescent device according to claim 8, wherein the functional layer comprises an organic electroluminescent layer, and the organic electroluminescent layer comprises the nitrogen-containing compound.
